SEQUENCE LISTING (53 sequences)

SEQ ID NO: 1 - protein, 747 aa, synthetic construct
MGSRIVINREHLMIDRPYVLLAVLFVMFLSLIGLLAIAGIRLHRAAIYTAEIHKSLSTNLDVTNSIEHQVKDVLTPLFKIIGDEVGLRTPQRFTDLVKFISDKIKFLNPDREYDFRDLTWCINPPERIKLDYDQYCADVAAEELMNALVNSTLLEARATNQFLAVSKGNCSGPTTIRGQFSNMSLSLLDLYLSRGYNVSSIVTMTSQGMYGGTYLVGKPNLSSKGSELSQLSMHRVFEVGVIRNPGLGAPVFHMTNYFEQPVSNDFSNCMVALGELKFAALCHREDSITIPYQGSGKGVSFQLVKLGVWKSPTDMRSWVPLSTDDPVIDRLYLSSHRGVIADNQAKWAVPTTRTDDKLRMETCFQQACKGKNQALCENPEWAPLKDNRIPSYGVLSVNLSLTVELKIKIASGFGPLITHGSGMDLYKTNHNNVYWLTIPPMKNLALGVINTLEWIPRFKVSPALFTVPIKEAGEDCHAPTYLPAEVDGDVKLSSNLVILPGQDLQYVLATYDTSRVEHAVVYYVYSPSRSFSYFYPFRLPIKGVPIELQVECFTWDKKLWCRHFCVLADSESGGHITHSGMVGMGVSCTVTREDGTGGGGSGGGGSGGGGSAAAQVQLVQSGGGLVQPGGSLGLSCAASGNIGSRDNMGWYRQAPGKQREWVATISGYGIATYRDSVKGRFTVAKDTAKNIVSLQMNYLTTEDTAVYYCYAYAVDSRNIFWSQGTQVTVSGGGSGGGSYPYDVPDYA

SEQ ID NO: 2 - protein, 749 aa, synthetic construct
MGSRIVINREHLMIDRPYVLLAVLFVMFLSLIGLLAIAGIRLHRAAIYTAEIHKSLSTNLDVTNSIEHQVKDVLTPLFKIIGDEVGLRTPQRFTDLVKFISDKIKFLNPDREYDFRDLTWCINPPERIKLDYDQYCADVAAEELMNALVNSTLLEARATNQFLAVSKGNCSGPTTIRGQFSNMSLSLLDLYLSRGYNVSSIVTMTSQGMYGGTYLVGKPNLSSKGSELSQLSMHRVFEVGVIRNPGLGAPVFHMTNYFEQPVSNDFSNCMVALGELKFAALCHREDSITIPYQGSGKGVSFQLVKLGVWKSPTDMRSWVPLSTDDPVIDRLYLSSHRGVIADNQAKWAVPTTRTDDKLRMETCFQQACKGKNQALCENPEWAPLKDNRIPSYGVLSVNLSLTVELKIKIASGFGPLITHGSGMDLYKTNHNNVYWLTIPPMKNLALGVINTLEWIPRFKVSPALFTVPIKEAGEDCHAPTYLPAEVDGDVKLSSNLVILPGQDLQYVLATYDTSRVEHAVVYYVYSPSRSFSYFYPFRLPIKGVPIELQVECFTWDKKLWCRHFCVLADSESGGHITHSGMVGMGVSCTVTREDGTGGGGSGGGGSGGGGSAAAQVQLVQSGGGLVQPGGSLRLSCAASGRAFNSAAMGWYRQAPGSQRELVASISAGTASYADAVKGRFTISRDYAKNIIYLQMNSLKPDDTAVYFCNYRTTYTSGYSEDYWGQGTQVTVSGGGSGGGSYPYDVPDYA

SEQ ID NO: 3 - protein, 757 aa, synthetic construct
MGSRIVINREHLMIDRPYVLLAVLFVMFLSLIGLLAIAGIRLHRAAIYTAEIHKSLSTNLDVTNSIEHQVKDVLTPLFKIIGDEVGLRTPQRFTDLVKFISDKIKFLNPDREYDFRDLTWCINPPERIKLDYDQYCADVAAEELMNALVNSTLLEARATNQFLAVSKGNCSGPTTIRGQFSNMSLSLLDLYLSRGYNVSSIVTMTSQGMYGGTYLVGKPNLSSKGSELSQLSMHRVFEVGVIRNPGLGAPVFHMTNYFEQPVSNDFSNCMVALGELKFAALCHREDSITIPYQGSGKGVSFQLVKLGVWKSPTDMRSWVPLSTDDPVIDRLYLSSHRGVIADNQAKWAVPTTRTDDKLRMETCFQQACKGKNQALCENPEWAPLKDNRIPSYGVLSVNLSLTVELKIKIASGFGPLITHGSGMDLYKTNHNNVYWLTIPPMKNLALGVINTLEWIPRFKVSPALFTVPIKEAGEDCHAPTYLPAEVDGDVKLSSNLVILPGQDLQYVLATYDTSRVEHAVVYYVYSPSRSFSYFYPFRLPIKGVPIELQVECFTWDKKLWCRHFCVLADSESGGHITHSGMVGMGVSCTVTREDGTGGGGSGGGGSGGGGSAAADVQLQESGGGSVQAGGSLRLSCAASGYTHSSYCMAWFRQAPGREREGVASIDSDGTTSYADSVKGRFTISQDNAKNTLYLQMNSLKPEDTAMYYCAARFGPMGCVDLSTLSFGHWGQGTQVTVSITGGGSGGGSYPYDVPDYA

SEQ ID NO: 4 - protein, 756 aa, synthetic construct
MGSRIVINREHLMIDRPYVLLAVLFVMFLSLIGLLAIAGIRLHRAAIYTAEIHKSLSTNLDVTNSIEHQVKDVLTPLFKIIGDEVGLRTPQRFTDLVKFISDKIKFLNPDREYDFRDLTWCINPPERIKLDYDQYCADVAAEELMNALVNSTLLEARATNQFLAVSKGNCSGPTTIRGQFSNMSLSLLDLYLSRGYNVSSIVTMTSQGMYGGTYLVGKPNLSSKGSELSQLSMHRVFEVGVIRNPGLGAPVFHMTNYFEQPVSNDFSNCMVALGELKFAALCHREDSITIPYQGSGKGVSFQLVKLGVWKSPTDMRSWVPLSTDDPVIDRLYLSSHRGVIADNQAKWAVPTTRTDDKLRMETCFQQACKGKNQALCENPEWAPLKDNRIPSYGVLSVNLSLTVELKIKIASGFGPLITHGSGMDLYKTNHNNVYWLTIPPMKNLALGVINTLEWIPRFKVSPALFTVPIKEAGEDCHAPTYLPAEVDGDVKLSSNLVILPGQDLQYVLATYDTSRVEHAVVYYVYSPSRSFSYFYPFRLPIKGVPIELQVECFTWDKKLWCRHFCVLADSESGGHITHSGMVGMGVSCTVTREDGTGGGGSGGGGSGGGGSAAAEVQLVESGGGSVQPGGSLTLSCGTSGRTFNVMGWFRQAPGKEREFVAAVRWSSTGIYYTQYADSVKSRFTISRDNAKNTVYLEMNSLKPEDTAVYYCAADTYNSNPARWDGYDFRGQGTQVTVSSGGGSGGGSYPYDVPDYA

SEQ ID NO: 5 - protein, 763 aa, synthetic construct
MGSRIVINREHLMIDRPYVLLAVLFVMFLSLIGLLAIAGIRLHRAAIYTAEIHKSLSTNLDVTNSIEHQVKDVLTPLFKIIGDEVGLRTPQRFTDLVKFISDKIKFLNPDREYDFRDLTWCINPPERIKLDYDQYCADVAAEELMNALVNSTLLEARATNQFLAVSKGNCSGPTTIRGQFSNMSLSLLDLYLSRGYNVSSIVTMTSQGMYGGTYLVGKPNLSSKGSELSQLSMHRVFEVGVIRNPGLGAPVFHMTNYFEQPVSNDFSNCMVALGELKFAALCHREDSITIPYQGSGKGVSFQLVKLGVWKSPTDMRSWVPLSTDDPVIDRLYLSSHRGVIADNQAKWAVPTTRTDDKLRMETCFQQACKGKNQALCENPEWAPLKDNRIPSYGVLSVNLSLTVELKIKIASGFGPLITHGSGMDLYKTNHNNVYWLTIPPMKNLALGVINTLEWIPRFKVSPALFTVPIKEAGEDCHAPTYLPAEVDGDVKLSSNLVILPGQDLQYVLATYDTSRVEHAVVYYVYSPSRSFSYFYPFRLPIKGVPIELQVECFTWDKKLWCRHFCVLADSESGGHITHSGMVGMGVSCTVTREDGTGGGGSGGGGSGGGGSAAAQVQLQESGGGSVQPGGSLRLSCAASGFTFDDYAMSWVRQVPGKGLEWVSTINWNGGSAEYAEPVKGRFTISRDNAKNTVYLQMNSLKLEDTAVYYCAKDADLVWYNLSTGQGTQVTVSSAAAYPYDVPDYGSGGGSGGGSYPYDVPDYA

SEQ ID NO: 6 - protein, 596 aa, Measles morbillivirus
MGSRIVINREHLMIDRPYVLLAVLFVMFLSLIGLLAIAGIRLHRAAIYTAEIHKSLSTNLDVTNSIEHQVKDVLTPLFKIIGDEVGLRTPQRFTDLVKFISDKIKFLNPDREYDFRDLTWCINPPERIKLDYDQYCADVAAEELMNALVNSTLLEARATNQFLAVSKGNCSGPTTIRGQFSNMSLSLLDLYLSRGYNVSSIVTMTSQGMYGGTYLVGKPNLSSKGSELSQLSMHRVFEVGVIRNPGLGAPVFHMTNYFEQPVSNDFSNCMVALGELKFAALCHREDSITIPYQGSGKGVSFQLVKLGVWKSPTDMRSWVPLSTDDPVIDRLYLSSHRGVIADNQAKWAVPTTRTDDKLRMETCFQQACKGKNQALCENPEWAPLKDNRIPSYGVLSVNLSLTVELKIKIASGFGPLITHGSGMDLYKTNHNNVYWLTIPPMKNLALGVINTLEWIPRFKVSPALFTVPIKEAGEDCHAPTYLPAEVDGDVKLSSNLVILPGQDLQYVLATYDTSRVEHAVVYYVYSPSRSFSYFYPFRLPIKGVPIELQVECFTWDKKLWCRHFCVLADSESGGHITHSGMVGMGVSCTVTREDGT

SEQ ID NO: 7 - protein, 116 aa, synthetic construct
QVQLVQSGGGLVQPGGSLGLSCAASGNIGSRDNMGWYRQAPGKQREWVATISGYGIATYRDSVKGRFTVAKDTAKNIVSLQMNYLTTEDTAVYYCYAYAVDSRNIFWSQGTQVTVS

SEQ ID NO: 8 - protein, 118 aa, synthetic construct
QVQLVQSGGGLVQPGGSLRLSCAASGRAFNSAAMGWYRQAPGSQRELVASISAGTASYADAVKGRFTISRDYAKNIIYLQMNSLKPDDTAVYFCNYRTTYTSGYSEDYWGQGTQVTVS

SEQ ID NO: 9 - protein, 126 aa, synthetic construct
DVQLQESGGGSVQAGGSLRLSCAASGYTHSSYCMAWFRQAPGREREGVASIDSDGTTSYADSVKGRFTISQDNAKNTLYLQMNSLKPEDTAMYYCAARFGPMGCVDLSTLSFGHWGQGTQVTVSIT

SEQ ID NO: 10 - protein, 125 aa, synthetic construct
EVQLVESGGGSVQPGGSLTLSCGTSGRTFNVMGWFRQAPGKEREFVAAVRWSSTGIYYTQYADSVKSRFTISRDNAKNTVYLEMNSLKPEDTAVYYCAADTYNSNPARWDGYDFRGQGTQVTVSS

SEQ ID NO: 11 - protein, 119 aa, synthetic construct
QVQLQESGGGSVQPGGSLRLSCAASGFTFDDYAMSWVRQVPGKGLEWVSTINWNGGSAEYAEPVKGRFTISRDNAKNTVYLQMNSLKLEDTAVYYCAKDADLVWYNLSTGQGTQVTVSS

SEQ ID NO: 12 - protein, 5 aa, synthetic construct
GGGGS

SEQ ID NO: 13 - protein, 4 aa, synthetic construct
GGGS

SEQ ID NO: 14 - protein, 15 aa, synthetic construct
GGGGSGGGGSGGGGS

SEQ ID NO: 15 - DNA, 348 bp, synthetic construct
  source 1..348: mol_type = other DNA
caggttcaactggtgcagagcggaggcggcctggtccagcccggcggaagcctgggcctgagctgtgccgcttctggcaacatcggcagcagagataatatgggctggtatagacaggcccctggaaaacagagagagtgggtggctacaatcagcggctacggcatcgccacataccgggacagcgtgaagggcagattcaccgtggccaaggacaccgccaagaacatcgtgtccctgcagatgaactacctgaccaccgaagatacagccgtgtactactgctacgcctacgccgtggacagccggaacatcttctggtcccagggcacccaggtgaccgtgtct

SEQ ID NO: 16 - DNA, 354 bp, synthetic construct
  source 1..354: mol_type = other DNA
caggtgcagctggtgcagagcggcggaggactggtgcaacctggaggcagcctgagactgagctgcgccgctagcggcagagcctttaacagcgccgccatgggctggtacagacaggcccctggctctcagagagagctggtcgcctctatctccgccggcaccgcctcttatgctgatgccgtgaaaggcagattcaccatcagccgggactacgccaagaacatcatctacctgcagatgaacagcctgaagcccgacgacacagctgtgtacttctgtaattaccggacaacctacaccagcggctacagcgaagattactggggccagggcacacaggtgaccgtgtcc

SEQ ID NO: 17 - DNA, 378 bp, synthetic construct
  source 1..378: mol_type = other DNA
gatgtgcagctgcaggagagcggcggcggatctgtgcaagctggcggaagcctgagactgtcttgcgccgcttctggctacacccacagcagctattgcatggcctggttccggcaggcccctggcagagaacgggaaggcgtggccagcatcgacagcgacggcaccaccagctacgccgatagcgtcaagggcagattcaccatcagccaggacaatgccaaaaacaccctgtacctgcagatgaacagcctgaagcccgaggacacagccatgtactactgtgccgccagatttggccctatgggctgcgtggacctgtccaccctctccttcggccactggggacagggcacacaggtgacagtgtccatcacc

SEQ ID NO: 18 - DNA, 375 bp, synthetic construct
  source 1..375: mol_type = other DNA
gaggtgcagctggtcgagagcggaggaggcagcgtgcagcccggcggatctctgaccctgagctgtggcacaagcggcagaacctttaacgtgatgggctggttcagacaggcccctggcaaggaaagagagttcgtggccgctgtgcggtggtcctctacaggcatctactatacacagtacgccgacagcgttaaaagcagattcaccatcagccgggacaacgccaagaacaccgtgtacctggaaatgaacagcctgaagccagaggatacagccgtgtactactgcgccgccgatacctacaactctaatcctgctagatgggacggctacgacttccggggccagggcacccaagtgaccgtgtccagc

SEQ ID NO: 19 - DNA, 396 bp, synthetic construct
  source 1..396: mol_type = other DNA
caagtgcagctgcaggagagcggcggaggcagcgtgcagcctggcggaagcctgagactgtcctgcgccgcttctggctttaccttcgacgactacgccatgagctgggtgcggcaggttccaggcaagggcctggaatgggtcagcacaatcaactggaacggcggatctgctgagtacgccgagcctgtgaagggcagattcacaatcagcagagataatgccaaaaacaccgtgtatctgcagatgaacagcctcaagctggaagatacagccgtgtactactgtgccaaggacgccgacctggtgtggtacaacctgagcaccggccagggcacccaggtgaccgtgtccagcgccgctgcctacccctacgacgtgcctgattacggctct

SEQ ID NO: 20 - protein, 520 aa, Measles morbillivirus
MGLKVNVSAIFMAVLLTLQTPTGQIHWGNLSKIGVVGIGSASYKVMTRSSHQSLVIKLMPNITLLNNCTRVEIAEYRRLLRTVLEPIRDALNAMTQNIRPVQSVASSRRHKRFAGVVLAGAALGVATAAQITAGIALHQSMLNSQAIDNLRASLETTNQAIEAIRQAGQEMILAVQGVQDYINNELIPSMNQLSCDLIGQKLGLKLLRYYTEILSLFGPSLRDPISAEISIQALSYALGGDINKVLEKLGYSGGDLLGILESRGIKARITHVDTESYFIVLSIAYPTLSEIKGVIVHRLEGVSYNIGSQEWYTTVPKYVATQGYLISNFDESSCTFMPEGTVCSQNALYPMSPLLQECLRGSTKSCARTLVSGSFGNRFILSQGNLIANCASILCKCYTTGTIINQDPDKILTYIAADHCPVVEVNGVTIQVGSRRYPDAVYLHRIDLGPPISLERLDVGTNLGNAIAKLEDAKELLESSDQILRSMKGLSSTSIVYILIAVCLGGLIGIPALICCCRGR

SEQ ID NO: 21 - DNA, 1563 bp, synthetic construct
  source 1..1563: mol_type = other DNA
atgggcctgaaggtgaacgtgagcgccatcttcatggccgtgctgctgaccctgcagaccccaacaggccagatccactggggcaatctgtctaagatcggagtggtgggaatcggatccgcctcttataaagtgatgacaaggagctcccaccagagcctggtcatcaagctgatgcctaacatcaccctgctgaacaattgtacacgcgtggagatcgccgagtaccggagactgctgaggaccgtgctggagccaatcagggacgccctgaacgccatgacacagaatatcaggccagtgcagagcgtggcatctagcaggcgccacaagagatttgccggcgtggtgctggcaggagccgccctgggagtggcaaccgccgcccagatcacagccggcatcgccctgcaccagtccatgctgaactctcaggccatcgataatctgagggcctccctggagacaacaaaccaggcaatcgaggcaatcaggcaggcaggacaggagatgatcctggccgtgcagggcgtgcaggactatatcaacaatgagctgatcccttctatgaatcagctgagctgcgatctgatcggccagaagctgggcctgaagctgctgcggtactataccgagatcctgtccctgttcggaccttctctgcgggaccccatcagcgccgagatctccatccaggccctgtcctatgcactgggaggcgacatcaacaaggtgctggagaagctgggctactctggcggcgatctgctgggcatcctggagagccggggcatcaaggccagaatcacccacgtggatacagagtcttacttcatcgtgctgagcatcgcctaccccacactgtccgagatcaagggcgtgatcgtgcacaggctggagggcgtgagctataatatcggctcccaggagtggtacaccacagtgcctaagtatgtggccacccagggctacctgatctctaacttcgacgagtcctcttgtacctttatgccagagggcacagtgtgctcccagaatgccctgtacccaatgtctcccctgctgcaggagtgtctgcggggctctaccaagagctgcgccagaacactggtgagcggctccttcggcaatcggttcatcctgtcccagggcaacctgatcgccaattgtgcctctatcctgtgcaagtgctataccacaggcaccatcatcaaccaggaccccgataagatcctgacatacatcgcagcagaccactgccctgtggtggaagtgaatggcgtgaccatccaagtgggcagccggagatacccagacgccgtgtatctgcacaggatcgatctgggaccacctatctccctggagcgcctggatgtgggcacaaacctgggcaatgccatcgccaagctggaggacgccaaggagctgctggagagctccgatcagatcctgaggagcatgaagggcctgtctagcacctccatcgtgtacatcctgatcgccgtgtgcctgggaggcctgatcggaatccccgccctgatctgctgttgcaggggccgctga

SEQ ID NO: 22 - DNA, 802 bp, synthetic construct
  source 1..802: mol_type = other DNA
gctgtacctgtcctctcacagaggcgtgatcgccgacaatcaggcaaagtgggcagtgccaaccacaaggaccgacgataagctgagaatggagacatgctttcagcaggcctgtaagggcaagaaccaggccctgtgcgagaatccagagtgggcacctctgaaggataacagaatccctagctacggcgtgctgagcgtgaatctgtccctgaccgtggagctgaagatcaagatcgccagcggcttcggcccactgatcacacacggctccggcatggacctgtacaagaccaaccacaacaacgtgtactggctgacaatcccacccatgaagaacctggccctgggcgtgatcaataccctggagtggatccccaggtttaaggtgtcccctgccctgttcaccgtgccaatcaaggaggccggcgaggattgtcacgccccaacatacctgcccgccgaggtggacggcgatgtgaagctgagctccaacctggtcatcctgcctggccaggacctgcagtacgtgctggccacctatgatacaagccgcgtggagcacgccgtggtgtactacgtgtactccccctctcggagcttctcctacttttatcctttcagactgccaatcaagggcgtgcctatcgagctgcaggtggagtgctttacctgggataagaagctgtggtgcaggcacttctgcgtgctggcagactctgagagcggaggacacatcacccactccggaatggtgggcatgggcgtgtcttgtaccgtgacacgcgaggacggcacatga

SEQ ID NO: 23 - protein, 872 aa, synthetic construct
MGSRIVINREHLMIDRPYVLLAVLFVMFLSLIGLLAIAGIRLHRAAIYTAEIHKSLSTNLDVTNSIEHQVKDVLTPLFKIIGDEVGLRTPQRFTDLVKFISDKIKFLNPDREYDFRDLTWCINPPERIKLDYDQYCADVAAEELMNALVNSTLLEARATNQFLAVSKGNCSGPTTIRGQFSNMSLSLLDLYLSRGYNVSSIVTMTSQGMYGGTYLVGKPNLSSKGSELSQLSMHRVFEVGVIRNPGLGAPVFHMTNYFEQPVSNDFSNCMVALGELKFAALCHREDSITIPYQGSGKGVSFQLVKLGVWKSPTDMRSWVPLSTDDPVIDRLYLSSHRGVIADNQAKWAVPTTRTDDKLRMETCFQQACKGKNQALCENPEWAPLKDNRIPSYGVLSVNLSLTVELKIKIASGFGPLITHGSGMDLYKTNHNNVYWLTIPPMKNLALGVINTLEWIPRFKVSPALFTVPIKEAGEDCHAPTYLPAEVDGDVKLSSNLVILPGQDLQYVLATYDTSRVEHAVVYYVYSPSRSFSYFYPFRLPIKGVPIELQVECFTWDKKLWCRHFCVLADSESGGHITHSGMVGMGVSCTVTREDGTNGGGGSGGGGSGGGGSAAQPAMAEVQLLESGGGLVQPGGSLRLSCAASGFTFSSYAMSWVRQAPGKGLEWVSAIYGSDGDTTYADSVKGRFTISRDNSKNTLYLQMNSLRAEDTAVYYCARVFYTAGFDYWGQGTLVTVSSGSDSNAGHASAGNTSDIELTQSPSSLSASVGDRVTITCRASQSISSSLNWYQQKPGKAPKLLIYAASSLQSGVPSRFSGSGSGTDFTLTISSLQPEDFATYYCQQAPAKPPTFGQGTKLEIKRAAARGSHHHHHH

SEQ ID NO: 24 - protein, 6 aa, synthetic construct
HHHHHH

SEQ ID NO: 25 - DNA, 2619 bp, synthetic construct
  source 1..2619: mol_type = other DNA
atgggctctaggatcgtgatcaaccgcgagcacctgatgatcgacaggccatacgtgctgctggccgtgctgttcgtgatgtttctgagcctgatcggcctgctggcaatcgcaggaatcaggctgcacagagccgccatctataccgccgagatccacaagagcctgtccaccaacctggacgtgacaaattccatcgagcaccaggtgaaggatgtgctgacaccactgttcaagatcatcggcgacgaagtgggcctgaggaccccacagcggttcaccgacctggtgaagttcatcagcgataagatcaagtttctgaaccctgatcgggagtacgacttcagagatctgacctggtgcatcaatccccctgagcggatcaagctggactacgatcagtattgtgccgacgtggccgccgaggagctgatgaacgccctggtgaattccaccctgctggaggccagagccacaaaccagtttctggccgtgagcaagggcaattgctccggcccaaccacaatccggggccagttctctaacatgtctctgagcctgctggatctgtacctgagcagaggctataatgtgagctccatcgtgaccatgacaagccagggcatgtacggcggcacctatctggtgggcaagcccaacctgtctagcaagggctccgagctgtctcagctgagcatgcacagggtgtttgaagtgggcgtgatcaggaacccaggcctgggagcccccgtgtttcacatgaccaattacttcgagcagcccgtgagcaacgatttttctaattgcatggtggccctgggagagctgaagttcgccgccctgtgccacagggaggacagcatcaccatcccatatcagggctctggcaagggcgtgagcttccagctggtgaagctgggcgtgtggaagtcccccaccgacatgagatcctgggtgcctctgtctacagacgatccagtgatcgatcggctgtacctgtcctctcacagaggcgtgatcgccgacaatcaggcaaagtgggcagtgccaaccacaaggaccgacgataagctgagaatggagacatgctttcagcaggcctgtaagggcaagaaccaggccctgtgcgagaatccagagtgggcacctctgaaggataacagaatccctagctacggcgtgctgagcgtgaatctgtccctgaccgtggagctgaagatcaagatcgccagcggcttcggcccactgatcacacacggctccggcatggacctgtacaagaccaaccacaacaacgtgtactggctgacaatcccacccatgaagaacctggccctgggcgtgatcaataccctggagtggatccccaggtttaaggtgtcccctgccctgttcaccgtgccaatcaaggaggccggcgaggattgtcacgccccaacatacctgcccgccgaggtggacggcgatgtgaagctgagctccaacctggtcatcctgcctggccaggacctgcagtacgtgctggccacctatgatacaagccgcgtggagcacgccgtggtgtactacgtgtactccccctctcggagcttctcctacttttatcctttcagactgccaatcaagggcgtgcctatcgagctgcaggtggagtgctttacctgggataagaagctgtggtgcaggcacttctgcgtgctggcagactctgagagcggaggacacatcacccactccggaatggtgggcatgggcgtgtcttgtaccgtgacacgcgaggacggcacaaacggaggaggaggaagcggaggaggaggatccggcggcggcggctctgccgcccagcctgccatggccgaggtgcagctgctggagtccggaggaggcctggtgcagccaggaggctctctgaggctgagctgcgcagcatccggcttcaccttttctagctacgccatgagctgggtgcgccaggcaccaggcaagggcctggagtgggtgagcgccatctacggatccgacggcgataccacatatgccgattccgtgaagggcaggttcaccatctctcgcgacaacagcaagaatacactgtatctgcagatgaactctctgagggccgaggataccgccgtgtactattgcgcccgcgtgttttacacagccggcttcgactattggggccagggcaccctggtgacagtgtcctctggctccgattctaacgcaggacacgcatctgccggaaataccagcgacatcgagctgacacagtccccaagctccctgagcgcctccgtgggcgacagggtgaccatcacatgtcgcgcctctcagagcatctctagctccctgaattggtaccagcagaagcccggcaaggcccctaagctgctgatctatgcagcatctagcctgcagtccggagtgccatctaggttttccggctctggcagcggcaccgatttcaccctgacaatctcctctctgcagcccgaggactttgccacatactattgtcagcaggcccctgccaagcctccaaccttcggccagggcacaaagctggagatcaagagggcagcagcaagaggcagccaccaccaccaccaccactga

SEQ ID NO: 26 - protein, 12 aa, synthetic construct
GGGSGGGSGGGS

SEQ ID NO: 27 - protein, 8 aa, synthetic construct
GGGSGGGS

SEQ ID NO: 28 - DNA, 2241 bp, synthetic construct
  source 1..2241: mol_type = other DNA
atgggctctaggatcgtgatcaaccgcgagcacctgatgatcgacaggccatacgtgctgctggccgtgctgttcgtgatgtttctgagcctgatcggcctgctggcaatcgcaggaatcaggctgcacagagccgccatctataccgccgagatccacaagagcctgtccaccaacctggacgtgacaaattccatcgagcaccaggtgaaggatgtgctgacaccactgttcaagatcatcggcgacgaagtgggcctgaggaccccacagcggttcaccgacctggtgaagttcatcagcgataagatcaagtttctgaaccctgatcgggagtacgacttcagagatctgacctggtgcatcaatccccctgagcggatcaagctggactacgatcagtattgtgccgacgtggccgccgaggagctgatgaacgccctggtgaattccaccctgctggaggccagagccacaaaccagtttctggccgtgagcaagggcaattgctccggcccaaccacaatccggggccagttctctaacatgtctctgagcctgctggatctgtacctgagcagaggctataatgtgagctccatcgtgaccatgacaagccagggcatgtacggcggcacctatctggtgggcaagcccaacctgtctagcaagggctccgagctgtctcagctgagcatgcacagggtgtttgaagtgggcgtgatcaggaacccaggcctgggagcccccgtgtttcacatgaccaattacttcgagcagcccgtgagcaacgatttttctaattgcatggtggccctgggagagctgaagttcgccgccctgtgccacagggaggacagcatcaccatcccatatcagggctctggcaagggcgtgagcttccagctggtgaagctgggcgtgtggaagtcccccaccgacatgagatcctgggtgcctctgtctacagacgatccagtgatcgatcggctgtacctgtcctctcacagaggcgtgatcgccgacaatcaggcaaagtgggcagtgccaaccacaaggaccgacgataagctgagaatggagacatgctttcagcaggcctgtaagggcaagaaccaggccctgtgcgagaatccagagtgggcacctctgaaggataacagaatccctagctacggcgtgctgagcgtgaatctgtccctgaccgtggagctgaagatcaagatcgccagcggcttcggcccactgatcacacacggctccggcatggacctgtacaagaccaaccacaacaacgtgtactggctgacaatcccacccatgaagaacctggccctgggcgtgatcaataccctggagtggatccccaggtttaaggtgtcccctgccctgttcaccgtgccaatcaaggaggccggcgaggattgtcacgccccaacatacctgcccgccgaggtggacggcgatgtgaagctgagctccaacctggtcatcctgcctggccaggacctgcagtacgtgctggccacctatgatacaagccgcgtggagcacgccgtggtgtactacgtgtactccccctctcggagcttctcctacttttatcctttcagactgccaatcaagggcgtgcctatcgagctgcaggtggagtgctttacctgggataagaagctgtggtgcaggcacttctgcgtgctggcagactctgagagcggaggacacatcacccactccggaatggtgggcatgggcgtgtcttgtaccgtgacacgcgaggacggcacagggggaggcggctctggaggtggggggtccggtggcggcggaagcgcggccgcgcaggttcaactggtgcagagcggaggcggcctggtccagcccggcggaagcctgggcctgagctgtgccgcttctggcaacatcggcagcagagataatatgggctggtatagacaggcccctggaaaacagagagagtgggtggctacaatcagcggctacggcatcgccacataccgggacagcgtgaagggcagattcaccgtggccaaggacaccgccaagaacatcgtgtccctgcagatgaactacctgaccaccgaagatacagccgtgtactactgctacgcctacgccgtggacagccggaacatcttctggtcccagggcacccaggtgaccgtgtctggcggcggctccggcggcggcagctatccctatgatgtgcccgactatgcg

SEQ ID NO: 29 - DNA, 2247 bp, synthetic construct
  source 1..2247: mol_type = other DNA
atgggctctaggatcgtgatcaaccgcgagcacctgatgatcgacaggccatacgtgctgctggccgtgctgttcgtgatgtttctgagcctgatcggcctgctggcaatcgcaggaatcaggctgcacagagccgccatctataccgccgagatccacaagagcctgtccaccaacctggacgtgacaaattccatcgagcaccaggtgaaggatgtgctgacaccactgttcaagatcatcggcgacgaagtgggcctgaggaccccacagcggttcaccgacctggtgaagttcatcagcgataagatcaagtttctgaaccctgatcgggagtacgacttcagagatctgacctggtgcatcaatccccctgagcggatcaagctggactacgatcagtattgtgccgacgtggccgccgaggagctgatgaacgccctggtgaattccaccctgctggaggccagagccacaaaccagtttctggccgtgagcaagggcaattgctccggcccaaccacaatccggggccagttctctaacatgtctctgagcctgctggatctgtacctgagcagaggctataatgtgagctccatcgtgaccatgacaagccagggcatgtacggcggcacctatctggtgggcaagcccaacctgtctagcaagggctccgagctgtctcagctgagcatgcacagggtgtttgaagtgggcgtgatcaggaacccaggcctgggagcccccgtgtttcacatgaccaattacttcgagcagcccgtgagcaacgatttttctaattgcatggtggccctgggagagctgaagttcgccgccctgtgccacagggaggacagcatcaccatcccatatcagggctctggcaagggcgtgagcttccagctggtgaagctgggcgtgtggaagtcccccaccgacatgagatcctgggtgcctctgtctacagacgatccagtgatcgatcggctgtacctgtcctctcacagaggcgtgatcgccgacaatcaggcaaagtgggcagtgccaaccacaaggaccgacgataagctgagaatggagacatgctttcagcaggcctgtaagggcaagaaccaggccctgtgcgagaatccagagtgggcacctctgaaggataacagaatccctagctacggcgtgctgagcgtgaatctgtccctgaccgtggagctgaagatcaagatcgccagcggcttcggcccactgatcacacacggctccggcatggacctgtacaagaccaaccacaacaacgtgtactggctgacaatcccacccatgaagaacctggccctgggcgtgatcaataccctggagtggatccccaggtttaaggtgtcccctgccctgttcaccgtgccaatcaaggaggccggcgaggattgtcacgccccaacatacctgcccgccgaggtggacggcgatgtgaagctgagctccaacctggtcatcctgcctggccaggacctgcagtacgtgctggccacctatgatacaagccgcgtggagcacgccgtggtgtactacgtgtactccccctctcggagcttctcctacttttatcctttcagactgccaatcaagggcgtgcctatcgagctgcaggtggagtgctttacctgggataagaagctgtggtgcaggcacttctgcgtgctggcagactctgagagcggaggacacatcacccactccggaatggtgggcatgggcgtgtcttgtaccgtgacacgcgaggacggcacagggggaggcggctctggaggtggggggtccggtggcggcggaagcgcggccgcgcaggtgcagctggtgcagagcggcggaggactggtgcaacctggaggcagcctgagactgagctgcgccgctagcggcagagcctttaacagcgccgccatgggctggtacagacaggcccctggctctcagagagagctggtcgcctctatctccgccggcaccgcctcttatgctgatgccgtgaaaggcagattcaccatcagccgggactacgccaagaacatcatctacctgcagatgaacagcctgaagcccgacgacacagctgtgtacttctgtaattaccggacaacctacaccagcggctacagcgaagattactggggccagggcacacaggtgaccgtgtccggcggcggctccggcggcggcagctatccctatgatgtgcccgactatgcg

SEQ ID NO: 30 - protein, 872 aa, synthetic construct
MGSRIVINREHLMIDRPYVLLAVLFVMFLSLIGLLAIAGIRLHRAAIYTAEIHKSLSTNLDVTNSIEHQVKDVLTPLFKIIGDEVGLRTPQRFTDLVKFISDKIKFLNPDREYDFRDLTWCINPPERIKLDYDQYCADVAAEELMNALVNSTLLEARATNQFLAVSKGNCSGPTTIRGQFSNMSLSLLDLYLSRGYNVSSIVTMTSQGMYGGTYLVGKPNLSSKGSELSQLSMHRVFEVGVIRNPGLGAPVFHMTNYFEQPVSNDFSNCMVALGELKFAALCHREDSITIPYQGSGKGVSFQLVKLGVWKSPTDMRSWVPLSTDDPVIDRLYLSSHRGVIADNQAKWAVPTTRTDDKLRMETCFQQACKGKNQALCENPEWAPLKDNRIPSYGVLSVNLSLTVELKIKIASGFGPLITHGSGMDLYKTNHNNVYWLTIPPMKNLALGVINTLEWIPRFKVSPALFTVPIKEAGEDCHAPTYLPAEVDGDVKLSSNLVILPGQDLQYVLATYDTSRVEHAVVYYVYSPSRSFSYFYPFRLPIKGVPIELQVECFTWDKKLWCRHFCVLADSESGGHITHSGMVGMGVSCTVTREDGTGGGGSGGGGSGGGGSAAAQVQLKESGPGLVKPSETLSLTCTVSGFSFTSYHISWVRQPPGKGLEWMGVIWGDGSTAYNSVFKSRLSISRDTSKSQVFLKMSSLKTEDTATYYCARDRDWELGNWFAYWGQGTLVTVSSGGGGSGGGGSGGGGSDIQMTQSPPSLSASLGDKVTITCQASQNINKYIAWYQQKPGKAPRQLIYYTSILVSGTPSRFSGSGSGRDYSFSISNVESEDIASYYCLQYDNLWTFGGGTKLELKGGGSGGGSYPYDVPDYA

SEQ ID NO: 31 - protein, 188 aa, Homo sapiens
TRPRFLWQLKFECHFFNGTERVRLLERCIYNQEESVRFDSDVGEYRAVTELGRPDAEYWNSQKDLLEQRRAAVDTYCRHNYGVGESFTVQRRVEPKVTVYPSKTQPLQHHNLLVCSVSGFYPGSIEVRWFRNGQEEKAGVVSTGLIQNGDWTFQTLVMLETVPRSGEVYTCQVEHPSVTSPLTVEWRA

SEQ ID NO: 32 - DNA, 2619 bp, synthetic construct
  source 1..2619: mol_type = other DNA
atgggctctaggatcgtgatcaaccgcgagcacctgatgatcgacaggccatacgtgctgctggccgtgctgttcgtgatgtttctgagcctgatcggcctgctggcaatcgcaggaatcaggctgcacagagccgccatctataccgccgagatccacaagagcctgtccaccaacctggacgtgacaaattccatcgagcaccaggtgaaggatgtgctgacaccactgttcaagatcatcggcgacgaagtgggcctgaggaccccacagcggttcaccgacctggtgaagttcatcagcgataagatcaagtttctgaaccctgatcgggagtacgacttcagagatctgacctggtgcatcaatccccctgagcggatcaagctggactacgatcagtattgtgccgacgtggccgccgaggagctgatgaacgccctggtgaattccaccctgctggaggccagagccacaaaccagtttctggccgtgagcaagggcaattgctccggcccaaccacaatccggggccagttctctaacatgtctctgagcctgctggatctgtacctgagcagaggctataatgtgagctccatcgtgaccatgacaagccagggcatgtacggcggcacctatctggtgggcaagcccaacctgtctagcaagggctccgagctgtctcagctgagcatgcacagggtgtttgaagtgggcgtgatcaggaacccaggcctgggagcccccgtgtttcacatgaccaattacttcgagcagcccgtgagcaacgatttttctaattgcatggtggccctgggagagctgaagttcgccgccctgtgccacagggaggacagcatcaccatcccatatcagggctctggcaagggcgtgagcttccagctggtgaagctgggcgtgtggaagtcccccaccgacatgagatcctgggtgcctctgtctacagacgatccagtgatcgatcggctgtacctgtcctctcacagaggcgtgatcgccgacaatcaggcaaagtgggcagtgccaaccacaaggaccgacgataagctgagaatggagacatgctttcagcaggcctgtaagggcaagaaccaggccctgtgcgagaatccagagtgggcacctctgaaggataacagaatccctagctacggcgtgctgagcgtgaatctgtccctgaccgtggagctgaagatcaagatcgccagcggcttcggcccactgatcacacacggctccggcatggacctgtacaagaccaaccacaacaacgtgtactggctgacaatcccacccatgaagaacctggccctgggcgtgatcaataccctggagtggatccccaggtttaaggtgtcccctgccctgttcaccgtgccaatcaaggaggccggcgaggattgtcacgccccaacatacctgcccgccgaggtggacggcgatgtgaagctgagctccaacctggtcatcctgcctggccaggacctgcagtacgtgctggccacctatgatacaagccgcgtggagcacgccgtggtgtactacgtgtactccccctctcggagcttctcctacttttatcctttcagactgccaatcaagggcgtgcctatcgagctgcaggtggagtgctttacctgggataagaagctgtggtgcaggcacttctgcgtgctggcagactctgagagcggaggacacatcacccactccggaatggtgggcatgggcgtgtcttgtaccgtgacacgcgaggacggcacagggggaggcggctctggaggtggggggtccggtggcggcggaagcgcggccgcgcaagtgcagctgaaggaatctggtcctggactggttaagcccagcgagacactgagcctgacctgtaccgtgagcggcttcagctttaccagctaccacatcagctgggtgcggcaacctcctggaaagggcctggagtggatgggcgtcatctggggcgacggctcaacagcttacaacagcgtgttcaagtctcggctgagcatcagcagagatacaagcaagagccaggtgtttctgaaaatgagcagccttaagaccgaggataccgccacctattactgcgccagagacagagactgggagctgggcaactggttcgcctactggggccagggcaccctggtgacagtgtccagcggcggcggaggctccggcggaggaggctctggcggcggcggctctgatatccagatgacacagagcccccccagcctgagcgccagcctgggcgacaaggtgaccatcacctgccaggcctcccagaacatcaacaagtacatcgcctggtaccagcagaaacctggcaaggcccctagacagctgatctactacacctctattctggtgtccggcacacctagccggttcagcggatctggcagcggcagagactacagcttcagcatcagcaatgtggaaagcgaggacatcgcttcttattactgtctgcagtacgacaacctgtggaccttcggcggtggcaccaagctggaactgaaaggaggcggatcaggaggtggatcctatccctatgatgtgcccgactatgcgtga

SEQ ID NO: 33 - DNA, 2274 bp, synthetic construct
  source 1..2274: mol_type = other DNA
atgggctctaggatcgtgatcaaccgcgagcacctgatgatcgacaggccatacgtgctgctggccgtgctgttcgtgatgtttctgagcctgatcggcctgctggcaatcgcaggaatcaggctgcacagagccgccatctataccgccgagatccacaagagcctgtccaccaacctggacgtgacaaattccatcgagcaccaggtgaaggatgtgctgacaccactgttcaagatcatcggcgacgaagtgggcctgaggaccccacagcggttcaccgacctggtgaagttcatcagcgataagatcaagtttctgaaccctgatcgggagtacgacttcagagatctgacctggtgcatcaatccccctgagcggatcaagctggactacgatcagtattgtgccgacgtggccgccgaggagctgatgaacgccctggtgaattccaccctgctggaggccagagccacaaaccagtttctggccgtgagcaagggcaattgctccggcccaaccacaatccggggccagttctctaacatgtctctgagcctgctggatctgtacctgagcagaggctataatgtgagctccatcgtgaccatgacaagccagggcatgtacggcggcacctatctggtgggcaagcccaacctgtctagcaagggctccgagctgtctcagctgagcatgcacagggtgtttgaagtgggcgtgatcaggaacccaggcctgggagcccccgtgtttcacatgaccaattacttcgagcagcccgtgagcaacgatttttctaattgcatggtggccctgggagagctgaagttcgccgccctgtgccacagggaggacagcatcaccatcccatatcagggctctggcaagggcgtgagcttccagctggtgaagctgggcgtgtggaagtcccccaccgacatgagatcctgggtgcctctgtctacagacgatccagtgatcgatcggctgtacctgtcctctcacagaggcgtgatcgccgacaatcaggcaaagtgggcagtgccaaccacaaggaccgacgataagctgagaatggagacatgctttcagcaggcctgtaagggcaagaaccaggccctgtgcgagaatccagagtgggcacctctgaaggataacagaatccctagctacggcgtgctgagcgtgaatctgtccctgaccgtggagctgaagatcaagatcgccagcggcttcggcccactgatcacacacggctccggcatggacctgtacaagaccaaccacaacaacgtgtactggctgacaatcccacccatgaagaacctggccctgggcgtgatcaataccctggagtggatccccaggtttaaggtgtcccctgccctgttcaccgtgccaatcaaggaggccggcgaggattgtcacgccccaacatacctgcccgccgaggtggacggcgatgtgaagctgagctccaacctggtcatcctgcctggccaggacctgcagtacgtgctggccacctatgatacaagccgcgtggagcacgccgtggtgtactacgtgtactccccctctcggagcttctcctacttttatcctttcagactgccaatcaagggcgtgcctatcgagctgcaggtggagtgctttacctgggataagaagctgtggtgcaggcacttctgcgtgctggcagactctgagagcggaggacacatcacccactccggaatggtgggcatgggcgtgtcttgtaccgtgacacgcgaggacggcacagggggaggcggctctggaggtggggggtccggtggcggcggaagcgcggccgcggatgtgcagctgcaggagagcggcggcggatctgtgcaagctggcggaagcctgagactgtcttgcgccgcttctggctacacccacagcagctattgcatggcctggttccggcaggcccctggcagagaacgggaaggcgtggccagcatcgacagcgacggcaccaccagctacgccgatagcgtcaagggcagattcaccatcagccaggacaatgccaaaaacaccctgtacctgcagatgaacagcctgaagcccgaggacacagccatgtactactgtgccgccagatttggccctatgggctgcgtggacctgtccaccctctccttcggccactggggacagggcacacaggtgacagtgtccatcaccggcggcggctccggcggcggcagctatccctatgatgtgcccgactatgcgtga

SEQ ID NO: 34 - DNA, 949 bp, synthetic construct
  source 1..949: mol_type = other DNA
gaagaacctggccctgggcgtgatcaataccctggagtggatccccaggtttaaggtgtcccctgccctgttcaccgtgccaatcaaggaggccggcgaggattgtcacgccccaacatacctgcccgccgaggtggacggcgatgtgaagctgagctccaacctggtcatcctgcctggccaggacctgcagtacgtgctggccacctatgatacaagccgcgtggagcacgccgtggtgtactacgtgtactccccctctcggagcttctcctacttttatcctttcagactgccaatcaagggcgtgcctatcgagctgcaggtggagtgctttacctgggataagaagctgtggtgcaggcacttctgcgtgctggcagactctgagagcggaggacacatcacccactccggaatggtgggcatgggcgtgtcttgtaccgtgacacgcgaggacggcacagggggaggcggctctggaggtggggggtccggtggcggcggaagcgcggccgcggaggtgcagctggtcgagagcggaggaggcagcgtgcagcccggcggatctctgaccctgagctgtggcacaagcggcagaacctttaacgtgatgggctggttcagacaggcccctggcaaggaaagagagttcgtggccgctgtgcggtggtcctctacaggcatctactatacacagtacgccgacagcgttaaaagcagattcaccatcagccgggacaacgccaagaacaccgtgtacctggaaatgaacagcctgaagccagaggatacagccgtgtactactgcgccgccgatacctacaactctaatcctgctagatgggacggctacgacttccggggccagggcacccaagtgaccgtgtccagcggcggcggctccggcggcggcagctatccctatgatgtgcccgactatgcgtga

SEQ ID NO: 35 - protein, 750 aa, synthetic construct
MGSRIVINREHLMIDRPYVLLAVLFVMFLSLIGLLAIAGIRLHRAAIYTAEIHKSLSTNLDVTNSIEHQVKDVLTPLFKIIGDEVGLRTPQRFTDLVKFISDKIKFLNPDREYDFRDLTWCINPPERIKLDYDQYCADVAAEELMNALVNSTLLEARATNQFLAVSKGNCSGPTTIRGQFSNMSLSLLDLYLSRGYNVSSIVTMTSQGMYGGTYLVGKPNLSSKGSELSQLSMHRVFEVGVIRNPGLGAPVFHMTNYFEQPVSNDFSNCMVALGELKFAALCHREDSITIPYQGSGKGVSFQLVKLGVWKSPTDMRSWVPLSTDDPVIDRLYLSSHRGVIADNQAKWAVPTTRTDDKLRMETCFQQACKGKNQALCENPEWAPLKDNRIPSYGVLSVNLSLTVELKIKIASGFGPLITHGSGMDLYKTNHNNVYWLTIPPMKNLALGVINTLEWIPRFKVSPALFTVPIKEAGEDCHAPTYLPAEVDGDVKLSSNLVILPGQDLQYVLATYDTSRVEHAVVYYVYSPSRSFSYFYPFRLPIKGVPIELQVECFTWDKKLWCRHFCVLADSESGGHITHSGMVGMGVSCTVTREDGTGGGGSGGGGSGGGGSAAAQVQLQESGGGSVQPGGSLRLSCAASGFTFDDYAMSWVRQVPGKGLEWVSTINWNGGSAEYAEPVKGRFTISRDNAKNTVYLQMNSLKLEDTAVYYCAKDADLVWYNLSTGQGTQVTVSSGGGSGGGSYPYDVPDYA

SEQ ID NO: 36 - DNA, 839 bp, synthetic construct
  source 1..839: mol_type = other DNA
aggtggacggcgatgtgaagctgagctccaacctggtcatcctgcctggccaggacctgcagtacgtgctggccacctatgatacaagccgcgtggagcacgccgtggtgtactacgtgtactccccctctcggagcttctcctacttttatcctttcagactgccaatcaagggcgtgcctatcgagctgcaggtggagtgctttacctgggataagaagctgtggtgcaggcacttctgcgtgctggcagactctgagagcggaggacacatcacccactccggaatggtgggcatgggcgtgtcttgtaccgtgacacgcgaggacggcacagggggaggcggctctggaggtggggggtccggtggcggcggaagcgcggccgcgcaagtgcagctgcaggagagcggcggaggcagcgtgcagcctggcggaagcctgagactgtcctgcgccgcttctggctttaccttcgacgactacgccatgagctgggtgcggcaggttccaggcaagggcctggaatgggtcagcacaatcaactggaacggcggatctgctgagtacgccgagcctgtgaagggcagattcacaatcagcagagataatgccaaaaacaccgtgtatctgcagatgaacagcctcaagctggaagatacagccgtgtactactgtgccaaggacgccgacctggtgtggtacaacctgagcaccggccagggcacccaggtgaccgtgtccagcgccgctgcctacccctacgacgtgcctgattacggctctggcggcggctccggcggcggcagctatccctatgatgtgcccgactatgcgtga

SEQ ID NO: 37 - protein, 363 aa, Homo sapiens
MEPPGRRECPFPSWRFPGLLLAAMVLLLYSFSDACEEPPTFEAMELIGKPKPYYEIGERVDYKCKKGYFYIPPLATHTICDRNHTWLPVSDDACYRETCPYIRDPLNGQAVPANGTYEFGYQMHFICNEGYYLIGEEILYCELKGSVAIWSGKPPICEKVLCTPPPKIKNGKHTFSEVEVFEYLDAVTYSCDPAPGPDPFSLIGESTIYCGDNSVWSRAAPECKVVKCRFPVVENGKQISGFGKKFYYKATVMFECDKGFYLDGSDTIVCDSNSTWDPPVPKCLKVSTSSTTKSPASSASGYPKPEEGILDSLDVWVIAVIVIAIVVGVAVICVVPYRYLQRRKKKGTYLTDETHREVKFTSL

SEQ ID NO: 38 - DNA, 3278 bp, Homo sapiens
  source 1..3278: mol_type = genomic DNA
gcggctcgggccacgcccacctgtcctgcagcactggatgctttgtgagttggggattgttgcgtcccatatctggacccagaagggacttccctgctcggctggctctcggtttctctgctttcctccggagaaataacagcgtcttccgcgccgcgcatggagcctcccggccgccgcgagtgtccctttccttcctggcgctttcctgggttgcttctggcggccatggtgttgctgctgtactccttctccgatgcctgtgaggagccaccaacatttgaagctatggagctcattggtaaaccaaaaccctactatgagattggtgaacgagtagattataagtgtaaaaaaggatacttctatatacctcctcttgccacccatactatttgtgatcggaatcatacatggctacctgtctcagatgacgcctgttatagagaaacatgtccatatatacgggatcctttaaatggccaagcagtccctgcaaatgggacttacgagtttggttatcagatgcactttatttgtaatgagggttattacttaattggtgaagaaattctatattgtgaacttaaaggatcagtagcaatttggagcggtaagcccccaatatgtgaaaaggttttgtgtacaccacctccaaaaataaaaaatggaaaacacacctttagtgaagtagaagtatttgagtatcttgatgcagtaacttatagttgtgatcctgcacctggaccagatccattttcacttattggagagagcacgatttattgtggtgacaattcagtgtggagtcgtgctgctccagagtgtaaagtggtcaaatgtcgatttccagtagtcgaaaatggaaaacagatatcaggatttggaaaaaaattttactacaaagcaacagttatgtttgaatgcgataagggtttttacctcgatggcagcgacacaattgtctgtgacagtaacagtacttgggatcccccagttccaaagtgtcttaaagtgtcgacttcttccactacaaaatctccagcgtccagtgcctcaggatatcctaaacctgaggaaggaatacttgacagtttggatgtttgggtcattgctgtgattgttattgccatagttgttggagttgcagtaatttgtgttgtcccgtacagatatcttcaaaggaggaagaagaaaggcacatacctaactgatgagacccacagagaagtaaaatttacttctctctgagaaggagagatgagagaaaggtttgcttttatcattaaaaggaaagcagatggtggagctgaatatgccacttaccagactaaatcaaccactccagcagagcagagaggctgaatagattccacaacctggtttgccagttcatcttttgactctattaaaatcttcaatagttgttattctgtagtttcactctcatgagtgcaactgtggcttagctaatattgcaatgtggcttgaatgtaggtagcatcctttgatgcttctttgaaacttgtatgaatttgggtatgaacagattgcctgctttcccttaaataacacttagatttattggaccagtcagcacagcatgcctggttgtattaaagcagggatatgctgtattttataaaattggcaaaattagagaaatatagttcacaatgaaattatattttctttgtaaagaaagtggcttgaaatcttttttgttcaaagattaatgccaactcttaagattattctttcaccaactatagaatgtattttatatatcgttcattgtaaaaagcccttaaaaatatgtgtatactactttggctcttgtgcataaaaacaagaacactgaaaattgggaatatgcacaaacttggcttctttaaccaagaatattattggaaaattctctaaaagttaatagggtaaattctctattttttgtaatgtgttcggtgatttcagaaagctagaaagtgtatgtgtggcatttgttttcactttttaaaacatccctaactgatcgaatatatcagtaatttcagaatcagatgcatcctttcataagaagtgagaggactctgacagccataacaggagtgccacttcatggtgcgaagtgaacactgtagtcttgttgttttcccaaagagaactccgtatgttctcttaggttgagtaacccactctgaattctggttacatgtgtttttctctccctccttaaataaagagaggggttaaacatgccctctaaaagtaggtggttttgaagagaataaattcatcagataacctcaagtcacatgagaatcttagtccatttacattgccttggctagtaaaagccatctatgtatatgtcttacctcatctcctaaaaggcagagtacaaagtaagccatgtatctcaggaaggtaacttcattttgtctatttgctgttgattgtaccaagggatggaagaagtaaatatagctcaggtagcactttatactcaggcagatctcagccctctactgagtcccttagccaagcagtttctttcaaagaagccagcaggcgaaaagcagggactgccactgcatttcatatcacactgttaaaagttgtgttttgaaattttatgtttagttgcacaaattgggccaaagaaacattgccttgaggaagatatgattggaaaatcaagagtgtagaagaataaatactgttttactgtccaaagacatgtttatagtgctctgtaaatgttcctttcctttgtagtctctggcaagatgctttaggaagataaaagtttgaggagaacaaacaggaattctgaattaagcacagagttgaagtttatacccgtttcacatgcttttcaagaatgtcgcaattactaagaagcagataatggtgttttttagaaacctaattgaagtatattcaaccaaatactttaatgtataaaataaatattatacaatatacttgtatagcagtttctgcttcacatttgattttttcaaatttaatatttatattagagatctatatatgtataaatatgtattttgtcaaatttgttacttaaatatatagagaccagttttctctggaagtttgtttaaatgacagaagcgtatatgaattcaagaaaatttaagctgcaaaaatgtatttgctataaaatgagaagtctcactgatagaggttctttattgctcattttttaaaaaatggactcttgaaatctgttaaaataaaattgtacatttgga

SEQ ID NO: 39 - protein, 323 aa, Homo sapiens
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 40 - DNA, 5292 bp, Homo sapiens
  source 1..5292: mol_type = genomic DNA
gcagcctgggcagtgggtcctgcctgtgacgcgcggcggcggtcggtcctgcctgtaacggcggcggcggctgctgctccggacacctgcggcggcggcggcgaccccgcggcgggcgcggagatgtggcccctggtagcggcgctgttgctgggctcggcgtgctgcggatcagctcagctactatttaataaaacaaaatctgtagaattcacgttttgtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttgtttcatggttttctccaaatgaaaatattcttattgttattttcccaatttttgctatactcctgttctggggacagtttggtattaaaacacttaaatatagatccggtggtatggatgagaaaacaattgctttacttgttgctggactagtgatcactgtcattgtcattgttggagccattcttttcgtcccaggtgaatattcattaaagaatgctactggccttggtttaattgtgacttctacagggatattaatattacttcactactatgtgtttagtacagcgattggattaacctccttcgtcattgccatattggttattcaggtgatagcctatatcctcgctgtggttggactgagtctctgtattgcggcgtgtataccaatgcatggccctcttctgatttcaggtttgagtatcttagctctagcacaattacttggactagtttatatgaaatttgtggcttccaatcagaagactatacaacctcctaggaaagctgtagaggaaccccttaatgcattcaaagaatcaaaaggaatgatgaatgatgaataactgaagtgaagtgatggactccgatttggagagtagtaagacgtgaaaggaatacacttgtgtttaagcaccatggccttgatgattcactgttggggagaagaaacaagaaaagtaactggttgtcacctatgagacccttacgtgattgttagttaagtttttattcaaagcagctgtaatttagttaataaaataattatgatctatgttgtttgcccaattgagatccagttttttgttgttatttttaatcaattaggggcaatagtagaatggacaatttccaagaatgatgcctttcaggtcctagggcctctggcctctaggtaaccagtttaaattggttcagggtgataactacttagcactgccctggtgattacccagagatatctatgaaaaccagtggcttccatcaaacctttgccaactcaggttcacagcagctttgggcagttatggcagtatggcattagctgagaggtgtctgccacttctgggtcaatggaataataaattaagtacaggcaggaatttggttgggagcatcttgtatgatctccgtatgatgtgatattgatggagatagtggtcctcattcttgggggttgccattcccacattcccccttcaacaaacagtgtaacaggtccttcccagatttagggtacttttattgatggatatgttttccttttattcacataaccccttgaaaccctgtcttgtcctcctgttacttgcttctgctgtacaagatgtagcaccttttctcctctttgaacatggtctagtgacacggtagcaccagttgcaggaaggagccagacttgttctcagagcactgtgttcacacttttcagcaaaaatagctatggttgtaacatatgtattcccttcctctgatttgaaggcaaaaatctacagtgtttcttcacttcttttctgatctggggcatgaaaaaagcaagattgaaatttgaactatgagtctcctgcatggcaacaaaatgtgtgtcaccatcaggccaacaggccagcccttgaatggggatttattactgttgtatctatgttgcatgataaacattcatcaccttcctcctgtagtcctgcctcgtactccccttcccctatgattgaaaagtaaacaaaacccacatttcctatcctggttagaagaaaattaatgttctgacagttgtgatcgcctggagtacttttagacttttagcattcgttttttacctgtttgtggatgtgtgtttgtatgtgcatacgtatgagataggcacatgcatcttctgtatggacaaaggtggggtacctacaggagagcaaaggttaattttgtgcttttagtaaaaacatttaaatacaaagttctttattgggtggaattatatttgatgcaaatatttgatcacttaaaacttttaaaacttctaggtaatttgccacgctttttgactgctcaccaataccctgtaaaaatacgtaattcttcctgtttgtgtaataagatattcatatttgtagttgcattaataatagttatttcttagtccatcagatgttcccgtgtgcctcttttatgccaaattgattgtcatatttcatgttgggaccaagtagtttgcccatggcaaacctaaatttatgacctgctgaggcctctcagaaaactgagcatactagcaagacagctcttcttgaaaaaaaaaatatgtatacacaaatatatacgtatatctatatatacgtatgtatatacacacatgtatattcttccttgattgtgtagctgtccaaaataataacatatatagagggagctgtattcctttatacaaatctgatggctcctgcagcactttttccttctgaaaatatttacattttgctaacctagtttgttactttaaaaatcagttttgatgaaaggagggaaaagcagatggacttgaaaaagatccaagctcctattagaaaaggtatgaaaatctttatagtaaaattttttataaactaaagttgtaccttttaatatgtagtaaactctcatttatttggggttcgctcttggatctcatccatccattgtgttctctttaatgctgcctgccttttgaggcattcactgccctagacaatgccaccagagatagtgggggaaatgccagatgaaaccaactcttgctctcactagttgtcagcttctctggataagtgaccacagaagcaggagtcctcctgcttgggcatcattgggccagttccttctctttaaatcagatttgtaatggctcccaaattccatcacatcacatttaaattgcagacagtgttttgcacatcatgtatctgttttgtcccataatatgctttttactccctgatcccagtttctgctgttgactcttccattcagttttatttattgtgtgttctcacagtgacaccatttgtccttttctgcaacaacctttccagctacttttgccaaattctatttgtcttctccttcaaaacattctcctttgcagttcctcttcatctgtgtagctgctcttttgtctcttaacttaccattcctatagtactttatgcatctctgcttagttctattagttttttggccttgctcttctccttgattttaaaattccttctatagctagagcttttctttctttcattctctcttcctgcagtgttttgcatacatcagaagctaggtacataagttaaatgattgagagttggctgtatttagatttatcactttttaatagggtgagcttgagagttttctttctttctgttttttttttttgttttttttttttttttttttttttttttttttgactaatttcacatgctctaaaaaccttcaaaggtgattatttttctcctggaaactccaggtccattctgtttaaatccctaagaatgtcagaattaaaataacagggctatcccgtaattggaaatatttcttttttcaggatgctatagtcaatttagtaagtgaccaccaaattgttatttgcactaacaaagctcaaaacacgataagtttactcctccatctcagtaataaaaattaagctgtaatcaaccttctaggtttctcttgtcttaaaatgggtattcaaaaatggggatctgtggtgtatgtatggaaacacatactccttaatttacctgttgttggaaactggagaaatgattgtcgggcaaccgtttattttttattgtattttatttggttgagggatttttttataaacagttttacttgtgtcatattttaaaattactaactgccatcacctgctggggtcctttgttaggtcattttcagtgactaatagggataatccaggtaactttgaagagatgagcagtgagtgaccaggcagtttttctgcctttagctttgacagttcttaattaagatcattgaagaccagctttctcataaatttctctttttgaaaaaaagaaagcatttgtactaagctcctctgtaagacaacatcttaaatcttaaaagtgttgttatcatgactggtgagagaagaaaacattttgtttttattaaatggagcattatttacaaaaagccattgttgagaattagatcccacatcgtataaatatctattaaccattctaaataaagagaactccagtgttgctatgtgcaagatcctctcttggagcttttttgcatagcaattaaaggtgtgctatttgtcagtagccatttttttgcagtgatttgaagaccaaagttgttttacagctgtgttaccgttaaaggtttttttttttatatgtattaaatcaatttatcactgtttaaagctttgaatatctgcaatctttgccaaggtacttttttatttaaaaaaaaacataactttgtaaatattaccctgtaatattatatatacttaataaaacattttaagctattttgttgggctatttctattgctgctacagcagaccacaagcacatttctgaaaaatttaatttattaatgtatttttaagttgcttatattctaggtaacaatgtaaagaatgatttaaaatattaattatgaattttttgagtataatacccaataagcttttaattagagcagagttttaattaaaagttttaaatcagtccaa

SEQ ID NO: 41 - protein, 440 aa, Homo sapiens
MTVARPSVPAALPLLGELPRLLLLVLLCLPAVWGDCGLPPDVPNAQPALEGRTSFPEDTVITYKCEESFVKIPGEKDSVICLKGSQWSDIEEFCNRSCEVPTRLNSASLKQPYITQNYFPVGTVVEYECRPGYRREPSLSPKLTCLQNLKWSTAVEFCKKKSCPNPGEIRNGQIDVPGGILFGATISFSCNTGYKLFGSTSSFCLISGSSVQWSDPLPECREIYCPAPPQIDNGIIQGERDHYGYRQSVTYACNKGFTMIGEHSIYCTVNNDEGEWSGPPPECRGKSLTSKVPPTVQKPTTVNVPTTEVSPTSQKTTTKTTTPNAQATRSTPVSRTTKHFHETTPNKGSGTTSGTTRLLSGSRPVTQAGMRWCDRSSLQSRTPGFKRSFHFSLPSSWYYRAHVFHVDRFAWDASNHGLADLAKEELRRKYTQVYRLFLVS

SEQ ID NO: 42 - DNA, 2708 bp, Homo sapiens
  source 1..2708: mol_type = genomic DNA
ctgcttactgcaactcgctccggccgctgggcgtagctgcgactcggcggagtcccggcggcgcgtccttgttctaacccggcgcgccatgaccgtcgcgcggccgagcgtgcccgcggcgctgcccctcctcggggagctgccccggctgctgctgctggtgctgttgtgcctgccggccgtgtggggtgactgtggccttcccccagatgtacctaatgcccagccagctttggaaggccgtacaagttttcccgaggatactgtaataacgtacaaatgtgaagaaagctttgtgaaaattcctggcgagaaggactcagtgatctgccttaagggcagtcaatggtcagatattgaagagttctgcaatcgtagctgcgaggtgccaacaaggctaaattctgcatccctcaaacagccttatatcactcagaattattttccagtcggtactgttgtggaatatgagtgccgtccaggttacagaagagaaccttctctatcaccaaaactaacttgccttcagaatttaaaatggtccacagcagtcgaattttgtaaaaagaaatcatgccctaatccgggagaaatacgaaatggtcagattgatgtaccaggtggcatattatttggtgcaaccatctccttctcatgtaacacagggtacaaattatttggctcgacttctagtttttgtcttatttcaggcagctctgtccagtggagtgacccgttgccagagtgcagagaaatttattgtccagcaccaccacaaattgacaatggaataattcaaggggaacgtgaccattatggatatagacagtctgtaacgtatgcatgtaataaaggattcaccatgattggagagcactctatttattgtactgtgaataatgatgaaggagagtggagtggcccaccacctgaatgcagaggaaaatctctaacttccaaggtcccaccaacagttcagaaacctaccacagtaaatgttccaactacagaagtctcaccaacttctcagaaaaccaccacaaaaaccaccacaccaaatgctcaagcaacacggagtacacctgtttccaggacaaccaagcattttcatgaaacaaccccaaataaaggaagtggaaccacttcaggtactacccgtcttctatctggttctcgtcctgtcacccaggctggtatgcggtggtgtgatcgtagctcactgcagtctcgaactcctgggttcaagcgatccttccacttcagcctcccaagtagctggtactacagggcacacgtgtttcacgttgacaggtttgcttgggacgctagtaaccatgggcttgctgacttagccaaagaagagttaagaagaaaatacacacaagtatacagactgttcctagtttcttagacttatctgcatattggataaaataaatgcaattgtgctcttcatttaggatgctttcattgtctttaagatgtgttaggaatgtcaacagagcaaggagaaaaaaggcagtcctggaatcacattcttagcacacctacacctcttgaaaatagaacaacttgcagaattgagagtgattcctttcctaaaagtgtaagaaagcatagagatttgttcgtatttagaatgggatcacgaggaaaagagaaggaaagtgatttttttccacaagatctgtaatgttatttccacttataaaggaaataaaaaatgaaaaacattatttggatatcaaaagcaaataaaaacccaattcagtctcttctaagcaaaattgctaaagagagatgaaccacattataaagtaatctttggctgtaaggcattttcatctttccttcgggttggcaaaatattttaaaggtaaaacatgctggtgaaccaggggtgttgatggtgataagggaggaatatagaatgaaagactgaatcttcctttgttgcacaaatagagtttggaaaaagcctgtgaaaggtgtcttctttgacttaatgtctttaaaagtatccagagatactacaatattaacataagaaaagattatatattatttctgaatcgagatgtccatagtcaaatttgtaaatcttattcttttgtaatatttatttatatttatttatgacagtgaacattctgattttacatgtaaaacaagaaaagttgaagaagatatgtgaagaaaaatgtatttttcctaaatagaaataaatgatcccattttttggtatcatgtagtatgtgaaatttattcttaaacgtgactactttatttctaaataagaaattccctacctgcttcctacaagcagttcagaatgccatgccttggttgtcctagtgtgaataattttcagctactttaaaattatattgtactttctcaagcatgtcatatcctttcctattagagtatctatattacttgttactgatttacctgaaggcaatctgattaatttctaggtttttaccatattcttgtcatcttgccaattacattttaagtgttagactagactaagatgtactagttgtatagaatataactagatttattatggcaatgtttattttgtcattttgcttcatctgttttgttgttgaagtactttaaatttcatacgttcatggcatttcactgtaaagactttaatgtgtatttcttaaaataaaactttttttcctccttaa

SEQ ID NO: 43 - protein, 128 aa, Homo sapiens
MGIQGGSVLFGLLLVLAVFCHSGHSLQCYNCPNPTADCKTAVNCSSDFDACLITKAGLQVYNKCWKFEHCNFNDVTTRLRENELTYYCCKKDLCNFNEQLENGGTSLSEKTVLLLVTPFLAAAWSLHP

SEQ ID NO: 44 - DNA, 7796 bp, Homo sapiens
  source 1..7796: mol_type = genomic DNA
ggggccggggggcggagccttgcgggctggagcgaaagaatgcgggggctgagcgcagaagcggctcgaggctggaagaggatcttgggcgccgccagtctctctctgttgcccaagctggagtgcagtggcacagtcttggctcactgcaacctccacctcctgggtgcaagcgattctcgtgtctcagcctctcaagtagctgggattacagtctttagcaccagttggtgtaggagttgagacctacttcacagtagttctgtggacaatcacaatgggaatccaaggagggtctgtcctgttcgggctgctgctcgtcctggctgtcttctgccattcaggtcatagcctgcagtgctacaactgtcctaacccaactgctgactgcaaaacagccgtcaattgttcatctgattttgatgcgtgtctcattaccaaagctgggttacaagtgtataacaagtgttggaagtttgagcattgcaatttcaacgacgtcacaacccgcttgagggaaaatgagctaacgtactactgctgcaagaaggacctgtgtaactttaacgaacagcttgaaaatggtgggacatccttatcagagaaaacagttcttctgctggtgactccatttctggcagcagcctggagccttcatccctaagtcaacaccaggagagcttctcccaaactccccgttcctgcgtagtccgctttctcttgctgccacattctaaaggcttgatattttccaaatggatcctgttgggaaagaataaaattagcttgagcaacctggctaagatagaggggctctgggagactttgaagaccagtcctgtttgcagggaagccccacttgaaggaagaagtctaagagtgaagtaggtgtgacttgaactagattgcatgcttcctcctttgctcttgggaagaccagctttgcagtgacagcttgagtgggttctctgcagccctcagattatttttcctctggctccttggatgtagtcagttagcatcattagtacatctttggagggtggggcaggagtatatgagcatcctctctcacatggaacgctttcataaacttcagggatcccgtgttgccatggaggcatgccaaatgttccatatgtgggtgtcagtcagggacaacaagatccttaatgcagagctagaggacttctggcagggaagtggggaagtgttccagatagcagggcatgaaaacttagagaggtacaagtggctgaaaatcgagtttttcctctgtctttaaattttatatgggctttgttatcttccactggaaaagtgtaatagcatacatcaatggtgtgttaaagctatttccttgccttttttttattggaatggtaggatatcttggctttgccacacacagttacagagtgaacactctactacatgtgactggcagtattaagtgtgcttattttaaatgttactggtagaaaggcagttcaggtatgtgtgtatatagtatgaatgcagtggggacaccctttgtggttacagtttgagacttccaaaggtcatccttaataacaacagatctgcaggggtatgttttaccatctgcatccagcctcctgctaactcctagctgactcagcatagattgtataaaatacctttgtaacggctcttagcacactcacagatgtttgaggctttcagaagctcttctaaaaaatgatacacacctttcacaagggcaaactttttccttttccctgtgtattctagtgaatgaatctcaagattcagtagacctaatgacatttgtattttatgatcttggctgtatttaatggcataggctgacttttgcagatggaggaatttcttgattaatgttgaaaaaaaacccttgattatactctgttggacaaaccgagtgcaatgaatgatgcttttctgaaaatgaaatataacaagtgggtgaatgtggttatggccgaaaaggatatgcagtatgcttaatggtagcaactgaaagaagacatcctgagcagtgccagctttcttctgttgatgccgttccctgaacataggaaaatagaaacttgcttatcaaaacttagcattaccttggtgctctgtgttctctgttagctcagtgtctttccttacatcaataggtttttttttttttttttggcctgaggaagtactgaccatgcccacagccaccggctgagcaaagaagctcatttcatgtgagttctaaggaatgagaaacaattttgatgaatttaagcagaaaatgaatttctgggaacttttttgggggcgggggggtggggaattcagccacactccagaaagccaggagtcgacagttttggaagcctctctcaggattgagattctaggatgagattggcttactgctatcttgtgtcatgtacccactttttggccagactacactgggaagaaggtagtcctctaaagcaaaatctgagtgccactaaatggggagatggggctgttaagctgtccaaatcaacaagggtcatataaatggccttaaactttggggttgctttctgcaaaaagttgctgtgactcatgccatagacaaggttgagtgcctggacccaaaggcaatactgtaatgtaaagacatttatagtactaggcaaacagcaccccaggtactccaggccctcctggctggagagggctgtggcaatagaaaattagtgccaactgcagtgagtcagcctaggttaaatagagagtgtaagagtgctggacaggaacctccaccctcatgtcacatttcttcaatgtgacccttctggcccctctcctcctgacagcggaacaatgactgccccgataggtgaggctggaggaagaatcagtcctgtccttggcaagctcttcactatgacagtaaaggctctctgcctgctgccaaggcctgtgactttctaacctggcctcacgctgggtaagcttaaggtagaggtgcaggattagcaagcccacctggctaccaggccgacagctacatcctccaactgaccctgatcaacgaagagggattcatgtgtctgtctcagttggttccaaatgaaaccagggagcaggggagttaggaatcgaacaccagtcatgcctactggctctctgctcgagagccaataccctgtgccctccactcatctggatttacaggaactgtcatagtgttcagtattgggtggtgataagcccattggattgtccccttggggggatgagctaggggtgcaaggaacacctgatgagtagataagtggagctcatggtatttcctgaaagatgctaatctatttgccaaacttggtcttgaatgtactgggggcttcaaggtatgggtatatttttcttgtgtccttgcagttagcccccatgtcttatgtgtgtcctgaaaaaataagagcctgcccaagactttgggcctcttgacagaattaaccacttttatacatctgagttctcttggtaagttctttagcagtgttcaaagtctactagctcgcattagtttctgttgctgccaacagatctgaactaatgctaacagatccccctgagggattcttgatgggctgagcagctggctggagctagtactgactgacattcattgtgatgagggcagctttctggtacaggattctaagctctatgttttatatacattttcatctgtacttgcacctcactttacacaagaggaaactatgcaaagttagctggatcgctcaaggtcacttaggtaagttggcaagtccatgcttcccactcagctcctcaggtcagcaagtctacttctctgcctattttgtatactctctttaatatgtgcctagctttggaaagtctagaatgggtccctggtgcctttttactttgaagaaatcagtttctgcctctttttggaaaagaaaacaaagtgcaattgttttttactggaaagttacccaatagcatgaggtgaacaggacgtagttaggccttcctgtaaacagaaaatcatatcaaaacactatcttcccatctgtttctcaatgcctgctacttcttgtagatatttcatttcaggagagcagcagttaaacccgtggattttgtagttaggaacctgggttcaaaccctcttccactaattggctatgtctctggacaagtttttttttttttttttttttaaaccctttctgaactttcactttctatgtctacctcaaagaattgttgtgaggcttgagataatgcatttgtaaagggtctgccagataggaagatgctagttatggatttacaaggttgttaaggctgtaagagtctaaaacctacagtgaatcacaatgcatttacccccactgacttggacataagtgaaaactagccagaagtctctttttcaaattacttacaggttattcaatataaaatttttgtaatggataatcttatttatctaaactaaagcttcctgtttatacacactcctgttattctgggataagataaatgaccacagtaccttaatttctaggtgggtgcctgtgatggttcattgtaggtaaggacattttctctttttcagcagctgtgtaggtccagagcctctgggagaggaggggggtagcatgcacccagcaggggactgaactgggaaactcaaggttctttttactgtggggtagtgagctgcctttctgtgatcggtttccctagggatgttgctgttcccctccttgctattcgcagctacatacaacgtggccaaccccagtaggctgatcctatatatgatcagtgctggtgctgactctcaatagccccacccaagctggctataggtttacagatacattaattaggcaacctaaaatattgatgctggtgttggtgtgacataatgctatggccagaactgaaacttagagttataattcatgtattagggttctccagagggacagaattagtaggatatatgtatatatgaaagggaggttattagggagaactggctcccacagttagaaggcgaagtcgcacaataggccgtctgcaagctgggttagagagaagccagtagtggctcagcctgagttcaaaaacctcaaaactggggaagctgacagtgcagccagccttcagtctgtggccaaaggcccaagagcccctggcaaccaacccactggtgcaagtcctagattccaaaggctgaagaacctggagtctgatgtccaagagcaggaagagtggaagaaagccagaagactcagcaaacaaggtagacagtgtctaccaccatagtggccataccaaagaggctaccgattccttcctgctacctggatccctgaagttgccctggtctctgcaccttctaaacctagttcttaagagctttccattacatgagctgtctcaaagccctccaataaattctcagtgtaagcttctgttgcttgtggacagaaaattctgacagacctaccctataagtgttactgtcaggataacatgagaacgcacaacagtaagtggtcactaagtgttagctacggttattttgcccaaggtagcatggctagttgatgccggttgatggggcttaaacccagctccctcatcttccaggcctctgtactccctattccactaaactacctctcaggtttatttttttaaattcttactctgcaagtacataggaccacatttacctgggaaaacaagaataaaggctgctctgcattttttagaaacttttttgaaagggagatgggaatgcctgcacccccaagtccagaccaacacaatggttaattgagatgaataataaaggaaagactgttctgggcttcccagaatagcttggtccttaaattgtggcacaaacaacctcctgtcagagccagcctcctgccaggaagaggggtaggagactagaggccgtgtgtgcagccttgccctgaaggctagggtgacaatttggaggctgtccaaacaccctggcctctagagctggcctgtctatttgaaatgccggctctgatgctaatcggcgaccctcaggcaagttacttaaccttacatgcctcagttttctcatctggaaaatgagaaccctaggtttagggttgttagaaaagttaaatgagttaagacaagtgcctgggacacagtagcctcttgtgtgtgtttatcattatgtcctcagcaggtcgtagaagcagcttctcaggtgtgaggctggcgcgattatctggagtgggttgggttttctaggatggaccccctgctgcattttcctcattcatccaccagggcttaatggggaatcaaggaatccatgtgtaactgtataataactgtagccacactccaatgaccacctactagttgtccctggcactgcttatacatatgtccatcaaatcaatcctatgaagtagatactgtcttcattttatagatcagagacaattggggttcagagagctgatgtgattttcccagggtcacagagagtcccagattcaggcacaactcttgtattccaagacacaaccactacatgtccaaaggctgcccagagccaccgggcacggcaaattgtgacatatccctaaagaggctgagcacctggtcaggatctgatggctgacagtgtgtccagatgcagagctggagtgggggaggggaaggggggctccttgggacagagaaggctttctgtgctttctctgaagggagcagtctgaggaccaagggaacccggcaaacagcacctcaggtactccaggccctcctggctggagagggctgtggcaatggaaaattagtgccaactgcaatgagtcagcctcggttaaatagagagtgaagaatgctggacaggaacctccaccctcatgtcacatttcttcagtgtgacccttctggcccctctcctcctgacagcggaacaatgactgccccgataggtgaggctggaggaagaatcagtcctgtccttggcaagctcttcactatgacagtaaaggctctctgcctgctgccaaggcctgtgactttctaacctggcctcacgctgggtaagcttaaggtagaggtgcaggattagcaagcccacctggctaccaggccgacagctacatctttcaactgaccctgatcaacgaagagggacttgtgtctctcagttggttccaaatgaaaccagggagcaggggcgttaggaagctccaacaggatggtacttaatggggcatttgagtggagaggtaggtgacatagtgctttggagcccagggagggaaaggttctgctgaagttgaattcaagactgttctttcatcacaaacttgagtttcctggacatttgtttgcagaaacaaccgtagggttttgccttaacctcgtgggtttattattacctcatagggactttgcctcctgacagcagtttatgggtgttcattgtggcacttgagttttcttgcatacttgttagagaaaccaagtttgtcatcaacttcttatttaaccccctggctataacttcatggattatgttataattaagccatccagagtaaaatctgtttagattatcttggagtaagggggaaaaaatctgtaattttttctcctcaactagatatatacataaaaaatgattgtattgcttcatttaaaaaatataacgcaaaatctcttttccttctaaaaaaaaaaaaaaaaaa

SEQ ID NO: 45 - protein, 458 aa, Homo sapiens
MNRGVPFRHLLLVLQLALLPAATQGKKVVLGKKGDTVELTCTASQKKSIQFHWKNSNQIKILGNQGSFLTKGPSKLNDRADSRRSLWDQGNFPLIIKNLKIEDSDTYICEVEDQKEEVQLLVFGLTANSDTHLLQGQSLTLTLESPPGSSPSVQCRSPRGKNIQGGKTLSVSQLELQDSGTWTCTVLQNQKKVEFKIDIVVLAFQKASSIVYKKEGEQVEFSFPLAFTVEKLTGSGELWWQAERASSSKSWITFDLKNKEVSVKRVTQDPKLQMGKKLPLHLTLPQALPQYAGSGNLTLALEAKTGKLHQEVNLVVMRATQLQKNLTCEVWGPTSPKLMLSLKLENKEAKVSKREKAVWVLNPEAGMWQCLLSDSGQVLLESNIKVLPTWSTPVQPMALIVLGGVAGLLLFIGLGIFFCVRCRHRRRQAERMSQIKRLLSEKKTCQCPHRFQKTCSPI

SEQ ID NO: 46 - DNA, 1377 bp, Homo sapiens
  source 1..1377: mol_type = genomic DNA
atgaaccggggagtcccttttaggcacttgcttctggtgctgcaactggcgctcctcccagcagccactcagggaaagaaagtggtgctgggcaaaaaaggggatacagtggaactgacctgtacagcttcccagaagaagagcatacaattccactggaaaaactccaaccagataaagattctgggaaatcagggctccttcttaactaaaggtccatccaagctgaatgatcgcgctgactcaagaagaagcctttgggaccaaggaaactttcccctgatcatcaagaatcttaagatagaagactcagatacttacatctgtgaagtggaggaccagaaggaggaggtgcaattgctagtgttcggattgactgccaactctgacacccacctgcttcaggggcagagcctgaccctgaccttggagagcccccctggtagtagcccctcagtgcaatgtaggagtccaaggggtaaaaacatacagggggggaagaccctctccgtgtctcagctggagctccaggatagtggcacctggacatgcactgtcttgcagaaccagaagaaggtggagttcaaaatagacatcgtggtgctagctttccagaaggcctccagcatagtctataagaaagagggggaacaggtggagttctccttcccactcgcctttacagttgaaaagctgacgggcagtggcgagctgtggtggcaggcggagagggcttcctcctccaagtcttggatcacctttgacctgaagaacaaggaagtgtctgtaaaacgggttacccaggaccctaagctccagatgggcaagaagctcccgctccacctcaccctgccccaggccttgcctcagtatgctggctctggaaacctcaccctggcccttgaagcgaaaacaggaaagttgcatcaggaagtgaacctggtggtgatgagagccactcagctccagaaaaatttgacctgtgaggtgtggggacccacctcccctaagctgatgctgagcttgaaactggagaacaaggaggcaaaggtctcgaagcgggagaaggcggtgtgggtgctgaaccctgaggcggggatgtggcagtgtctgctgagtgactcgggacaggtcctgctggaatccaacatcaaggttctgcccacatggtccaccccggtgcagccaatggccctgattgtgctggggggcgtcgccggcctcctgcttttcattgggctaggcatcttcttctgtgtcaggtgccggcaccgaaggcgccaagcagagcggatgtctcagatcaagagactcctcagtgagaagaagacctgccagtgccctcaccggtttcagaagacatgtagccccatttag

SEQ ID NO: 47 - protein, 240 aa, Homo sapiens
MAGPPRLLLLPLLLALARGLPGALAAQEVQQSPHCTTVPVGASVNITCSTSGGLRGIYLRQLGPQPQDIIYYEDGVVPTTDRRFRGRIDFSGSQDNLTITMHRLQLSDTGTYTCQAITEVNVYGSGTLVLVTEEQSQGWHRCSDAPPRASALPAPPTGSALPDPQTASALPDPPAASALPAALAVISFLLGLGLGVACVLARTQIKKLCSWRDKNSAACVVYEDMSHSRCNTLSSPNQYQ

SEQ ID NO: 48 - DNA, 723 bp, Homo sapiens
  source 1..723: mol_type = genomic DNA
atggccgggcctccgaggctcctgctgctgcccctgcttctggcgctggctcgcggcctgcctggggccctggctgcccaagaggtgcagcagtctccccactgcacgactgtccccgtgggagcctccgtcaacatcacctgctccaccagcgggggcctgcgtgggatctacctgaggcagctcgggccacagccccaagacatcatttactacgaggacggggtggtgcccactacggacagacggttccggggccgcatcgacttctcagggtcccaggacaacctgactatcaccatgcaccgcctgcagctgtcggacactggcacctacacctgccaggccatcacggaggtcaatgtctacggctccggcaccctggtcctggtgacagaggaacagtcccaaggatggcacagatgctcggacgccccaccaagggcctctgccctccctgccccaccgacaggctccgccctccctgacccgcagacagcctctgccctccctgacccgccagcagcctctgccctccctgcggccctggcggtgatctccttcctcctcgggctgggcctgggggtggcgtgtgtgctggcgaggacacagataaagaaactgtgctcgtggcgggataagaattcggcggcatgtgtggtgtacgaggacatgtcgcacagccgctgcaacacgctgtcctcccccaaccagtaccagtga

SEQ ID NO: 49 - protein, 235 aa, Homo sapiens
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 50 - DNA, 708 bp, Homo sapiens
  source 1..708: mol_type = genomic DNA
atggccttaccagtgaccgccttgctcctgccgctggccttgctgctccacgccgccaggccgagccagttccgggtgtcgccgctggatcggacctggaacctgggcgagacagtggagctgaagtgccaggtgctgctgtccaacccgacgtcgggctgctcgtggctcttccagccgcgcggcgccgccgccagtcccaccttcctcctatacctctcccaaaacaagcccaaggcggccgaggggctggacacccagcggttctcgggcaagaggttgggggacaccttcgtcctcaccctgagcgacttccgccgagagaacgagggctactatttctgctcggccctgagcaactccatcatgtacttcagccacttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaccacaggaaccgaagacgtgtttgcaaatgtccccggcctgtggtcaaatcgggagacaagcccagcctttcggcgagatacgtctaa

SEQ ID NO: 51 - protein, 1306 aa, Homo sapiens
MTMYLWLKLLAFGFAFLDTEVFVTGQSPTPSPTGLTTAKMPSVPLSSDPLPTHTTAFSPASTFERENDFSETTTSLSPDNTSTQVSPDSLDNASAFNTTGVSSVQTPHLPTHADSQTPSAGTDTQTFSGSAANAKLNPTPGSNAISDVPGERSTASTFPTDPVSPLTTTLSLAHHSSAALPARTSNTTITANTSDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDLHKKRSCNLDEQQELVERDDEKQLMNVEPIHADILLETYKRKIADEGRLFLAEFQSIPRVFSKFPIKEARKPFNQNKNRYVDILPYDYNRVELSEINGDAGSNYINASYIDGFKEPRKYIAAQGPRDETVDDFWRMIWEQKATVIVMVTRCEEGNRNKCAEYWPSMEEGTRAFGDVVVKINQHKRCPDYIIQKLNIVNKKEKATGREVTHIQFTSWPDHGVPEDPHLLLKLRRRVNAFSNFFSGPIVVHCSAGVGRTGTYIGIDAMLEGLEAENKVDVYGYVVKLRRQRCLMVQVEAQYILIHQALVEYNQFGETEVNLSELHPYLHNMKKRDPPSEPSPLEAEFQRLPSYRSWRTQHIGNQEENKSKNRNSNVIPYDYNRVPLKHELEMSKESEHDSDESSDDDSDSEEPSKYINASFIMSYWKPEVMIAAQGPLKETIGDFWQMIFQRKVKVIVMLTELKHGDQEICAQYWGEGKQTYGDIEVDLKDTDKSSTYTLRVFELRHSKRKDSRTVYQYQYTNWSVEQLPAEPKELISMIQVVKQKLPQKNSSEGNKHHKSTPLLIHCRDGSQQTGIFCALLNLLESAETEEVVDIFQVVKALRKARPGMVSTFEQYQFLYDVIASTYPAQNGQVKKNNHQEDKIEFDNEVDKVKQDANCVNPLGAPEKLPEAKEQAEGSEPTSGTEGPEHSVNGPASPALNQGS

SEQ ID NO: 52 - DNA, 3921 bp, Homo sapiens
  source 1..3921: mol_type = genomic DNA
atgaccatgtatttgtggcttaaactcttggcatttggctttgcctttctggacacagaagtatttgtgacagggcaaagcccaacaccttcccccactggattgactacagcaaagatgcccagtgttccactttcaagtgaccccttacctactcacaccactgcattctcacccgcaagcacctttgaaagagaaaatgacttctcagagaccacaacttctcttagtccagacaatacttccacccaagtatccccggactctttggataatgctagtgcttttaataccacaggtgtttcatcagtacagacgcctcaccttcccacgcacgcagactcgcagacgccctctgctggaactgacacgcagacattcagcggctccgccgccaatgcaaaactcaaccctaccccaggcagcaatgctatctcagatgtcccaggagagaggagtacagccagcacctttcctacagacccagtttccccattgacaaccaccctcagccttgcacaccacagctctgctgccttacctgcacgcacctccaacaccaccatcacagcgaacacctcagatgcctaccttaatgcctctgaaacaaccactctgagcccttctggaagcgctgtcatttcaaccacaacaatagctactactccatctaagccaacatgtgatgaaaaatatgcaaacatcactgtggattacttatataacaaggaaactaaattatttacagcaaagctaaatgttaatgagaatgtggaatgtggaaacaatacttgcacaaacaatgaggtgcataaccttacagaatgtaaaaatgcgtctgtttccatatctcataattcatgtactgctcctgataagacattaatattagatgtgccaccaggggttgaaaagtttcagttacatgattgtacacaagttgaaaaagcagatactactatttgtttaaaatggaaaaatattgaaacctttacttgtgatacacagaatattacctacagatttcagtgtggtaatatgatatttgataataaagaaattaaattagaaaaccttgaacccgaacatgagtataagtgtgactcagaaatactctataataaccacaagtttactaacgcaagtaaaattattaaaacagattttgggagtccaggagagcctcagattattttttgtagaagtgaagctgcacatcaaggagtaattacctggaatccccctcaaagatcatttcataattttaccctctgttatataaaagagacagaaaaagattgcctcaatctggataaaaacctgatcaaatatgatttgcaaaatttaaaaccttatacgaaatatgttttatcattacatgcctacatcattgcaaaagtgcaacgtaatggaagtgctgcaatgtgtcatttcacaactaaaagtgctcctccaagccaggtctggaacatgactgtctccatgacatcagataatagtatgcatgtcaagtgtaggcctcccagggaccgtaatggcccccatgaacgttaccatttggaagttgaagctggaaatactctggttagaaatgagtcgcataagaattgcgatttccgtgtaaaagatcttcaatattcaacagactacacttttaaggcctattttcacaatggagactatcctggagaaccctttattttacatcattcaacatcttataattctaaggcactgatagcatttctggcatttctgattattgtgacatcaatagccctgcttgttgttctctacaaaatctatgatctacataagaaaagatcctgcaatttagatgaacagcaggagcttgttgaaagggatgatgaaaaacaactgatgaatgtggagccaatccatgcagatattttgttggaaacttataagaggaagattgctgatgaaggaagactttttctggctgaatttcagagcatcccgcgggtgttcagcaagtttcctataaaggaagctcgaaagccctttaaccagaataaaaaccgttatgttgacattcttccttatgattataaccgtgttgaactctctgagataaacggagatgcagggtcaaactacataaatgccagctatattgatggtttcaaagaacccaggaaatacattgctgcacaaggtcccagggatgaaactgttgatgatttctggaggatgatttgggaacagaaagccacagttattgtcatggtcactcgatgtgaagaaggaaacaggaacaagtgtgcagaatactggccgtcaatggaagagggcactcgggcttttggagatgttgttgtaaagatcaaccagcacaaaagatgtccagattacatcattcagaaattgaacattgtaaataaaaaagaaaaagcaactggaagagaggtgactcacattcagttcaccagctggccagaccacggggtgcctgaggatcctcacttgctcctcaaactgagaaggagagtgaatgccttcagcaatttcttcagtggtcccattgtggtgcactgcagtgctggtgttgggcgcacaggaacctatatcggaattgatgccatgctagaaggcctggaagccgagaacaaagtggatgtttatggttatgttgtcaagctaaggcgacagagatgcctgatggttcaagtagaggcccagtacatcttgatccatcaggctttggtggaatacaatcagtttggagaaacagaagtgaatttgtctgaattacatccatatctacataacatgaagaaaagggatccacccagtgagccgtctccactagaggctgaattccagagacttccttcatataggagctggaggacacagcacattggaaatcaagaagaaaataaaagtaaaaacaggaattctaatgtcatcccatatgactataacagagtgccacttaaacatgagctggaaatgagtaaagagagtgagcatgattcagatgaatcctctgatgatgacagtgattcagaggaaccaagcaaatacatcaatgcatcttttataatgagctactggaaacctgaagtgatgattgctgctcagggaccactgaaggagaccattggtgacttttggcagatgatcttccaaagaaaagtcaaagttattgttatgctgacagaactgaaacatggagaccaggaaatctgtgctcagtactggggagaaggaaagcaaacatatggagatattgaagttgacctgaaagacacagacaaatcttcaacttatacccttcgtgtctttgaactgagacattccaagaggaaagactctcgaactgtgtaccagtaccaatatacaaactggagtgtggagcagcttcctgcagaacccaaggaattaatctctatgattcaggtcgtcaaacaaaaacttccccagaagaattcctctgaagggaacaagcatcacaagagtacacctctactcattcactgcagggatggatctcagcaaacgggaatattttgtgctttgttaaatctcttagaaagtgcggaaacagaagaggtagtggatatttttcaagtggtaaaagctctacgcaaagctaggccaggcatggtttccacattcgagcaatatcaattcctatatgacgtcattgccagcacctaccctgctcagaatggacaagtaaagaaaaacaaccatcaagaagataaaattgaatttgataatgaagtggacaaagtaaagcaggatgctaattgtgttaatccacttggtgccccagaaaagctccctgaagcaaaggaacaggctgaaggttctgaacccacgagtggcactgaggggccagaacattctgtcaatggtcctgcaagtccagctttaaatcaaggttcatag

SEQ ID NO: 53 - protein, 180 aa, Homo sapiens
EEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDA

(19) United States
(12) Patent Application Publication
(10) Pub. No.: US 2024/0294942 A1
(43) Pub. Date: Sep. 5, 2024

(54) RETARGETED RETROVIRAL VECTORS AND COMPOSITIONS OR METHODS OF USE THEREOF

(71) Applicants: The Broad Institute, Inc., Cambridge, MA (US); The General Hospital Corporation, Boston, MA (US)

(72) Inventors: Kepler MEARS, Cambridge, MA (US); Robert MANGUSO, Cambridge, MA (US); Kathleen YATES, Cambridge, MA (US)

(73) Assignees: The Broad Institute, Inc., Cambridge, MA (US); The General Hospital Corporation, Boston, MA (US)

(21) Appl. No.: 18/667,718

(22) Filed: May 17, 2024

Related U.S. Application Data
(63) Continuation of application No. PCT/US2022/080152, filed on Nov. 18, 2022.
(60) Provisional application No. 63/280,919, filed on Nov. 18, 2021.
(60) Provisional application No. 63/280,926, filed on Nov. 18, 2021.
(60) Provisional application No. 63/359,027, filed on Jul. 7, 2022.

(51) Int. Cl. C12N 15/86; A61K 48/00; C07K 14/005; C07K 16/28

(52) U.S. Cl. CPC C12N 15/86; A61K 48/0058; C07K 14/005; C07K 16/2803; C07K 16/2812; C07K 16/2815; C07K 16/2833; C07K 16/289; C07K 2317/569; C07K 2317/622; C07K 2319/33; C12N 2740/15042; C12N 2740/15045; C12N 2740/15052; C12N 2740/15062; C12N 2740/15071; C12N 2760/18422; C12N 2760/18471

(57) ABSTRACT

The invention features pseudotyped viral particles (e.g., lentiviral or gammaretroviral particles) and compositions and methods of use thereof, where the viral particles comprise a VHH domain.

### CROSS-REFERENCE TO RELATED APPLICATIONS

This application is a continuation under 35 U.S.C. §111(a) of PCT International Patent Application No. PCT/US2022/080152, filed Nov. 18, 2022, designating the United States and published in English, which claims priority to and the benefit of U.S. Provisional Applications No. 63/359,027, filed Jul. 7, 2022, 63/280,919, filed Nov. 18, 2021, and 63/280,926, filed Nov. 18, 2021, the entire contents of which are incorporated herein by reference.

### SEQUENCE LISTING

The present application contains a Sequence Listing which has been submitted electronically in XML format following conversion from the originally filed TXT format.

The content of the electronic XML Sequence Listing, (Date of creation: Jan. 11, 2023; Size: 102,400 bytes: Name: 167741-032001PCT-SL.xml), is herein incorporated by reference in its entirety.

### BACKGROUND OF THE INVENTION

Efforts for altering somatic cells in a subject have been hindered by inefficient vector delivery and an inability to target the desired cells specifically. One approach that overcomes this obstacle is the genetic manipulation of cells ex vivo, as is done for T cells in adoptive cell therapy or chimeric antigen receptor (CAR) therapy. T cells are activated using anti-CD3/anti-CD28 and/or cytokine stimulation, followed by lentiviral transduction and transfer into a new animal. However, this process of in vitro expansion changes the T cell state and affects differentiation: moreover, this approach is not easily extendable to other cell types which cannot be expanded ex vivo. This approach also suffers from inefficient engraftment of gene modified cells after transplantation in vivo.

In vivo approaches for altering cells would benefit from effective methods for retargeting vectors to be specific for particular cell types in a subject. However, such methods remain inefficient and/or poorly developed.

Thus, there is a need for improved methods for in vivo delivery of vectors to a target cell.

### SUMMARY OF THE INVENTION

As described below, the present invention features pseudotyped viral particles (e.g., lentiviral or gammaretroviral particles) and compositions and methods of use thereof, where the viral particles comprise a VHH domain.

In one aspect, the invention features a pseudotyped viral particle. The viral particle contains (a) an envelope containing a fusion protein. The fusion protein contains a viral envelope glycoprotein domain or fragment thereof fused to a single variable domain on a heavy chain (VHH) antibody (VHH) domain or antigen binding fragment thereof. The VHH domain or antigen binding fragment thereof specifically binds an antigen present on a target cell. The pseudotyped viral particle also contains (b) a heterologous polynucleotide.

In another aspect, the invention features a lentiviral particle. The lentiviral particle contains (a) an envelope containing a fusion protein. The fusion protein contains a VHH domain or antigen binding fragment thereof and a Morbillivirus hemagglutinin domain or fragment thereof. The VHH specifically binds αMHCII. The lentiviral particle also contains (b) a polynucleotide encoding a guide polynucleotide and/or a Cas9 or another component of a genome editing system.

In another aspect, the invention features a lentiviral particle. The lentiviral particle contains (a) an envelope containing a fusion protein. The fusion protein contains a VHH domain or antigen binding fragment thereof and a Morbillivirus hemagglutinin domain or fragment thereof. The VHH specifically binds αCD7, αCD8, or αCD4. The lentiviral particle also contains (b) a polynucleotide encoding a guide polynucleotide and/or a Cas9

In another aspect, the invention features a method for delivering a heterologous polynucleotide to a target cell. The method involves contacting a target cell with the viral particle of any of the above aspects, thereby delivering the heterologous polynucleotide to the target cell.

In another aspect, the invention features a method of treating a subject having a cancer. The method involves administering to the subject a composition containing the pseudotyped viral particle of any of the above aspects.

In another aspect, the invention features a method for generating a pseudotyped viral particle for delivering a heterologous polynucleotide to a target cell. The method involves (a) displaying on the cell membrane of a eukaryotic cell a viral envelope glycoprotein domain or fragment thereof fused to a VHH domain or fragment thereof. The VHH domain or fragment thereof specifically binds an antigen present on the target cell. The method further involves, (b) transfecting the eukaryotic cell with a viral transfer vector and one or more additional vectors encoding one or more viral polypeptides, thereby generating the pseudotyped viral particle for delivering a heterologous polynucleotide to the target cell.

In another aspect, the invention features a eukaryotic cell for generating a pseudotyped viral particle. The eukaryotic cell contains (a) a cell membrane containing a viral envelope glycoprotein domain or fragment thereof fused to a VHH domain or fragment thereof. The VHH domain or fragment thereof specifically binds an antigen present on a target cell. The eukaryotic cell also contains (b) a viral transfer vector: and (c) one or more additional vectors encoding one or more viral polypeptides.

In another aspect, the invention features a mammalian expression vector. The mammalian expression vector contains a polynucleotide encoding a polypeptide. The polypeptide contains a viral envelope glycoprotein domain or fragment thereof fused to a VHH domain or fragment thereof. The VHH domain or fragment thereof specifically binds an antigen present on a target cell.

In another aspect, the invention features a pharmaceutical composition containing the pseudotyped viral particle of any of the above aspects, and a pharmaceutically acceptable excipient.

In another aspect, the invention features a kit for use in the method of any of the above aspects. The kit contains the pseudotyped viral particle of any one of the above aspects, the mammalian expression vector of any of the above aspects, or the pharmaceutical composition of any of the above aspects. The pseudotyped viral particle contains a heterologous polynucleotide containing a polypeptide-encoding sequence under the control of a promoter. The kit further contains instructions for the use of the kit in the method of any of the above aspects.

In another aspect, the invention features a fusion protein suitable for pseudotyping a viral particle. The fusion protein contains a viral envelope glycoprotein domain fused to a VHH domain. The VHH domain or fragment thereof specifically binds an antigen present on a target cell. The fusion protein contains a sequence with at least 85% sequence identity to a sequence selected from one or more of:

MeV-Hwtc 18-MHCII (N11)
MGSRIVINREHLMIDRPYVLLAVLFVMFLSLIGLLAIAGIRLHRAAIYTAEIHKSLSTNLDVTN
SIEHQVKDVLTPLFKIIGDEVGLRTPQRFTDLVKFISDKIKFLNPDREYDFRDLTWCINPPERI
KLDYDQYCADVAAEELMNALVNSTLLEARATNQFLAVSKGNCSGPTTIRGQFSNMSLSLLDLYL
SRGYNVSSIVTMTSQGMYGGTYLVGKPNLSSKGSELSQLSMHRVFEVGVIRNPGLGAPVFHMTN
YFEQPVSNDFSNCMVALGELKFAALCHREDSITIPYQGSGKGVSFQLVKLGVWKSPTDMRSWVP
LSTDDPVIDRLYLSSHRGVIADNQAKWAVPTTRTDDKLRMETCFQQACKGKNQALCENPEWAPL
KDNRIPSYGVLSVNLSLTVELKIKIASGFGPLITHGSGMDLYKTNHNNVYWLTIPPMKNLALGV
INTLEWIPRFKVSPALFTVPIKEAGEDCHAPTYLPAEVDGDVKLSSNLVILPGQDLQYVLATYD
TSRVEHAVVYYVYSPSRSFSYFYPFRLPIKGVPIELQVECFTWDKKLWCRHFCVLADSESGGHI
THSGMVGMGVSCTVTREDGTGGGGSGGGGSGGGGSAAAQVQLVQSGGGLVQPGGSLGLSCAASG
NIGSRDNMGWYRQAPGKQREWVATISGYGIATYRDSVKGRFTVAKDTAKNIVSLQMNYLTTEDT
AVYYCYAYAVDSRNIFWSQGTQVTVSGGGSGGGSYPYDVPDYA;
MeV-Hwtc 18-CD45 (32)
MGSRIVINREHLMIDRPYVLLAVLFVMFLSLIGLLAIAGIRLHRAAIYTAEIHKSLSTNLDVTN
SIEHQVKDVLTPLFKIIGDEVGLRTPQRFTDLVKFISDKIKFLNPDREYDERDLTWCINPPERI
KLDYDQYCADVAAEELMNALVNSTLLEARATNQFLAVSKGNCSGPTTIRGQFSNMSLSLLDLYL
SRGYNVSSIVTMTSQGMYGGTYLVGKPNLSSKGSELSQLSMHRVFEVGVIRNPGLGAPVFHMTN
YFEQPVSNDFSNCMVALGELKFAALCHREDSITIPYQGSGKGVSFQLVKLGVWKSPTDMRSWVP
LSTDDPVIDRLYLSSHRGVIADNQAKWAVPTTRTDDKLRMETCFQQACKGKNQALCENPEWAPL
KDNRIPSYGVLSVNLSLTVELKIKIASGFGPLITHGSGMDLYKTNHNNVYWLTIPPMKNLALGV
INTLEWIPRFKVSPALFTVPIKEAGEDCHAPTYLPAEVDGDVKLSSNLVILPGQDLQYVLATYD
TSRVEHAVVYYVYSPSRSFSYFYPFRLPIKGVPIELQVECFTWDKKLWCRHFCVLADSESGGHI
THSGMVGMGVSCTVTREDGTGGGGSGGGGSGGGGSAAAQVQLVQSGGGLVQPGGSLRLSCAASG
RAFNSAAMGWYRQAPGSQRELVASISAGTASYADAVKGRFTISRDYAKNIIYLQMNSLKPDDTA
VYFCNYRTTYTSGYSEDYWGQGTQVTVSGGGSGGGSYPYDVPDYA;
MeV-Hwtc 18-CD7 (humanized VHH10)
MGSRIVINREHLMIDRPYVLLAVLFVMFLSLIGLLAIAGIRLHRAAIYTAEIHKSLSTNLDVTN
SIEHQVKDVLTPLFKIIGDEVGLRTPQRFTDLVKFISDKIKFLNPDREYDERDLTWCINPPERI
KLDYDQYCADVAAEELMNALVNSTLLEARATNQFLAVSKGNCSGPTTIRGQFSNMSLSLLDLYL
SRGYNVSSIVTMTSQGMYGGTYLVGKPNLSSKGSELSQLSMHRVFEVGVIRNPGLGAPVFHMTN
YFEQPVSNDFSNCMVALGELKFAALCHREDSITIPYQGSGKGVSFQLVKLGVWKSPTDMRSWVP
LSTDDPVIDRLYLSSHRGVIADNQAKWAVPTTRTDDKLRMETCFQQACKGKNQALCENPEWAPL
KDNRIPSYGVLSVNLSLTVELKIKIASGFGPLITHGSGMDLYKTNHNNVYWLTIPPMKNLALGV
INTLEWIPRFKVSPALFTVPIKEAGEDCHAPTYLPAEVDGDVKLSSNLVILPGQDLQYVLATYD
TSRVEHAVVYYVYSPSRSFSYFYPFRLPIKGVPIELQVECFTWDKKLWCRHFCVLADSESGGHI
THSGMVGMGVSCTVTREDGTGGGGSGGGGSGGGGSAAADVQLQESGGGSVQAGGSLRLSCAASG
YTHSSYCMAWFRQAPGREREGVASIDSDGTTSYADSVKGRFTISQDNAKNTLYLQMNSLKPEDT
AMYYCAARFGPMGCVDLSTLSFGHWGQGTQVTVSITGGGSGGGSYPYDVPDYA;
MeV-Hwtc18-CD4 (03F11)
MGSRIVINREHLMIDRPYVLLAVLFVMFLSLIGLLAIAGIRLHRAAIYTAEIHKSLSTNLDVTN
SIEHQVKDVLTPLFKIIGDEVGLRTPQRFTDLVKFISDKIKFLNPDREYDFRDLTWCINPPERI
KLDYDQYCADVAAEELMNALVNSTLLEARATNQFLAVSKGNCSGPTTIRGQFSNMSLSLLDLYL
SRGYNVSSIVTMTSQGMYGGTYLVGKPNLSSKGSELSQLSMHRVFEVGVIRNPGLGAPVFHMTN
YFEQPVSNDFSNCMVALGELKFAALCHREDSITIPYQGSGKGVSFQLVKLGVWKSPTDMRSWVP
LSTDDPVIDRLYLSSHRGVIADNQAKWAVPTTRTDDKLRMETCFQQACKGKNQALCENPEWAPL
KDNRIPSYGVLSVNLSLTVELKIKIASGFGPLITHGSGMDLYKTNHNNVYWLTIPPMKNLALGV
INTLEWIPRFKVSPALFTVPIKEAGEDCHAPTYLPAEVDGDVKLSSNLVILPGQDLQYVLATYD
TSRVEHAVVYYVYSPSRSFSYFYPFRLPIKGVPIELQVECFTWDKKLWCRHFCVLADSESGGHI
THSGMVGMGVSCTVTREDGTGGGGSGGGGSGGGGSAAAEVOLVESGGGSVQPGGSLTLSCGTSG
RTFNVMGWFRQAPGKEREFVAAVRWSSTGIYYTQYADSVKSRFTISRDNAKNTVYLEMNSLKPE
DTAVYYCAADTYNSNPARWDGYDFRGQGTQVTVSSGGGSGGGSYPYDVPDYA;
and
MeV-Hwtc18-CD8a (R3HCD27)
MGSRIVINREHLMIDRPYVLLAVLFVMFLSLIGLLAIAGIRLHRAAIYTAEIHKSLSTNLDVTN
SIEHQVKDVLTPLFKIIGDEVGLRTPQRFTDLVKFISDKIKFLNPDREYDERDLTWCINPPERI
KLDYDQYCADVAAEELMNALVNSTLLEARATNQFLAVSKGNCSGPTTIRGQFSNMSLSLLDLYL
SRGYNVSSIVTMTSQGMYGGTYLVGKPNLSSKGSELSQLSMHRVFEVGVIRNPGLGAPVFHMTN
YFEQPVSNDFSNCMVALGELKFAALCHREDSITIPYQGSGKGVSFQLVKLGVWKSPTDMRSWVP
LSTDDPVIDRLYLSSHRGVIADNQAKWAVPTTRTDDKLRMETCFQQACKGKNQALCENPEWAPL
KDNRIPSYGVLSVNLSLTVELKIKIASGFGPLITHGSGMDLYKTNHNNVYWLTIPPMKNLALGV
INTLEWIPRFKVSPALFTVPIKEAGEDCHAPTYLPAEVDGDVKLSSNLVILPGQDLQYVLATYD
TSRVEHAVVYYVYSPSRSFSYFYPFRLPIKGVPIELQVECFTWDKKLWCRHFCVLADSESGGHI
THSGMVGMGVSCTVTREDGTGGGGSGGGGSGGGGSAAAQVQLQESGGGSVQPGGSLRLSCAASG
FTFDDYAMSWVRQVPGKGLEWVSTINWNGGSAEYAEPVKGRFTISRDNAKNTVYLQMNSLKLED
TAVYYCAKDADLVWYNLSTGQGTQVTVSSAAAYPYDVPDYGSGGGSGGGSYPYDVPDYA.

In any of the above aspects, or embodiments thereof, the viral envelope glycoprotein domain or fragment thereof contains a viral hemagglutinin domain or fragment thereof. In any of the above aspects, or embodiments thereof, viral envelope glycoprotein domain or fragment thereof is derived from a hemagglutinin polypeptide of the measles virus.

In any of the above aspects, or embodiments thereof, the viral envelope glycoprotein domain or fragment thereof contains a sequence with at least 85% amino acid sequence identity to the following sequence:

MeV-Hwtc18 polypeptide
MGSRIVINREHLMIDRPYVLLAVLFVMFLSLIGLLAIAGIRLHRAAIYT
AEIHKSLSTNLDVTNSIEHQVKDVLTPLFKIIGDEVGLRTPQRFTDLVK
FISDKIKFLNPDREYDFRDLTWCINPPERIKLDYDQYCADVAAEELMNA
LVNSTLLEARATNQFLAVSKGNCSGPTTIRGQFSNMSLSLLDLYLSRGY
NVSSIVTMTSQGMYGGTYLVGKPNLSSKGSELSQLSMHRVFEVGVIRNP
GLGAPVFHMTNYFEQPVSNDFSNCMVALGELKFAALCHREDSITIPYQG
SGKGVSFQLVKLGVWKSPTDMRSWVPLSTDDPVIDRLYLSSHRGVIADN
QAKWAVPTTRTDDKLRMETCFQQACKGKNQALCENPEWAPLKDNRIPSY
GVLSVNLSLTVELKIKIASGFGPLITHGSGMDLYKTNHNNVYWLTIPPM
KNLALGVINTLEWIPRFKVSPALFTVPIKEAGEDCHAPTYLPAEVDGDV
KLSSNLVILPGQDLQYVLATYDTSRVEHAVVYYVYSPSRSFSYFYPFRL
PIKGVPIELQVECFTWDKKLWCRHFCVLADSESGGHITHSGMVGMGVSC
TVTREDGT.

In any of the above aspects, or embodiments thereof, the VHH domain or antigen binding fragment thereof contains a VHH amino acid sequence with at least 85% sequence identity to a sequence selected from one or more of:

Anti-MHCIIVHH (N11)
QVQLVQSGGGLVQPGGSLGLSCAASGNIGSRDNMGWYRQAPGKQREWVA
TISGYGIATYRDSVKGRFTVAKDTAKNIVSLQMNYLTTEDTAVYYCYAY
AVDSRNIFWSQGTQVTVS;
Anti-CD45 (32) VHH
QVQLVQSGGGLVQPGGSLRLSCAASGRAFNSAAMGWYRQAPGSQRELVA
SISAGTASYADAVKGRFTISRDYAKNIIYLQMNSLKPDDTAVYFCNYRT
TYTSGYSEDYWGQGTQVTVS;
Anti-CD7 (VHH10) VHH
DVQLQESGGGSVQAGGSLRLSCAASGYTHSSYCMAWFRQAPGREREGVA
SIDSDGTTSYADSVKGRFTISQDNAKNTLYLQMNSLKPEDTAMYYCAAR
FGPMGCVDLSTLSFGHWGQGTQVTVSIT;
Anti-CD4 (03F11) VHH
EVOLVESGGGSVQPGGSLTLSCGTSGRTFNVMGWFRQAPGKEREFVAAV
RWSSTGIYYTQYADSVKSRFTISRDNAKNTVYLEMNSLKPEDTAVYYCA
ADTYNSNPARWDGYDFRGQGTQVTVSS;
and
Anti-CD8 (R3HCD27) VHH
QVQLQESGGGSVQPGGSLRLSCAASGFTFDDYAMSWVRQVPGKGLEWVS
TINWNGGSAEYAEPVKGRFTISRDNAKNTVYLQMNSLKLEDTAVYYCAK
DADLVWYNLSTGQGTQVTVSS.

In any of the above aspects, or embodiments thereof, the viral envelope glycoprotein domain or fragment thereof and the VHH domain or fragment thereof are separated by a linker. In embodiments, the linker containing a G4S and/or a G3S amino acid sequence. In embodiments, the linker contains the sequence GGGGSGGGGSGGGGS.

In any of the above aspects, or embodiments thereof, the viral envelope glycoprotein domain or fragment thereof fused to the VHH domain or antigen binding fragment thereof fragment thereof contains a sequence with at least 85% sequence identity to a sequence selected one or more of:

MeV-Hwtc 18-MHCII(N11)
MGSRIVINREHLMIDRPYVLLAVLFVMFLSLIGLLAIAGIRLHRAAIYTAEIHKSLSTNLDVTN
SIEHQVKDVLTPLFKIIGDEVGLRTPQRFTDLVKFISDKIKFLNPDREYDFRDLTWCINPPERI
KLDYDQYCADVAAEELMNALVNSTLLEARATNQFLAVSKGNCSGPTTIRGQFSNMSLSLLDLYL
SRGYNVSSIVTMTSQGMYGGTYLVGKPNLSSKGSELSQLSMHRVFEVGVIRNPGLGAPVFHMTN
YFEQPVSNDFSNCMVALGELKFAALCHREDSITIPYQGSGKGVSFQLVKLGVWKSPTDMRSWVP
LSTDDPVIDRLYLSSHRGVIADNQAKWAVPTTRTDDKLRMETCFQQACKGKNQALCENPEWAPL
KDNRIPSYGVLSVNLSLTVELKIKIASGFGPLITHGSGMDLYKTNHNNVYWLTIPPMKNLALGV
INTLEWIPRFKVSPALFTVPIKEAGEDCHAPTYLPAEVDGDVKLSSNLVILPGQDLQYVLATYD
TSRVEHAVVYYVYSPSRSFSYFYPFRLPIKGVPIELQVECFTWDKKLWCRHFCVLADSESGGHI
THSGMVGMGVSCTVTREDGTGGGGSGGGGSGGGGSAAAQVQLVQSGGGLVQPGGSLGLSCAASG
NIGSRDNMGWYRQAPGKQREWVATISGYGIATYRDSVKGRFTVAKDTAKNIVSLQMNYLTTEDT
AVYYCYAYAVDSRNIFWSQGTQVTVSGGGSGGGSYPYDVPDYA;
MeV-Hwtc 18-CD45 (32)
MGSRIVINREHLMIDRPYVLLAVLFVMFLSLIGLLAIAGIRLHRAAIYTAEIHKSLSTNLDVTN
SIEHQVKDVLTPLFKIIGDEVGLRTPQRFTDLVKFISDKIKFLNPDREYDERDLTWCINPPERI
KLDYDQYCADVAAEELMNALVNSTLLEARATNQFLAVSKGNCSGPTTIRGQFSNMSLSLLDLYL
SRGYNVSSIVTMTSQGMYGGTYLVGKPNLSSKGSELSQLSMHRVFEVGVIRNPGLGAPVFHMTN
YFEQPVSNDFSNCMVALGELKFAALCHREDSITIPYQGSGKGVSFQLVKLGVWKSPTDMRSWVP
LSTDDPVIDRLYLSSHRGVIADNQAKWAVPTTRTDDKLRMETCFQQACKGKNQALCENPEWAPL
KDNRIPSYGVLSVNLSLTVELKIKIASGFGPLITHGSGMDLYKTNHNNVYWLTIPPMKNLALGV
INTLEWIPRFKVSPALFTVPIKEAGEDCHAPTYLPAEVDGDVKLSSNLVILPGQDLQYVLATYD
TSRVEHAVVYYVYSPSRSFSYFYPFRLPIKGVPIELQVECFTWDKKLWCRHFCVLADSESGGHI
THSGMVGMGVSCTVTREDGTGGGGSGGGGSGGGGSAAAQVQLVQSGGGLVQPGGSLRLSCAASG
RAFNSAAMGWYRQAPGSQRELVASISAGTASYADAVKGRFTISRDYAKNIIYLQMNSLKPDDTA
VYFCNYRTTYTSGYSEDYWGQGTQVTVSGGGSGGGSYPYDVPDYA;
MeV-Hwtc 18-CD7 (humanized VHH10)
MGSRIVINREHLMIDRPYVLLAVLFVMFLSLIGLLAIAGIRLHRAAIYTAEIHKSLSTNLDVTN
SIEHQVKDVLTPLFKIIGDEVGLRTPQRFTDLVKFISDKIKFLNPDREYDERDLTWCINPPERI
KLDYDQYCADVAAEELMNALVNSTLLEARATNQFLAVSKGNCSGPTTIRGOFSNMSLSLLDLYL
SRGYNVSSIVTMTSQGMYGGTYLVGKPNLSSKGSELSQLSMHRVFEVGVIRNPGLGAPVFHMTN
YFEQPVSNDFSNCMVALGELKFAALCHREDSITIPYQGSGKGVSFQLVKLGVWKSPTDMRSWVP
LSTDDPVIDRLYLSSHRGVIADNQAKWAVPTTRTDDKLRMETCFQQACKGKNQALCENPEWAPL
KDNRIPSYGVLSVNLSLTVELKIKIASGFGPLITHGSGMDLYKTNHNNVYWLTIPPMKNLALGV
INTLEWIPRFKVSPALFTVPIKEAGEDCHAPTYLPAEVDGDVKLSSNLVILPGQDLQYVLATYD
TSRVEHAVVYYVYSPSRSFSYFYPFRLPIKGVPIELQVECFTWDKKLWCRHFCVLADSESGGHI
THSGMVGMGVSCTVTREDGTGGGGSGGGGSGGGGSAAADVQLQESGGGSVQAGGSLRLSCAASG
YTHSSYCMAWFRQAPGREREGVASIDSDGTTSYADSVKGRFTISQDNAKNTLYLQMNSLKPEDT
AMYYCAARFGPMGCVDLSTLSFGHWGQGTQVTVSITGGGSGGGSYPYDVPDYA;
MeV-Hwtc18-CD4 (03F11)
MGSRIVINREHLMIDRPYVLLAVLFVMFLSLIGLLAIAGIRLHRAAIYTAEIHKSLSTNLDVTN
SIEHQVKDVLTPLFKIIGDEVGLRTPQRFTDLVKFISDKIKFLNPDREYDFRDLTWCINPPERI
KLDYDQYCADVAAEELMNALVNSTLLEARATNQFLAVSKGNCSGPTTIRGQFSNMSLSLLDLYL
SRGYNVSSIVTMTSQGMYGGTYLVGKPNLSSKGSELSQLSMHRVFEVGVIRNPGLGAPVFHMTN
YFEQPVSNDFSNCMVALGELKFAALCHREDSITIPYQGSGKGVSFQLVKLGVWKSPTDMRSWVP
LSTDDPVIDRLYLSSHRGVIADNQAKWAVPTTRTDDKLRMETCFQQACKGKNQALCENPEWAPL
KDNRIPSYGVLSVNLSLTVELKIKIASGFGPLITHGSGMDLYKTNHNNVYWLTIPPMKNLALGV
INTLEWIPRFKVSPALFTVPIKEAGEDCHAPTYLPAEVDGDVKLSSNLVILPGQDLQYVLATYD
TSRVEHAVVYYVYSPSRSFSYFYPFRLPIKGVPIELQVECFTWDKKLWCRHFCVLADSESGGHI
THSGMVGMGVSCTVTREDGTGGGGSGGGGSGGGGSAAAEVOLVESGGGSVQPGGSLTLSCGTSG
RTFNVMGWFRQAPGKEREFVAAVRWSSTGIYYTQYADSVKSRFTISRDNAKNTVYLEMNSLKPE
DTAVYYCAADTYNSNPARWDGYDFRGQGTQVTVSSGGGSGGGSYPYDVPDYA;
and
MeV-Hwtc18-CD8a (R3HCD27)
MGSRIVINREHLMIDRPYVLLAVLFVMFLSLIGLLAIAGIRLHRAAIYTAEIHKSLSTNLDVTN
SIEHQVKDVLTPLFKIIGDEVGLRTPQRFTDLVKFISDKIKFLNPDREYDFRDLTWCINPPERI
KLDYDQYCADVAAEELMNALVNSTLLEARATNQFLAVSKGNCSGPTTIRGQFSNMSLSLLDLYL
SRGYNVSSIVTMTSQGMYGGTYLVGKPNLSSKGSELSQLSMHRVFEVGVIRNPGLGAPVFHMTN
YFEQPVSNDFSNCMVALGELKFAALCHREDSITIPYQGSGKGVSFQLVKLGVWKSPTDMRSWVP
LSTDDPVIDRLYLSSHRGVIADNQAKWAVPTTRTDDKLRMETCFQQACKGKNQALCENPEWAPL
KDNRIPSYGVLSVNLSLTVELKIKIASGFGPLITHGSGMDLYKTNHNNVYWLTIPPMKNLALGV
INTLEWIPRFKVSPALFTVPIKEAGEDCHAPTYLPAEVDGDVKLSSNLVILPGQDLQYVLATYD
TSRVEHAVVYYVYSPSRSFSYFYPFRLPIKGVPIELQVECFTWDKKLWCRHFCVLADSESGGHI
THSGMVGMGVSCTVTREDGTGGGGSGGGGSGGGGSAAAQVQLQESGGGSVQPGGSLRLSCAASG
FTFDDYAMSWVRQVPGKGLEWVSTINWNGGSAEYAEPVKGRFTISRDNAKNTVYLQMNSLKLED
TAVYYCAKDADLVWYNLSTGQGTQVTVSSAAAYPYDVPDYGSGGGSGGGSYPYDVPDYA.

In any of the above aspects, or embodiments thereof, the pseudotyped viral particle is a pseudotyped retroviral viral particle. In any of the above aspects, or embodiments thereof, the pseudotyped retroviral viral particle is a pseudotyped lentiviral viral particle. In any of the above aspects, or embodiments thereof, the pseudotyped retroviral viral particle is a pseudotyped Gammaretrovirus viral particle. In any of the above aspects, or embodiments thereof, the Gammaretrovirus viral particle is a pseudotyped leukemia virus particle. In any of the above aspects, or embodiments thereof, the pseudotyped viral particle is a pseudotyped retroviral viral particle and/or the viral transfer vector is a retroviral transfer vector. In any of the above aspects, or embodiments thereof, the pseudotyped retroviral viral particle is a pseudotyped lentiviral viral particle and/or the viral transfer vector is lentiviral transfer vector. In any of the above aspects, or embodiments thereof, the pseudotyped retroviral viral particle is a pseudotyped

Gammaretrovirus viral particle and/or the viral transfer vector is a Gammaretrovirus transfer vector. In any of the above aspects, or embodiments thereof, the Gammaretrovirus is a pseudotyped murine leukemia virus particle and/or the Gammaretrovirus transfer vector is a murine leukemia virus transfer vector.

In any of the above aspects, or embodiments thereof, the pseudotyped viral particle is self-replicating. In any of the above aspects, or embodiments thereof, the pseudotyped viral particle is not self-replicating.

In any of the above aspects, or embodiments thereof, the target cell is an immune cell. In embodiments, the immune cell is a professional antigen-presenting cell. In any of the above aspects, or embodiments thereof, the target cell is a splenocyte or a thymocyte. In any of the above aspects, or embodiments thereof, the target cell is selected from one or more of a B cell, a dendritic cell, an eosinophil, a granulocyte, an iNKT cell, a macrophage, a monocyte, a natural killer cell, a neutrophil, a cancer or tumor cell, a regulatory T cell, and a T cell. In any of the above aspects, or embodiments thereof, the target cell is CD4 and/or CD8.

In any of the above aspects, or embodiments thereof, the antigen is selected from one or more of BCR/Ig, CD3, CD4, CD7, CD8, CD11, CD19, CD20, CD30, CD34, CD38, CD45, CD133, CD103, CD105, CD110, CD117, CTLA-4, CXCR4, DC-SIGN, EGFR, Emr1, EpCAM, GluA4, Her2/neu, IL3R, IL7R, Mac, MHCII, Mucin 4, NK1.1, P-glycoprotein, TIM3, Thy1, and Thy1.2.

In any of the above aspects, or embodiments thereof, the VHH or fragment thereof is derived from a VHH selected from one or more of 03F11, 6QRM, aCD8 VHH, aCD11b VHH, Anti-CD3 VHH, DCI, DC1.8, DC2.1, DC8, DC14, DC15, hH6, 281F12, mH2, MU375, MU551, MU1053, R2HCD26, R3HCD27, R3HCD129, VHH4, VHH6, VHH6 Humanized 1, VHH6 Humanized 2, VHH7, VHH10, VHH10 Humanized 1, VHH10 Humanized 2, VHH32, VHH49, VHH51, VHH81, VHHDC13, VHHG7, VHHN11, and VHHV36.

In any of the above aspects, or embodiments thereof, the cell membrane contains a viral fusion polypeptide.

In any of the above aspects, or embodiments thereof, the cell membrane contains a phagocytosis inhibitor.

In any of the above aspects, or embodiments thereof, the envelope contains a phagocytosis inhibitor. In embodiments, the phagocytosis inhibitor is CD47.

In any of the above aspects, or embodiments thereof, the envelope contains a complement regulatory polypeptide. In any of the above aspects, or embodiments thereof, the cell membrane contains a complement regulatory polypeptide. In embodiments, the complement regulatory polypeptide is selected from one or more of CD46, CD55, and CD59.

In any of the above aspects, or embodiments thereof, the heterologous polynucleotide encodes a heterologous polypeptide to be delivered to the target cell. In any of the above aspects, or embodiments thereof, the envelope further contains a heterologous polypeptide to be delivered to the target cell. In any of the above aspects, or embodiments thereof, the viral transfer vector contains a polynucleotide sequence encoding, and/or the cell membrane further contains, a heterologous polypeptide to be delivered to the target cell. In any of the above aspects, or embodiments thereof, the cell membrane contains the heterologous polypeptide.

In any of the above aspects, or embodiments thereof, the heterologous polypeptide is a chemokine or a cytokine. In any of the above aspects, or embodiments thereof, the heterologous polypeptide is selected from one or more of a CD3, Ccl14, CD28, CD40L, Cxcl10, IL-2, IL-12, and a gene-editing polypeptide. In any of the above aspects, or embodiments thereof, the heterologous polypeptide is a cytomegalovirus antigen, a flu virus antigen, or a coronavirus antigen. In embodiments, the coronavirus antigen is a SARS-COV2 antigen.

In any of the above aspects, or embodiments thereof, the method further involves integrating the heterologous polynucleotide into the genome of the target cell. In any of the above aspects, or embodiments thereof, the heterologous polypeptide is a gene-editing polypeptide. In any of the above aspects, or embodiments thereof, the heterologous polypeptide is a cytomegalovirus antigen, a flu virus antigen, or a coronavirus antigen. In any of the above aspects, or embodiments thereof, the heterologous polynucleotide encodes a chimeric antigen receptor.

In any of the above aspects, or embodiments thereof, the viral transfer vector contains a polynucleotide encoding a chimeric antigen receptor.

In any of the above aspects, or embodiments thereof, the pseudotyped viral particle is administered systemically or locally. In any of the above aspects, or embodiments thereof, target cell is a mammalian cell. In any of the above aspects, or embodiments thereof, where the target cell is a human cell. In any of the above aspects, or embodiments thereof, the target cell is a cancer cell. In any of the above aspects, or embodiments thereof, the cell is a splenocyte, peripheral blood mononuclear cell, or immune cell. In embodiments, the immune cell is a T cell or NK cell. In embodiments, the cancer cell is a B cell lymphoma cell.

In any of the above aspects, or embodiments thereof, the target cell is in vitro. In any of the above aspects, or embodiments thereof, the target cell is in vivo.

In any of the above aspects, or embodiments thereof, the subject is a mammal. In any of the above aspects, or embodiments thereof, the subject is a human. In any of the above aspects, or embodiments thereof, the cancer is a leukemia or a lymphoma.

In any of the above aspects, or embodiments thereof, expression of the polypeptide is under the control of a promoter. In any of the above aspects, or embodiments thereof, the eukaryotic cell is selected from one or more of a 293T cell, a Jurkat T cell, a primary human T cell, a SupTI cell, a CHO cell, a HepG2 cell, an MCF-7 cell, and an MEF cell.

The invention provides pseudotyped viral particles (e.g., lentiviral or gammaretroviral particles) and compositions and methods of use thereof, where the viral particles comprise a VHH domain. Compositions and articles defined by the invention were isolated or otherwise manufactured in connection with the examples provided below. Other features and advantages of the invention will be apparent from the detailed description, and from the claims.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994): The Cambridge Dictionary of Science and Technology (Walker ed., 1988): The Glossary of Genetics, 5th Ed., R. Rieger et al. (eds.), Springer Verlag (1991): and Hale & Marham, The Harper Collins Dictionary of Biology (1991). As used herein, the following terms have the meanings ascribed to them below, unless specified otherwise.

By “viral envelope glycoprotein domain” or “glycoprotein domain” is meant a domain that binds a receptor site on the surface of a target cell and/or mediates insertion into a target cell. In embodiments, the viral envelope glycoprotein domain or fragment thereof is fused to a VHH domain or fragment thereof. Exemplary glycoprotein domains include the MeV-Fc30 polypeptide and the MeV-Hwtc18 polypeptide.

By “VHH domain” is meant an antigen binding domain of a heavy chain only antibody. Exemplary VHH domains and their respective targets are provided at Table 2.

By “anti-major histocompatibility complex II (MHCII) VHH (N11) polypeptide” is meant a polypeptide or fragment thereof with at least about 85% amino acid sequence identity to the sequence provided below, and that is capable of binding a MHCII polypeptide.

QVQLVQSGGGLVQPGGSLGLSCAASGNIGSRDNMGWYRQAPGKQREWVA
TISGYGIATYRDSVKGRFTVAKDTAKNIVSLQMNYLTTEDTAVYYCYAY
AVDSRNIFWSQGTQVTVS.

By “anti-major histocompatibility II (MHCII) VHH (N11) polynucleotide” is meant a polynucleotide encoding an anti-major histocompatibility complex II (MHCII) VHH (N11) polypeptide. An exemplary anti-MHCII VHH polynucleotide is provided below.

CAGGTTCAACTGGTGCAGAGCGGAGGCGGCCTGGTCCAGCCCGGCGGAA
GCCTGGGCCTGAGCTGTGCCGCTTCTGGCAACATCGGCAGCAGAGATAA
TATGGGCTGGTATAGACAGGCCCCTGGAAAACAGAGAGAGTGGGTGGCT
ACAATCAGCGGCTACGGCATCGCCACATACCGGGACAGCGTGAAGGGCA
GATTCACCGTGGCCAAGGACACCGCCAAGAACATCGTGTCCCTGCAGAT
GAACTACCTGACCACCGAAGATACAGCCGTGTACTACTGCTACGCCTAC
GCCGTGGACAGCCGGAACATCTTCTGGTCCCAGGGCACCCAGGTGACCG
TGTCT.

By “anti-CD45 (32) VHH polypeptide” is meant a polypeptide or fragment thereof with at least about 85% amino acid sequence identity to the sequence provided below, and that is capable of binding a CD45 polypeptide.

QVQLVQSGGGLVQPGGSLRLSCAASGRAFNSAAMGWYRQAPGSQRELVA
SISAGTASYADAVKGRFTISRDYAKNIIYLQMNSLKPDDTAVYFCNYRT
TYTSGYSEDYWGQGTQVTVS.

By “anti-CD45 (32) VHH polynucleotide” is meant a polynucleotide encoding an anti-CD45 (32) VHH polypeptide. An exemplary anti-CD45 VHH polynucleotide is provided below.

CAGGTGCAGCTGGTGCAGAGCGGCGGAGGACTGGTGCAACCTGGAGGCA
GCCTGAGACTGAGCTGCGCCGCTAGCGGCAGAGCCTTTAACAGCGCCGC
CATGGGCTGGTACAGACAGGCCCCTGGCTCTCAGAGAGAGCTGGTCGCC
TCTATCTCCGCCGGCACCGCCTCTTATGCTGATGCCGTGAAAGGCAGAT
TCACCATCAGCCGGGACTACGCCAAGAACATCATCTACCTGCAGATGAA
CAGCCTGAAGCCCGACGACACAGCTGTGTACTTCTGTAATTACCGGACA
ACCTACACCAGCGGCTACAGCGAAGATTACTGGGGCCAGGGCACACAGG
TGACCGTGTCC.

By “anti-CD7 (VHH10) VHH polypeptide” is meant a polypeptide or fragment thereof with at least about 85% amino acid sequence identity to the sequence provided below, and that is capable of binding a CD7 polypeptide.

DVQLQESGGGSVQAGGSLRLSCAASGYTHSSYCMAWFRQAPGREREGVA
SIDSDGTTSYADSVKGRFTISQDNAKNTLYLQMNSLKPEDTAMYYCAAR
FGPMGCVDLSTLSFGHWGQGTQVTVSIT.

By “anti-CD7 (VHH10) VHH polynucleotide” is meant a polynucleotide encoding an anti-CD7 (VHH10) VHH polypeptide. An exemplary anti-CD7 VHH polynucleotide is provided below.

GATGTGCAGCTGCAGGAGAGCGGCGGCGGATCTGTGCAAGCTGGCGGAA
GCCTGAGACTGTCTTGCGCCGCTTCTGGCTACACCCACAGCAGCTATTG
CATGGCCTGGTTCCGGCAGGCCCCTGGCAGAGAACGGGAAGGCGTGGCC
AGCATCGACAGCGACGGCACCACCAGCTACGCCGATAGCGTCAAGGGCA
GATTCACCATCAGCCAGGACAATGCCAAAAACACCCTGTACCTGCAGAT
GAACAGCCTGAAGCCCGAGGACACAGCCATGTACTACTGTGCCGCCAGA
TTTGGCCCTATGGGCTGCGTGGACCTGTCCACCCTCTCCTTCGGCCACT
GGGGACAGGGCACACAGGTGACAGTGTCCATCACC.

By “anti-CD4 (03F11) VHH polypeptide” is meant a polypeptide or fragment thereof with at least about 85% amino acid sequence identity to the sequence provided below, and that is capable of binding a CD4 polypeptide.

EVOLVESGGGSVQPGGSLTLSCGTSGRTFNVMGWFRQAPGKEREFVAAV
RWSSTGIYYTQYADSVKSRFTISRDNAKNTVYLEMNSLKPEDTAVYYCA
ADTYNSNPARWDGYDFRGQGTQVTVSS.

By “anti-CD4 (03F11) VHH polynucleotide” is meant a polynucleotide encoding an anti-CD4 (03F11) VHH polypeptide. An exemplary anti-CD4 VHH polynucleotide is provided below.

GAGGTGCAGCTGGTCGAGAGCGGAGGAGGCAGCGTGCAGCCCGGCGGAT
CTCTGACCCTGAGCTGTGGCACAAGCGGCAGAACCTTTAACGTGATGGG
CTGGTTCAGACAGGCCCCTGGCAAGGAAAGAGAGTTCGTGGCCGCTGTG
CGGTGGTCCTCTACAGGCATCTACTATACACAGTACGCCGACAGCGTTA
AAAGCAGATTCACCATCAGCCGGGACAACGCCAAGAACACCGTGTACCT
GGAAATGAACAGCCTGAAGCCAGAGGATACAGCCGTGTACTACTGCGCC
GCCGATACCTACAACTCTAATCCTGCTAGATGGGACGGCTACGACTTCC
GGGGCCAGGGCACCCAAGTGACCGTGTCCAGC.

By “anti-CD8 (R3HCD27) VHH polypeptide” is meant a polypeptide or fragment thereof with at least about 85% amino acid sequence identity to the sequence provided below, and that is capable of binding a CD8 polypeptide.

QVQLQESGGGSVQPGGSLRLSCAASGFTFDDYAMSWVRQVPGKGLEWVS
TINWNGGSAEYAEPVKGRFTISRDNAKNTVYLQMNSLKLEDTAVYYCAK
DADLVWYNLSTGQGTQVTVSS.

By “anti-CD8 (R3HCD27) VHH polynucleotide” is meant a polynucleotide encoding an anti-CD8 (R3HCD27) VHH polypeptide. An exemplary anti-CD8 VHH polynucleotide is provided below.

CAAGTGCAGCTGCAGGAGAGCGGCGGAGGCAGCGTGCAGCCTGGCGGAA
GCCTGAGACTGTCCTGCGCCGCTTCTGGCTTTACCTTCGACGACTACGC
CATGAGCTGGGTGCGGCAGGTTCCAGGCAAGGGCCTGGAATGGGTCAGC
ACAATCAACTGGAACGGCGGATCTGCTGAGTACGCCGAGCCTGTGAAGG
GCAGATTCACAATCAGCAGAGATAATGCCAAAAACACCGTGTATCTGCA
GATGAACAGCCTCAAGCTGGAAGATACAGCCGTGTACTACTGTGCCAAG
GACGCCGACCTGGTGTGGTACAACCTGAGCACCGGCCAGGGCACCCAGG
TGACCGTGTCCAGCGCCGCTGCCTACCCCTACGACGTGCCTGATTACGG
CTCT.

By “MeV-Fc30 polypeptide” is meant a polypeptide or fragment thereof with at least about 85% amino acid sequence identity to the sequence provided below, and that has activity associated with fusion of a Lentivirus envelope containing an H protein domain with a membrane. In embodiments, the Mev-Fc30 polypeptide functions in combination with an MeV-Hwtc18, MeV-Hwtc18-hCD105, MeV-Hwtc18-N11, MeV-Hwtc18-32, MeV-Hwtc18-Thy1.1 (154.7.7.10), MeV-Hwtc18-CD7 (humanized VHH10), MeV-Hwtc18-CD4 (03F11), or MeV-Hwtc18-CD8a (R3HCD27) polypeptide to fuse a Lentivirus lipid envelope with a membrane (e.g., a cell membrane). An exemplary MeV-Fc30 polypeptide sequence is provided below.

MGLKVNVSAIFMAVLLTLQTPTGQIHWGNLSKIGVVGIGSASYKVMTRS
SHQSLVIKLMPNITLLNNCTRVEIAEYRRLLRTVLEPIRDALNAMTQNI
RPVQSVASSRRHKRFAGVVLAGAALGVATAAQITAGIALHQSMLNSQAI
DNLRASLETTNQAIEAIRQAGQEMILAVQGVODYINNELIPSMNQLSCD
LIGQKLGLKLLRYYTEILSLFGPSLRDPISAEISIQALSYALGGDINKV
LEKLGYSGGDLLGILESRGIKARITHVDTESYFIVLSIAYPTLSEIKGV
IVHRLEGVSYNIGSQEWYTTVPKYVATQGYLISNFDESSCTFMPEGTVC
SQNALYPMSPLLQECLRGSTKSCARTLVSGSFGNRFILSQGNLIANCAS
ILCKCYTTGTIINQDPDKILTYIAADHCPVVEVNGVTIQVGSRRYPDAV
YLHRIDLGPPISLERLDVGTNLGNAIAKLEDAKELLESSDQILRSMKGL
SSTSIVYILIAVCLGGLIGIPALICCCRGR.

In the above polypeptide sequence, the signal peptide sequence is underlined, the transmembrane domain is in italics, the intracellular domain (which includes a 30 amino acid truncation) is in bold, and the extracellular domain is in plain text.

By “MeV-Fc30 polynucleotide” is meant a polynucleotide encoding an MeV-Fc30 polypeptide. An exemplary MeV-Fc30 polynucleotide sequence is provided below.

ATGGGCCTGAAGGTGAACGTGAGCGCCATCTTCATGGCCGTGCTGCTGA
CCCTGCAGACCCCAACAGGCCAGATCCACTGGGGCAATCTGTCTAAGAT
CGGAGTGGTGGGAATCGGATCCGCCTCTTATAAAGTGATGACAAGGAGC
TCCCACCAGAGCCTGGTCATCAAGCTGATGCCTAACATCACCCTGCTGA
ACAATTGTACACGCGTGGAGATCGCCGAGTACCGGAGACTGCTGAGGAC
CGTGCTGGAGCCAATCAGGGACGCCCTGAACGCCATGACACAGAATATC
AGGCCAGTGCAGAGCGTGGCATCTAGCAGGCGCCACAAGAGATTTGCCG
GCGTGGTGCTGGCAGGAGCCGCCCTGGGAGTGGCAACCGCCGCCCAGAT
CACAGCCGGCATCGCCCTGCACCAGTCCATGCTGAACTCTCAGGCCATC
GATAATCTGAGGGCCTCCCTGGAGACAACAAACCAGGCAATCGAGGCAA
TCAGGCAGGCAGGACAGGAGATGATCCTGGCCGTGCAGGGCGTGCAGGA
CTATATCAACAATGAGCTGATCCCTTCTATGAATCAGCTGAGCTGCGAT
CTGATCGGCCAGAAGCTGGGCCTGAAGCTGCTGCGGTACTATACCGAGA
TCCTGTCCCTGTTCGGACCTTCTCTGCGGGACCCCATCAGCGCCGAGAT
CTCCATCCAGGCCCTGTCCTATGCACTGGGAGGCGACATCAACAAGGTG
CTGGAGAAGCTGGGCTACTCTGGCGGCGATCTGCTGGGCATCCTGGAGA
GCCGGGGCATCAAGGCCAGAATCACCCACGTGGATACAGAGTCTTACTT
CATCGTGCTGAGCATCGCCTACCCCACACTGTCCGAGATCAAGGGCGTG
ATCGTGCACAGGCTGGAGGGCGTGAGCTATAATATCGGCTCCCAGGAGT
GGTACACCACAGTGCCTAAGTATGTGGCCACCCAGGGCTACCTGATCTC
TAACTTCGACGAGTCCTCTTGTACCTTTATGCCAGAGGGCACAGTGTGC
TCCCAGAATGCCCTGTACCCAATGTCTCCCCTGCTGCAGGAGTGTCTGC
GGGGCTCTACCAAGAGCTGCGCCAGAACACTGGTGAGCGGCTCCTTCGG
CAATCGGTTCATCCTGTCCCAGGGCAACCTGATCGCCAATTGTGCCTCT
ATCCTGTGCAAGTGCTATACCACAGGCACCATCATCAACCAGGACCCCG
ATAAGATCCTGACATACATCGCAGCAGACCACTGCCCTGTGGTGGAAGT
GAATGGCGTGACCATCCAAGTGGGCAGCCGGAGATACCCAGACGCCGTG
TATCTGCACAGGATCGATCTGGGACCACCTATCTCCCTGGAGCGCCTGG
ATGTGGGCACAAACCTGGGCAATGCCATCGCCAAGCTGGAGGACGCCAA
GGAGCTGCTGGAGAGCTCCGATCAGATCCTGAGGAGCATGAAGGGCCTG
TCTAGCACCTCCATCGTGTACATCCTGATCGCCGTGTGCCTGGGAGGCC
TGATCGGAATCCCCGCCCTGATCTGCTGTTGCAGGGGCCGCTGA.

By “MeV-Hwtc18 polypeptide” is meant a polypeptide or fragment thereof having at least about 85% amino acid sequence identity to the sequence provided below, and that has activity associated with fusion of a Lentivirus envelope containing an F protein domain with a membrane. In embodiments, the MeV-Hwtc18 polypeptide functions in combination with an MeV-Fc30 polypeptide to fuse a Lentivirus lipid envelope with a membrane (e.g., a cell membrane). An exemplary MeV-Hwtc18 polypeptide sequence is provided below.

MGSRIVINREHLMIDRPYVLLAVLFVMFLSLIGLLAIAGIRLHRAAIYT
AEIHKSLSTNLDVTNSIEHQVKDVLTPLFKIIGDEVGLRTPQRFTDLVK
FISDKIKFLNPDREYDERDLTWCINPPERIKLDYDQYCADVAAEELMNA
LVNSTLLEARATNQFLAVSKGNCSGPTTIRGQFSNMSLSLLDLYLSRGY
NVSSIVTMTSQGMYGGTYLVGKPNLSSKGSELSQLSMHRVFEVGVIRNP
GLGAPVFHMTNYFEQPVSNDFSNCMVALGELKFAALCHREDSITIPYQG
SGKGVSFQLVKLGVWKSPTDMRSWVPLSTDDPVIDRLYLSSHRGVIADN
QAKWAVPTTRTDDKLRMETCFQQACKGKNQALCENPEWAPLKDNRIPSY
GVLSVNLSLTVELKIKIASGFGPLITHGSGMDLYKTNHNNVYWLTIPPM
KNLALGVINTLEWIPRFKVSPALFTVPIKEAGEDCHAPTYLPAEVDGDV
KLSSNLVILPGQDLQYVLATYDTSRVEHAVVYYVYSPSRSFSYFYPFRL
PIKGVPIELQVECFTWDKKLWCRHFCVLADSESGGHITHSGMVGMGVSC
TVTREDGT.

In the above polypeptide sequence, the cytoplasmic domain (which includes an 18 amino acid truncation) is underlined, the transmembrane domain is in italics, and the extracellular domain is in plain text. By “MeV-Hwtc18 polynucleotide” is meant a polynucleotide encoding an MeV-Hwtc18 polypeptide. An exemplary MeV-Fc30 polynucleotide sequence is provided below.

GCTGTACCTGTCCTCTCACAGAGGCGTGATCGCCGACAATCAGGCAAAG
TGGGCAGTGCCAACCACAAGGACCGACGATAAGCTGAGAATGGAGACAT
GCTTTCAGCAGGCCTGTAAGGGCAAGAACCAGGCCCTGTGCGAGAATCC
AGAGTGGGCACCTCTGAAGGATAACAGAATCCCTAGCTACGGCGTGCTG
AGCGTGAATCTGTCCCTGACCGTGGAGCTGAAGATCAAGATCGCCAGCG
GCTTCGGCCCACTGATCACACACGGCTCCGGCATGGACCTGTACAAGAC
CAACCACAACAACGTGTACTGGCTGACAATCCCACCCATGAAGAACCTG
GCCCTGGGCGTGATCAATACCCTGGAGTGGATCCCCAGGTTTAAGGTGT
CCCCTGCCCTGTTCACCGTGCCAATCAAGGAGGCCGGCGAGGATTGTCA
CGCCCCAACATACCTGCCCGCCGAGGTGGACGGCGATGTGAAGCTGAGC
TCCAACCTGGTCATCCTGCCTGGCCAGGACCTGCAGTACGTGCTGGCCA
CCTATGATACAAGCCGCGTGGAGCACGCCGTGGTGTACTACGTGTACTC
CCCCTCTCGGAGCTTCTCCTACTTTTATCCTTTCAGACTGCCAATCAAG
GGCGTGCCTATCGAGCTGCAGGTGGAGTGCTTTACCTGGGATAAGAAGC
TGTGGTGCAGGCACTTCTGCGTGCTGGCAGACTCTGAGAGCGGAGGACA
CATCACCCACTCCGGAATGGTGGGCATGGGCGTGTCTTGTACCGTGACA
CGCGAGGACGGCACATGA.

By “MeV-Hwtc18-hCD105 polypeptide” is meant a polypeptide or fragment thereof having at least about 85% amino acid sequence identity to the sequence provided below, and that has activity associated with fusion of a Lentivirus envelope containing an F protein domain with a membrane of a cell surface-expressing a CD105 polypeptide. In some instances the CD105 polypeptide is a murine or human CD105 polypeptide. In embodiments, the MeV-Hwtc18-hCD105 polypeptide functions in combination with an MeV-Fc30 polypeptide to fuse a Lentivirus lipid envelope with a membrane (e.g., a cell membrane). An exemplary MeV-Hwtc18-hCD105 polypeptide sequence is provided below.

MGSRIVINREHLMIDRPYVLLAVLFVMFLSLIGLLAIAGIRLHRAAIYT
AEIHKSLSTNLDVTNSIEHQVKDVLTPLFKIIGDEVGLRTPQRFTDLVK
FISDKIKFLNPDREYDFRDLTWCINPPERIKLDYDQYCADVAAEELMNA
LVNSTLLEARATNQFLAVSKGNCSGPTTIRGQFSNMSLSLLDLYLSRGY
NVSSIVTMTSQGMYGGTYLVGKPNLSSKGSELSQLSMHRVFEVGVIRNP
GLGAPVFHMTNYFEQPVSNDFSNCMVALGELKFAALCHREDSITIPYQG
SGKGVSFQLVKLGVWKSPTDMRSWVPLSTDDPVIDRLYLSSHRGVIADN
QAKWAVPTTRTDDKLRMETCFQQACKGKNQALCENPEWAPLKDNRIPSY
GVLSVNLSLTVELKIKIASGFGPLITHGSGMDLYKTNHNNVYWLTIPPM
KNLALGVINTLEWIPRFKVSPALFTVPIKEAGEDCHAPTYLPAEVDGDV
KLSSNLVILPGQDLQYVLATYDTSRVEHAVVYYVYSPSRSFSYFYPFRL
PIKGVPIELQVECFTWDKKLWCRHFCVLADSESGGHITHSGMVGMGVSC
TVTREDGTNGGGGSGGGGSGGGGSAAQPAMAEVQLLESGGGLVQPGGSL
RLSCAASGFTFSSYAMSWVRQAPGKGLEWVSAIYGSDGDTTYADSVKGR
FTISRDNSKNTLYLQMNSLRAEDTAVYYCARVFYTAGFDYWGQGTLVTV
SSGSDSNAGHASAGNTSDIELTQSPSSLSASVGDRVTITCRASQSISSS
LNWYQQKPGKAPKLLIYAASSLQSGVPSRFSGSGSGTDFTLTISSLQPE
DFATYYCQQAPAKPPTFGQGTKLEIKRAAARGSHHHHHH.

In the above polypeptide sequence, the cytoplasmic domain is underlined, the transmembrane domain is in italics, the extracellular domain is in plain text, the linker is in bold, the SfiI site is in bold underline, the anti-hCD105 scFv domain is in bold italic, the NotI site is in italic underline, and the 6His tag is double-underlined.

By “MeV-Hwtc18-hCD105 polynucleotide” is meant a polynucleotide encoding an MeV-Hwtc18-hCD105 polypeptide. An exemplary MeV-Hwtc18-hCD105 polynucleotide sequence is provided below.

ATGGGCTCTAGGATCGTGATCAACCGCGAGCACCTGATGATCGACAGGC
CATACGTGCTGCTGGCCGTGCTGTTCGTGATGTTTCTGAGCCTGATCGG
CCTGCTGGCAATCGCAGGAATCAGGCTGCACAGAGCCGCCATCTATACC
GCCGAGATCCACAAGAGCCTGTCCACCAACCTGGACGTGACAAATTCCA
TCGAGCACCAGGTGAAGGATGTGCTGACACCACTGTTCAAGATCATCGG
CGACGAAGTGGGCCTGAGGACCCCACAGCGGTTCACCGACCTGGTGAAG
TTCATCAGCGATAAGATCAAGTTTCTGAACCCTGATCGGGAGTACGACT
TCAGAGATCTGACCTGGTGCATCAATCCCCCTGAGCGGATCAAGCTGGA
CTACGATCAGTATTGTGCCGACGTGGCCGCCGAGGAGCTGATGAACGCC
CTGGTGAATTCCACCCTGCTGGAGGCCAGAGCCACAAACCAGTTTCTGG
CCGTGAGCAAGGGCAATTGCTCCGGCCCAACCACAATCCGGGGCCAGTT
CTCTAACATGTCTCTGAGCCTGCTGGATCTGTACCTGAGCAGAGGCTAT
AATGTGAGCTCCATCGTGACCATGACAAGCCAGGGCATGTACGGCGGCA
CCTATCTGGTGGGCAAGCCCAACCTGTCTAGCAAGGGCTCCGAGCTGTC
TCAGCTGAGCATGCACAGGGTGTTTGAAGTGGGCGTGATCAGGAACCCA
GGCCTGGGAGCCCCCGTGTTTCACATGACCAATTACTTCGAGCAGCCCG
TGAGCAACGATTTTTCTAATTGCATGGTGGCCCTGGGAGAGCTGAAGTT
CGCCGCCCTGTGCCACAGGGAGGACAGCATCACCATCCCATATCAGGGC
TCTGGCAAGGGCGTGAGCTTCCAGCTGGTGAAGCTGGGCGTGTGGAAGT
CCCCCACCGACATGAGATCCTGGGTGCCTCTGTCTACAGACGATCCAGT
GATCGATCGGCTGTACCTGTCCTCTCACAGAGGCGTGATCGCCGACAAT
CAGGCAAAGTGGGCAGTGCCAACCACAAGGACCGACGATAAGCTGAGAA
TGGAGACATGCTTTCAGCAGGCCTGTAAGGGCAAGAACCAGGCCCTGTG
CGAGAATCCAGAGTGGGCACCTCTGAAGGATAACAGAATCCCTAGCTAC
GGCGTGCTGAGCGTGAATCTGTCCCTGACCGTGGAGCTGAAGATCAAGA
TCGCCAGCGGCTTCGGCCCACTGATCACACACGGCTCCGGCATGGACCT
GTACAAGACCAACCACAACAACGTGTACTGGCTGACAATCCCACCCATG
AAGAACCTGGCCCTGGGCGTGATCAATACCCTGGAGTGGATCCCCAGGT
TTAAGGTGTCCCCTGCCCTGTTCACCGTGCCAATCAAGGAGGCCGGCGA
GGATTGTCACGCCCCAACATACCTGCCCGCCGAGGTGGACGGCGATGTG
AAGCTGAGCTCCAACCTGGTCATCCTGCCTGGCCAGGACCTGCAGTACG
TGCTGGCCACCTATGATACAAGCCGCGTGGAGCACGCCGTGGTGTACTA
CGTGTACTCCCCCTCTCGGAGCTTCTCCTACTTTTATCCTTTCAGACTG
CCAATCAAGGGCGTGCCTATCGAGCTGCAGGTGGAGTGCTTTACCTGGG
ATAAGAAGCTGTGGTGCAGGCACTTCTGCGTGCTGGCAGACTCTGAGAG
CGGAGGACACATCACCCACTCCGGAATGGTGGGCATGGGCGTGTCTTGT
ACCGTGACACGCGAGGACGGCACAAACGGAGGAGGAGGAAGCGGAGGAG
GAGGATCCGGCGGCGGCGGCTCTGCCGCCCAGCCTGCCATGGCCGAGGT
GCAGCTGCTGGAGTCCGGAGGAGGCCTGGTGCAGCCAGGAGGCTCTCTG
AGGCTGAGCTGCGCAGCATCCGGCTTCACCTTTTCTAGCTACGCCATGA
GCTGGGTGCGCCAGGCACCAGGCAAGGGCCTGGAGTGGGTGAGCGCCAT
CTACGGATCCGACGGCGATACCACATATGCCGATTCCGTGAAGGGCAGG
TTCACCATCTCTCGCGACAACAGCAAGAATACACTGTATCTGCAGATGA
ACTCTCTGAGGGCCGAGGATACCGCCGTGTACTATTGCGCCCGCGTGTT
TTACACAGCCGGCTTCGACTATTGGGGCCAGGGCACCCTGGTGACAGTG
TCCTCTGGCTCCGATTCTAACGCAGGACACGCATCTGCCGGAAATACCA
GCGACATCGAGCTGACACAGTCCCCAAGCTCCCTGAGCGCCTCCGTGGG
CGACAGGGTGACCATCACATGTCGCGCCTCTCAGAGCATCTCTAGCTCC
CTGAATTGGTACCAGCAGAAGCCCGGCAAGGCCCCTAAGCTGCTGATCT
ATGCAGCATCTAGCCTGCAGTCCGGAGTGCCATCTAGGTTTTCCGGCTC
TGGCAGCGGCACCGATTTCACCCTGACAATCTCCTCTCTGCAGCCCGAG
GACTTTGCCACATACTATTGTCAGCAGGCCCCTGCCAAGCCTCCAACCT
TCGGCCAGGGCACAAAGCTGGAGATCAAGAGGGCAGCAGCAAGAGGCAG
CCACCACCACCACCACCACTGA.

By “MeV-Hwtc18-MHCII (N11) polypeptide” is meant a polypeptide or fragment thereof having at least about 85% amino acid sequence identity to the sequence provided below, and that has activity associated with fusion of a Lentivirus envelope containing an F protein domain with a membrane of a cell surface-expressing an MHC-II polypeptide. In some instances the MHC-II polypeptide is a murine or human MHC-II polypeptide. In embodiments, the MeV-Hwtc18-MHCII (N11) polypeptide functions in combination with an MeV-Fc30 polypeptide to fuse a Lentivirus lipid envelope with a membrane (e.g., a cell membrane). An exemplary MeV-Hwtc18-MHCII (N11) polypeptide sequence is provided below.

MGSRIVINREHLMIDRPYVLLAVLFVMFLSLIGLLAIAGIRLHRAAIYT
AEIHKSLSTNLDVTNSIEHQVKDVLTPLFKIIGDEVGLRTPQRFTDLVK
FISDKIKFLNPDREYDERDLTWCINPPERIKLDYDQYCADVAAEELMNA
LVNSTLLEARATNQFLAVSKGNCSGPTTIRGQFSNMSLSLLDLYLSRGY
NVSSIVTMTSQGMYGGTYLVGKPNLSSKGSELSQLSMHRVFEVGVIRNP
GLGAPVFHMTNYFEQPVSNDFSNCMVALGELKFAALCHREDSITIPYQG
SGKGVSFQLVKLGVWKSPTDMRSWVPLSTDDPVIDRLYLSSHRGVIADN
QAKWAVPTTRTDDKLRMETCFQQACKGKNQALCENPEWAPLKDNRIPSY
GVLSVNLSLTVELKIKIASGFGPLITHGSGMDLYKTNHNNVYWLTIPPM
KNLALGVINTLEWIPRFKVSPALFTVPIKEAGEDCHAPTYLPAEVDGDV
KLSSNLVILPGQDLQYVLATYDTSRVEHAVVYYVYSPSRSFSYFYPFRL
PIKGVPIELQVECFTWDKKLWCRHFCVLADSESGGHITHSGMVGMGVSC
TVTREDGTGGGGSGGGGSGGGGSAAAQVQLVQSGGGLVQPGGSLGLSCA
ASGNIGSRDNMGWYRQAPGKQREWVATISGYGIATYRDSVKGRFTVAKD
TAKNIVSLQMNYLTTEDTAVYYCYAYAVDSRNIFWSQGTQVTVSGGGSG
GGSYPYDVPDYA.

In the above polypeptide sequence, the cytoplasmic domain is underlined, the transmembrane domain is in italics, the extracellular domain is in plain text, the (G3S)3 linker is in bold, the NotI site is in bold underline, the N11 VHH domain is in bold italic, the (G3S)2 linker is in italic underline, and the HA tag is double-underlined.

By “MeV-Hwtc18-MHCII (N11) polynucleotide” is meant a polynucleotide encoding an MeV-Hwtc18-MHCII (N11) polypeptide. An exemplary MeV-Hwtc18-MHCII (N11) polynucleotide sequence is provided below.

ATGGGCTCTAGGATCGTGATCAACCGCGAGCACCTGATGATCGACAGGC
CATACGTGCTGCTGGCCGTGCTGTTCGTGATGTTTCTGAGCCTGATCGG
CCTGCTGGCAATCGCAGGAATCAGGCTGCACAGAGCCGCCATCTATACC
GCCGAGATCCACAAGAGCCTGTCCACCAACCTGGACGTGACAAATTCCA
TCGAGCACCAGGTGAAGGATGTGCTGACACCACTGTTCAAGATCATCGG
CGACGAAGTGGGCCTGAGGACCCCACAGCGGTTCACCGACCTGGTGAAG
TTCATCAGCGATAAGATCAAGTTTCTGAACCCTGATCGGGAGTACGACT
TCAGAGATCTGACCTGGTGCATCAATCCCCCTGAGCGGATCAAGCTGGA
CTACGATCAGTATTGTGCCGACGTGGCCGCCGAGGAGCTGATGAACGCC
CTGGTGAATTCCACCCTGCTGGAGGCCAGAGCCACAAACCAGTTTCTGG
CCGTGAGCAAGGGCAATTGCTCCGGCCCAACCACAATCCGGGGCCAGTT
CTCTAACATGTCTCTGAGCCTGCTGGATCTGTACCTGAGCAGAGGCTAT
AATGTGAGCTCCATCGTGACCATGACAAGCCAGGGCATGTACGGCGGCA
CCTATCTGGTGGGCAAGCCCAACCTGTCTAGCAAGGGCTCCGAGCTGTC
TCAGCTGAGCATGCACAGGGTGTTTGAAGTGGGCGTGATCAGGAACCCA
GGCCTGGGAGCCCCCGTGTTTCACATGACCAATTACTTCGAGCAGCCCG
TGAGCAACGATTTTTCTAATTGCATGGTGGCCCTGGGAGAGCTGAAGTT
CGCCGCCCTGTGCCACAGGGAGGACAGCATCACCATCCCATATCAGGGC
TCTGGCAAGGGCGTGAGCTTCCAGCTGGTGAAGCTGGGCGTGTGGAAGT
CCCCCACCGACATGAGATCCTGGGTGCCTCTGTCTACAGACGATCCAGT
GATCGATCGGCTGTACCTGTCCTCTCACAGAGGCGTGATCGCCGACAAT
CAGGCAAAGTGGGCAGTGCCAACCACAAGGACCGACGATAAGCTGAGAA
TGGAGACATGCTTTCAGCAGGCCTGTAAGGGCAAGAACCAGGCCCTGTG
CGAGAATCCAGAGTGGGCACCTCTGAAGGATAACAGAATCCCTAGCTAC
GGCGTGCTGAGCGTGAATCTGTCCCTGACCGTGGAGCTGAAGATCAAGA
TCGCCAGCGGCTTCGGCCCACTGATCACACACGGCTCCGGCATGGACCT
GTACAAGACCAACCACAACAACGTGTACTGGCTGACAATCCCACCCATG
AAGAACCTGGCCCTGGGCGTGATCAATACCCTGGAGTGGATCCCCAGGT
TTAAGGTGTCCCCTGCCCTGTTCACCGTGCCAATCAAGGAGGCCGGCGA
GGATTGTCACGCCCCAACATACCTGCCCGCCGAGGTGGACGGCGATGTG
AAGCTGAGCTCCAACCTGGTCATCCTGCCTGGCCAGGACCTGCAGTACG
TGCTGGCCACCTATGATACAAGCCGCGTGGAGCACGCCGTGGTGTACTA
CGTGTACTCCCCCTCTCGGAGCTTCTCCTACTTTTATCCTTTCAGACTG
CCAATCAAGGGCGTGCCTATCGAGCTGCAGGTGGAGTGCTTTACCTGGG
ATAAGAAGCTGTGGTGCAGGCACTTCTGCGTGCTGGCAGACTCTGAGAG
CGGAGGACACATCACCCACTCCGGAATGGTGGGCATGGGCGTGTCTTGT
ACCGTGACACGCGAGGACGGCACAGGGGGAGGCGGCTCTGGAGGTGGGG
GGTCCGGTGGCGGCGGAAGCGCGGCCGCGCAGGTTCAACTGGTGCAGAG
CGGAGGCGGCCTGGTCCAGCCCGGCGGAAGCCTGGGCCTGAGCTGTGCC
GCTTCTGGCAACATCGGCAGCAGAGATAATATGGGCTGGTATAGACAGG
CCCCTGGAAAACAGAGAGAGTGGGTGGCTACAATCAGCGGCTACGGCAT
CGCCACATACCGGGACAGCGTGAAGGGCAGATTCACCGTGGCCAAGGAC
ACCGCCAAGAACATCGTGTCCCTGCAGATGAACTACCTGACCACCGAAG
ATACAGCCGTGTACTACTGCTACGCCTACGCCGTGGACAGCCGGAACAT
CTTCTGGTCCCAGGGCACCCAGGTGACCGTGTCTGGCGGCGGCTCCGGC
GGCGGCAGCTATCCCTATGATGTGCCCGACTATGCG.

By “MeV-Hwtc18-CD45 (32) polypeptide” is meant a polypeptide or fragment thereof having at least about 85% amino acid sequence identity to the sequence provided below, and that has activity associated with fusion of a Lentivirus envelope containing an F protein domain with a membrane of a cell surface-expressing a CD45 polypeptide. In some instances the CD45 polypeptide is a murine or human CD45 polypeptide. In embodiments, the MeV-Hwtc18-CD45 (32) polypeptide functions in combination with an MeV-Fc30 polypeptide to fuse a lentivirus lipid envelope with a membrane (e.g., a cell membrane). An exemplary MeV-Hwtc18-CD45 (32) polypeptide sequence is provided below.

MGSRIVINREHLMIDRPYVLLAVLFVMFLSLIGLLAIAGIRLHRAAIYT
AEIHKSLSTNLDVTNSIEHQVKDVLTPLFKIIGDEVGLRTPQRFTDLVK
FISDKIKFLNPDREYDFRDLTWCINPPERIKLDYDQYCADVAAEELMNA
LVNSTLLEARATNQFLAVSKGNCSGPTTIRGQFSNMSLSLLDLYLSRGY
NVSSIVTMTSQGMYGGTYLVGKPNLSSKGSELSQLSMHRVFEVGVIRNP
GLGAPVFHMTNYFEQPVSNDFSNCMVALGELKFAALCHREDSITIPYQG
SGKGVSFQLVKLGVWKSPTDMRSWVPLSTDDPVIDRLYLSSHRGVIADN
QAKWAVPTTRTDDKLRMETCFQQACKGKNQALCENPEWAPLKDNRIPSY
GVLSVNLSLTVELKIKIASGFGPLITHGSGMDLYKTNHNNVYWLTIPPM
KNLALGVINTLEWIPRFKVSPALFTVPIKEAGEDCHAPTYLPAEVDGDV
KLSSNLVILPGQDLQYVLATYDTSRVEHAVVYYVYSPSRSFSYFYPFRL
PIKGVPIELQVECFTWDKKLWCRHFCVLADSESGGHITHSGMVGMGVSC
TVTREDGTGGGGSGGGGSGGGGSAAAQVQLVQSGGGLVQPGGSLRLSCA
ASGRAFNSAAMGWYRQAPGSQRELVASISAGTASYADAVKGRFTISRDY
AKNIIYLQMNSLKPDDTAVYFCNYRTTYTSGYSEDYWGQGTQVTVSGGG
SGGGSYPYDVPDYA.

In the above polypeptide sequence, the cytoplasmic domain is underlined, the transmembrane domain is in italics, the extracellular domain is in plain text, the (G3S)3 linker is in bold, the NotI site is in bold underline, the VHH domain is in bold italic, the (G3S)2 linker is in italic underline, and the HA tag is double-underlined.

By “MeV-Hwtc18-CD45 (32) polynucleotide” is meant a polynucleotide encoding an MeV-Hwtc18-CD45 (32) polypeptide. An exemplary MeV-Hwtc18-CD45 (32) polynucleotide sequence is provided below.

ATGGGCTCTAGGATCGTGATCAACCGCGAGCACCTGATGATCGACAGGC
CATACGTGCTGCTGGCCGTGCTGTTCGTGATGTTTCTGAGCCTGATCGG
CCTGCTGGCAATCGCAGGAATCAGGCTGCACAGAGCCGCCATCTATACC
GCCGAGATCCACAAGAGCCTGTCCACCAACCTGGACGTGACAAATTCCA
TCGAGCACCAGGTGAAGGATGTGCTGACACCACTGTTCAAGATCATCGG
CGACGAAGTGGGCCTGAGGACCCCACAGCGGTTCACCGACCTGGTGAAG
TTCATCAGCGATAAGATCAAGTTTCTGAACCCTGATCGGGAGTACGACT
TCAGAGATCTGACCTGGTGCATCAATCCCCCTGAGCGGATCAAGCTGGA
CTACGATCAGTATTGTGCCGACGTGGCCGCCGAGGAGCTGATGAACGCC
CTGGTGAATTCCACCCTGCTGGAGGCCAGAGCCACAAACCAGTTTCTGG
CCGTGAGCAAGGGCAATTGCTCCGGCCCAACCACAATCCGGGGCCAGTT
CTCTAACATGTCTCTGAGCCTGCTGGATCTGTACCTGAGCAGAGGCTAT
AATGTGAGCTCCATCGTGACCATGACAAGCCAGGGCATGTACGGCGGCA
CCTATCTGGTGGGCAAGCCCAACCTGTCTAGCAAGGGCTCCGAGCTGTC
TCAGCTGAGCATGCACAGGGTGTTTGAAGTGGGCGTGATCAGGAACCCA
GGCCTGGGAGCCCCCGTGTTTCACATGACCAATTACTTCGAGCAGCCCG
TGAGCAACGATTTTTCTAATTGCATGGTGGCCCTGGGAGAGCTGAAGTT
CGCCGCCCTGTGCCACAGGGAGGACAGCATCACCATCCCATATCAGGGC
TCTGGCAAGGGCGTGAGCTTCCAGCTGGTGAAGCTGGGCGTGTGGAAGT
CCCCCACCGACATGAGATCCTGGGTGCCTCTGTCTACAGACGATCCAGT
GATCGATCGGCTGTACCTGTCCTCTCACAGAGGCGTGATCGCCGACAAT
CAGGCAAAGTGGGCAGTGCCAACCACAAGGACCGACGATAAGCTGAGAA
TGGAGACATGCTTTCAGCAGGCCTGTAAGGGCAAGAACCAGGCCCTGTG
CGAGAATCCAGAGTGGGCACCTCTGAAGGATAACAGAATCCCTAGCTAC
GGCGTGCTGAGCGTGAATCTGTCCCTGACCGTGGAGCTGAAGATCAAGA
TCGCCAGCGGCTTCGGCCCACTGATCACACACGGCTCCGGCATGGACCT
GTACAAGACCAACCACAACAACGTGTACTGGCTGACAATCCCACCCATG
AAGAACCTGGCCCTGGGCGTGATCAATACCCTGGAGTGGATCCCCAGGT
TTAAGGTGTCCCCTGCCCTGTTCACCGTGCCAATCAAGGAGGCCGGCGA
GGATTGTCACGCCCCAACATACCTGCCCGCCGAGGTGGACGGCGATGTG
AAGCTGAGCTCCAACCTGGTCATCCTGCCTGGCCAGGACCTGCAGTACG
TGCTGGCCACCTATGATACAAGCCGCGTGGAGCACGCCGTGGTGTACTA
CGTGTACTCCCCCTCTCGGAGCTTCTCCTACTTTTATCCTTTCAGACTG
CCAATCAAGGGCGTGCCTATCGAGCTGCAGGTGGAGTGCTTTACCTGGG
ATAAGAAGCTGTGGTGCAGGCACTTCTGCGTGCTGGCAGACTCTGAGAG
CGGAGGACACATCACCCACTCCGGAATGGTGGGCATGGGCGTGTCTTGT
ACCGTGACACGCGAGGACGGCACAGGGGGAGGCGGCTCTGGAGGTGGGG
GGTCCGGTGGCGGCGGAAGCGCGGCCGCGCAGGTGCAGCTGGTGCAGAG
CGGCGGAGGACTGGTGCAACCTGGAGGCAGCCTGAGACTGAGCTGCGCC
GCTAGCGGCAGAGCCTTTAACAGCGCCGCCATGGGCTGGTACAGACAGG
CCCCTGGCTCTCAGAGAGAGCTGGTCGCCTCTATCTCCGCCGGCACCGC
CTCTTATGCTGATGCCGTGAAAGGCAGATTCACCATCAGCCGGGACTAC
GCCAAGAACATCATCTACCTGCAGATGAACAGCCTGAAGCCCGACGACA
CAGCTGTGTACTTCTGTAATTACCGGACAACCTACACCAGCGGCTACAG
CGAAGATTACTGGGGCCAGGGCACACAGGTGACCGTGTCCGGCGGCGGC
TCCGGCGGCGGCAGCTATCCCTATGATGTGCCCGACTATGCG.

By “MeV-Hwtc18-Thy1.1 (154.7.7.10) polypeptide” is meant a polypeptide or fragment thereof having at least about 85% amino acid sequence identity to the sequence provided below, and that has activity associated with fusion of a Lentivirus envelope containing an F protein domain with a membrane of a cell surface-expressing a Thy1 polypeptide. In some instances the Thy1 polypeptide is a murine or human Thy1 polypeptide. In embodiments, the MeV-Hwtc18-Thy1.1 (154.7.7.10) polypeptide functions in combination with an MeV-Fc30 polypeptide to fuse a Lentivirus lipid envelope with a membrane (e.g., a cell membrane). An exemplary MeV-Hwtc18-Thy1.1 (154.7.7.10) polypeptide sequence is provided below.

MGSRIVINREHLMIDRPYVLLAVLFVMFLSLIGLLAIAGIRLHRAAIYT
AEIHKSLSTNLDVTNSIEHQVKDVLTPLFKIIGDEVGLRTPQRFTDLVK
FISDKIKFLNPDREYDFRDLTWCINPPERIKLDYDQYCADVAAEELMNA
LVNSTLLEARATNQFLAVSKGNCSGPTTIRGQFSNMSLSLLDLYLSRGY
NVSSIVTMTSQGMYGGTYLVGKPNLSSKGSELSQLSMHRVFEVGVIRNP
GLGAPVFHMTNYFEQPVSNDFSNCMVALGELKFAALCHREDSITIPYQG
SGKGVSFQLVKLGVWKSPTDMRSWVPLSTDDPVIDRLYLSSHRGVIADN
QAKWAVPTTRTDDKLRMETCFQQACKGKNQALCENPEWAPLKDNRIPSY
GVLSVNLSLTVELKIKIASGFGPLITHGSGMDLYKTNHNNVYWLTIPPM
KNLALGVINTLEWIPRFKVSPALFTVPIKEAGEDCHAPTYLPAEVDGDV
KLSSNLVILPGQDLQYVLATYDTSRVEHAVVYYVYSPSRSFSYFYPFRL
PIKGVPIELQVECFTWDKKLWCRHFCVLADSESGGHITHSGMVGMGVSC
TVTREDGTGGGGSGGGGSGGGGSAAAQVQLKESGPGLVKPSETLSLTCT
VSGFSFTSYHISWVRQPPGKGLEWMGVIWGDGSTAYNSVFKSRLSISRD
TSKSQVFLKMSSLKTEDTATYYCARDRDWELGNWFAYWGQGTLVTVSSG
GGGSGGGGSGGGGSDIQMTQSPPSLSASLGDKVTITCQASQNINKYIAW
YQQKPGKAPRQLIYYTSILVSGTPSRFSGSGSGRDYSFSISNVESEDIA
SYYCLQYDNLWTFGGGTKLELKGGGSGGGSYPYDVPDYA.

In the above polypeptide sequence, the cytoplasmic domain is underlined, the transmembrane domain is in italics, the extracellular domain is in plain text, the (G3S)3 linkers are in bold, the NotI site is in bold underline, the variable light (VL) chain sequence of the scFv is in bold italic, the variable heavy (VH) chain sequence of the scFv is in italic underline, the (G3S)2 linker is in bold underlined italics, and the HA tag is double-underlined.

By “MeV-Hwtc18-Thy1.1 (154.7.7.10) polynucleotide” is meant a polynucleotide encoding an MeV-Hwtc18-Thy1.1 (154.7.7.10) polypeptide. An exemplary MeV-Hwtc18-Thy1.1 (154.7.7.10) polynucleotide sequence is provided below.

ATGGGCTCTAGGATCGTGATCAACCGCGAGCACCTGATGATCGACAGGC
CATACGTGCTGCTGGCCGTGCTGTTCGTGATGTTTCTGAGCCTGATCGG
CCTGCTGGCAATCGCAGGAATCAGGCTGCACAGAGCCGCCATCTATACC
GCCGAGATCCACAAGAGCCTGTCCACCAACCTGGACGTGACAAATTCCA
TCGAGCACCAGGTGAAGGATGTGCTGACACCACTGTTCAAGATCATCGG
CGACGAAGTGGGCCTGAGGACCCCACAGCGGTTCACCGACCTGGTGAAG
TTCATCAGCGATAAGATCAAGTTTCTGAACCCTGATCGGGAGTACGACT
TCAGAGATCTGACCTGGTGCATCAATCCCCCTGAGCGGATCAAGCTGGA
CTACGATCAGTATTGTGCCGACGTGGCCGCCGAGGAGCTGATGAACGCC
CTGGTGAATTCCACCCTGCTGGAGGCCAGAGCCACAAACCAGTTTCTGG
CCGTGAGCAAGGGCAATTGCTCCGGCCCAACCACAATCCGGGGCCAGTT
CTCTAACATGTCTCTGAGCCTGCTGGATCTGTACCTGAGCAGAGGCTAT
AATGTGAGCTCCATCGTGACCATGACAAGCCAGGGCATGTACGGCGGCA
CCTATCTGGTGGGCAAGCCCAACCTGTCTAGCAAGGGCTCCGAGCTGTC
TCAGCTGAGCATGCACAGGGTGTTTGAAGTGGGCGTGATCAGGAACCCA
GGCCTGGGAGCCCCCGTGTTTCACATGACCAATTACTTCGAGCAGCCCG
TGAGCAACGATTTTTCTAATTGCATGGTGGCCCTGGGAGAGCTGAAGTT
CGCCGCCCTGTGCCACAGGGAGGACAGCATCACCATCCCATATCAGGGC
TCTGGCAAGGGCGTGAGCTTCCAGCTGGTGAAGCTGGGCGTGTGGAAGT
CCCCCACCGACATGAGATCCTGGGTGCCTCTGTCTACAGACGATCCAGT
GATCGATCGGCTGTACCTGTCCTCTCACAGAGGCGTGATCGCCGACAAT
CAGGCAAAGTGGGCAGTGCCAACCACAAGGACCGACGATAAGCTGAGAA
TGGAGACATGCTTTCAGCAGGCCTGTAAGGGCAAGAACCAGGCCCTGTG
CGAGAATCCAGAGTGGGCACCTCTGAAGGATAACAGAATCCCTAGCTAC
GGCGTGCTGAGCGTGAATCTGTCCCTGACCGTGGAGCTGAAGATCAAGA
TCGCCAGCGGCTTCGGCCCACTGATCACACACGGCTCCGGCATGGACCT
GTACAAGACCAACCACAACAACGTGTACTGGCTGACAATCCCACCCATG
AAGAACCTGGCCCTGGGCGTGATCAATACCCTGGAGTGGATCCCCAGGT
TTAAGGTGTCCCCTGCCCTGTTCACCGTGCCAATCAAGGAGGCCGGCGA
GGATTGTCACGCCCCAACATACCTGCCCGCCGAGGTGGACGGCGATGTG
AAGCTGAGCTCCAACCTGGTCATCCTGCCTGGCCAGGACCTGCAGTACG
TGCTGGCCACCTATGATACAAGCCGCGTGGAGCACGCCGTGGTGTACTA
CGTGTACTCCCCCTCTCGGAGCTTCTCCTACTTTTATCCTTTCAGACTG
CCAATCAAGGGCGTGCCTATCGAGCTGCAGGTGGAGTGCTTTACCTGGG
ATAAGAAGCTGTGGTGCAGGCACTTCTGCGTGCTGGCAGACTCTGAGAG
CGGAGGACACATCACCCACTCCGGAATGGTGGGCATGGGCGTGTCTTGT
ACCGTGACACGCGAGGACGGCACAGGGGGAGGCGGCTCTGGAGGTGGGG
GGTCCGGTGGCGGCGGAAGCGCGGCCGCGCAAGTGCAGCTGAAGGAATC
TGGTCCTGGACTGGTTAAGCCCAGCGAGACACTGAGCCTGACCTGTACC
GTGAGCGGCTTCAGCTTTACCAGCTACCACATCAGCTGGGTGCGGCAAC
CTCCTGGAAAGGGCCTGGAGTGGATGGGCGTCATCTGGGGCGACGGCTC
AACAGCTTACAACAGCGTGTTCAAGTCTCGGCTGAGCATCAGCAGAGAT
ACAAGCAAGAGCCAGGTGTTTCTGAAAATGAGCAGCCTTAAGACCGAGG
ATACCGCCACCTATTACTGCGCCAGAGACAGAGACTGGGAGCTGGGCAA
CTGGTTCGCCTACTGGGGCCAGGGCACCCTGGTGACAGTGTCCAGCGGC
GGCGGAGGCTCCGGCGGAGGAGGCTCTGGCGGCGGCGGCTCTGATATCC
AGATGACACAGAGCCCCCCCAGCCTGAGCGCCAGCCTGGGCGACAAGGT
GACCATCACCTGCCAGGCCTCCCAGAACATCAACAAGTACATCGCCTGG
TACCAGCAGAAACCTGGCAAGGCCCCTAGACAGCTGATCTACTACACCT
CTATTCTGGTGTCCGGCACACCTAGCCGGTTCAGCGGATCTGGCAGCGG
CAGAGACTACAGCTTCAGCATCAGCAATGTGGAAAGCGAGGACATCGCT
TCTTATTACTGTCTGCAGTACGACAACCTGTGGACCTTCGGCGGTGGCA
CCAAGCTGGAACTGAAAGGAGGCGGATCAGGAGGTGGATCCTATCCCTA
TGATGTGCCCGACTATGCGTGA.

By “MeV-Hwtc18-CD7 (humanized VHH10) polypeptide” is meant a polypeptide or fragment thereof having at least about 85% amino acid sequence identity to the sequence provided below, and that has activity associated with fusion of a Lentivirus envelope containing an F protein domain with a membrane of a cell surface-expressing a CD7 polypeptide. In some instances the CD7 polypeptide is a murine or human CD7 polypeptide. In embodiments, the MeV-Hwtc18-CD7 (humanized VHH10) polypeptide functions in combination with an MeV-Fc30 polypeptide to fuse a Lentivirus lipid envelope with a membrane (e.g., a cell membrane). An exemplary MeV-Hwtc18-CD7 (humanized VHH10) polypeptide sequence is provided below.

MGSRIVINREHLMIDRPYVLLAVLFVMFLSLIGLLAIAGIRLHRAAIYT
AEIHKSLSTNLDVTNSIEHQVKDVLTPLFKIIGDEVGLRTPQRFTDLVK
FISDKIKFLNPDREYDFRDLTWCINPPERIKLDYDQYCADVAAEELMNA
LVNSTLLEARATNQFLAVSKGNCSGPTTIRGQFSNMSLSLLDLYLSRGY
NVSSIVTMTSQGMYGGTYLVGKPNLSSKGSELSQLSMHRVFEVGVIRNP
GLGAPVFHMTNYFEQPVSNDFSNCMVALGELKFAALCHREDSITIPYQG
SGKGVSFQLVKLGVWKSPTDMRSWVPLSTDDPVIDRLYLSSHRGVIADN
QAKWAVPTTRTDDKLRMETCFQQACKGKNQALCENPEWAPLKDNRIPSY
GVLSVNLSLTVELKIKIASGFGPLITHGSGMDLYKTNHNNVYWLTIPPM
KNLALGVINTLEWIPRFKVSPALFTVPIKEAGEDCHAPTYLPAEVDGDV
KLSSNLVILPGQDLQYVLATYDTSRVEHAVVYYVYSPSRSFSYFYPFRL
PIKGVPIELQVECFTWDKKLWCRHFCVLADSESGGHITHSGMVGMGVSC
TVTREDGTGGGGSGGGGSGGGGSAAADVQLQESGGGSVQAGGSLRLSCA
ASGYTHSSYCMAWFRQAPGREREGVASIDSDGTTSYADSVKGRFTISQD
NAKNTLYLQMNSLKPEDTAMYYCAARFGPMGCVDLSTLSFGHWGQGTQV
TVSITGGGSGGGSYPYDVPDYA.

In the above polypeptide sequence, the cytoplasmic domain is underlined, the transmembrane domain is in italics, the extracellular domain is in plain text, the (G3S)3 linker is in bold, the NotI site is in bold underline, humanized VHH10 is in bold italic, the (G3S)2 linker is in underlined italics, and the HA tag is double-underlined.

By “MeV-Hwtc18-CD7 (humanized VHH10) polynucleotide” is meant a polynucleotide encoding an MeV-Hwtc18-CD7 (humanized VHH10) polypeptide. An exemplary MeV-Hwtc18-CD7 (humanized VHH10) polynucleotide sequence is provided below.

ATGGGCTCTAGGATCGTGATCAACCGCGAGCACCTGATGATCGACAGGC
CATACGTGCTGCTGGCCGTGCTGTTCGTGATGTTTCTGAGCCTGATCGG
CCTGCTGGCAATCGCAGGAATCAGGCTGCACAGAGCCGCCATCTATACC
GCCGAGATCCACAAGAGCCTGTCCACCAACCTGGACGTGACAAATTCCA
TCGAGCACCAGGTGAAGGATGTGCTGACACCACTGTTCAAGATCATCGG
CGACGAAGTGGGCCTGAGGACCCCACAGCGGTTCACCGACCTGGTGAAG
TTCATCAGCGATAAGATCAAGTTTCTGAACCCTGATCGGGAGTACGACT
TCAGAGATCTGACCTGGTGCATCAATCCCCCTGAGCGGATCAAGCTGGA
CTACGATCAGTATTGTGCCGACGTGGCCGCCGAGGAGCTGATGAACGCC
CTGGTGAATTCCACCCTGCTGGAGGCCAGAGCCACAAACCAGTTTCTGG
CCGTGAGCAAGGGCAATTGCTCCGGCCCAACCACAATCCGGGGCCAGTT
CTCTAACATGTCTCTGAGCCTGCTGGATCTGTACCTGAGCAGAGGCTAT
AATGTGAGCTCCATCGTGACCATGACAAGCCAGGGCATGTACGGCGGCA
CCTATCTGGTGGGCAAGCCCAACCTGTCTAGCAAGGGCTCCGAGCTGTC
TCAGCTGAGCATGCACAGGGTGTTTGAAGTGGGCGTGATCAGGAACCCA
GGCCTGGGAGCCCCCGTGTTTCACATGACCAATTACTTCGAGCAGCCCG
TGAGCAACGATTTTTCTAATTGCATGGTGGCCCTGGGAGAGCTGAAGTT
CGCCGCCCTGTGCCACAGGGAGGACAGCATCACCATCCCATATCAGGGC
TCTGGCAAGGGCGTGAGCTTCCAGCTGGTGAAGCTGGGCGTGTGGAAGT
CCCCCACCGACATGAGATCCTGGGTGCCTCTGTCTACAGACGATCCAGT
GATCGATCGGCTGTACCTGTCCTCTCACAGAGGCGTGATCGCCGACAAT
CAGGCAAAGTGGGCAGTGCCAACCACAAGGACCGACGATAAGCTGAGAA
TGGAGACATGCTTTCAGCAGGCCTGTAAGGGCAAGAACCAGGCCCTGTG
CGAGAATCCAGAGTGGGCACCTCTGAAGGATAACAGAATCCCTAGCTAC
GGCGTGCTGAGCGTGAATCTGTCCCTGACCGTGGAGCTGAAGATCAAGA
TCGCCAGCGGCTTCGGCCCACTGATCACACACGGCTCCGGCATGGACCT
GTACAAGACCAACCACAACAACGTGTACTGGCTGACAATCCCACCCATG
AAGAACCTGGCCCTGGGCGTGATCAATACCCTGGAGTGGATCCCCAGGT
TTAAGGTGTCCCCTGCCCTGTTCACCGTGCCAATCAAGGAGGCCGGCGA
GGATTGTCACGCCCCAACATACCTGCCCGCCGAGGTGGACGGCGATGTG
AAGCTGAGCTCCAACCTGGTCATCCTGCCTGGCCAGGACCTGCAGTACG
TGCTGGCCACCTATGATACAAGCCGCGTGGAGCACGCCGTGGTGTACTA
CGTGTACTCCCCCTCTCGGAGCTTCTCCTACTTTTATCCTTTCAGACTG
CCAATCAAGGGCGTGCCTATCGAGCTGCAGGTGGAGTGCTTTACCTGGG
ATAAGAAGCTGTGGTGCAGGCACTTCTGCGTGCTGGCAGACTCTGAGAG
CGGAGGACACATCACCCACTCCGGAATGGTGGGCATGGGCGTGTCTTGT
ACCGTGACACGCGAGGACGGCACAGGGGGAGGCGGCTCTGGAGGTGGGG
GGTCCGGTGGCGGCGGAAGCGCGGCCGCGGATGTGCAGCTGCAGGAGAG
CGGCGGCGGATCTGTGCAAGCTGGCGGAAGCCTGAGACTGTCTTGCGCC
GCTTCTGGCTACACCCACAGCAGCTATTGCATGGCCTGGTTCCGGCAGG
CCCCTGGCAGAGAACGGGAAGGCGTGGCCAGCATCGACAGCGACGGCAC
CACCAGCTACGCCGATAGCGTCAAGGGCAGATTCACCATCAGCCAGGAC
AATGCCAAAAACACCCTGTACCTGCAGATGAACAGCCTGAAGCCCGAGG
ACACAGCCATGTACTACTGTGCCGCCAGATTTGGCCCTATGGGCTGCGT
GGACCTGTCCACCCTCTCCTTCGGCCACTGGGGACAGGGCACACAGGTG
ACAGTGTCCATCACCGGCGGCGGCTCCGGCGGCGGCAGCTATCCCTATG
ATGTGCCCGACTATGCGTGA.

By “MeV-Hwtc18-CD4 (03F11) polypeptide” is meant a polypeptide or fragment thereof having at least about 85% amino acid sequence identity to the sequence provided below, and that has activity associated with fusion of a Lentivirus envelope containing an F protein domain with a membrane of a cell surface-expressing a CD4 polypeptide. In some instances the CD4 polypeptide is a murine or human CD4 polypeptide. In embodiments, the MeV-Hwtc18-CD4 (03F11) polypeptide functions in combination with an MeV-Fc30 polypeptide to fuse a lentivirus lipid envelope with a membrane (e.g., a cell membrane). An exemplary MeV-Hwtc18-CD4 (03F11) polypeptide sequence is provided below.

MGSRIVINREHLMIDRPYVLLAVLFVMFLSLIGLLAIAGIRLHRAAIYT
AEIHKSLSTNLDVTNSIEHQVKDVLTPLFKIIGDEVGLRTPQRFTDLVK
FISDKIKFLNPDREYDFRDLTWCINPPERIKLDYDQYCADVAAEELMNA
LVNSTLLEARATNQFLAVSKGNCSGPTTIRGQFSNMSLSLLDLYLSRGY
NVSSIVTMTSQGMYGGTYLVGKPNLSSKGSELSQLSMHRVFEVGVIRNP
GLGAPVFHMTNYFEQPVSNDFSNCMVALGELKFAALCHREDSITIPYQG
SGKGVSFQLVKLGVWKSPTDMRSWVPLSTDDPVIDRLYLSSHRGVIADN
QAKWAVPTTRTDDKLRMETCFQQACKGKNQALCENPEWAPLKDNRIPSY
GVLSVNLSLTVELKIKIASGFGPLITHGSGMDLYKTNHNNVYWLTIPPM
KNLALGVINTLEWIPRFKVSPALFTVPIKEAGEDCHAPTYLPAEVDGDV
KLSSNLVILPGQDLQYVLATYDTSRVEHAVVYYVYSPSRSFSYFYPFRL
PIKGVPIELQVECFTWDKKLWCRHFCVLADSESGGHITHSGMVGMGVSC
TVTREDGTGGGGSGGGGSGGGGSAAAEVQLVESGGGSVQPGGSLTLSCG
TSGRTFNVMGWFRQAPGKEREFVAAVRWSSTGIYYTQYADSVKSRFTIS
RDNAKNTVYLEMNSLKPEDTAVYYCAADTYNSNPARWDGYDFRGQGTQV
TVSSGGGSGGGSYPYDVPDYA.

In the above polypeptide sequence, the cytoplasmic domain is underlined, the transmembrane domain is in italics, the extracellular domain is in plain text, the (G3S)3 linker is in bold, the NotI site is in bold underline, the VHH domain is in bold italic, the (G3S)2 linker is in underlined italics, and the HA tag is double-underlined. By “MeV-Hwtc18-CD4 (03F11) polynucleotide” is meant a polynucleotide encoding an MeV-Hwtc18-CD4 (03F11) polypeptide. An exemplary MeV-Hwtc18-CD4 (03F11) polynucleotide sequence is provided below.

GAAGAACCTGGCCCTGGGCGTGATCAATACCCTGGAGTGGATCCCCAGG
TTTAAGGTGTCCCCTGCCCTGTTCACCGTGCCAATCAAGGAGGCCGGCG
AGGATTGTCACGCCCCAACATACCTGCCCGCCGAGGTGGACGGCGATGT
GAAGCTGAGCTCCAACCTGGTCATCCTGCCTGGCCAGGACCTGCAGTAC
GTGCTGGCCACCTATGATACAAGCCGCGTGGAGCACGCCGTGGTGTACT
ACGTGTACTCCCCCTCTCGGAGCTTCTCCTACTTTTATCCTTTCAGACT
GCCAATCAAGGGCGTGCCTATCGAGCTGCAGGTGGAGTGCTTTACCTGG
GATAAGAAGCTGTGGTGCAGGCACTTCTGCGTGCTGGCAGACTCTGAGA
GCGGAGGACACATCACCCACTCCGGAATGGTGGGCATGGGCGTGTCTTG
TACCGTGACACGCGAGGACGGCACAGGGGGAGGCGGCTCTGGAGGTGGG
GGGTCCGGTGGCGGCGGAAGCGCGGCCGCGGAGGTGCAGCTGGTCGAGA
GCGGAGGAGGCAGCGTGCAGCCCGGCGGATCTCTGACCCTGAGCTGTGG
CACAAGCGGCAGAACCTTTAACGTGATGGGCTGGTTCAGACAGGCCCCT
GGCAAGGAAAGAGAGTTCGTGGCCGCTGTGCGGTGGTCCTCTACAGGCA
TCTACTATACACAGTACGCCGACAGCGTTAAAAGCAGATTCACCATCAG
CCGGGACAACGCCAAGAACACCGTGTACCTGGAAATGAACAGCCTGAAG
CCAGAGGATACAGCCGTGTACTACTGCGCCGCCGATACCTACAACTCTA
ATCCTGCTAGATGGGACGGCTACGACTTCCGGGGCCAGGGCACCCAAGT
GACCGTGTCCAGCGGCGGCGGCTCCGGCGGCGGCAGCTATCCCTATGAT
GTGCCCGACTATGCGTGA.

By “MeV-Hwtc18-CD8a (R3HCD27) polypeptide” is meant a polypeptide or fragment thereof having at least about 85% amino acid sequence identity to the sequence provided below, and that has activity associated with fusion of a Lentivirus envelope containing an F protein domain with a membrane of a cell surface-expressing a CD8 polypeptide. In some instances the CD8 polypeptide is a murine or human CD8 polypeptide. In embodiments, the MeV-Hwtc18-CD8a (R3HCD27) polypeptide functions in combination with an MeV-Fc30 polypeptide to fuse a Lentivirus lipid envelope with a membrane (e.g., a cell membrane). An exemplary MeV-Hwtc18-CD8a (R3HCD27) polypeptide sequence is provided below.

MGSRIVINREHLMIDRPYVLLAVLFVMFLSLIGLLAIAGIRLHRAAIYT
AEIHKSLSTNLDVTNSIEHQVKDVLTPLFKIIGDEVGLRTPQRFTDLVK
FISDKIKFLNPDREYDFRDLTWCINPPERIKLDYDQYCADVAAEELMNA
LVNSTLLEARATNQFLAVSKGNCSGPTTIRGQFSNMSLSLLDLYLSRGY
NVSSIVTMTSQGMYGGTYLVGKPNLSSKGSELSQLSMHRVFEVGVIRNP
GLGAPVFHMTNYFEQPVSNDFSNCMVALGELKFAALCHREDSITIPYQG
SGKGVSFQLVKLGVWKSPTDMRSWVPLSTDDPVIDRLYLSSHRGVIADN
QAKWAVPTTRTDDKLRMETCFQQACKGKNQALCENPEWAPLKDNRIPSY
GVLSVNLSLTVELKIKIASGFGPLITHGSGMDLYKTNHNNVYWLTIPPM
KNLALGVINTLEWIPRFKVSPALFTVPIKEAGEDCHAPTYLPAEVDGDV
KLSSNLVILPGQDLQYVLATYDTSRVEHAVVYYVYSPSRSFSYFYPFRL
PIKGVPIELQVECFTWDKKLWCRHFCVLADSESGGHITHSGMVGMGVSC
TVTREDGTGGGGSGGGGSGGGGSAAAQVQLQESGGGSVQPGGSLRLSCA
ASGFTFDDYAMSWVRQVPGKGLEWVSTINWNGGSAEYAEPVKGRFTISR
DNAKNTVYLQMNSLKLEDTAVYYCAKDADLVWYNLSTGQGTQVTVSSGG
GSGGGSYPYDVPDYA.

In the above polypeptide sequence, the cytoplasmic domain is underlined, the transmembrane domain is in italics, the extracellular domain is in plain text, the (G3S)3 linker is in bold, the NotI site is in bold underline, the VHH domain is in bold italic, the (G3S)2 linker is in underlined italics, and the HA tag is double-underlined.

By “MeV-Hwtc18-CD8a (R3HCD27) polynucleotide” is meant a polynucleotide encoding an MeV-Hwtc18-CD8a (R3HCD27) polypeptide. An exemplary MeV-Hwtc18-CD8a (R3HCD27) polynucleotide sequence is provided below.

AGGTGGACGGCGATGTGAAGCTGAGCTCCAACCTGGTCATCCTGCCTGG
CCAGGACCTGCAGTACGTGCTGGCCACCTATGATACAAGCCGCGTGGAG
CACGCCGTGGTGTACTACGTGTACTCCCCCTCTCGGAGCTTCTCCTACT
TTTATCCTTTCAGACTGCCAATCAAGGGCGTGCCTATCGAGCTGCAGGT
GGAGTGCTTTACCTGGGATAAGAAGCTGTGGTGCAGGCACTTCTGCGTG
CTGGCAGACTCTGAGAGCGGAGGACACATCACCCACTCCGGAATGGTGG
GCATGGGCGTGTCTTGTACCGTGACACGCGAGGACGGCACAGGGGGAGG
CGGCTCTGGAGGTGGGGGGTCCGGTGGCGGCGGAAGCGCGGCCGCGCAA
GTGCAGCTGCAGGAGAGCGGCGGAGGCAGCGTGCAGCCTGGCGGAAGCC
TGAGACTGTCCTGCGCCGCTTCTGGCTTTACCTTCGACGACTACGCCAT
GAGCTGGGTGCGGCAGGTTCCAGGCAAGGGCCTGGAATGGGTCAGCACA
ATCAACTGGAACGGCGGATCTGCTGAGTACGCCGAGCCTGTGAAGGGCA
GATTCACAATCAGCAGAGATAATGCCAAAAACACCGTGTATCTGCAGAT
GAACAGCCTCAAGCTGGAAGATACAGCCGTGTACTACTGTGCCAAGGAC
GCCGACCTGGTGTGGTACAACCTGAGCACCGGCCAGGGCACCCAGGTGA
CCGTGTCCAGCGCCGCTGCCTACCCCTACGACGTGCCTGATTACGGCTC
TGGCGGCGGCTCCGGCGGCGGCAGCTATCCCTATGATGTGCCCGACTAT
GCGTGA.

By “CD46 polypeptide” is meant a polypeptide or fragment thereof having at least about 85% amino acid sequence identity to NCBI Reference Sequence No. NP_758865.1, and that extends viral half-life in a subject. An exemplary CD46 polypeptide sequence is provided below:

MEPPGRRECPFPSWRFPGLLLAAMVLLLYSFSDACEEPPTFEAMELIGK
PKPYYEIGERVDYKCKKGYFYIPPLATHTICDRNHTWLPVSDDACYRET
CPYIRDPLNGQAVPANGTYEFGYQMHFICNEGYYLIGEEILYCELKGSV
AIWSGKPPICEKVLCTPPPKIKNGKHTFSEVEVFEYLDAVTYSCDPAPG
PDPFSLIGESTIYCGDNSVWSRAAPECKVVKCRFPVVENGKQISGFGKK
FYYKATVMFECDKGFYLDGSDTIVCDSNSTWDPPVPKCLKVSTSSTTKS
PASSASGYPKPEEGILDSLDVWVIAVIVIAIVVGVAVICVVPYRYLQRR
KKKGTYLTDETHREVKFTSL.

By “CD46 polynucleotide” is meant a polynucleotide that encodes a CD46 polypeptide or a fragment thereof. An exemplary CD46 polynucleotide sequence is provided at base pairs 160 to 1251 of NCBI Reference Sequence No.: NM_172355.3. An exemplary CD46 polynucleotide sequence is provided below:

GCGGCTCGGGCCACGCCCACCTGTCCTGCAGCACTGGATGCTTTGTGAG
TTGGGGATTGTTGCGTCCCATATCTGGACCCAGAAGGGACTTCCCTGCT
CGGCTGGCTCTCGGTTTCTCTGCTTTCCTCCGGAGAAATAACAGCGTCT
TCCGCGCCGCGCATGGAGCCTCCCGGCCGCCGCGAGTGTCCCTTTCCTT
CCTGGCGCTTTCCTGGGTTGCTTCTGGCGGCCATGGTGTTGCTGCTGTA
CTCCTTCTCCGATGCCTGTGAGGAGCCACCAACATTTGAAGCTATGGAG
CTCATTGGTAAACCAAAACCCTACTATGAGATTGGTGAACGAGTAGATT
ATAAGTGTAAAAAAGGATACTTCTATATACCTCCTCTTGCCACCCATAC
TATTTGTGATCGGAATCATACATGGCTACCTGTCTCAGATGACGCCTGT
TATAGAGAAACATGTCCATATATACGGGATCCTTTAAATGGCCAAGCAG
TCCCTGCAAATGGGACTTACGAGTTTGGTTATCAGATGCACTTTATTTG
TAATGAGGGTTATTACTTAATTGGTGAAGAAATTCTATATTGTGAACTT
AAAGGATCAGTAGCAATTTGGAGCGGTAAGCCCCCAATATGTGAAAAGG
TTTTGTGTACACCACCTCCAAAAATAAAAAATGGAAAACACACCTTTAG
TGAAGTAGAAGTATTTGAGTATCTTGATGCAGTAACTTATAGTTGTGAT
CCTGCACCTGGACCAGATCCATTTTCACTTATTGGAGAGAGCACGATTT
ATTGTGGTGACAATTCAGTGTGGAGTCGTGCTGCTCCAGAGTGTAAAGT
GGTCAAATGTCGATTTCCAGTAGTCGAAAATGGAAAACAGATATCAGGA
TTTGGAAAAAAATTTTACTACAAAGCAACAGTTATGTTTGAATGCGATA
AGGGTTTTTACCTCGATGGCAGCGACACAATTGTCTGTGACAGTAACAG
TACTTGGGATCCCCCAGTTCCAAAGTGTCTTAAAGTGTCGACTTCTTCC
ACTACAAAATCTCCAGCGTCCAGTGCCTCAGGATATCCTAAACCTGAGG
AAGGAATACTTGACAGTTTGGATGTTTGGGTCATTGCTGTGATTGTTAT
TGCCATAGTTGTTGGAGTTGCAGTAATTTGTGTTGTCCCGTACAGATAT
CTTCAAAGGAGGAAGAAGAAAGGCACATACCTAACTGATGAGACCCACA
GAGAAGTAAAATTTACTTCTCTCTGAGAAGGAGAGATGAGAGAAAGGTT
TGCTTTTATCATTAAAAGGAAAGCAGATGGTGGAGCTGAATATGCCACT
TACCAGACTAAATCAACCACTCCAGCAGAGCAGAGAGGCTGAATAGATT
CCACAACCTGGTTTGCCAGTTCATCTTTTGACTCTATTAAAATCTTCAA
TAGTTGTTATTCTGTAGTTTCACTCTCATGAGTGCAACTGTGGCTTAGC
TAATATTGCAATGTGGCTTGAATGTAGGTAGCATCCTTTGATGCTTCTT
TGAAACTTGTATGAATTTGGGTATGAACAGATTGCCTGCTTTCCCTTAA
ATAACACTTAGATTTATTGGACCAGTCAGCACAGCATGCCTGGTTGTAT
TAAAGCAGGGATATGCTGTATTTTATAAAATTGGCAAAATTAGAGAAAT
ATAGTTCACAATGAAATTATATTTTCTTTGTAAAGAAAGTGGCTTGAAA
TCTTTTTTGTTCAAAGATTAATGCCAACTCTTAAGATTATTCTTTCACC
AACTATAGAATGTATTTTATATATCGTTCATTGTAAAAAGCCCTTAAAA
ATATGTGTATACTACTTTGGCTCTTGTGCATAAAAACAAGAACACTGAA
AATTGGGAATATGCACAAACTTGGCTTCTTTAACCAAGAATATTATTGG
AAAATTCTCTAAAAGTTAATAGGGTAAATTCTCTATTTTTTGTAATGTG
TTCGGTGATTTCAGAAAGCTAGAAAGTGTATGTGTGGCATTTGTTTTCA
CTTTTTAAAACATCCCTAACTGATCGAATATATCAGTAATTTCAGAATC
AGATGCATCCTTTCATAAGAAGTGAGAGGACTCTGACAGCCATAACAGG
AGTGCCACTTCATGGTGCGAAGTGAACACTGTAGTCTTGTTGTTTTCCC
AAAGAGAACTCCGTATGTTCTCTTAGGTTGAGTAACCCACTCTGAATTC
TGGTTACATGTGTTTTTCTCTCCCTCCTTAAATAAAGAGAGGGGTTAAA
CATGCCCTCTAAAAGTAGGTGGTTTTGAAGAGAATAAATTCATCAGATA
ACCTCAAGTCACATGAGAATCTTAGTCCATTTACATTGCCTTGGCTAGT
AAAAGCCATCTATGTATATGTCTTACCTCATCTCCTAAAAGGCAGAGTA
CAAAGTAAGCCATGTATCTCAGGAAGGTAACTTCATTTTGTCTATTTGC
TGTTGATTGTACCAAGGGATGGAAGAAGTAAATATAGCTCAGGTAGCAC
TTTATACTCAGGCAGATCTCAGCCCTCTACTGAGTCCCTTAGCCAAGCA
GTTTCTTTCAAAGAAGCCAGCAGGCGAAAAGCAGGGACTGCCACTGCAT
TTCATATCACACTGTTAAAAGTTGTGTTTTGAAATTTTATGTTTAGTTG
CACAAATTGGGCCAAAGAAACATTGCCTTGAGGAAGATATGATTGGAAA
ATCAAGAGTGTAGAAGAATAAATACTGTTTTACTGTCCAAAGACATGTT
TATAGTGCTCTGTAAATGTTCCTTTCCTTTGTAGTCTCTGGCAAGATGC
TTTAGGAAGATAAAAGTTTGAGGAGAACAAACAGGAATTCTGAATTAAG
CACAGAGTTGAAGTTTATACCCGTTTCACATGCTTTTCAAGAATGTCGC
AATTACTAAGAAGCAGATAATGGTGTTTTTTAGAAACCTAATTGAAGTA
TATTCAACCAAATACTTTAATGTATAAAATAAATATTATACAATATACT
TGTATAGCAGTTTCTGCTTCACATTTGATTTTTTCAAATTTAATATTTA
TATTAGAGATCTATATATGTATAAATATGTATTTTGTCAAATTTGTTAC
TTAAATATATAGAGACCAGTTTTCTCTGGAAGTTTGTTTAAATGACAGA
AGCGTATATGAATTCAAGAAAATTTAAGCTGCAAAAATGTATTTGCTAT
AAAATGAGAAGTCTCACTGATAGAGGTTCTTTATTGCTCATTTTTTAAA
AAATGGACTCTTGAAATCTGTTAAAATAAAATTGTACATTTGGA.

By “CD47 polypeptide” is meant a polypeptide or fragment thereof having at least about 85% amino acid sequence identity to NCBI Reference Sequence No. NP_001768.1, and that that extends viral half-life in a subject. An exemplary CD47 polypeptide sequence is provided below:

MWPLVAALLLGSACCGSAQLLFNKTKSVEFTFCNDTVVIPCFVTNMEAQ
NTTEVYVKWKFKGRDIYTFDGALNKSTVPTDFSSAKIEVSQLLKGDASL
KMDKSDAVSHTGNYTCEVTELTREGETIIELKYRVVSWFSPNENILIVI
FPIFAILLFWGQFGIKTLKYRSGGMDEKTIALLVAGLVITVIVIVGAIL
FVPGEYSLKNATGLGLIVTSTGILILLHYYVFSTAIGLTSFVIAILVIQ
VIAYILAVVGLSLCIAACIPMHGPLLISGLSILALAQLLGLVYMKFVAS
NQKTIQPPRKAVEEPLNAFKESKGMMNDE.

By “CD47 polynucleotide” is meant a polynucleotide that encodes a CD47 polypeptide or a fragment thereof. An exemplary CD47 polynucleotide sequence is provided at base pairs 124 to 1095 of NCBI Reference Sequence No.: NM_001777.4. An exemplary CD47 polynucleotide sequence is provided below:

GCAGCCTGGGCAGTGGGTCCTGCCTGTGACGCGCGGCGGCGGTCGGTCCTGCCTGTAACGGCGG
CGGCGGCTGCTGCTCCGGACACCTGCGGCGGCGGCGGCGACCCCGCGGCGGGCGCGGAGATGTG
GCCCCTGGTAGCGGCGCTGTTGCTGGGCTCGGCGTGCTGCGGATCAGCTCAGCTACTATTTAAT
AAAACAAAATCTGTAGAATTCACGTTTTGTAATGACACTGTCGTCATTCCATGCTTTGTTACTA
ATATGGAGGCACAAAACACTACTGAAGTATACGTAAAGTGGAAATTTAAAGGAAGAGATATTTA
CACCTTTGATGGAGCTCTAAACAAGTCCACTGTCCCCACTGACTTTAGTAGTGCAAAAATTGAA
GTCTCACAATTACTAAAAGGAGATGCCTCTTTGAAGATGGATAAGAGTGATGCTGTCTCACACA
CAGGAAACTACACTTGTGAAGTAACAGAATTAACCAGAGAAGGTGAAACGATCATCGAGCTAAA
ATATCGTGTTGTTTCATGGTTTTCTCCAAATGAAAATATTCTTATTGTTATTTTCCCAATTTTT
GCTATACTCCTGTTCTGGGGACAGTTTGGTATTAAAACACTTAAATATAGATCCGGTGGTATGG
ATGAGAAAACAATTGCTTTACTTGTTGCTGGACTAGTGATCACTGTCATTGTCATTGTTGGAGC
CATTCTTTTCGTCCCAGGTGAATATTCATTAAAGAATGCTACTGGCCTTGGTTTAATTGTGACT
TCTACAGGGATATTAATATTACTTCACTACTATGTGTTTAGTACAGCGATTGGATTAACCTCCT
TCGTCATTGCCATATTGGTTATTCAGGTGATAGCCTATATCCTCGCTGTGGTTGGACTGAGTCT
CTGTATTGCGGCGTGTATACCAATGCATGGCCCTCTTCTGATTTCAGGTTTGAGTATCTTAGCT
CTAGCACAATTACTTGGACTAGTTTATATGAAATTTGTGGCTTCCAATCAGAAGACTATACAAC
CTCCTAGGAAAGCTGTAGAGGAACCCCTTAATGCATTCAAAGAATCAAAAGGAATGATGAATGA
TGAATAACTGAAGTGAAGTGATGGACTCCGATTTGGAGAGTAGTAAGACGTGAAAGGAATACAC
TTGTGTTTAAGCACCATGGCCTTGATGATTCACTGTTGGGGAGAAGAAACAAGAAAAGTAACTG
GTTGTCACCTATGAGACCCTTACGTGATTGTTAGTTAAGTTTTTATTCAAAGCAGCTGTAATTT
AGTTAATAAAATAATTATGATCTATGTTGTTTGCCCAATTGAGATCCAGTTTTTTGTTGTTATT
TTTAATCAATTAGGGGCAATAGTAGAATGGACAATTTCCAAGAATGATGCCTTTCAGGTCCTAG
GGCCTCTGGCCTCTAGGTAACCAGTTTAAATTGGTTCAGGGTGATAACTACTTAGCACTGCCCT
GGTGATTACCCAGAGATATCTATGAAAACCAGTGGCTTCCATCAAACCTTTGCCAACTCAGGTT
CACAGCAGCTTTGGGCAGTTATGGCAGTATGGCATTAGCTGAGAGGTGTCTGCCACTTCTGGGT
CAATGGAATAATAAATTAAGTACAGGCAGGAATTTGGTTGGGAGCATCTTGTATGATCTCCGTA
TGATGTGATATTGATGGAGATAGTGGTCCTCATTCTTGGGGGTTGCCATTCCCACATTCCCCCT
TCAACAAACAGTGTAACAGGTCCTTCCCAGATTTAGGGTACTTTTATTGATGGATATGTTTTCC
TTTTATTCACATAACCCCTTGAAACCCTGTCTTGTCCTCCTGTTACTTGCTTCTGCTGTACAAG
ATGTAGCACCTTTTCTCCTCTTTGAACATGGTCTAGTGACACGGTAGCACCAGTTGCAGGAAGG
AGCCAGACTTGTTCTCAGAGCACTGTGTTCACACTTTTCAGCAAAAATAGCTATGGTTGTAACA
TATGTATTCCCTTCCTCTGATTTGAAGGCAAAAATCTACAGTGTTTCTTCACTTCTTTTCTGAT
CTGGGGCATGAAAAAAGCAAGATTGAAATTTGAACTATGAGTCTCCTGCATGGCAACAAAATGT
GTGTCACCATCAGGCCAACAGGCCAGCCCTTGAATGGGGATTTATTACTGTTGTATCTATGTTG
CATGATAAACATTCATCACCTTCCTCCTGTAGTCCTGCCTCGTACTCCCCTTCCCCTATGATTG
AAAAGTAAACAAAACCCACATTTCCTATCCTGGTTAGAAGAAAATTAATGTTCTGACAGTTGTG
ATCGCCTGGAGTACTTTTAGACTTTTAGCATTCGTTTTTTACCTGTTTGTGGATGTGTGTTTGT
ATGTGCATACGTATGAGATAGGCACATGCATCTTCTGTATGGACAAAGGTGGGGTACCTACAGG
AGAGCAAAGGTTAATTTTGTGCTTTTAGTAAAAACATTTAAATACAAAGTTCTTTATTGGGTGG
AATTATATTTGATGCAAATATTTGATCACTTAAAACTTTTAAAACTTCTAGGTAATTTGCCACG
CTTTTTGACTGCTCACCAATACCCTGTAAAAATACGTAATTCTTCCTGTTTGTGTAATAAGATA
TTCATATTTGTAGTTGCATTAATAATAGTTATTTCTTAGTCCATCAGATGTTCCCGTGTGCCTC
TTTTATGCCAAATTGATTGTCATATTTCATGTTGGGACCAAGTAGTTTGCCCATGGCAAACCTA
AATTTATGACCTGCTGAGGCCTCTCAGAAAACTGAGCATACTAGCAAGACAGCTCTTCTTGAAA
AAAAAAATATGTATACACAAATATATACGTATATCTATATATACGTATGTATATACACACATGT
ATATTCTTCCTTGATTGTGTAGCTGTCCAAAATAATAACATATATAGAGGGAGCTGTATTCCTT
TATACAAATCTGATGGCTCCTGCAGCACTTTTTCCTTCTGAAAATATTTACATTTTGCTAACCT
AGTTTGTTACTTTAAAAATCAGTTTTGATGAAAGGAGGGAAAAGCAGATGGACTTGAAAAAGAT
CCAAGCTCCTATTAGAAAAGGTATGAAAATCTTTATAGTAAAATTTTTTATAAACTAAAGTTGT
ACCTTTTAATATGTAGTAAACTCTCATTTATTTGGGGTTCGCTCTTGGATCTCATCCATCCATT
GTGTTCTCTTTAATGCTGCCTGCCTTTTGAGGCATTCACTGCCCTAGACAATGCCACCAGAGAT
AGTGGGGGAAATGCCAGATGAAACCAACTCTTGCTCTCACTAGTTGTCAGCTTCTCTGGATAAG
TGACCACAGAAGCAGGAGTCCTCCTGCTTGGGCATCATTGGGCCAGTTCCTTCTCTTTAAATCA
GATTTGTAATGGCTCCCAAATTCCATCACATCACATTTAAATTGCAGACAGTGTTTTGCACATC
ATGTATCTGTTTTGTCCCATAATATGCTTTTTACTCCCTGATCCCAGTTTCTGCTGTTGACTCT
TCCATTCAGTTTTATTTATTGTGTGTTCTCACAGTGACACCATTTGTCCTTTTCTGCAACAACC
TTTCCAGCTACTTTTGCCAAATTCTATTTGTCTTCTCCTTCAAAACATTCTCCTTTGCAGTTCC
TCTTCATCTGTGTAGCTGCTCTTTTGTCTCTTAACTTACCATTCCTATAGTACTTTATGCATCT
CTGCTTAGTTCTATTAGTTTTTTGGCCTTGCTCTTCTCCTTGATTTTAAAATTCCTTCTATAGC
TAGAGCTTTTCTTTCTTTCATTCTCTCTTCCTGCAGTGTTTTGCATACATCAGAAGCTAGGTAC
ATAAGTTAAATGATTGAGAGTTGGCTGTATTTAGATTTATCACTTTTTAATAGGGTGAGCTTGA
GAGTTTTCTTTCTTTCTGTTTTTTTTTTTTGTTTTTTTTTTTTTTTTTTTTTTTTTTTTTTTTG
ACTAATTTCACATGCTCTAAAAACCTTCAAAGGTGATTATTTTTCTCCTGGAAACTCCAGGTCC
ATTCTGTTTAAATCCCTAAGAATGTCAGAATTAAAATAACAGGGCTATCCCGTAATTGGAAATA
TTTCTTTTTTCAGGATGCTATAGTCAATTTAGTAAGTGACCACCAAATTGTTATTTGCACTAAC
AAAGCTCAAAACACGATAAGTTTACTCCTCCATCTCAGTAATAAAAATTAAGCTGTAATCAACC
TTCTAGGTTTCTCTTGTCTTAAAATGGGTATTCAAAAATGGGGATCTGTGGTGTATGTATGGAA
ACACATACTCCTTAATTTACCTGTTGTTGGAAACTGGAGAAATGATTGTCGGGCAACCGTTTAT
TTTTTATTGTATTTTATTTGGTTGAGGGATTTTTTTATAAACAGTTTTACTTGTGTCATATTTT
AAAATTACTAACTGCCATCACCTGCTGGGGTCCTTTGTTAGGTCATTTTCAGTGACTAATAGGG
ATAATCCAGGTAACTTTGAAGAGATGAGCAGTGAGTGACCAGGCAGTTTTTCTGCCTTTAGCTT
TGACAGTTCTTAATTAAGATCATTGAAGACCAGCTTTCTCATAAATTTCTCTTTTTGAAAAAAA
GAAAGCATTTGTACTAAGCTCCTCTGTAAGACAACATCTTAAATCTTAAAAGTGTTGTTATCAT
GACTGGTGAGAGAAGAAAACATTTTGTTTTTATTAAATGGAGCATTATTTACAAAAAGCCATTG
TTGAGAATTAGATCCCACATCGTATAAATATCTATTAACCATTCTAAATAAAGAGAACTCCAGT
GTTGCTATGTGCAAGATCCTCTCTTGGAGCTTTTTTGCATAGCAATTAAAGGTGTGCTATTTGT
CAGTAGCCATTTTTTTGCAGTGATTTGAAGACCAAAGTTGTTTTACAGCTGTGTTACCGTTAAA
GGTTTTTTTTTTTATATGTATTAAATCAATTTATCACTGTTTAAAGCTTTGAATATCTGCAATC
TTTGCCAAGGTACTTTTTTATTTAAAAAAAAACATAACTTTGTAAATATTACCCTGTAATATTA
TATATACTTAATAAAACATTTTAAGCTATTTTGTTGGGCTATTTCTATTGCTGCTACAGCAGAC
CACAAGCACATTTCTGAAAAATTTAATTTATTAATGTATTTTTAAGTTGCTTATATTCTAGGTA
ACAATGTAAAGAATGATTTAAAATATTAATTATGAATTTTTTGAGTATAATACCCAATAAGCTT
TTAATTAGAGCAGAGTTTTAATTAAAAGTTTTAAATCAGTCCAA.

By “CD55 polypeptide” is meant a polypeptide or fragment thereof having at least about 85% amino acid sequence identity to NCBI Reference Sequence No. NP_001108224.1, and that extends viral half-life in a subject. An exemplary CD55 polypeptide sequence is provided below:

MTVARPSVPAALPLLGELPRLLLLVLLCLPAVWGDCGLPPDVPNAQPAL
EGRTSFPEDTVITYKCEESFVKIPGEKDSVICLKGSQWSDIEEFCNRSC
EVPTRLNSASLKQPYITQNYFPVGTVVEYECRPGYRREPSLSPKLTCLQ
NLKWSTAVEFCKKKSCPNPGEIRNGQIDVPGGILFGATISFSCNTGYKL
FGSTSSFCLISGSSVQWSDPLPECREIYCPAPPQIDNGIIQGERDHYGY
RQSVTYACNKGFTMIGEHSIYCTVNNDEGEWSGPPPECRGKSLTSKVPP
TVQKPTTVNVPTTEVSPTSQKTTTKTTTPNAQATRSTPVSRTTKHFHET
TPNKGSGTTSGTTRLLSGSRPVTQAGMRWCDRSSLQSRTPGFKRSFHFS
LPSSWYYRAHVFHVDRFAWDASNHGLADLAKEELRRKYTQVYRLFLVS.

By “CD55 polynucleotide” is meant a polynucleotide that encodes a CD55 polypeptide or a fragment thereof. An exemplary CD55 polynucleotide sequence is provided at base pairs 89 to 1411 of NCBI Reference Sequence No.: NM_001114752.3. An exemplary CD55 polynucleotide sequence is provided below:

CTGCTTACTGCAACTCGCTCCGGCCGCTGGGCGTAGCTGCGACTCGGCG
GAGTCCCGGCGGCGCGTCCTTGTTCTAACCCGGCGCGCCATGACCGTCG
CGCGGCCGAGCGTGCCCGCGGCGCTGCCCCTCCTCGGGGAGCTGCCCCG
GCTGCTGCTGCTGGTGCTGTTGTGCCTGCCGGCCGTGTGGGGTGACTGT
GGCCTTCCCCCAGATGTACCTAATGCCCAGCCAGCTTTGGAAGGCCGTA
CAAGTTTTCCCGAGGATACTGTAATAACGTACAAATGTGAAGAAAGCTT
TGTGAAAATTCCTGGCGAGAAGGACTCAGTGATCTGCCTTAAGGGCAGT
CAATGGTCAGATATTGAAGAGTTCTGCAATCGTAGCTGCGAGGTGCCAA
CAAGGCTAAATTCTGCATCCCTCAAACAGCCTTATATCACTCAGAATTA
TTTTCCAGTCGGTACTGTTGTGGAATATGAGTGCCGTCCAGGTTACAGA
AGAGAACCTTCTCTATCACCAAAACTAACTTGCCTTCAGAATTTAAAAT
GGTCCACAGCAGTCGAATTTTGTAAAAAGAAATCATGCCCTAATCCGGG
AGAAATACGAAATGGTCAGATTGATGTACCAGGTGGCATATTATTTGGT
GCAACCATCTCCTTCTCATGTAACACAGGGTACAAATTATTTGGCTCGA
CTTCTAGTTTTTGTCTTATTTCAGGCAGCTCTGTCCAGTGGAGTGACCC
GTTGCCAGAGTGCAGAGAAATTTATTGTCCAGCACCACCACAAATTGAC
AATGGAATAATTCAAGGGGAACGTGACCATTATGGATATAGACAGTCTG
TAACGTATGCATGTAATAAAGGATTCACCATGATTGGAGAGCACTCTAT
TTATTGTACTGTGAATAATGATGAAGGAGAGTGGAGTGGCCCACCACCT
GAATGCAGAGGAAAATCTCTAACTTCCAAGGTCCCACCAACAGTTCAGA
AACCTACCACAGTAAATGTTCCAACTACAGAAGTCTCACCAACTTCTCA
GAAAACCACCACAAAAACCACCACACCAAATGCTCAAGCAACACGGAGT
ACACCTGTTTCCAGGACAACCAAGCATTTTCATGAAACAACCCCAAATA
AAGGAAGTGGAACCACTTCAGGTACTACCCGTCTTCTATCTGGTTCTCG
TCCTGTCACCCAGGCTGGTATGCGGTGGTGTGATCGTAGCTCACTGCAG
TCTCGAACTCCTGGGTTCAAGCGATCCTTCCACTTCAGCCTCCCAAGTA
GCTGGTACTACAGGGCACACGTGTTTCACGTTGACAGGTTTGCTTGGGA
CGCTAGTAACCATGGGCTTGCTGACTTAGCCAAAGAAGAGTTAAGAAGA
AAATACACACAAGTATACAGACTGTTCCTAGTTTCTTAGACTTATCTGC
ATATTGGATAAAATAAATGCAATTGTGCTCTTCATTTAGGATGCTTTCA
TTGTCTTTAAGATGTGTTAGGAATGTCAACAGAGCAAGGAGAAAAAAGG
CAGTCCTGGAATCACATTCTTAGCACACCTACACCTCTTGAAAATAGAA
CAACTTGCAGAATTGAGAGTGATTCCTTTCCTAAAAGTGTAAGAAAGCA
TAGAGATTTGTTCGTATTTAGAATGGGATCACGAGGAAAAGAGAAGGAA
AGTGATTTTTTTCCACAAGATCTGTAATGTTATTTCCACTTATAAAGGA
AATAAAAAATGAAAAACATTATTTGGATATCAAAAGCAAATAAAAACCC
AATTCAGTCTCTTCTAAGCAAAATTGCTAAAGAGAGATGAACCACATTA
TAAAGTAATCTTTGGCTGTAAGGCATTTTCATCTTTCCTTCGGGTTGGC
AAAATATTTTAAAGGTAAAACATGCTGGTGAACCAGGGGTGTTGATGGT
GATAAGGGAGGAATATAGAATGAAAGACTGAATCTTCCTTTGTTGCACA
AATAGAGTTTGGAAAAAGCCTGTGAAAGGTGTCTTCTTTGACTTAATGT
CTTTAAAAGTATCCAGAGATACTACAATATTAACATAAGAAAAGATTAT
ATATTATTTCTGAATCGAGATGTCCATAGTCAAATTTGTAAATCTTATT
CTTTTGTAATATTTATTTATATTTATTTATGACAGTGAACATTCTGATT
TTACATGTAAAACAAGAAAAGTTGAAGAAGATATGTGAAGAAAAATGTA
TTTTTCCTAAATAGAAATAAATGATCCCATTTTTTGGTATCATGTAGTA
TGTGAAATTTATTCTTAAACGTGACTACTTTATTTCTAAATAAGAAATT
CCCTACCTGCTTCCTACAAGCAGTTCAGAATGCCATGCCTTGGTTGTCC
TAGTGTGAATAATTTTCAGCTACTTTAAAATTATATTGTACTTTCTCAA
GCATGTCATATCCTTTCCTATTAGAGTATCTATATTACTTGTTACTGAT
TTACCTGAAGGCAATCTGATTAATTTCTAGGTTTTTACCATATTCTTGT
CATCTTGCCAATTACATTTTAAGTGTTAGACTAGACTAAGATGTACTAG
TTGTATAGAATATAACTAGATTTATTATGGCAATGTTTATTTTGTCATT
TTGCTTCATCTGTTTTGTTGTTGAAGTACTTTAAATTTCATACGTTCAT
GGCATTTCACTGTAAAGACTTTAATGTGTATTTCTTAAAATAAAACTTT
TTTTCCTCCTTAA.

By “CD59 polypeptide” is meant a polypeptide or fragment thereof having at least about 85% amino acid sequence identity to NCBI Reference Sequence No. NP_976075.1. and that extends viral half-life in a subject. An exemplary CD59 polypeptide sequence is provided below:

MGIQGGSVLFGLLLVLAVFCHSGHSLQCYNCPNPTADCKTAVNCSSDFD
ACLITKAGLQVYNKCWKFEHCNFNDVTTRLRENELTYYCCKKDLCNFNE
QLENGGTSLSEKTVLLLVTPFLAAAWSLHP.

By “CD59 polynucleotide” is meant a polynucleotide that encodes a CD59 polypeptide or a fragment thereof. An exemplary CD59 polynucleotide sequence is provided at base pairs 278 to 664 of NCBI Reference Sequence No.: NM_203330.2. An exemplary CD59 polynucleotide sequence is provided below:

GGGGCCGGGGGGCGGAGCCTTGCGGGCTGGAGCGAAAGAATGCGGGGGCTGAGCGCAGAAGCGG
CTCGAGGCTGGAAGAGGATCTTGGGCGCCGCCAGTCTCTCTCTGTTGCCCAAGCTGGAGTGCAG
TGGCACAGTCTTGGCTCACTGCAACCTCCACCTCCTGGGTGCAAGCGATTCTCGTGTCTCAGCC
TCTCAAGTAGCTGGGATTACAGTCTTTAGCACCAGTTGGTGTAGGAGTTGAGACCTACTTCACA
GTAGTTCTGTGGACAATCACAATGGGAATCCAAGGAGGGTCTGTCCTGTTCGGGCTGCTGCTCG
TCCTGGCTGTCTTCTGCCATTCAGGTCATAGCCTGCAGTGCTACAACTGTCCTAACCCAACTGC
TGACTGCAAAACAGCCGTCAATTGTTCATCTGATTTTGATGCGTGTCTCATTACCAAAGCTGGG
TTACAAGTGTATAACAAGTGTTGGAAGTTTGAGCATTGCAATTTCAACGACGTCACAACCCGCT
TGAGGGAAAATGAGCTAACGTACTACTGCTGCAAGAAGGACCTGTGTAACTTTAACGAACAGCT
TGAAAATGGTGGGACATCCTTATCAGAGAAAACAGTTCTTCTGCTGGTGACTCCATTTCTGGCA
GCAGCCTGGAGCCTTCATCCCTAAGTCAACACCAGGAGAGCTTCTCCCAAACTCCCCGTTCCTG
CGTAGTCCGCTTTCTCTTGCTGCCACATTCTAAAGGCTTGATATTTTCCAAATGGATCCTGTTG
GGAAAGAATAAAATTAGCTTGAGCAACCTGGCTAAGATAGAGGGGCTCTGGGAGACTTTGAAGA
CCAGTCCTGTTTGCAGGGAAGCCCCACTTGAAGGAAGAAGTCTAAGAGTGAAGTAGGTGTGACT
TGAACTAGATTGCATGCTTCCTCCTTTGCTCTTGGGAAGACCAGCTTTGCAGTGACAGCTTGAG
TGGGTTCTCTGCAGCCCTCAGATTATTTTTCCTCTGGCTCCTTGGATGTAGTCAGTTAGCATCA
TTAGTACATCTTTGGAGGGTGGGGCAGGAGTATATGAGCATCCTCTCTCACATGGAACGCTTTC
ATAAACTTCAGGGATCCCGTGTTGCCATGGAGGCATGCCAAATGTTCCATATGTGGGTGTCAGT
CAGGGACAACAAGATCCTTAATGCAGAGCTAGAGGACTTCTGGCAGGGAAGTGGGGAAGTGTTC
CAGATAGCAGGGCATGAAAACTTAGAGAGGTACAAGTGGCTGAAAATCGAGTTTTTCCTCTGTC
TTTAAATTTTATATGGGCTTTGTTATCTTCCACTGGAAAAGTGTAATAGCATACATCAATGGTG
TGTTAAAGCTATTTCCTTGCCTTTTTTTTATTGGAATGGTAGGATATCTTGGCTTTGCCACACA
CAGTTACAGAGTGAACACTCTACTACATGTGACTGGCAGTATTAAGTGTGCTTATTTTAAATGT
TACTGGTAGAAAGGCAGTTCAGGTATGTGTGTATATAGTATGAATGCAGTGGGGACACCCTTTG
TGGTTACAGTTTGAGACTTCCAAAGGTCATCCTTAATAACAACAGATCTGCAGGGGTATGTTTT
ACCATCTGCATCCAGCCTCCTGCTAACTCCTAGCTGACTCAGCATAGATTGTATAAAATACCTT
TGTAACGGCTCTTAGCACACTCACAGATGTTTGAGGCTTTCAGAAGCTCTTCTAAAAAATGATA
CACACCTTTCACAAGGGCAAACTTTTTCCTTTTCCCTGTGTATTCTAGTGAATGAATCTCAAGA
TTCAGTAGACCTAATGACATTTGTATTTTATGATCTTGGCTGTATTTAATGGCATAGGCTGACT
TTTGCAGATGGAGGAATTTCTTGATTAATGTTGAAAAAAAACCCTTGATTATACTCTGTTGGAC
AAACCGAGTGCAATGAATGATGCTTTTCTGAAAATGAAATATAACAAGTGGGTGAATGTGGTTA
TGGCCGAAAAGGATATGCAGTATGCTTAATGGTAGCAACTGAAAGAAGACATCCTGAGCAGTGC
CAGCTTTCTTCTGTTGATGCCGTTCCCTGAACATAGGAAAATAGAAACTTGCTTATCAAAACTT
AGCATTACCTTGGTGCTCTGTGTTCTCTGTTAGCTCAGTGTCTTTCCTTACATCAATAGGTTTT
TTTTTTTTTTTTTGGCCTGAGGAAGTACTGACCATGCCCACAGCCACCGGCTGAGCAAAGAAGC
TCATTTCATGTGAGTTCTAAGGAATGAGAAACAATTTTGATGAATTTAAGCAGAAAATGAATTT
CTGGGAACTTTTTTGGGGGGGGGGGGGTGGGGAATTCAGCCACACTCCAGAAAGCCAGGAGTCG
ACAGTTTTGGAAGCCTCTCTCAGGATTGAGATTCTAGGATGAGATTGGCTTACTGCTATCTTGT
GTCATGTACCCACTTTTTGGCCAGACTACACTGGGAAGAAGGTAGTCCTCTAAAGCAAAATCTG
AGTGCCACTAAATGGGGAGATGGGGCTGTTAAGCTGTCCAAATCAACAAGGGTCATATAAATGG
CCTTAAACTTTGGGGTTGCTTTCTGCAAAAAGTTGCTGTGACTCATGCCATAGACAAGGTTGAG
TGCCTGGACCCAAAGGCAATACTGTAATGTAAAGACATTTATAGTACTAGGCAAACAGCACCCC
AGGTACTCCAGGCCCTCCTGGCTGGAGAGGGCTGTGGCAATAGAAAATTAGTGCCAACTGCAGT
GAGTCAGCCTAGGTTAAATAGAGAGTGTAAGAGTGCTGGACAGGAACCTCCACCCTCATGTCAC
ATTTCTTCAATGTGACCCTTCTGGCCCCTCTCCTCCTGACAGCGGAACAATGACTGCCCCGATA
GGTGAGGCTGGAGGAAGAATCAGTCCTGTCCTTGGCAAGCTCTTCACTATGACAGTAAAGGCTC
TCTGCCTGCTGCCAAGGCCTGTGACTTTCTAACCTGGCCTCACGCTGGGTAAGCTTAAGGTAGA
GGTGCAGGATTAGCAAGCCCACCTGGCTACCAGGCCGACAGCTACATCCTCCAACTGACCCTGA
TCAACGAAGAGGGATTCATGTGTCTGTCTCAGTTGGTTCCAAATGAAACCAGGGAGCAGGGGAG
TTAGGAATCGAACACCAGTCATGCCTACTGGCTCTCTGCTCGAGAGCCAATACCCTGTGCCCTC
CACTCATCTGGATTTACAGGAACTGTCATAGTGTTCAGTATTGGGTGGTGATAAGCCCATTGGA
TTGTCCCCTTGGGGGGATGAGCTAGGGGTGCAAGGAACACCTGATGAGTAGATAAGTGGAGCTC
ATGGTATTTCCTGAAAGATGCTAATCTATTTGCCAAACTTGGTCTTGAATGTACTGGGGGCTTC
AAGGTATGGGTATATTTTTCTTGTGTCCTTGCAGTTAGCCCCCATGTCTTATGTGTGTCCTGAA
AAAATAAGAGCCTGCCCAAGACTTTGGGCCTCTTGACAGAATTAACCACTTTTATACATCTGAG
TTCTCTTGGTAAGTTCTTTAGCAGTGTTCAAAGTCTACTAGCTCGCATTAGTTTCTGTTGCTGC
CAACAGATCTGAACTAATGCTAACAGATCCCCCTGAGGGATTCTTGATGGGCTGAGCAGCTGGC
TGGAGCTAGTACTGACTGACATTCATTGTGATGAGGGCAGCTTTCTGGTACAGGATTCTAAGCT
CTATGTTTTATATACATTTTCATCTGTACTTGCACCTCACTTTACACAAGAGGAAACTATGCAA
AGTTAGCTGGATCGCTCAAGGTCACTTAGGTAAGTTGGCAAGTCCATGCTTCCCACTCAGCTCC
TCAGGTCAGCAAGTCTACTTCTCTGCCTATTTTGTATACTCTCTTTAATATGTGCCTAGCTTTG
GAAAGTCTAGAATGGGTCCCTGGTGCCTTTTTACTTTGAAGAAATCAGTTTCTGCCTCTTTTTG
GAAAAGAAAACAAAGTGCAATTGTTTTTTACTGGAAAGTTACCCAATAGCATGAGGTGAACAGG
ACGTAGTTAGGCCTTCCTGTAAACAGAAAATCATATCAAAACACTATCTTCCCATCTGTTTCTC
AATGCCTGCTACTTCTTGTAGATATTTCATTTCAGGAGAGCAGCAGTTAAACCCGTGGATTTTG
TAGTTAGGAACCTGGGTTCAAACCCTCTTCCACTAATTGGCTATGTCTCTGGACAAGTTTTTTT
TTTTTTTTTTTTTTAAACCCTTTCTGAACTTTCACTTTCTATGTCTACCTCAAAGAATTGTTGT
GAGGCTTGAGATAATGCATTTGTAAAGGGTCTGCCAGATAGGAAGATGCTAGTTATGGATTTAC
AAGGTTGTTAAGGCTGTAAGAGTCTAAAACCTACAGTGAATCACAATGCATTTACCCCCACTGA
CTTGGACATAAGTGAAAACTAGCCAGAAGTCTCTTTTTCAAATTACTTACAGGTTATTCAATAT
AAAATTTTTGTAATGGATAATCTTATTTATCTAAACTAAAGCTTCCTGTTTATACACACTCCTG
TTATTCTGGGATAAGATAAATGACCACAGTACCTTAATTTCTAGGTGGGTGCCTGTGATGGTTC
ATTGTAGGTAAGGACATTTTCTCTTTTTCAGCAGCTGTGTAGGTCCAGAGCCTCTGGGAGAGGA
GGGGGGTAGCATGCACCCAGCAGGGGACTGAACTGGGAAACTCAAGGTTCTTTTTACTGTGGGG
TAGTGAGCTGCCTTTCTGTGATCGGTTTCCCTAGGGATGTTGCTGTTCCCCTCCTTGCTATTCG
CAGCTACATACAACGTGGCCAACCCCAGTAGGCTGATCCTATATATGATCAGTGCTGGTGCTGA
CTCTCAATAGCCCCACCCAAGCTGGCTATAGGTTTACAGATACATTAATTAGGCAACCTAAAAT
ATTGATGCTGGTGTTGGTGTGACATAATGCTATGGCCAGAACTGAAACTTAGAGTTATAATTCA
TGTATTAGGGTTCTCCAGAGGGACAGAATTAGTAGGATATATGTATATATGAAAGGGAGGTTAT
TAGGGAGAACTGGCTCCCACAGTTAGAAGGCGAAGTCGCACAATAGGCCGTCTGCAAGCTGGGT
TAGAGAGAAGCCAGTAGTGGCTCAGCCTGAGTTCAAAAACCTCAAAACTGGGGAAGCTGACAGT
GCAGCCAGCCTTCAGTCTGTGGCCAAAGGCCCAAGAGCCCCTGGCAACCAACCCACTGGTGCAA
GTCCTAGATTCCAAAGGCTGAAGAACCTGGAGTCTGATGTCCAAGAGCAGGAAGAGTGGAAGAA
AGCCAGAAGACTCAGCAAACAAGGTAGACAGTGTCTACCACCATAGTGGCCATACCAAAGAGGC
TACCGATTCCTTCCTGCTACCTGGATCCCTGAAGTTGCCCTGGTCTCTGCACCTTCTAAACCTA
GTTCTTAAGAGCTTTCCATTACATGAGCTGTCTCAAAGCCCTCCAATAAATTCTCAGTGTAAGC
TTCTGTTGCTTGTGGACAGAAAATTCTGACAGACCTACCCTATAAGTGTTACTGTCAGGATAAC
ATGAGAACGCACAACAGTAAGTGGTCACTAAGTGTTAGCTACGGTTATTTTGCCCAAGGTAGCA
TGGCTAGTTGATGCCGGTTGATGGGGCTTAAACCCAGCTCCCTCATCTTCCAGGCCTCTGTACT
CCCTATTCCACTAAACTACCTCTCAGGTTTATTTTTTTAAATTCTTACTCTGCAAGTACATAGG
ACCACATTTACCTGGGAAAACAAGAATAAAGGCTGCTCTGCATTTTTTAGAAACTTTTTTGAAA
GGGAGATGGGAATGCCTGCACCCCCAAGTCCAGACCAACACAATGGTTAATTGAGATGAATAAT
AAAGGAAAGACTGTTCTGGGCTTCCCAGAATAGCTTGGTCCTTAAATTGTGGCACAAACAACCT
CCTGTCAGAGCCAGCCTCCTGCCAGGAAGAGGGGTAGGAGACTAGAGGCCGTGTGTGCAGCCTT
GCCCTGAAGGCTAGGGTGACAATTTGGAGGCTGTCCAAACACCCTGGCCTCTAGAGCTGGCCTG
TCTATTTGAAATGCCGGCTCTGATGCTAATCGGCGACCCTCAGGCAAGTTACTTAACCTTACAT
GCCTCAGTTTTCTCATCTGGAAAATGAGAACCCTAGGTTTAGGGTTGTTAGAAAAGTTAAATGA
GTTAAGACAAGTGCCTGGGACACAGTAGCCTCTTGTGTGTGTTTATCATTATGTCCTCAGCAGG
TCGTAGAAGCAGCTTCTCAGGTGTGAGGCTGGCGCGATTATCTGGAGTGGGTTGGGTTTTCTAG
GATGGACCCCCTGCTGCATTTTCCTCATTCATCCACCAGGGCTTAATGGGGAATCAAGGAATCC
ATGTGTAACTGTATAATAACTGTAGCCACACTCCAATGACCACCTACTAGTTGTCCCTGGCACT
GCTTATACATATGTCCATCAAATCAATCCTATGAAGTAGATACTGTCTTCATTTTATAGATCAG
AGACAATTGGGGTTCAGAGAGCTGATGTGATTTTCCCAGGGTCACAGAGAGTCCCAGATTCAGG
CACAACTCTTGTATTCCAAGACACAACCACTACATGTCCAAAGGCTGCCCAGAGCCACCGGGCA
CGGCAAATTGTGACATATCCCTAAAGAGGCTGAGCACCTGGTCAGGATCTGATGGCTGACAGTG
TGTCCAGATGCAGAGCTGGAGTGGGGGAGGGGAAGGGGGGCTCCTTGGGACAGAGAAGGCTTTC
TGTGCTTTCTCTGAAGGGAGCAGTCTGAGGACCAAGGGAACCCGGCAAACAGCACCTCAGGTAC
TCCAGGCCCTCCTGGCTGGAGAGGGCTGTGGCAATGGAAAATTAGTGCCAACTGCAATGAGTCA
GCCTCGGTTAAATAGAGAGTGAAGAATGCTGGACAGGAACCTCCACCCTCATGTCACATTTCTT
CAGTGTGACCCTTCTGGCCCCTCTCCTCCTGACAGCGGAACAATGACTGCCCCGATAGGTGAGG
CTGGAGGAAGAATCAGTCCTGTCCTTGGCAAGCTCTTCACTATGACAGTAAAGGCTCTCTGCCT
GCTGCCAAGGCCTGTGACTTTCTAACCTGGCCTCACGCTGGGTAAGCTTAAGGTAGAGGTGCAG
GATTAGCAAGCCCACCTGGCTACCAGGCCGACAGCTACATCTTTCAACTGACCCTGATCAACGA
AGAGGGACTTGTGTCTCTCAGTTGGTTCCAAATGAAACCAGGGAGCAGGGGCGTTAGGAAGCTC
CAACAGGATGGTACTTAATGGGGCATTTGAGTGGAGAGGTAGGTGACATAGTGCTTTGGAGCCC
AGGGAGGGAAAGGTTCTGCTGAAGTTGAATTCAAGACTGTTCTTTCATCACAAACTTGAGTTTC
CTGGACATTTGTTTGCAGAAACAACCGTAGGGTTTTGCCTTAACCTCGTGGGTTTATTATTACC
TCATAGGGACTTTGCCTCCTGACAGCAGTTTATGGGTGTTCATTGTGGCACTTGAGTTTTCTTG
CATACTTGTTAGAGAAACCAAGTTTGTCATCAACTTCTTATTTAACCCCCTGGCTATAACTTCA
TGGATTATGTTATAATTAAGCCATCCAGAGTAAAATCTGTTTAGATTATCTTGGAGTAAGGGGG
AAAAAATCTGTAATTTTTTCTCCTCAACTAGATATATACATAAAAAATGATTGTATTGCTTCAT
TTAAAAAATATAACGCAAAATCTCTTTTCCTTCTAAAAAAAAAAAAAAAAAA.

By “CD4 polypeptide” is meant a polypeptide or fragment thereof having at least about 85% amino acid sequence identity to GenBank accession no. AAV38594.1. provided below, and that functions as a co-receptor for a T-cell receptor (TCR).

MNRGVPFRHLLLVLQLALLPAATQGKKVVLGKKGDTVELTCTASQKKS
IQFHWKNSNQIKILGNQGSFLTKGPSKLNDRADSRRSLWDQGNFPLII
KNLKIEDSDTYICEVEDQKEEVQLLVFGLTANSDTHLLQGQSLTLTLE
SPPGSSPSVQCRSPRGKNIQGGKTLSVSQLELQDSGTWTCTVLQNQKK
VEFKIDIVVLAFQKASSIVYKKEGEQVEFSFPLAFTVEKLTGSGELWW
QAERASSSKSWITFDLKNKEVSVKRVTQDPKLQMGKKLPLHLTLPQAL
PQYAGSGNLTLALEAKTGKLHQEVNLVVMRATQLQKNLTCEVWGPTSP
KLMLSLKLENKEAKVSKREKAVWVLNPEAGMWQCLLSDSGQVLLESNI
KVLPTWSTPVQPMALIVLGGVAGLLLFIGLGIFFCVRCRHRRRQAERM
SQIKRLLSEKKTCQCPHRFQKTCSPI.

By “CD4 polynucleotide” is meant a polynucleotide that encodes a CD4 polypeptide or a fragment thereof. An exemplary CD4 polynucleotide sequence is provided at GenBank accession no. BT019791.1, provided below.

ATGAACCGGGGAGTCCCTTTTAGGCACTTGCTTCTGGTGCTGCAACTGG
CGCTCCTCCCAGCAGCCACTCAGGGAAAGAAAGTGGTGCTGGGCAAAAA
AGGGGATACAGTGGAACTGACCTGTACAGCTTCCCAGAAGAAGAGCATA
CAATTCCACTGGAAAAACTCCAACCAGATAAAGATTCTGGGAAATCAGG
GCTCCTTCTTAACTAAAGGTCCATCCAAGCTGAATGATCGCGCTGACTC
AAGAAGAAGCCTTTGGGACCAAGGAAACTTTCCCCTGATCATCAAGAAT
CTTAAGATAGAAGACTCAGATACTTACATCTGTGAAGTGGAGGACCAGA
AGGAGGAGGTGCAATTGCTAGTGTTCGGATTGACTGCCAACTCTGACAC
CCACCTGCTTCAGGGGCAGAGCCTGACCCTGACCTTGGAGAGCCCCCCT
GGTAGTAGCCCCTCAGTGCAATGTAGGAGTCCAAGGGGTAAAAACATAC
AGGGGGGGAAGACCCTCTCCGTGTCTCAGCTGGAGCTCCAGGATAGTGG
CACCTGGACATGCACTGTCTTGCAGAACCAGAAGAAGGTGGAGTTCAAA
ATAGACATCGTGGTGCTAGCTTTCCAGAAGGCCTCCAGCATAGTCTATA
AGAAAGAGGGGGAACAGGTGGAGTTCTCCTTCCCACTCGCCTTTACAGT
TGAAAAGCTGACGGGCAGTGGCGAGCTGTGGTGGCAGGCGGAGAGGGCT
TCCTCCTCCAAGTCTTGGATCACCTTTGACCTGAAGAACAAGGAAGTGT
CTGTAAAACGGGTTACCCAGGACCCTAAGCTCCAGATGGGCAAGAAGCT
CCCGCTCCACCTCACCCTGCCCCAGGCCTTGCCTCAGTATGCTGGCTCT
GGAAACCTCACCCTGGCCCTTGAAGCGAAAACAGGAAAGTTGCATCAGG
AAGTGAACCTGGTGGTGATGAGAGCCACTCAGCTCCAGAAAAATTTGAC
CTGTGAGGTGTGGGGACCCACCTCCCCTAAGCTGATGCTGAGCTTGAAA
CTGGAGAACAAGGAGGCAAAGGTCTCGAAGCGGGAGAAGGCGGTGTGGG
TGCTGAACCCTGAGGCGGGGATGTGGCAGTGTCTGCTGAGTGACTCGGG
ACAGGTCCTGCTGGAATCCAACATCAAGGTTCTGCCCACATGGTCCACC
CCGGTGCAGCCAATGGCCCTGATTGTGCTGGGGGGCGTCGCCGGCCTCC
TGCTTTTCATTGGGCTAGGCATCTTCTTCTGTGTCAGGTGCCGGCACCG
AAGGCGCCAAGCAGAGCGGATGTCTCAGATCAAGAGACTCCTCAGTGAG
AAGAAGACCTGCCAGTGCCCTCACCGGTTTCAGAAGACATGTAGCCCCA
TTTAG.

By “CD7 polypeptide” is meant a polypeptide or fragment thereof having at least about 85% amino acid sequence identity to GenBank accession no. AAA51953.1, provided below, and that functions in T-cell or B-cell interactions during early lymphoid development.

MAGPPRLLLLPLLLALARGLPGALAAQEVQQSPHCTTVPVGASVNITCS
TSGGLRGIYLRQLGPQPQDIIYYEDGVVPTTDRRFRGRIDFSGSQDNLT
ITMHRLQLSDTGTYTCQAITEVNVYGSGTLVLVTEEQSQGWHRCSDAPP
RASALPAPPTGSALPDPQTASALPDPPAASALPAALAVISFLLGLGLGV
ACVLARTQIKKLCSWRDKNSAACVVYEDMSHSRCNTLSSPNQYQ.

By “CD7 polynucleotide” is meant a polynucleotide that encodes a CD7 polypeptide or a fragment thereof. An exemplary CD7 polynucleotide sequence is provided at GenBank accession no. M37271.1, provided below.

ATGGCCGGGCCTCCGAGGCTCCTGCTGCTGCCCCTGCTTCTGGCGCTGG
CTCGCGGCCTGCCTGGGGCCCTGGCTGCCCAAGAGGTGCAGCAGTCTCC
CCACTGCACGACTGTCCCCGTGGGAGCCTCCGTCAACATCACCTGCTCC
ACCAGCGGGGGCCTGCGTGGGATCTACCTGAGGCAGCTCGGGCCACAGC
CCCAAGACATCATTTACTACGAGGACGGGGTGGTGCCCACTACGGACAG
ACGGTTCCGGGGCCGCATCGACTTCTCAGGGTCCCAGGACAACCTGACT
ATCACCATGCACCGCCTGCAGCTGTCGGACACTGGCACCTACACCTGCC
AGGCCATCACGGAGGTCAATGTCTACGGCTCCGGCACCCTGGTCCTGGT
GACAGAGGAACAGTCCCAAGGATGGCACAGATGCTCGGACGCCCCACCA
AGGGCCTCTGCCCTCCCTGCCCCACCGACAGGCTCCGCCCTCCCTGACC
CGCAGACAGCCTCTGCCCTCCCTGACCCGCCAGCAGCCTCTGCCCTCCC
TGCGGCCCTGGCGGTGATCTCCTTCCTCCTCGGGCTGGGCCTGGGGGTG
GCGTGTGTGCTGGCGAGGACACAGATAAAGAAACTGTGCTCGTGGCGGG
ATAAGAATTCGGCGGCATGTGTGGTGTACGAGGACATGTCGCACAGCCG
CTGCAACACGCTGTCCTCCCCCAACCAGTACCAGTGA.

By “CD8 polypeptide” is meant a polypeptide or fragment thereof having at least about 85% amino acid sequence identity to GenBank accession no. AAA79217.1, provided below, and that functions in T cell signaling and aids with cytotoxic T cell antigen interactions.

MALPVTALLLPLALLLHAARPSQFRVSPLDRTWNLGETVELKCQVLLSN
PTSGCSWLFQPRGAAASPTFLLYLSQNKPKAAEGLDTQRFSGKRLGDTF
VLTLSDFRRENEGYYFCSALSNSIMYFSHFVPVFLPAKPTTTPAPRPPT
PAPTIASQPLSLRPEACRPAAGGAVHTRGLDFACDIYIWAPLAGTCGVL
LLSLVITLYCNHRNRRRVCKCPRPVVKSGDKPSLSARYV.

By “CD8 polynucleotide” is meant a polynucleotide that encodes a CD8 polypeptide or a fragment thereof. An exemplary CD8 polynucleotide sequence is provided at GenBank accession no. AH003215.2, provided below.

ATGGCCTTACCAGTGACCGCCTTGCTCCTGCCGCTGGCCTTGCTGCTCC
ACGCCGCCAGGCCGAGCCAGTTCCGGGTGTCGCCGCTGGATCGGACCTG
GAACCTGGGCGAGACAGTGGAGCTGAAGTGCCAGGTGCTGCTGTCCAAC
CCGACGTCGGGCTGCTCGTGGCTCTTCCAGCCGCGCGGCGCCGCCGCCA
GTCCCACCTTCCTCCTATACCTCTCCCAAAACAAGCCCAAGGCGGCCGA
GGGGCTGGACACCCAGCGGTTCTCGGGCAAGAGGTTGGGGGACACCTTC
GTCCTCACCCTGAGCGACTTCCGCCGAGAGAACGAGGGCTACTATTTCT
GCTCGGCCCTGAGCAACTCCATCATGTACTTCAGCCACTTCGTGCCGGT
CTTCCTGCCAGCGAAGCCCACCACGACGCCAGCGCCGCGACCACCAACA
CCGGCGCCCACCATCGCGTCGCAGCCCCTGTCCCTGCGCCCAGAGGCGT
GCCGGCCAGCGGCGGGGGGCGCAGTGCACACGAGGGGGCTGGACTTCGC
CTGTGATATCTACATCTGGGCGCCCTTGGCCGGGACTTGTGGGGTCCTT
CTCCTGTCACTGGTTATCACCCTTTACTGCAACCACAGGAACCGAAGAC
GTGTTTGCAAATGTCCCCGGCCTGTGGTCAAATCGGGAGACAAGCCCAG
CCTTTCGGCGAGATACGTCTAA.

By “CD45 polypeptide” is meant a polypeptide or fragment thereof having at least about 85% amino acid sequence identity to NCBI Reference Sequence no. NP_002829.3, provided below, and that functions in T cell signaling and aids with cytotoxic T cell antigen interactions.

MTMYLWLKLLAFGFAFLDTEVFVTGQSPTPSPTGLTTAKMPSVPLSSDP
LPTHTTAFSPASTFERENDFSETTTSLSPDNTSTQVSPDSLDNASAFNT
TGVSSVQTPHLPTHADSQTPSAGTDTQTFSGSAANAKLNPTPGSNAISD
VPGERSTASTFPTDPVSPLTTTLSLAHHSSAALPARTSNTTITANTSDA
YLNASETTTLSPSGSAVISTTTIATTPSKPTCDEKYANITVDYLYNKET
KLFTAKLNVNENVECGNNTCTNNEVHNLTECKNASVSISHNSCTAPDKT
LILDVPPGVEKFQLHDCTQVEKADTTICLKWKNIETFTCDTQNITYRFQ
CGNMIFDNKEIKLENLEPEHEYKCDSEILYNNHKFTNASKIIKTDFGSP
GEPQIIFCRSEAAHQGVITWNPPQRSFHNFTLCYIKETEKDCLNLDKNL
IKYDLQNLKPYTKYVLSLHAYIIAKVQRNGSAAMCHFTTKSAPPSQVWN
MTVSMTSDNSMHVKCRPPRDRNGPHERYHLEVEAGNTLVRNESHKNCDF
RVKDLQYSTDYTFKAYFHNGDYPGEPFILHHSTSYNSKALIAFLAFLII
VTSIALLVVLYKIYDLHKKRSCNLDEQQELVERDDEKQLMNVEPIHADI
LLETYKRKIADEGRLFLAEFQSIPRVFSKFPIKEARKPFNQNKNRYVDI
LPYDYNRVELSEINGDAGSNYINASYIDGFKEPRKYIAAQGPRDETVDD
FWRMIWEQKATVIVMVTRCEEGNRNKCAEYWPSMEEGTRAFGDVVVKIN
QHKRCPDYIIQKLNIVNKKEKATGREVTHIQFTSWPDHGVPEDPHLLLK
LRRRVNAFSNFFSGPIVVHCSAGVGRTGTYIGIDAMLEGLEAENKVDVY
GYVVKLRRQRCLMVQVEAQYILIHQALVEYNQFGETEVNLSELHPYLHN
MKKRDPPSEPSPLEAEFQRLPSYRSWRTQHIGNQEENKSKNRNSNVIPY
DYNRVPLKHELEMSKESEHDSDESSDDDSDSEEPSKYINASFIMSYWKP
EVMIAAQGPLKETIGDFWQMIFQRKVKVIVMLTELKHGDQEICAQYWGE
GKQTYGDIEVDLKDTDKSSTYTLRVFELRHSKRKDSRTVYQYQYTNWSV
EQLPAEPKELISMIQVVKQKLPQKNSSEGNKHHKSTPLLIHCRDGSQQT
GIFCALLNLLESAETEEVVDIFQVVKALRKARPGMVSTFEQYQFLYDVI
ASTYPAQNGQVKKNNHQEDKIEFDNEVDKVKQDANCVNPLGAPEKLPEA
KEQAEGSEPTSGTEGPEHSVNGPASPALNQGS.

By “CD45 polynucleotide” is meant a polynucleotide that encodes a CD45 polypeptide or a fragment thereof. An exemplary CD45 polynucleotide sequence is provided at NCBI Reference Sequence no. NM_002838.5, provided below.

ATGACCATGTATTTGTGGCTTAAACTCTTGGCATTTGGCTTTGCCTTTC
TGGACACAGAAGTATTTGTGACAGGGCAAAGCCCAACACCTTCCCCCAC
TGGATTGACTACAGCAAAGATGCCCAGTGTTCCACTTTCAAGTGACCCC
TTACCTACTCACACCACTGCATTCTCACCCGCAAGCACCTTTGAAAGAG
AAAATGACTTCTCAGAGACCACAACTTCTCTTAGTCCAGACAATACTTC
CACCCAAGTATCCCCGGACTCTTTGGATAATGCTAGTGCTTTTAATACC
ACAGGTGTTTCATCAGTACAGACGCCTCACCTTCCCACGCACGCAGACT
CGCAGACGCCCTCTGCTGGAACTGACACGCAGACATTCAGCGGCTCCGC
CGCCAATGCAAAACTCAACCCTACCCCAGGCAGCAATGCTATCTCAGAT
GTCCCAGGAGAGAGGAGTACAGCCAGCACCTTTCCTACAGACCCAGTTT
CCCCATTGACAACCACCCTCAGCCTTGCACACCACAGCTCTGCTGCCTT
ACCTGCACGCACCTCCAACACCACCATCACAGCGAACACCTCAGATGCC
TACCTTAATGCCTCTGAAACAACCACTCTGAGCCCTTCTGGAAGCGCTG
TCATTTCAACCACAACAATAGCTACTACTCCATCTAAGCCAACATGTGA
TGAAAAATATGCAAACATCACTGTGGATTACTTATATAACAAGGAAACT
AAATTATTTACAGCAAAGCTAAATGTTAATGAGAATGTGGAATGTGGAA
ACAATACTTGCACAAACAATGAGGTGCATAACCTTACAGAATGTAAAAA
TGCGTCTGTTTCCATATCTCATAATTCATGTACTGCTCCTGATAAGACA
TTAATATTAGATGTGCCACCAGGGGTTGAAAAGTTTCAGTTACATGATT
GTACACAAGTTGAAAAAGCAGATACTACTATTTGTTTAAAATGGAAAAA
TATTGAAACCTTTACTTGTGATACACAGAATATTACCTACAGATTTCAG
TGTGGTAATATGATATTTGATAATAAAGAAATTAAATTAGAAAACCTTG
AACCCGAACATGAGTATAAGTGTGACTCAGAAATACTCTATAATAACCA
CAAGTTTACTAACGCAAGTAAAATTATTAAAACAGATTTTGGGAGTCCA
GGAGAGCCTCAGATTATTTTTTGTAGAAGTGAAGCTGCACATCAAGGAG
TAATTACCTGGAATCCCCCTCAAAGATCATTTCATAATTTTACCCTCTG
TTATATAAAAGAGACAGAAAAAGATTGCCTCAATCTGGATAAAAACCTG
ATCAAATATGATTTGCAAAATTTAAAACCTTATACGAAATATGTTTTAT
CATTACATGCCTACATCATTGCAAAAGTGCAACGTAATGGAAGTGCTGC
AATGTGTCATTTCACAACTAAAAGTGCTCCTCCAAGCCAGGTCTGGAAC
ATGACTGTCTCCATGACATCAGATAATAGTATGCATGTCAAGTGTAGGC
CTCCCAGGGACCGTAATGGCCCCCATGAACGTTACCATTTGGAAGTTGA
AGCTGGAAATACTCTGGTTAGAAATGAGTCGCATAAGAATTGCGATTTC
CGTGTAAAAGATCTTCAATATTCAACAGACTACACTTTTAAGGCCTATT
TTCACAATGGAGACTATCCTGGAGAACCCTTTATTTTACATCATTCAAC
ATCTTATAATTCTAAGGCACTGATAGCATTTCTGGCATTTCTGATTATT
GTGACATCAATAGCCCTGCTTGTTGTTCTCTACAAAATCTATGATCTAC
ATAAGAAAAGATCCTGCAATTTAGATGAACAGCAGGAGCTTGTTGAAAG
GGATGATGAAAAACAACTGATGAATGTGGAGCCAATCCATGCAGATATT
TTGTTGGAAACTTATAAGAGGAAGATTGCTGATGAAGGAAGACTTTTTC
TGGCTGAATTTCAGAGCATCCCGCGGGTGTTCAGCAAGTTTCCTATAAA
GGAAGCTCGAAAGCCCTTTAACCAGAATAAAAACCGTTATGTTGACATT
CTTCCTTATGATTATAACCGTGTTGAACTCTCTGAGATAAACGGAGATG
CAGGGTCAAACTACATAAATGCCAGCTATATTGATGGTTTCAAAGAACC
CAGGAAATACATTGCTGCACAAGGTCCCAGGGATGAAACTGTTGATGAT
TTCTGGAGGATGATTTGGGAACAGAAAGCCACAGTTATTGTCATGGTCA
CTCGATGTGAAGAAGGAAACAGGAACAAGTGTGCAGAATACTGGCCGTC
AATGGAAGAGGGCACTCGGGCTTTTGGAGATGTTGTTGTAAAGATCAAC
CAGCACAAAAGATGTCCAGATTACATCATTCAGAAATTGAACATTGTAA
ATAAAAAAGAAAAAGCAACTGGAAGAGAGGTGACTCACATTCAGTTCAC
CAGCTGGCCAGACCACGGGGTGCCTGAGGATCCTCACTTGCTCCTCAAA
CTGAGAAGGAGAGTGAATGCCTTCAGCAATTTCTTCAGTGGTCCCATTG
TGGTGCACTGCAGTGCTGGTGTTGGGCGCACAGGAACCTATATCGGAAT
TGATGCCATGCTAGAAGGCCTGGAAGCCGAGAACAAAGTGGATGTTTAT
GGTTATGTTGTCAAGCTAAGGCGACAGAGATGCCTGATGGTTCAAGTAG
AGGCCCAGTACATCTTGATCCATCAGGCTTTGGTGGAATACAATCAGTT
TGGAGAAACAGAAGTGAATTTGTCTGAATTACATCCATATCTACATAAC
ATGAAGAAAAGGGATCCACCCAGTGAGCCGTCTCCACTAGAGGCTGAAT
TCCAGAGACTTCCTTCATATAGGAGCTGGAGGACACAGCACATTGGAAA
TCAAGAAGAAAATAAAAGTAAAAACAGGAATTCTAATGTCATCCCATAT
GACTATAACAGAGTGCCACTTAAACATGAGCTGGAAATGAGTAAAGAGA
GTGAGCATGATTCAGATGAATCCTCTGATGATGACAGTGATTCAGAGGA
ACCAAGCAAATACATCAATGCATCTTTTATAATGAGCTACTGGAAACCT
GAAGTGATGATTGCTGCTCAGGGACCACTGAAGGAGACCATTGGTGACT
TTTGGCAGATGATCTTCCAAAGAAAAGTCAAAGTTATTGTTATGCTGAC
AGAACTGAAACATGGAGACCAGGAAATCTGTGCTCAGTACTGGGGAGAA
GGAAAGCAAACATATGGAGATATTGAAGTTGACCTGAAAGACACAGACA
AATCTTCAACTTATACCCTTCGTGTCTTTGAACTGAGACATTCCAAGAG
GAAAGACTCTCGAACTGTGTACCAGTACCAATATACAAACTGGAGTGTG
GAGCAGCTTCCTGCAGAACCCAAGGAATTAATCTCTATGATTCAGGTCG
TCAAACAAAAACTTCCCCAGAAGAATTCCTCTGAAGGGAACAAGCATCA
CAAGAGTACACCTCTACTCATTCACTGCAGGGATGGATCTCAGCAAACG
GGAATATTTTGTGCTTTGTTAAATCTCTTAGAAAGTGCGGAAACAGAAG
AGGTAGTGGATATTTTTCAAGTGGTAAAAGCTCTACGCAAAGCTAGGCC
AGGCATGGTTTCCACATTCGAGCAATATCAATTCCTATATGACGTCATT
GCCAGCACCTACCCTGCTCAGAATGGACAAGTAAAGAAAAACAACCATC
AAGAAGATAAAATTGAATTTGATAATGAAGTGGACAAAGTAAAGCAGGA
TGCTAATTGTGTTAATCCACTTGGTGCCCCAGAAAAGCTCCCTGAAGCA
AAGGAACAGGCTGAAGGTTCTGAACCCACGAGTGGCACTGAGGGGCCAG
AACATTCTGTCAATGGTCCTGCAAGTCCAGCTTTAAATCAAGGTTCATA
G.

By “major histocompatibility complex II (MHCII) polypeptide” is meant a polypeptide or fragment thereof having at least about 85% amino acid sequence identity to the MHCII alpha chain or MHCII beta chain amino acid sequence provided below, and that is capable of functioning in antigen presentation.

> MHCII alpha chain
EEHVIIQAEFYLNPDQSGEFMFDFDGDEIFHVDMAKKETVWRLEEFGRF
ASFEAQGALANIAVDKANLEIMTKRSNYTPITNVPPEVTVLINSPVELR
EPNVLICFIDKFTPPVVNVTWLRNGKPVTTGVSETVFLPREDHLFRKFH
YLPFLPSTEDVYDCRVEHWGLDEPLLKHWEFDA.
> MHCII beta chain
TRPRFLWQLKFECHFFNGTERVRLLERCIYNQEESVRFDSDVGEYRAVT
ELGRPDAEYWNSQKDLLEQRRAAVDTYCRHNYGVGESFTVQRRVEPKVT
VYPSKTQPLQHHNLLVCSVSGFYPGSIEVRWFRNGQEEKAGVVSTGLIQ
NGDWTFQTLVMLETVPRSGEVYTCQVEHPSVTSPLTVEWRA.

By “MHCII polynucleotide” is meant a polynucleotide that encodes a MHCII polypeptide or a fragment thereof.

By “administering” is meant giving, supplying, dispensing a composition, agent, therapeutic product, and the like to a subject, or applying or bringing the composition and the like into contact with the subject. Administering or administration may be accomplished by any of a number of routes, such as, for example, without limitation, parenteral or systemic, intravenous (IV), (injection), subcutaneous, intrathecal, intracranial, intramuscular, dermal, intradermal, inhalation, rectal, intravaginal, topical, oral, subcutaneous, intramuscular, or intraocular. In embodiments, administration is systemic, such as by inoculation, injection, or intravenous injection.

By “agent” is meant any small molecule chemical compound, antibody, nucleic acid molecule, or polypeptide, or fragments thereof.

By “alteration” is meant a change (increase or decrease) in the structure, expression levels or activity of a gene or polypeptide as detected by standard art known methods such as those described herein. As used herein, an alteration includes a 10% change in expression levels, preferably a 25% change, more preferably a 40% change, and most preferably a 50% or greater change in expression levels.”

By “ameliorate” is meant decrease, suppress, attenuate, diminish, arrest, or stabilize the development or progression of a disease.

By “analog” is meant a molecule that is not identical, but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

By “antigen” is meant an agent to which an antibody or other polypeptide capture molecule specifically binds. Exemplary antigens include small molecules, carbohydrates, proteins, and polynucleotides. In embodiments, the polypeptide capture molecule is a VHH.

By “Chimeric Antigen Receptor” or alternatively a “CAR” is meant a polypeptide capable of providing an immune effector cell with specificity for a target cell, typically a cancer cell. In some embodiments, a CAR comprises at least an extracellular antigen binding domain, a transmembrane domain and a cytoplasmic signaling domain comprising a functional signaling domain derived from a stimulatory molecule and/or costimulatory molecule. In embodiments, the stimulatory molecule is the zeta chain associated with the T cell receptor complex. In one embodiment, the cytoplasmic signaling domain further comprises one or more functional signaling domains derived from at least one costimulatory molecule. In one embodiment, the costimulatory molecule is 4-1BB (i.e., CD137). CD27 and/or CD28. In one embodiment, the CAR comprises a chimeric fusion protein comprising an extracellular antigen binding domain, a transmembrane domain and an intracellular signaling domain comprising a functional signaling domain derived from a stimulatory molecule. In one embodiment, the CAR comprises a chimeric fusion protein comprising an extracellular antigen binding domain, a transmembrane domain and an intracellular signaling domain comprising a functional signaling domain derived from a costimulatory molecule and a functional signaling domain derived from a stimulatory molecule. In one embodiment, the CAR comprises a chimeric fusion protein comprising an extracellular antigen binding domain, a transmembrane domain and an intracellular signaling domain comprising two functional signaling domains derived from one or more costimulatory molecule(s) and a functional signaling domain derived from a stimulatory molecule. In one embodiment, the CAR comprises a chimeric fusion protein comprising an extracellular antigen binding domain, a transmembrane domain and an intracellular signaling domain comprising at least two functional signaling domains derived from one or more costimulatory molecule(s) and a functional signaling domain derived from a stimulatory molecule. In one embodiment the CAR comprises an optional leader sequence at the amino-terminus (N-ter) of the CAR fusion protein. In one embodiment, the CAR further comprises a leader sequence at the N-terminus of the extracellular antigen binding domain. wherein the leader sequence is optionally cleaved from the antigen binding domain (e.g., a scFv) during cellular processing and localization of the CAR to the cellular membrane.

The term a “costimulatory molecule” refers to a cognate binding partner on a T cell that specifically binds with a costimulatory ligand, thereby mediating a costimulatory response by the T cell, such as, but not limited to, proliferation. Costimulatory molecules are cell surface molecules other than antigen receptors or their ligands that contribute to an efficient immune response. Costimulatory molecules include, but are not limited to an MHC class I molecule, BTLA and a Toll ligand receptor, as well as OX40, CD27, CD28, CDS, ICAM-1, LFA-1 (CD11a/CD18), ICOS (CD278), and 4-1BB (CD137). Further examples of such costimulatory molecules include CDS, ICAM-1, GITR, BAFFR, HVEM (LIGHTR), SLAMF7, NKp80 (KLRF1), NKp44, NKp30, NKp46, CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD1la, LFA-1, ITGAM, CD11 b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83.

In this disclosure, “comprises,” “comprising,” “containing” and “having” and the like can have the meaning ascribed to them in U.S. Patent law and can mean “ includes,” “including,” and the like: “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments. Any embodiments specified as “comprising” a particular component(s) or element(s) are also contemplated as “consisting of” or “consisting essentially of” the particular component(s) or element(s) in some embodiments.

By “consist essentially” it is meant that the ingredients include only the listed components along with the normal impurities present in commercial materials and with any other additives present at levels which do not affect the operation of the disclosure, for instance at levels less than 5% by weight or less than 1% or even 0.5% by weight.

By “disease” is meant any condition or disorder that damages or interferes with the normal function of a cell, tissue, or organ. Non-limiting examples of diseases include a cancer or tumor. In embodiments, the disease is a cytomegalovirus (CMV) infection, an influenza infection, a cancer or tumor, a lymphoma (e.g., a B-cell lymphoma), a neoplasia, or coronavirus disease of 2019 (COVID-19).

By “effective amount” is meant the amount of an agent required to ameliorate the symptoms of a disease relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1,000 nucleotides or amino acids.

As used herein, “heterologous” is used to refer to a gene, polynucleotide, or polypeptide experimentally put into a cell or viral particle that does not normally comprise that polynucleotide or polypeptide. In various embodiments “heterologous” is used to refer to a sequence derived from a different cell or virus from that virus or cell into which the sequence has been introduced.

By “increases” is meant a positive alteration of at least 10%, 25%, 50%, 75%, or 100%. The terms “isolated,” “purified.” or “biologically pure” refer to material that is free to varying degrees From components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid (e.g., a DNA) that is free of the genes which, in the naturally-occurring genome of the organism from which the nucleic acid molecule of the invention is derived, flank the gene. The term therefore includes, for example, a recombinant DNA that is incorporated into a vector: into an autonomously replicating plasmid or virus: or into the genomic DNA of a prokaryote or eukaryote: or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide: or by chemically synthesizing the protein. Purity can be measured by any appropriate method, for example, column chromatography, polyacrylamide gel electrophoresis, or by HPLC analysis.

As used herein, “obtaining” as in “obtaining an agent” includes synthesizing, purchasing, or otherwise acquiring the agent.

in As used herein, the term “pharmaceutically acceptable” refers to molecular entities, biological products and compositions that are physiologically tolerable and do not typically produce an allergic or other adverse reaction, such as gastric upset, dizziness and the like, when administered to a subject.

By “polypeptide” or “amino acid sequence” is meant any chain of amino acids, regardless of length or post-translational modification. In various embodiments, the post-translational modification is glycosylation or phosphorylation. In various embodiments, conservative amino acid substitutions may be made to a polypeptide to provide functionally equivalent variants, or homologs of the polypeptide. In some embodiments the invention embraces sequence alterations that result in conservative amino acid substitutions. In some embodiments, a “conservative amino acid substitution” refers to an amino acid substitution that does not alter the relative charge or size characteristics of the protein in which the conservative amino acid substitution is made. Variants can be prepared according to methods for altering polypeptide sequence known to one of ordinary skill in the art such as are found in references that compile such methods, e.g. Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. Non-limiting examples of conservative substitutions of amino acids include substitutions made among amino acids within the following groups: (a) M, I, L, V; (b) F, Y, W; (c) K, R; H; (d) A, G; (c) S, T; (f) Q, N; and (g) E, D. In various embodiments, conservative amino acid substitutions can be made to the amino acid sequence of the proteins and polypeptides disclosed herein.

The term “promoter” refers to a DNA sequence recognized by polypeptides required to initiate the transcription of a polynucleotide sequence in a cell.

As used herein, the term “pseudotyped” refers to a viral particle that contains one or more heterologous viral proteins. In embodiments the heterologous viral protein is an envelope glycoprotein. A pseudotyped virus may be one in which the envelope glycoproteins of an enveloped virus or the capsid proteins of a non-enveloped virus originate from a virus that differs from the source of the original virus genome and the genome replication apparatus. (D. A. Sanders, 2002, Curr. Opin. Biotechnol., 13:437-442). The foreign viral envelope proteins of a pseudotyped virus can be utilized to alter host tropism or to increase or decrease the stability of the virus particles. FIG. 1 provides a representative list of envelope glycoproteins. Examples of pseudotyped viral particles include a virus that contains one or more envelope glycoproteins that do not naturally occur on the exterior of the wild-type virus. Pseudotyped viral particles can infect cells and express and produce proteins or molecules encoded by polynucleotides contained within the viral particles.

By “reduces” is meant a negative alteration of at least 10%, 25%, 50%, 75%, or 100%.

By “reference” is meant a healthy subject or a subject prior to administration of a pseudotyped viral particle of the invention. In embodiments, the reference has never been administered a pseudotyped viral particle of the invention. In embodiments, the reference is a healthy subject prior to a particular instance of administration of a pseudotyped viral particle of the invention. A healthy subject is a subject free from a disease treated using a pseudotyped viral particle of the invention.

A “reference sequence” is a defined sequence used as a basis for sequence comparison. A reference sequence may be a subset of or the entirety of a specified sequence: for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or therebetween.

The term “signaling domain” refers to the functional portion of a protein which acts by transmitting information within the cell to regulate cellular activity via defined signaling pathways by generating second messengers or functioning as effectors by responding to such messengers.

An “intracellular signaling domain,” refers to an intracellular portion of a molecule. The intracellular signaling domain generates a signal that promotes an immune effector function of a CAR containing cell, e.g., a CART cell. Examples of immune effector function, e.g., in a CART cell, include cytolytic activity and helper activity, including the secretion of cytokines.

By “specifically binds” is meant in the context of an antibody or other polypeptide capture molecule recognizes and binds a polypeptide of the invention, but which does not substantially recognize and bind other molecules in a sample, for example, a biological sample, which naturally includes a polypeptide of the invention. In embodiments, the capture molecule is a VHH domain or a fragment thereof. A VHH domain or fragment thereof that specifically binds to an antigen will bind to the antigen with a K_{D} of less than 100 nM. For example, a VHH domain or fragment thereof that specifically binds to an antigen will bind to the antigen with a K_{D} of up to 100 nM (e.g., between 1 pM and 100 nM). A VHH domain or fragment thereof that does not exhibit specific binding to a particular antigen or epitope thereof will exhibit a K_{D} of greater than 100 nM (e.g., greater than 500 nm, 1 uM, 100 uM, 500 uM, or 1 mM) for that particular antigen or epitope thereof. A variety of immunoassay formats may be used to select a VHH domain or fragment thereof that specifically immunoreactive with a particular protein or carbohydrate. For example, solid-phase ELISA immunoassays are routinely used to select VHH domains or fragments thereof specifically immunoreactive with a protein or carbohydrate. See, Harlow & Lane, Antibodies, A Laboratory Manual, Cold Spring Harbor Press, New York (1988) and Harlow & Lane, Using Antibodies, A Laboratory Manual, Cold Spring Harbor Press, New York (1999), for a description of immunoassay formats and conditions that can be used to determine specific immunoreactivity.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399: Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C. and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 g/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C. and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42 C in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York): and Sambrook et al., Molecular Cloning: A Laboratory Manual, Cold Spring Harbor Laboratory Press, New York.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine: valine, isoleucine, leucine: aspartic acid, glutamic acid, asparagine, glutamine: serine, threonine: lysine, arginine: and phenylalanine, tyrosine. In an exemplary approach to determining the degree of identity, a BLAST program may be used, with a probability score between e⁻³ and e⁻¹⁰⁰ indicating a closely related sequence.

By “subject” is meant a mammal, including, but not limited to, a human or non-human mammal, such as a murine, bovine, equine, canine, ovine, or feline.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

As used herein, the term “vector” refers to a polynucleotide suitable for delivery of a gene sequence to a cell, or to a pseudotyped virus particle. Non-limiting examples of vectors include plasmids and cosmids. A “vector” further refers to a nucleic acid (polynucleotide) molecule into which foreign nucleic acid can be inserted without disrupting the ability of the vector to be expressed in, replicate in, and/or integrate into a host cell. A variety of vectors have been developed for the delivery of polynucleotides encoding exogenous proteins into a prokaryotic or eukaryotic cell. A vector may contain a polynucleotide sequence that includes gene of interest (e.g., a heterologous gene, such as a therapeutic gene, or a reporter gene) as well as, for example, additional sequence elements capable of regulating transcription, translation, and/or the integration of these polynucleotide sequences into the genome of a cell. A vector may contain regulatory sequences, such as a promoter, e.g., a subgenomic promoter, region and an enhancer region, which direct gene transcription. A vector may contain polynucleotide sequences (enhancer sequences) that enhance the rate of translation of these genes or improve the stability or nuclear export of the mRNA that results from gene transcription. These sequence elements may include, e.g., 5′ and 3′ untranslated regions, an internal ribosomal entry site (IRES), and/or a polyadenylation signal site in order to direct efficient transcription of a gene carried on the expression vector. Vectors, such as the pseudotyped viral particles described herein, may also be referred to as expression vectors.

By “viral transfer vector” is meant a vector comprising a polynucleotide encoding a heterologous polypeptide and containing viral cis-elements required for packaging into a viral particle and insertion into host genome.

“Transduction” refers to a process by which DNA or polynucleotide, e.g., one or more heterologous genes, contained in a virus or pseudotyped viral particle is introduced or transferred into a cell by the virus or pseudotyped viral particle, wherein the DNA or polynucleotide is expressed. In an embodiment, the DNA or polynucleotide transduced into a cell is stably expressed in the cell. In some cases, the virus or virus vector is said to infect a cell.

As used herein, the term “vehicle” refers to a solvent, diluent, or carrier component of a pharmaceutical composition.

By “viral particle” is meant an agent capable of infecting a cell and that exists as an independent particle containing a core viral genome or polynucleotide, a capsid, which surrounds the genetic material and protects it, and an envelope of lipids surrounding the capsid. A viral particle may refer to the form of a virus before it infects a cell and becomes intracellular, or to the form of the virus that infects a cell.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIG. 1 is a chart providing an overview of information relating to different lentiviral vectors. The chart of FIG. 1 is taken from Frank and Bucholz, “Surface-Engineered Lentiviral Vectors for Selective Gene Transfer into Subtypes of Lymphocytes,” Molecular Therapy—Methods & Clinical Development, 12:19-31 (2019), doi: 10.1016/j.omtm.2018.10.006.

FIGS. 2A and 2B provide a schematic depicting the domain architecture of MeV-scFv and MeV-VHH fusions and a collection of overlaid flow cytometry histograms. In FIG. 2A, the term “Hwt” designates a wild-type Morbillivirus H envelope protein domain: the term “H” designates an H envelope protein domain: the term “MeV” designates “Morbillivirus”: the term “418” designates a cytoplasmic tail truncation of 18 cytoplasmic nucleotides from the Morbillivirus H envelope protein domain: “Hmut” designates a Morbillivirus envelope protein domain containing an N481A alteration: “HA” designates a humanized influenza hemagglutinin tag: the term “3(G4S)” designates a peptide linker with the sequence GGGGSGGGGSGGGGS: and the term “2(G3S)” designates a peptide linker with the sequence GGGGSGGGGS. The flow cytometry histograms of FIG. 2B show that MV-H-scFv fusions were variably expressed on 293 producer cells. In FIG. 2B. The grey lines correspond to the MV-H scFv fusions, the black lines correspond to MV-H wild-type, and the greyed area corresponds to untransfected 293 cells. The rightmost peak of the black lines correspond to surface-expressed VH-H wild-type or MV-H scFv fusions.

FIGS. 3A and 3B provide stacked flow cytometry histograms and a bar graph demonstrating that poor surface-expression of MV-H-scFv fusions resulted in poor infection of receptor-expressing cells.

FIG. 4 is a collection of flow cytometry scatter plots demonstrating that, despite poor cell surface expression, a mThy1-scFv-H fusion can mediate some stable T cell transduction. Cells were infected on day 0. In FIG. 4, the numbers in each quadrant of the scatter plots (Q1-Q4) indicate the percent of total counted cells falling within each respective quadrant.

FIGS. 5A and 5B provide a collection of overlaid flow cytometry histograms and a bar graph all demonstrating that surface expression of VHH-H fusions was superior to scFv-H fusions. In the flow cytometry histograms of FIGS. 5A, the grey lines correspond to the MV-H VHH fusions, the black lines correspond to MV-H wild-type, and the greyed area corresponds to untransfected 293 cells. The rightmost peak of the black and grey lines correspond to surface-expressed VH-H wild-type, or MV-H VHH fusions, respectively. FIG. 5B provides a bar graph demonstrating that surface expression of MV-H VHH fusions (middle and right bars shown in medium and light grey, respectively) targeting the indicated antigens listed on the x-axis showed higher surface expression than MV-H scFv fusions (left bars shown in the darkest shade of grey in the figure) targeting the indicated antigens listed on the x-axis. The expression levels shown in the bar graph of FIG. 5B are shown as a ratio of the mean fluorescent intensity (MFI) (i.e., level of surface expression) measured for the indicated fusion (alternatively “chimera”) to MFI measured for the wild-type Morbillivirus H protein.

FIG. 6 provides a collection of flow cytometry scatter plots demonstrating that the indicated VHH-H fusions targeting CD45 and MHC-II efficiently and selectively infected primary splenocytes. At day 0, splenocytes were stimulated with anti-CD3/CD28 antibody and IL-2: virus was added at the same time. FACS from Day 2. In FIG. 6, the numbers in each quadrant of the scatter plots (Q1-Q4) indicate the percent of total counted cells falling within each respective quadrant.

FIG. 7 provides flow cytometry scatter plots and a plot of editing efficiency demonstrating that CRISPR guides delivered by aCD45-VHH-H fusions efficiently edited MHC-I in primary CD8 T cells. In FIG. 7, the numbers in each quadrant of the scatter plots indicate the percent of total counted cells falling within each respective quadrant.

FIGS. 8A and 8B provide a collection of flow cytometry scatter plots demonstrating that the indicated VHH-H fusions targeting mouse targets (e.g., MHC-II) or human targets (e.g., CD7) efficiently and selectively infected primary cells activated in vitro. In FIGS. 8A and 8B, the term “VsVg” designates the vesicular stomatitis virus glycoprotein (VSVg), and the term “PBMC” designates peripheral blood mononuclear cells. In FIGS. 8A and 8B, the numbers within each quadrant indicate the percent of total counted cells falling within each respective quadrant.

FIG. 9 provides a collection of flow cytometry scatter plots demonstrating that lentivirus (LV) pseudotyped with VHH-H fusions targeting human cell surface proteins efficiently and selectively infected primary human cells activated in vitro. 10E5 hPBMC cells were stimulated with αCD3/αCD28 beads and hIL-2. Cells were infected 3-days post-stimulation with lentivirus pseudotyped as indicated. Flow cytometry measurements were taken on day five post-infection. In FIG. 9, the numbers within each quadrant indicate the percent of total counted cells falling within each respective quadrant.

FIG. 10 provides a collection of flow cytometry scatter plots demonstrating that αMHCII-VHH and αCD45-VHH MeV-LVs infected A20 cells (A20 mouse B cell lymphoma model, which is CD45+and MHCII+) more efficiently than VsVg-LVs. 10,000 cells were infected with Lentivirus pseudotyped as indicated. All viruses contained a GFP reporter. Infected cells were analyzed via flow cytometry. In FIG. 10, the numbers adjacent to the outlined regions represent the percentage of total cells counted that fall within the outlined region. In FIG. 10, the numbers above the outlined regions represent the number of total counted cells falling within the indicated region.

FIGS. 11A and 11B provide a schematic presenting an experimental design and a collection of flow cytometry scatter plots. FIG. 11A provides a schematic presenting the experimental design used in the collection of the data presented in FIG. 11B. The plots of FIG. 11B demonstrate successful in vivo infection of A20 cells (A20 mouse B cell lymphoma model, which is CD45+, MHCII+, and CD19+) in immunodeficient mice (NOD scid gamma (NSG) mice) using Lentivirus pseudotyped with MV-H VHH fusions targeting αMHCII. Each plot in FIG. 11B represents results obtained using a different mouse. The viruses all contained a GFP reporter. In FIG. 11B, the numbers in each quadrant of the scatter plots indicate the percent of total counted cells falling within each respective quadrant and the number within the outlined regions represent the number of the total counted cells falling within the outlined region.

### DETAILED DESCRIPTION OF THE INVENTION

The invention features pseudotyped viral particles (e.g., lentiviral or gammaretroviral particles) and compositions and methods of use thereof, where the viral particles comprise a VHH domain. The pseudotyped viral particles are useful for, among other things, the in vivo delivery of a polynucleotide and/or polypeptide to a cell to treat a disease or condition (e.g., cancer) in a subject.

The invention is based, at least in part, upon the discovery that viral fusion proteins containing a VHH domain and a Morbillivirus hemagglutinin domain (VHH-MV-HA fusions) showed higher levels of surface expression in producer cells than fusions containing a single-chain variable fragment (scFv) domain and the hemagglutinin domain (scFv-MV-HA fusions). Lentiviral particles pseudotyped with the VHH-MV-HA fusions effectively targeted and transfected cells displaying the VHH antigen.

In embodiments, pseudotyped viral particles of the invention can be used in methods for in vivo cellular reprogramming of target cells. In various embodiments, such methods allow for a dramatic reduction ion manufacturing costs and time required for cell therapy and an increase in the number of patients that can benefit from cell therapy. The methods can have the advantage of allowing for in vivo editing of cells that are difficult to expand ex vivo, such as macrophage and NK cells. The lentiviral particles of the present invention have the advantage of having a large packaging unit and, thus, enable delivery of larger payloads than possible using adeno-associated virus (AAV) vectors or some nanoparticle approaches.

### Pseudotyped Viral Particles

The present invention features pseudotyped viral particles. In embodiments, the viral particle is a retroviral particle (e.g., a lentiviral particle or a gammaretroviral particle). In embodiments, the retroviral particle comprises a viral glycoprotein (e.g., a Morbillivirus H protein) or fragment thereof fused to a VHH domain or fragment thereof. Retroviral particles comprise an lipid envelope surrounding a viral capsid, where the viral capsid encapsidates (i.e., surrounds) a polynucleotide (e.g., single or double-stranded RNA). A retrovirus is a type of virus that inserts a copy of its genome (i.e., the encapsidated polynucleotide) into the genome of a host cell that it invades/infects. Once inside the host cell's cytoplasm, a retrovirus uses its own reverse transcriptase enzyme to produce DNA from the virus' own RNA genome. The DNA produced by the reverse transcriptase is then incorporated into the host cell genome by an integrase enzyme. Such incorporation results in stable expression of a gene(s) encoded by the polynucleotide in the infected cell and its progeny. There are three basic groups of retroviruses: oncoretroviruses, lentiviruses, and spumaviruses. Human retroviruses include HIV-1, HIV-2, and the human T-lymphotrophic virus. Mouse retroviruses include the murine leukemia virus.

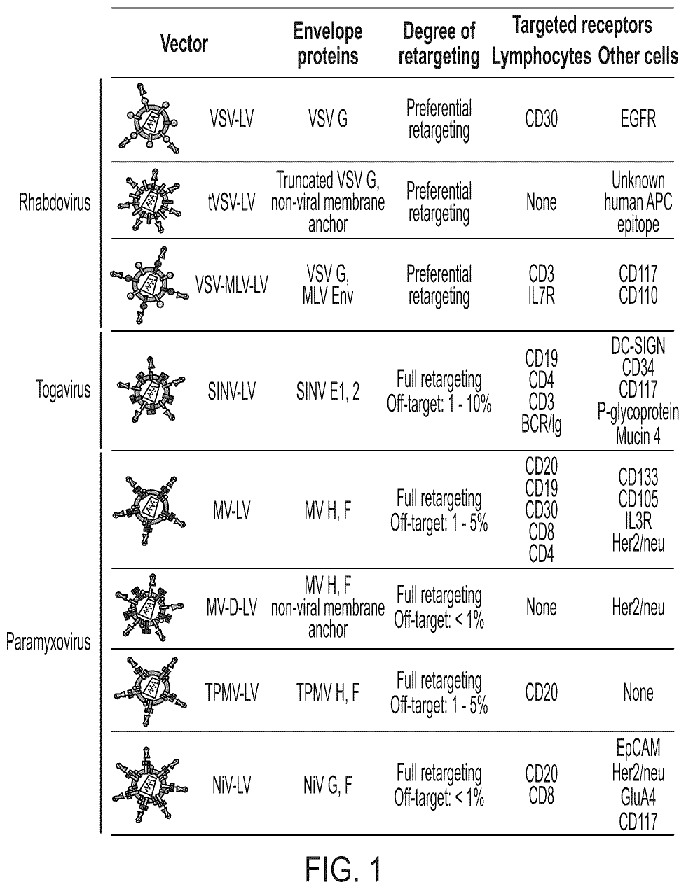

Retrovirus particles comprise a lipid envelope and are about 75-125 nm in diameter. The outer lipid envelope contains glycoprotein. Examples of glycoproteins contained in the lipid envelope of different retroviral particles are provided in FIG. 1. A retroviral particle can be pseudotyped by replacing the retroviral particle's endemic envelope proteins (e.g., a glycoprotein) with a heterologous envelope protein(s) (e.g., those listed in FIG. 1). In embodiments, the retroviral particle is pseudotyped with a glycoprotein from a Morbilllivirus. Glycoproteins facilitate targeting of the viral particle to a target cell. In embodiments, the glycoprotein of the invention is fused to a VHH domain. In embodiments, the glycoprotein or fragment thereof is mutated so as to no longer target a surface protein of a cell. Retroviruses typically have a genome comprising two single-stranded RNA molecules 7-10 kb in length. The two molecules can exist as a dimer formed through complementary base-pairing. In embodiments, a retrovirus genome encodes group-specific antigen (gag) proteins, protease (pro) proteins, polymerase (pol) proteins, and envelope (env) proteins. Gag proteins in embodiments are a major component of the viral capsid, and a viral capsid can comprise from about 2000 to about 4000 gag proteins. Gag proteins contain nucleic acid binding domains, including matrix (MA) and nucleocapsid (NC), that assist in packaging the polynucleotide into the capsid. Gag proteins are important for many aspects of virion assembly. Protease assists in virion maturation by, for example, assisting in proper gag protein and pol protein processing. Pol proteins are responsible for synthesis of viral DNA and integration into host DNA following infection. Env proteins (e.g., a glycoprotein) facilitate cell targeting and entry of the encapsidated polynucleotide into the target cell.

Lentiviruses and Gammaretrovirusesare genuses of retroviruses. In embodiments, the pseudotyped viral particles of the invention are pseudotyped lentiviral or gammaretroviral particles.

Retroviral particles have the advantage of being comparatively large (e.g., in comparison to adeno-associated virus (AAV) particles) and, therefore, capable of delivering larger polynucleotide sequences and/or a larger number of polypeptide sequences to a target cell than would be possible using alternative viral particles. Retroviral particles have the further advantage of possessing a viral envelope within which may be displayed a variety of polypeptides for delivery to a target cell. Delivering polypeptides to a target cell, as opposed to a polynucleotide, can have the advantage of facilitating the temporal introduction of an activity (e.g., an enzymatic or stimulatory activity) to a cell rather than constitutive activity (e.g., through integration of a polynucleotide sequence encoding a heterologous polypeptide into the genome of the target cell). A further advantage of retroviral particles is that, by virtue of containing a viral envelope, the surface of the viral particles (i.e., the envelope) may be altered to alter targeting of the retroviral particle or to alter interactions between the retroviral particle and the target cell.

The pseudotyped viral particles of the invention contain a polynucleotide. In embodiments, the polynucleotide encodes a heterologous gene. In embodiments, the heterologous gene is a chimeric antigen receptor, or a component thereof.

In embodiments, the viral envelope displays a polypeptide facilitating evasion of a subject's immune system by the viral particle. In embodiments, the viral envelope contains a polypeptide that inhibits phagocytosis. In embodiments, the viral envelope comprises a CD47 polypeptide. In embodiments, the viral envelope contains a complement regulatory polypeptide. Non-limiting examples of complement regulatory polypeptides include CD46, CD55, and CD59.

In embodiments, the viral particle contains (e.g., as displayed on the viral envelope) polypeptides that activate a physiological response (e.g., proliferation, survival, intracellular signaling, changes in gene expression, apoptosis, or differentiation) in the target cell (e.g., through introduction of a cytokine or a chemokine to the target cell). Non-limiting examples of cytokines or chemokines that can be included in the viral envelope include of aCD3, Ccl14, CD28, CD40L, Cxcl10, IL-2, IL-7, IL-12, IL-15, IL-18, and IL-21.

Methods for displaying polypeptides in a viral envelope are known and are suitable for use in embodiments of the invention. See, for example, Taube, et al., “Lentivirus Display: Stable Expression of Human Antibodies on the Surface of Human Cells and Virus Particles”, PLOS ONE, 3: e3181 (2008).

In embodiments, the viral particle is not capable of self-replication. In embodiments, the viral particle is capable of self-replication.

### VHH Domains

In embodiments, pseudotyped viral particles of the invention comprise VHH domains. In embodiments, the VHH domain binds an antigen selected from, as non-limiting examples, BCR/Ig, CD3, CD4, CD7, CD8, CD11, CD19, CD20, CD30, CD34, CD38, CD45, CD133, CD103, CD105, CD110, CD117, CTLA-4, CXCR4, DC-SIGN, EGFR, Emr1, EpCAM, GluA4, Her2/neu, IL3R, IL7R, Mac, MHCII, Mucin 4, NK1.1, P-glycoprotein, TIM3, Thy1, and Thy1.2. In embodiments, the VHH binds an antigen associated with a target cell. In embodiments, the target cell is an immune cell. As non-liming examples, the target cell can be a B cell, a dendritic cell, an eosinophil, a granulocyte, an iNKT cell, a macrophage, a monocyte, a natural killer cell, a neutrophil, a lymphoma cell, a regulatory T cell, or a T cell. In embodiments, the immune cell is CD4+and/or CD8+.

VHH domains are derived from nanobodies. Nanobodies are antibody-derived therapeutic proteins that contain the unique structural and functional properties of naturally-occurring heavy-chain antibodies. These heavy-chain antibodies contain a single variable domain (VHH) and two constant domains (CH2 and CH3). Importantly, the cloned and isolated VHH domain is a stable polypeptide harboring the full antigen-binding capacity of the original heavy-chain antibody. Nanobodies have a high homology with the VH domains of human antibodies and can be further humanized without any loss of activity. Importantly, Nanobodies have a low immunogenic potential, which has been confirmed in primate studies with Nanobody lead compounds.

Nanobodies combine the advantages of conventional antibodies with important features of small molecule drugs. Like conventional antibodies, Nanobodies show high target specificity, high affinity for their target and low inherent toxicity. However, like small molecule drugs they can inhibit enzymes and readily access receptor clefts. Furthermore, Nanobodies are stable, can be administered by means other than injection (see, e.g., WO2004041867A2, which is herein incorporated by reference in its entirety) and are easy to manufacture. Other advantages of Nanobodies include recognizing uncommon or hidden epitopes as a result of their small size, binding into cavities or active sites of protein targets with high affinity and selectivity due to their unique 3-dimensional, drug format flexibility, tailoring of half-life and ease and speed of drug discovery.

Nanobodies are encoded by single genes and are efficiently produced in almost all prokaryotic and eukaryotic hosts, e.g., E. coli (see, e.g., U.S. Pat. No. 6,765,087, which is herein incorporated by reference in its entirety), molds (for example Aspergillus or Trichoderma) and yeast (for example Saccharomyces, Kluyveromyces, Hansenula, or Pichia) (see, e.g., U.S. Pat. No. 6,838,254, which is herein incorporated by reference in its entirety). Methods known in the art may be used to generate nanobodies. These nanobodies may then serve as the basis for the generation of a library which may be produced and selected from according using methods such as, for example, the Nanoclone method (see, e.g., WO 06/079372, which is herein incorporated by reference in its entirety), which is a proprietary method for generating Nanobodies against a desired target, based on automated high-throughput selection of B-cells and could be used in the context of the invention. The successful selection of nanobodies using the Nanoclone method may provide an initial set of nanobodies, which are then used to discover bispecific molecules comprising nanobodies using the methods described herein.

A variety of VHH domains are commercially available, any of which may be used in embodiments of the present invention. A list of VHH domains that may be used in connection with embodiments of the invention is provided in Table 2 of the Examples.

Method of producing pseudotyped viral particles

A method of producing a pseudotyped viral (e.g., lentiviral or gammaretroviral) particle described herein will generally involve introducing a viral transfer vector and one or more additional vectors (e.g., a retroviral packaging vector) into a cell. A variety of methods suitable for production of pseudotyped viral vectors of the invention are known, such as those presented in Merten, et al., “Production of lentiviral vectors”, Mol Ther Methods Clin Dev, 3:16017 (2016) and in Nasri, et al., “Production, purification and titration of a Lentivirus-based vector for gene delivery purposes”, Cytotechnology, 66:1031-1038 (2014), the disclosures of which are incorporated herein by reference in their entireties for all purposes.

In embodiments, the production of a pseudotyped viral particle involves introducing into a cell (i.e., a producer cell) a viral transfer vector containing a heterologous gene sequence, a packaging vector, and an envelope vector (e.g., a vector encoding a glycoprotein or fragment thereof fused to a VHH or fragment thereof). In embodiments, the viral transfer vector contains a heterologous polynucleotide sequence containing a heterologous gene flanked by long terminal repeat (LTR) sequences, which facilitate integration of the heterologous gene sequence into the genome of a target cell. In embodiments, the transfer vector may contain a deletion in a 3′LTR to render the pseudotyped viral particle self-inactivating (SIN) after integration of the polynucleotide into the genome of the target cell.

The vectors may be introduced into the cell using transfection methods well known in the art. After transfection, the cell may be permitted to express viral proteins encoded by the viral transfer vector and/or the one or more additional vectors (e.g., by incubating the cell under standard conditions known in the art for inducing viral gene expression). In embodiments, the viral genes are expressed under the control of a constitutive or inducible promoter. In the latter case, viral gene expression may be selectively induced by incubating the cell under conditions suitable for activating the inducible promoter. Viral proteins produced by the cell may subsequently form a viral particle, which buds from the cell surface and can be isolated from the solution (e.g., according to methods well known in the art). When the viral particle buds from the cell surface and obtains a viral envelope containing a portion of the lipid membrane of the cell from which it budded as well as associated membrane proteins (e.g., a hemagglutinin) that were contained within the lipid membrane of the cell. During formation of the virus, a polynucleotide encoding a heterologous polypeptide may be incorporated into the viral particle. Thus, this process yields a pseudotyped retroviral particle that includes a polynucleotide encoding a heterologous gene (e.g., a heterologous polypeptide), where the polynucleotide sequence originated from the viral transfer vector.

The heterologous gene may include a gene encoding a polypeptide or a gene for a noncoding RNA that is to be expressed in a target cell. In some instances, the heterologous protein ORF is positioned downstream of a Kozak sequence. In some instances, the polynucleotide of the viral transfer vector will be present in a retroviral particle produced in a cell transfected with the viral transfer vector and, optionally, one or more additional vectors (e.g., packaging vectors). In certain instances, the polynucleotide may be integrated into the genome of a cell infected with the pseudotyped retroviral particle. Integration of the heterologous nucleic acid into the genome of such a cell may permit the cell and its progeny to express the heterologous gene of interest. The gene of interest may be any gene known in the art. Exemplary genes of interest include, without limitation, genes encoding chimeric antigen receptors (CARs), binding moieties (e.g., antibodies and antibody fragments), signaling proteins, cell surface proteins (e.g., T cell receptors), proteins involved in disease (e.g., cancers, autoimmune diseases, neurological disorders, or any other disease known in the art), or any derivative or combination thereof. In embodiments, the heterologous polypeptide is an antigen (e.g., an influenza, coronavirus, cancer, or cytomegalovirus antigen). In embodiments, the heterologous polypeptide is a therapeutic polypeptide (e.g., a chimeric antigen receptor (CAR)).

A viral transfer vector of the invention may be introduced into a cell (producer cell). The viral transfer vector is generally co-transfected into the cell together with one or more additional vectors (e.g., one or more packaging vectors). The one or more additional vectors may encode viral proteins and/or regulatory proteins. Co-transfection of the viral transfer vector and the one or more additional vectors (e.g., a vector encoding a glycoprotein fused to a VHH) enables the host cell to produce a pseudotyped viral particle (e.g., a Lentivirus or Gammaretrovirus containing a polynucleotide from the lentiviral transfer vector). Pseudotyped retroviral particles produced by a cell as described herein may be used to infect another cell. The polynucleotide containing a heterologous gene sequence (e.g., encoding a polypeptide of interest) and/or one or more additional elements (e.g., promoters and viral elements) may be integrated into the genome of the infected cell, thereby permitting the cell and its progeny to express gene(s) originating from the viral transfer vector.

A producer cell suitable for transfection with the lentiviral transfer vector (and one or more packaging vectors) may be a eukaryotic cell, such as a mammalian cell. The host cell may originate from a cell line (e.g., an immortalized cell line). For example, the host cell may be a HEK 293 cell.

Target cell is the cell that is infected (transduced) with a pseudotyped viral particle containing a polynucleotide encoding a gene of interest. After transduction, the heterologous gene of interest is stably inserted into target cell genome and can be detected by molecular biology methods such as PCR and Southern blot. Transgene can be expressed in target cell and detected by flow cytometry or Western blot. In some instances, target cell is a human cell. In certain instances, the host cell is a particular cell type of interest, e.g., a primary T cell, SupTI cell, Jurkat cell, or 293T cell.

The viral transfer vectors may include one or more of the following: a promoter (e.g., a CMV, RSV, or EF la promoter) driving expression of one or more viral sequences, long terminal repeat (LTR) regions (e.g., an R region or an U5 region), optionally flanking a heterologous gene sequence, a primer binding site (PBS), a packaging signal (psi) (e.g., a packaging signal including a major splice donor site (SD)), acPPT element, a Kozak sequence positioned upstream (e.g., immediately upstream) of a heterologous gene sequence to be transferred to a cell), a Rev-response element (RRE), a subgenomic promoter (e.g., P-EF1a), a heterologous gene (e.g., a heterologous gene encoding a CAR gene), a post-transcriptional regulatory element (e.g., a WPRE or HPRE), a polyA sequence, a selectable marker (e.g., a kanamycin resistance gene (nptII), ampicilin resistance gene, or a chloramphenicol resistance gene), and an origin of replication (e.g., a pUC origin of replication, an SV40 origin of replication, or an f1 origin of replication).

The viral transfer vector may also include elements suitable for driving expression of a heterologous protein in a cell. In certain instances, a Kozak sequence is positioned upstream of the heterologous protein open reading frame. For example, the viral transfer vector may include a promoter (e.g., a CMV, RSV, or EF1a promoter) that controls the expression of the heterologous nucleic acid. Other promoters suitable for use in the lentiviral transfer vector include, for example, constitutive promoters or tissue/cell type-specific promoters. In some instances, the lentiviral transfer vector includes a means of selectively marking a gene product (e.g., a polypeptide or RNA) encoded by at least a portion of the polynucleotide (e.g., a polynucleotide encoding a gene product of interest). For example, the viral transfer vector may include a marker gene (e.g., a gene encoding a selectable marker, such as a fluorescent protein (e.g., GFP, YFP, RFP, dsRed, mCherry, or any derivative thereof)). The marker gene may be expressed independently of the gene product of interest. Alternatively, the marker gene may be co-expressed with the gene product of interest. For example, the marker gene may be under the control of the same or different promoter as the gene product of interest. In another example, the marker gene may be fused to the gene product of interest. The elements of the viral transfer vectors of the invention are, in general, in operable association with one another, to enable the transfer vectors together with one or more packaging vectors to participate in the formation of a pesudotyped viral particle in a transfected cell.

The viral transfer vectors of the invention may be co-transfected into a cell together with one or more additional vectors. In some instances, the one or more additional vectors may include lentiviral packaging vectors and/or envelop vectors. In certain instances, the one or more additional vectors may include an envelope vector (e.g., an envelope vector encoding a glycoprotein fused to a VHH). Generally, a packaging vector includes one or more polynucleotide sequences encoding viral proteins (e.g., gag, pol, env, tat, rev, vif, vpu, vpr, and/or nef protein, or a derivative, combination, or portion thereof). A packaging vector to be co-transfected into a cell with a viral transfer vector of the invention may include sequence(s) encoding one or more viral proteins not encoded by the transfer vector. For example, a viral transfer vector may be co-transfected with a first packaging vector encoding gag and pol and a second packaging vector encoding rev. Thus, co-transfection of a viral transfer vector with such packaging vector(s) may result in the introduction of all genes required for viral particle formation into the cell, thereby enabling the cell to produce viral particles that may be isolated. Further, the viral particles produced by the cell lack genes critical for viral particle formation and are, thus, incapable of self-replication. For various safety reasons, it can be advantageous to produce pseudotyped viral particles and are incapable of self-replication. Appropriate packaging vectors for use in the invention can be selected by those of skill in the art based on, for example, consideration of the features selected for a viral transfer vector of the invention. For examples of packaging vectors that can be used or adapted for use in the invention see, e.g., WO 03/064665, WO 2009/153563, U.S. Pat. No. 7,419,829, WO 2004/022761, U.S. Pat. No. 5,817,491, WO 99/41397, U.S. Pat. Nos. 6,924,123, 7,056,699, WO 99/32646, WO 98/51810, and WO 98/17815. In some instances, a packaging vector may encode a gag and/or pol protein, and may optionally include an RRE sequence (e.g., an pMDLgpRRE vector: see, e.g., Dull et al., J. Virol. 72(11): 8463-8471, 1998). In certain instances, a packaging vector may encode a rev protein (e.g., a pRSV-Rev vector).

### Genome Editing

Therapeutic gene editing is a major focus of biomedical research, embracing the interface between basic and clinical science. An immune cell may be treated according to the methods of the present invention by knocking out (e.g., by deletion) or inhibiting expression of a target gene(s). The development of novel “gene editing” tools provides the ability to manipulate the DNA sequence of a cell (e.g., to delete a target gene) at a specific chromosomal locus, without introducing mutations at other sites of the genome. This technology effectively enables the researcher to manipulate the genome of a subject's cells in vitro or in vivo.

In one embodiment, gene editing involves targeting an endonuclease (an enzyme that causes DNA breaks internally within a DNA molecule) to a specific site of the genome and thereby triggering formation of a chromosomal double strand break (DSB) at the chosen site. If, concomitant with the introduction of the chromosome breaks, a donor DNA molecule may be introduced (for example, by plasmid or oligonucleotide introduction), interactions between the broken chromosome and the introduced DNA can occur, especially if the two sequences share homology. In this instance, a process termed “gene targeting” can occur, in which the DNA ends of the chromosome invade homologous sequences of the donor DNA by homologous recombination (HR). By using the donor plasmid sequence as a template for HR, a seamless repair of the chromosomal DSB can be accomplished. In some embodiments, no donor DNA molecule is introduced and the double-stranded break is repaired by the error-prone non-homologous end joining NHEJ pathway leading to knock-out or deletion of the target gene (e.g., through the introduction of indels or nonsense mutations). In some embodiments, an endonuclease(s) can be targeted to at least two distinct chosen sites located within a gene sequence so that chromosomal double strand breaks at the distinct sites leads to excision and deletion of a nucleotide sequence flanked by the two distinct sites.

Current genome editing tools use the induction of double strand breaks (DSBs) to enhance gene manipulation of cells, including the deletion or knockout of genes. Such methods include zinc finger nucleases (ZFNs: described for example in U.S. Pat. Nos. 6,534,261, 6,607,882, 6,746,838, 6,794,136, 6,824,978, 6,866,997, 6,933, 113, 6,979,539, 7,013,219, 7,030,215, 7,220,719, 7,241,573, 7,241,574, 7,585,849, 7,595,376, 6,903,185, and 6,479,626, and U.S. Pat. Publ. Nos. 20030232410 and U.S. Pat. No. 20,090,20314, which are incorporated herein by reference), Transcription Activator-Like Effector Nucleases (TALENs: described for example in U.S. Pat. Nos. 8,440,431, 8,440,432, 8,450,471, 8,586,363, and 8,697,853, and U.S. Pat. Publ. Nos. 20110145940, 20120178131, 20120178169, 20120214228, 20130122581, 20140335592, and 20140335618, which are incorporated herein by reference), and the CRISPR (Clustered Regularly Interspaced Short Palindromic Repeats)/Cas9 system (described for example in U.S. Pat. Nos. 8,697,359, 8,771,945, 8,795,965, 8,871,445, 8,889,356, 8,906,616, 8,932,814, 8,945,839, 8,993,233, and 8,999,641, and U.S. Pat. Publ. Nos. 20140170753, 20140227787, 20140179006, 20140189896, 20140273231, 20140242664, 20140273232, 20150184139, 20150203872, 20150031134, 20150079681, 20150232882, and 20150247150, which are incorporated herein by reference). In some embodiments a CRISPR/Cas 12 system can be used for gene editing. In some embodiments, the Cas 12 polypeptide is Cas12b. In some embodiments any Cas polypeptide can be used for gene editing (e.g., CasX). In various embodiments, the Cas polypeptide is selected so that a nucleotide encoding the Cas polypeptide can fit within an adeno-associated virus (AAV) capsid. For example, ZFN DNA sequence recognition capabilities and specificity can be unpredictable. Similarly, TALENs and CRISPR/Cas9 cleave not only at the desired site, but often at other “off-target” sites, as well. These methods have significant issues connected with off-target double-stranded break induction and the potential for deleterious mutations, including indels, genomic rearrangements, and chromosomal rearrangements, associated with these off-target effects. ZFNs and TALENs entail use of modular sequence-specific DNA binding proteins to generate specificity for ˜18 bp sequences in the genome. CRISPR/Cas9, TALENs, and ZFNs have all been used in clinical trials (see, e.g., Li., H, et al., “Applications of genome editing technology in the targeted therapy of human diseases: mechanisms, advances and prospects”, Signal Transduct Target Ther., 5:1 (2020), DOI: 10.1038/s41392-019-0089-y).

RNA-guided nucleases-mediated genome editing, based on Type 2 CRISPR (Clustered Regularly Interspaced Short Palindromic Repeat)/Cas (CRISPR Associated) systems, offers a valuable approach to alter the genome. In brief, Cas9, a nuclease guided by single-guide RNA (sgRNA), binds to a targeted genomic locus next to the protospacer adjacent motif (PAM) and generates a double-strand break (DSB). The DSB is then repaired either by non-homologous end joining (NHEJ), which leads to insertion/deletion (indel) mutations, or by homology-directed repair (HDR), which requires an exogenous template and can generate a precise modification at a target locus (Mali et al., Science. 2013 Feb. 15:339(6121): 823-6). Genetic manipulation using engineered nucleases has been demonstrated in tissue culture cells and rodent models of diseases.

CRISPR has been used in a wide range of organisms including baker's yeast (S. cerevisiae), zebra fish, nematodes (C. elegans), plants, mice, and several other organisms. Additionally, CRISPR has been modified to make programmable transcription factors that allow scientists to target and activate or silence specific genes. Libraries of tens of thousands of guide RNAs are now available.

Since 2012, the CRISPR/Cas system has been used for gene editing (silencing, enhancing or changing specific genes) that even works in eukaryotes like mice and primates. By inserting a plasmid containing Cas genes and specifically designed CRISPRs, an organism's genome can be cut at any desired location.

CRISPR repeats range in size from 24 to 48 base pairs. They usually show some dyad symmetry, implying the formation of a secondary structure such as a hairpin, but are not truly palindromic. Repeats are separated by spacers of similar length. Some CRISPR spacer sequences exactly match sequences from plasmids and phages, although some spacers match the prokaryote's genome (self-targeting spacers). New spacers can be added rapidly in response to phage infection.

CRISPR-associated (cas) genes are often associated with CRISPR repeat-spacer arrays. As of 2013, more than forty different Cas protein families had been described. Of these protein families, Casl appears to be ubiquitous among different CRISPR/Cas systems. Particular combinations of Cas genes and repeat structures have been used to define 8 CRISPR subtypes (E. coli, Y. pest, Nmeni, Dvulg. Tneap, Hmari, Apern, and Mtube), some of which are associated with an additional gene module encoding repeat-associated mysterious proteins (RAMPs). More than one CRISPR subtype may occur in a single genome. The sporadic distribution of the CRISPR/Cas subtypes suggests that the system is subject to horizontal gene transfer during microbial evolution.

Exogenous DNA is apparently processed by proteins encoded by Cas genes into small elements (about 30 base pairs in length), which are then somehow inserted into the CRISPR locus near the leader sequence. RNAs from the CRISPR loci are constitutively expressed and are processed by Cas proteins to small RNAs composed of individual, exogenously-derived sequence elements with a flanking repeat sequence. The RNAs guide other Cas proteins to silence exogenous genetic elements at the RNA or DNA level. Evidence suggests functional diversity among CRISPR subtypes. The Cse (Cas subtype E. coli) proteins (called CasA-E in E. coli) form a functional complex, Cascade, that processes CRISPR RNA transcripts into spacer-repeat units that Cascade retains. In other prokaryotes, Cas6 processes the CRISPR transcripts. Interestingly, CRISPR-based phage inactivation in E. coli requires Cascade and Cas3, but not Cas1 and Cas2. The Cmr (Cas RAMP module) proteins found in Pyrococcus furiosus and other prokaryotes form a functional complex with small CRISPR RNAs that recognizes and cleaves complementary target RNAs. RNA-guided CRISPR enzymes are classified as type V restriction enzymes. See also U.S. Patent Publication 2014/0068797, which is incorporated by reference in its entirety.

### Cas9

Cas9 is a nuclease, an enzyme specialized for cutting DNA, with two active cutting sites, one for each strand of the double helix. The team demonstrated that they could disable one or both sites while preserving Cas9′s ability to home located its target DNA. Jinek et al. (2012) combined tracrRNA and spacer RNA into a “single-guide RNA” molecule that, mixed with Cas9, could find and cut the correct DNA targets. It has been proposed that such synthetic guide RNAs might be able to be used for gene editing (Jinek et al., Science. 2012 Aug. 17:337(6096):816-21).

Cas9 proteins are highly enriched in pathogenic and commensal bacteria. CRISPR/Cas-mediated gene regulation may contribute to the regulation of endogenous bacterial genes, particularly during bacterial interaction with eukaryotic hosts. For example, Cas protein Cas9 of Francisella novicida uses a unique, small, CRISPR/Cas-associated RNA (scaRNA) to repress an endogenous transcript encoding a bacterial lipoprotein that is critical for F. novicida to dampen host response and promote virulence. Coinjection of Cas9 mRNA and sgRNAs into the germline (zygotes) generated mice with mutations. Delivery of Cas9 DNA sequences also is contemplated.

gRNA

As an RNA guided protein, Cas9 requires a short RNA to direct the recognition of DNA targets. Though Cas9 preferentially interrogates DNA sequences containing a PAM sequence NGG it can bind here without a protospacer target. However, the Cas9-gRNA complex requires a close match to the gRNA to create a double strand break. CRISPR sequences in bacteria are expressed in multiple RNAs and then processed to create guide strands for RNA. Because Eukaryotic systems lack some of the proteins required to process CRISPR RNAs the synthetic construct gRNA was created to combine the essential pieces of RNA for Cas9 targeting into a single RNA expressed with the RNA polymerase type 21 promoter U6). Synthetic gRNAs are slightly over 100 bp at the minimum length and contain a portion which is targets the 20 protospacer nucleotides immediately preceding the PAM sequence NGG: gRNAs do not contain a PAM sequence.

### Pharmaceutical Compositions

In some aspects, the present invention provides pharmaceutical compositions. To prepare the pharmaceutical compositions of this invention, an effective amount of an agent (e.g., a pseudotyped viral particle) is combined with a pharmaceutically acceptable carrier, which carrier may take a wide variety of forms depending on the form of preparation desired for administration. In some embodiments, the pharmaceutical composition comprises a cell that can be used to produce pseudotyped viral particles of the invention. These pharmaceutical compositions are desirable in unitary dosage form suitable, particularly, for administration percutaneously, or by parenteral injection. Any of the usual pharmaceutical media may be employed such as, for example, water, glycols, oils, alcohols and the like in the case of oral liquid preparations such as suspensions, syrups, elixirs and solutions: or solid carriers such as starches, sugars, kaolin, lubricants, binders, disintegrating agents and the like in the case of powders, pills, capsules, and tablets. For parenteral compositions, the carrier will usually comprise sterile water, at least in large part, though other ingredients, for example, to aid solubility and cell viability, may be included. Other ingredients may include antioxidants, viscosity stabilizers, chelating agents, buffers, preservatives. If desired, further ingredients may be incorporated in the compositions, e.g. anti-inflammatory agents, antibacterials, antifungals, disinfectants, vitamins, antibiotics.

Agents of the invention may be administered as part of a pharmaceutical composition. The compositions should be sterile and contain a therapeutically effective amount of the polypeptides or nucleic acid molecules in a unit of weight or volume suitable for administration to a subject.

Agents of the invention (e.g., a pseudotyped viral particle) may be administered within a pharmaceutically-acceptable diluent, carrier, or excipient, in unit dosage form. Conventional pharmaceutical practice may be employed to provide suitable formulations or compositions to administer the compounds to patients suffering from a neurological condition. Administration may begin before the patient is symptomatic. Any appropriate route of administration may be employed, for example, administration may be parenteral, intravenous, intraarterial, subcutaneous, intratumoral, intramuscular, intracranial, intraorbital, ophthalmic, intraventricular, intrahepatic, intracapsular, intrathecal, intracisternal, intraperitoneal, intranasal, aerosol, suppository, or oral administration. For example, therapeutic formulations may be in the form of liquid solutions or suspensions: for oral administration, formulations may be in the form of tablets or capsules: and for intranasal formulations, in the form of powders, nasal drops, or aerosols. In some embodiments, the composition is administered locally to a patient (e.g., proximal to a tumor) and not systemically. In some embodiment, the composition is administered systemically.

Methods well known in the art for making formulations are found, for example, in “Remington: The Science and Practice of Pharmacy” Ed. A. R. Gennaro, Lippincourt Williams & Wilkins, Philadelphia, Pa., 2000. Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful parenteral delivery systems include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel. The formulations can be administered to human patients in therapeutically effective amounts (e.g., amounts which prevent, eliminate, or reduce a pathological condition) to provide therapy for a neoplastic disease or condition. The preferred dosage of an agent of the invention is likely to depend on such variables as the type and extent of the disorder, the overall health status of the particular subject, the formulation of the compound excipients, and its route of administration.

Generally, doses of pseudotyped viral particles of the present invention can be from about or at least about 1×10e7 transduction units (TU), 1×10e8 TU, 1×10e9 TU, 1×10e10 TU, or 1×10e11 TU. In embodiments, the dose of the pseudotyped viral particle of the present invention is about or at least about 1×10e7 TU/kg, 1×10e8 TU/kg, 1×10e9 TU/kg, 1×10e10 TU/kg, or 1×10e11 TU/kg. Lower doses will result from certain forms of administration, such as intravenous administration. In the event that a response in a subject is insufficient at the initial doses applied, higher doses (or effectively higher doses by a different, more localized delivery route) may be employed to the extent that patient tolerance permits. Multiple doses per day are contemplated to achieve appropriate systemic levels of an agent of the invention.

A variety of administration routes are available. The methods of the invention, generally speaking, may be practiced using any mode of administration that is medically acceptable, meaning any mode that produces effective levels of the active compounds without causing clinically unacceptable adverse effects. Other modes of administration include oral, rectal, topical, intraocular, buccal, intravaginal, intracisternal, intracerebroventricular, intratracheal, nasal, transdermal, within/on implants, e.g., fibers such as collagen, osmotic pumps, or grafts comprising appropriately transformed cells, etc., or parenteral routes.

### Methods of Treatment

The present invention provides methods of treating disease and/or disorders or symptoms thereof which comprise administering a therapeutically effective amount of a pharmaceutical composition comprising a pseudotyped viral particle (e.g., a pseudotyped lentiviral particle or a psedudotyped gammaretroviral particle). Thus, one embodiment is a method of treating a subject suffering from or susceptible to a cancer or infection (e.g., cytomegalovirus (CMV), influenza, or coronavirus disease of 2019 (COVID-19)). The method includes the step of administering to the mammal a therapeutic amount of a pseudotyped viral particle sufficient to treat the disease or disorder or symptom thereof, under conditions such that the disease or disorder is treated.

The methods herein include administering to the subject (including a subject identified as in need of such treatment) an effective amount of a pesudotyped viral particle described herein, or a composition described herein to produce such effect. Identifying a subject in need of such treatment can be in the judgment of a subject or a health care professional and can be subjective (e.g. opinion) or objective (e.g. measurable by a test or diagnostic method).

The therapeutic methods of the invention (which include prophylactic treatment) in general comprise administration of a therapeutically effective amount of the pseudotyped viral particle herein to a subject (e.g., animal, human) in need thereof, including a mammal, particularly a human. Such treatment will be suitably administered to subjects, particularly humans, suffering from, having, susceptible to, or at risk for a disease (e.g., a cancer, cytomegalovirus (CMV), influenza, or coronavirus disease of 2019 (COVID-19)), disorder, or symptom thereof. Determination of those subjects “at risk” can be made by any objective or subjective determination by a diagnostic test or opinion of a subject or health care provider (e.g., genetic test, enzyme or protein marker, Marker (as defined herein), family history, and the like).

The cancer can be a hematologic cancer, e.g., a cancer chosen from one or more of chronic lymphocytic leukemia (CLL), acute leukemias, acute lymphoid leukemia (ALL), B-cell acute lymphoid leukemia (B-ALL), T-cell acute lymphoid leukemia (T-ALL), chronic myelogenous leukemia (CML), B cell prolymphocytic leukemia, blastic plasmacytoid dendritic cell neoplasm, Burkitt's lymphoma, diffuse large B cell lymphoma, follicular lymphoma, hairy cell leukemia, small cell-or a large cell-follicular lymphoma, malignant lymphoproliferative conditions, MALT lymphoma, mantle cell lymphoma, marginal zone lymphoma, multiple myeloma, myelodysplasia and myelodysplastic syndrome, non-Hodgkin's lymphoma. Hodgkin's lymphoma. plasmablastic lymphoma, plasmacytoid dendritic cell neoplasm, Waldenstrom macroglobulinemia, or pre-leukemia.

The cancer can also be chosen from colon cancer, rectal cancer, renal-cell carcinoma, liver cancer, non-small cell carcinoma of the lung, cancer of the small intestine, cancer of the esophagus, melanoma, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin's lymphoma, cancer of the endocrine system, cancer of the thyroid gland, cancer of the parathyroid gland, cancer of the adrenal gland, sarcoma of soft tissue, cancer of the urethra, cancer of the penis, solid tumors of childhood, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer. T-cell lymphoma, environmentally induced cancers, combinations of said cancers, and metastatic lesions of said cancers.

In one embodiment, the invention provides a method of monitoring treatment progress. The method includes the step of determining a level of diagnostic marker (Marker) or diagnostic measurement (e.g., screen, assay) in a subject suffering from or susceptible to a disorder or symptoms thereof associated with a disease (e.g., a cancer), in which the subject has been administered a therapeutic amount of a compound herein sufficient to treat the disease or symptoms thereof. The level of Marker determined in the method can be compared to known levels of Marker in either healthy normal controls or in other afflicted patients to establish the subject's disease status. In preferred embodiments, a second level of Marker in the subject is determined at a time point later than the determination of the first level, and the two levels are compared to monitor the course of disease or the efficacy of the therapy. In certain preferred embodiments, a pre-treatment level of Marker in the subject is determined prior to beginning treatment according to this invention: this pre-treatment level of Marker can then be compared to the level of Marker in the subject after the treatment commences, to determine the efficacy of the treatment.

The pharmaceutical compositions of this invention can be administered by any suitable routes including, by way of illustration, oral, topical, rectal, transdermal, subcutaneous, intravenous, intramuscular, intranasal, intracranial, intracerebral, intraventricular, intrathecal, and the like. In some embodiments, the administration modalities as described in U.S. Pat. Nos. 5,543,158: 5,641,515 and 5,399,363 may be used to deliver compositions of the present invention.

For therapeutic uses, the compositions and agents disclosed herein may be administered by any convenient method: for example, parenterally, conveniently in a pharmaceutically or physiologically acceptable carrier, e.g., phosphate buffered saline, saline, deionized water, or the like. The compositions may be added to a retained physiological fluid such as blood or synovial fluid. For central nervous system (CNS) administration, a variety of techniques are available for promoting transfer of an agent across the blood brain barrier including disruption by surgery or injection, drugs which transiently open adhesion contact between central nervous system (CNS) vasculature endothelial cells, and compounds which facilitate translocation through such cells. As examples, many of the disclosed compositions are amenable to be directly injected or infused or contained within implants e.g. osmotic pumps, grafts comprising appropriately transformed cells. Compositions of the present invention may also be amenable to direct injection or infusion, topical, intratracheal/nasal administration e.g. through aerosol, intraocularly, or within/on implants e.g. fibers e.g. collagen, osmotic pumps, or grafts comprising appropriately transformed cells. Generally, the amount administered will be empirically determined. Other additives may be included, such as stabilizers, bactericides, etc. In various embodiments, these additives can be present in conventional amounts.

In various embodiments, the agents of the present invention are administered in sufficient amounts to provide sufficient levels of the agent in a subject without undue adverse effects. Conventional and pharmaceutically acceptable routes of administration include, but are not limited to, direct delivery to a selected organ or tissue (e.g., the spinal cord or brain), oral, inhalation (including intranasal and intratracheal delivery), intraocular, intravenous, intramuscular, subcutaneous, intradermal, intratumoral, and other parental routes of administration. Routes of administration may be combined, if desired.

The dose of an agent used to achieve a particular “therapeutic effect” will vary based on several factors including, but not limited to: the route of administration, the level of gene or RNA expression used to achieve a therapeutic effect, the specific disease or disorder being treated, and the stability of the agent. One of skill in the art can readily determine a dose range to treat a patient having a particular disease, injury, or condition based on the aforementioned factors, as well as other factors that are well known in the art.

Administration of agents of the present invention to a subject may be by. for example, intramuscular injection or by administration into the bloodstream of the subject. Administration into the bloodstream may be by injection into a vein, an artery, or any other vascular conduit. Agents of the present invention can be inserted into a delivery device which facilitates introduction by injection or implantation into a subject. Such delivery devices include tubes, e.g., catheters, for injecting cells and fluids into the body of a recipient subject. In a preferred embodiment, the tubes additionally have a needle, e.g., a syringe, through which the contents of the invention can be introduced into the subject at a desired location. Agents of the invention can be inserted into such a delivery device, e.g., a syringe, in different forms. For example, an agent can be suspended in a solution or embedded in a support matrix when contained in such a delivery device. As used herein, the term “solution” includes a pharmaceutically acceptable carrier or diluent in which the agent of the invention remain functional and/or viable. Pharmaceutically acceptable carriers and diluents include saline, aqueous buffer solutions, solvents and/or dispersion media. The use of such carriers and diluents is well known in the art. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. In some embodiments, the selection of the carrier is not a limitation of the present invention. The solution is preferably sterile and fluid. Preferably, the solution is stable under the conditions of manufacture and storage and preserved against the contaminating action of microorganisms such as bacteria and fungi through the use of, for example, parabens, chlorobutanol, phenol, ascorbic acid, thimerosal, and the like. Solutions of the invention can be prepared by incorporating recombinant adeno-associated virus particles, nucleotide molecules, and/or vectors as described herein in a pharmaceutically acceptable carrier or diluent and, as other ingredients enumerated herein, followed by filtered sterilization. Optionally, an agent may be administered on support matrices. Support matrices in which an agent can be incorporated or embedded include matrices which are recipient-compatible and which degrade into products which are not harmful to the recipient. Natural and/or synthetic biodegradable matrices are examples of such matrices. Natural biodegradable matrices include plasma clots, e.g., derived from a mammal, and collagen matrices. Synthetic biodegradable matrices include synthetic polymers such as polyanhydrides, polyorthoesters, and polylactic acid. Other examples of synthetic polymers and methods of incorporating or embedding cells into these matrices are known in the art. These matrices provide support and protection for the cells in vivo.

Methods of introduction may also be provided by rechargeable or biodegradable devices. Various slow release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a bioactive factor at a particular target site.

One feature of certain embodiments of an implant can be the linear release of an agent of the present invention, which can be achieved through the manipulation of the polymer composition and form. By choice of monomer composition or polymerization technique, the amount of water, porosity and consequent permeability characteristics can be controlled. The selection of the shape, size, polymer, and method for implantation can be determined on an individual basis according to the disorder, injury, or disease to be treated and the individual patient response. The generation of such implants is generally known in the art. In another embodiment of an implant an agent of the invention is encapsulated in implantable hollow fibers or the like. Such fibers can be pre-spun and subsequently loaded with the agent, or can be co-extruded with a polymer which acts to form a polymeric coat about the agent.

In addition to the methods of delivery described above, the following techniques are also contemplated as alternative methods of delivering an agent to a subject. Ultrasound has been used as a device for enhancing the rate and efficacy of drug permeation into and through a circulatory system. Other drug delivery alternatives contemplated are intraosseous injection (see, e.g., U.S. Pat. No. 5,779,708), microchip devices (see, e.g., U.S. Pat. No. 5,797,898), ophthalmic formulations, transdermal matrices (see, e.g., U.S. Pat. Nos. 5,770,219 and 5,783,208), and feedback-controlled delivery (see, e.g., U.S. Pat. No. 5,697,899).

### Kits

Also provided are kits for preventing or treating a disease (e.g., a cancer, an influenza infection, a coronavirus disease, or a cytomegalovirus infection), condition, or pathology in a subject in need thereof. In one embodiment, the kit provides a therapeutic or prophylactic composition containing an effective amount of a pseudotyped viral particle as described herein, which contains a glycoprotein domain or fragment thereof fused to a VHH domain or fragment thereof, where the kit is for use in administering the pseudotyped viral particle to a subject. In embodiments, the pseudotyped viral particle targets an immune cell (e.g., a B cell, a dendritic cell, an cosinophil, a granulocyte, an iNKT cell, a macrophage, a monocyte, a natural killer cell, a neutrophil, a lymphoma cell, a regulatory T cell, and a T cell).

In another embodiment, the kit provides a therapeutic or prophylactic composition containing an effective amount of a pseudotyped viral particle as described herein.

In some embodiments, the kit comprises a sterile container which contains the therapeutic or prophylactic composition: such containers can be boxes, ampoules, bottles, vials, tubes, bags, pouches, blister-packs, or other suitable container forms known in the art. The containers can be made of plastic, glass, laminated paper, metal foil, or other materials suitable for holding medicaments.

A composition comprising a viral particle pseudotyped with a glycoprotein domain or fragment thereof fused to a VHH domain or fragment thereof, as described herein, is provided together with instructions for administering the composition to a subject having or at risk of developing a disease. The instructions will generally include information about the use of the composition for the treatment of the disease. In other embodiments, the instructions include at least one of the following: description of the therapeutic agent: dosage schedule and administration for treatment or prevention of a disease (e.g., cancer) or symptoms thereof: precautions: warnings: indications: counter-indications: overdosage information: adverse reactions: animal pharmacology: clinical studies: and/or references. The instructions may be printed directly on the container (when present), or as a label applied to the container, as information stored on a remotely-accessible server, or as a separate sheet, pamphlet, card, or folder supplied in or with the container.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989): “Oligonucleotide Synthesis” (Gait, 1984): “Animal Cell Culture” (Freshney, 1987): “Methods in Enzymology” “Handbook of Experimental Immunology” (Weir, 1996); “Gene Transfer Vectors for Mammalian Cells” (Miller and Calos, 1987): “Current Protocols in Molecular Biology” (Ausubel, 1987): “PCR: The Polymerase Chain Reaction”, (Mullis, 1994): “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1: Using MV-H scFv Fusions for Targeting Lentiviral Vectors In Vitro

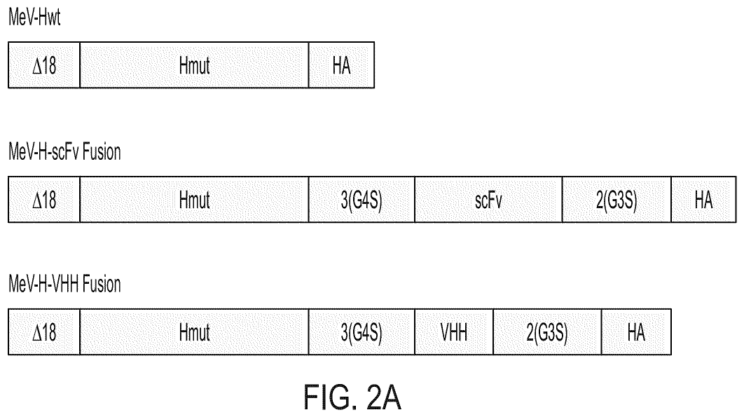

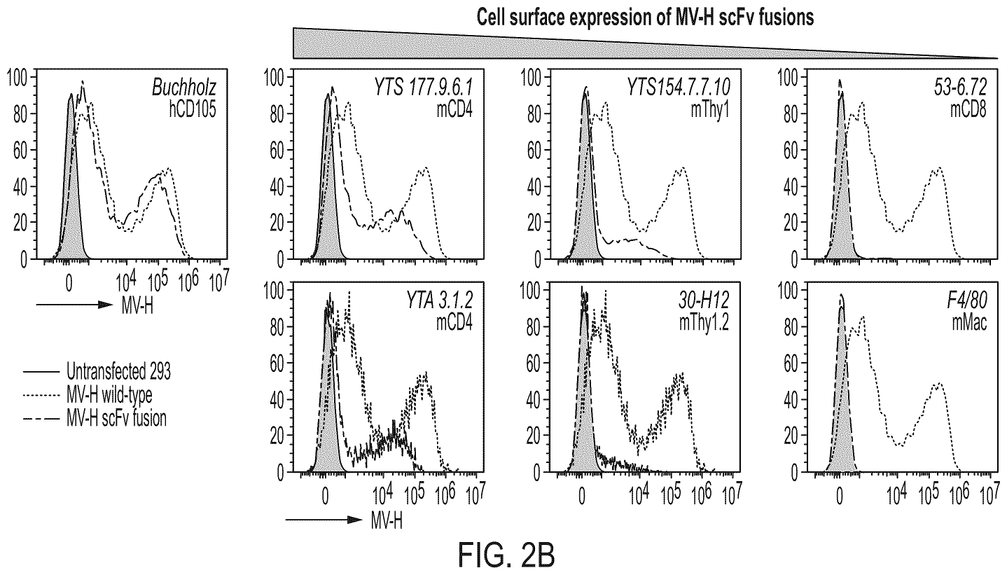

Many lentiviral vectors (LVs) are pseudotyped with the vesicular stomatitis virus glycoprotein (VSVg) (FIG. 1), due to its broad tissue tropism. However, LDL-R, the cellular receptor mediating VSVg LV entry, is poorly expressed on immune cells. To overcome this challenge, the effectiveness of retargeting Morbillivirus-pseudotyped lentiviral vectors using single chain variable fragments (scFvs) was evaluated. This was done by fusing a Lentivirus envelope glycoprotein to single chain variable fragments (scFvs) selectively targeted to specific cell surface antigens. Non-limiting examples of lentiviral envelope glycoproteins are presented in FIG. 1. In particular, scFvs targeting mouse cell surface antigens were fused to Morbillivirus hemagglutinin domain (MV-HA) to form a (scFv-MV-HA fusion (see FIG. 2A) for targeted lentiviral delivery of CRISPR sgRNAs to cell subsets of interest.

Hybridomas for antibodies targeting murine cell markers, Table 1, were identified and the leader & variable domains were sequenced to design scFv-MV-HA fusion polypeptides.

TABLE 1
Hybridomal antibodies used to design scFvs for targeted gene delivery.

| | | | | | | |
|---|---|---|---|---|---|---|
| Target | CD4 | CD8a | CD45 | NK1.1 | mThy1 | mEmr1 |
| Cell subset | CD4⁺ T cells | CD8⁺ T cells | Immune cells | NK cells | T cells | Macrophages |
| Hybridomas | GK1.5 | 53-6.72 | MB23G2 | PK136 | YTS | F4/80 |
| | | YTS 169.4.2.1 | YW 62.3.20 | | 154.7.7.10 | |
| | YTS 191.1.1.2 | | | | YBM 29.2.1 | |
| | YTS 177.9.6.1 | | | | 30-H12 | |
| | YTS 3.1.2 | | | | | |

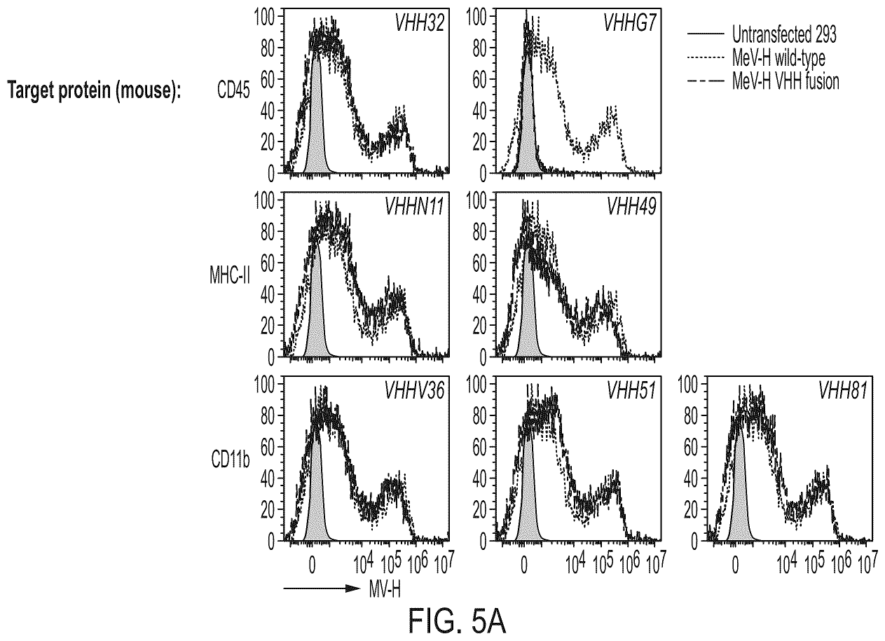

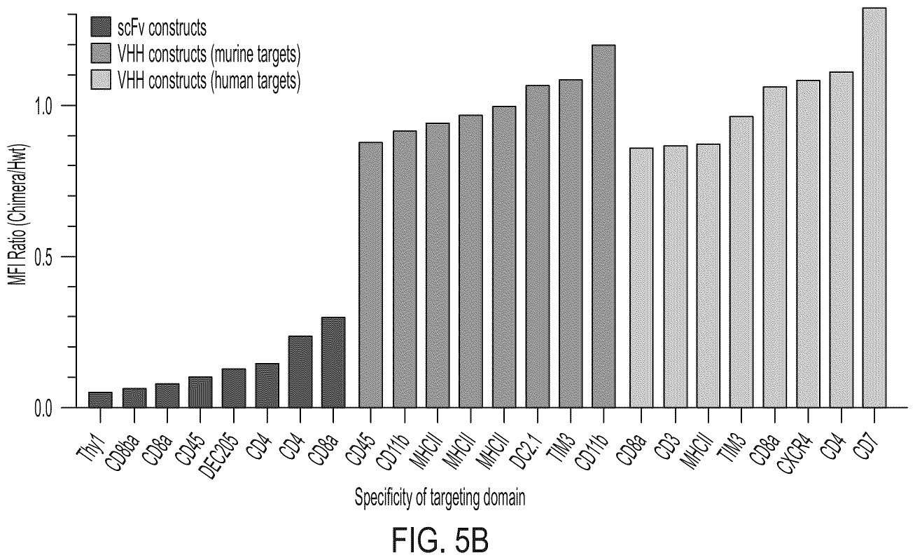

It is known that scFv fusion polypeptides are poorly expressed by viral producer cells. Therefore, to identify scFvs fusion polypeptides capable of high expression, multiple unique scFvs were evaluated. As a positive control, the MV-H-scFv fusion targeting hCD105 (Bucholtz hCD105) from Kays, et al. was used (see Kays, et al., “CD105 Is a Surface Marker for Receptor-Targeted Gene Transfer into Human Long-Term Repopulating Hematopoietic Stem Cells,” Stem Cells and Development, vol. 24, no. 6 (2014), DOI: 10.1089/scd.2014.0455, which is incorporated herein by reference in its entirety). Some scFv amino acid sequences were taken from Anliker B, et al. “Specific gene transfer to neurons, endothelial cells and hematopoietic progenitors with lentiviral vectors.” Nat Methods. 2010 November:7(11):929-35. doi: 10.1038/nmeth. 1514. Epub 2010 Oct. 10. PMID: 20935652; and/or Völkel, et al., “Isolation of endothelial cell-specific human antibodies from a novel fully synthetic scFv library,” Biochemical and Biophysical Research Communications, Volume 317, Issue 2, 2004, Pages 515-521, ISSN 0006-291X, DOI: 10.1016/j.bbrc.2004.03.074, the disclosures of which are incorporated herein by reference in its entirety for all purposes. The MV-H-scFv fusions were variably expressed on producer cells (293T cells), and generally showed low levels of surface-display (FIGS. 2B and 5B).

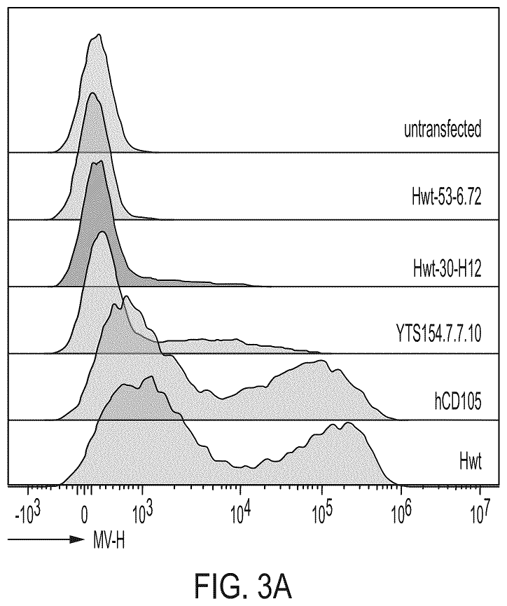

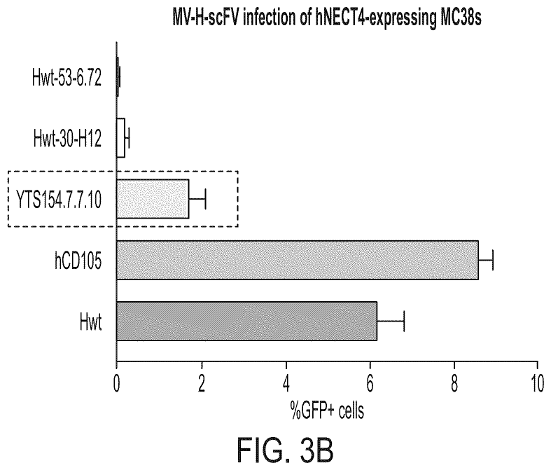

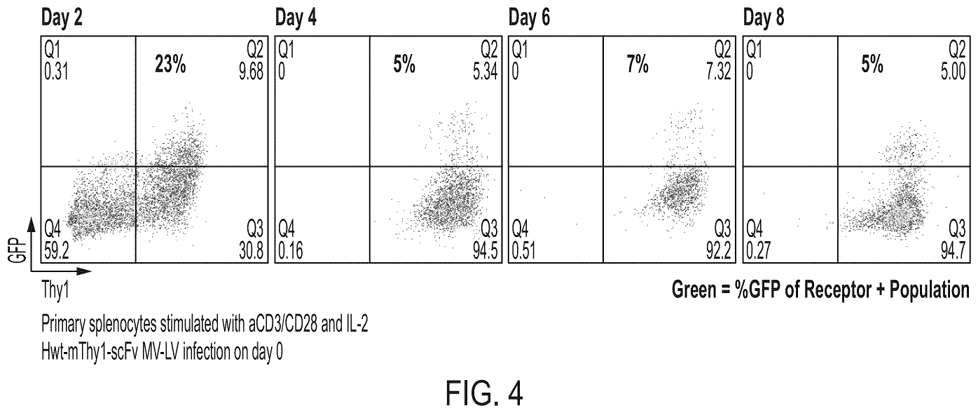

Lentiviral vectors comprising scFv-MV-HA fusions designed based upon antibodies from the YTS 154.7.7.10 and 30-H12 hybridomas were tested in vivo for infection selectivity and efficiency (FIGS. 3A and 3B). These scFv-MV-HA fusions, which showed poor levels of surface-display in producer cells, were associated with poor infection of receptor-expressing cells (FIGS. 3A and 3B). Nevertheless, despite the poor cell surface expression, the mThy1-scFv-H fusion designed based upon an antibody from YTS 154.7.7.10 mediated some stable T cell transduction (primary splenocytes stimulated with aCD3/CD28 and IL-2) (FIG. 4).

Without intending to be bound by theory, the poor surface expression and resulting limited infectious ability of the lentiviral vectors comprising the scFv-MV-HA fusions may have been due to aggregation or improper folding of the scFv-MV-HA fusions.

### Example 2: Using VHH-MV-HA Fusions for Targeting Lentiviral Vectors In Vitro

Given the poor expression and limited infectious ability of the scFv-MV-HA fusions, an alternative approach was taken to retarget the lentiviral vectors. In particular, the efficacy of using various VHH domains (Table 2 provides a list of VHH domains) in place of the scFv domains in the scFv-MV-HA fusions was evaluated (see FIG. 2A). VHH-MV-HA fusions were prepared using VHH domains listed in Table 2. Table 2 also includes a list of VHH domains suitable for targeting human antigens.

TABLE 2
VHH domains considered for evaluation and VHH domains targeting
human antigens. VHH domains in bold were evaluated.

| Target | VHH domains |
|---|---|
| CD8a (subset of T cells) | aCD8 VHH, R2HCD26, |
| | R3HCD27, R3HCD129 |
| CD11b (Monocyte) | 51, 81, V36, VHHDC13, |
| | aCD11b VHH |
| CD45 (immune cells) | 32, G7 |
| MHCII | VHH7, 49, N11, DC1, DC8, |
| | DC14, DC15, VHH4 |
| TIM3 | mH2 |
| Dendritic cell | DC1.8, DC2.1 (antigen |
| | unknown) |
| CD7 | VHH6, VHH10, VHH6 |
| | Humanized 1, VHH10 |
| | Humanized 1, VHH6 |
| | Humanized 2, VHH10 |
| | Humanized 2 |
| CD3 | Anti-CD3 VHH |
| CD4 | 03F11 |
| CD38 | MU375, MU551, MU1053 |
| TIM3 | hH6 |
| CXCR4 | 281F12 |
| CTLA-4 | 6RQM |

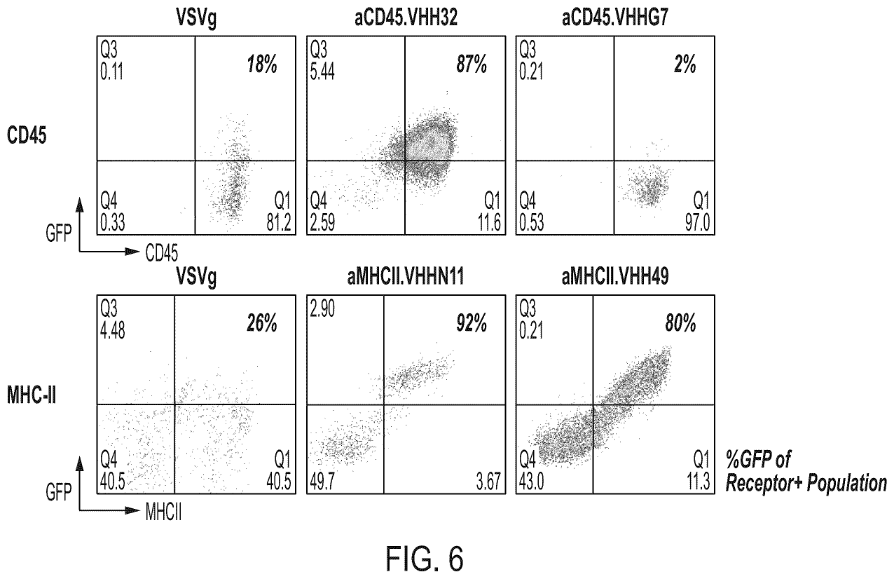

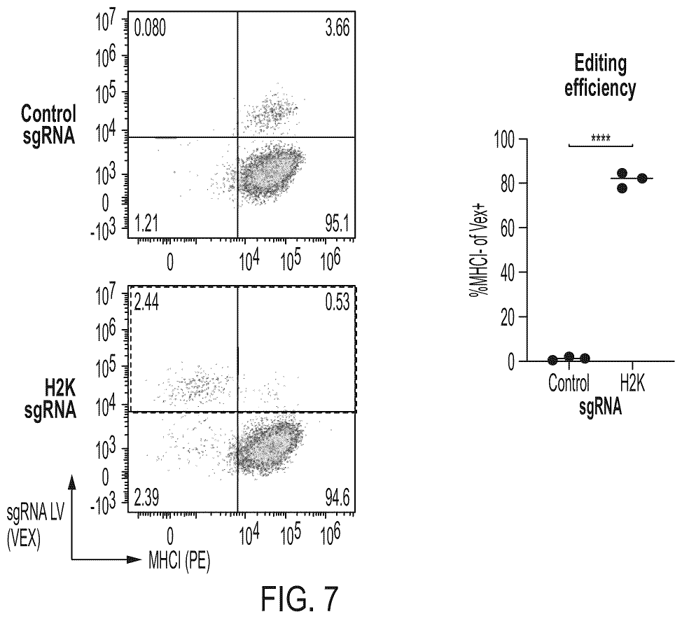

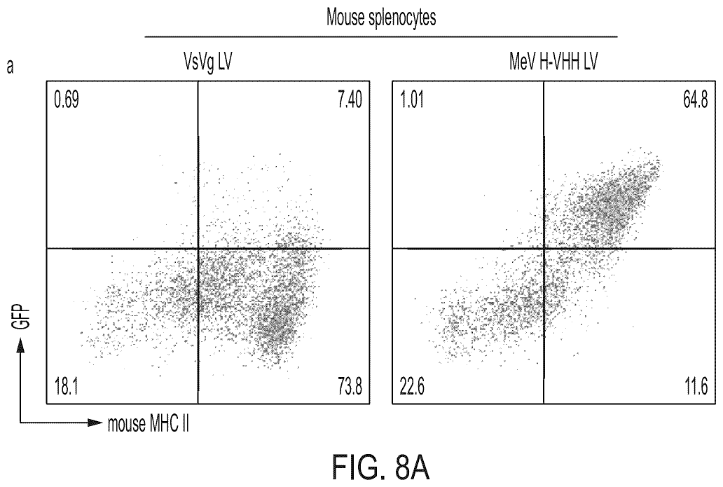

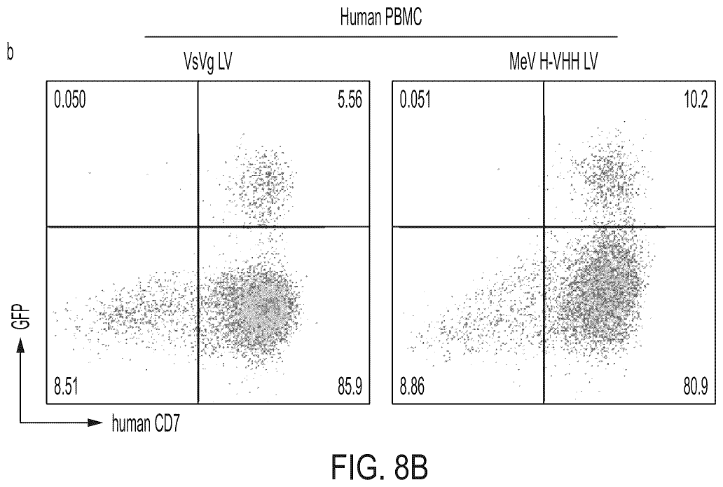

Surface expression of the VHH-MV-HA fusions in 293T cells was superior to that of the scFv-MV-HA fusions (FIGS. 5A and 5B). Lentiviral vectors containing VHH-MV-HA fusions targeting CD45 or MHC-II efficiently and selectively infected primary splenocytes (FIG. 6). Lentiviral vectors containing VHH-MV-HA fusions targeting mouse MHCII successfully infected mouse splendocytes and lentiviral vectors containing VHH-MV-HA fusions targeting human CD7 successfully infected human peripheral blood mononuclear cells (PBMC), see FIGS. 8A and 8B, respectively. Infected cells expressed GFP. The VHH-MV-HA fusions were associated with higher infection rates than VsVg-pseudotyped Lentivirus. Also, CRISPR guides delivered by lentiviral vectors containing CD45-VHH-H fusions efficiently edited MHC-I in primary CD8 T cells (FIG. 7).

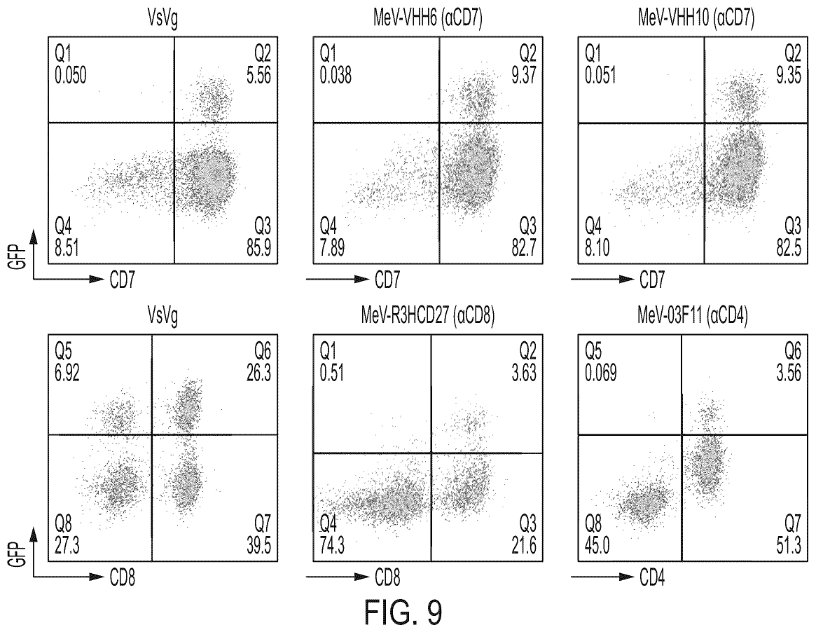

Lentiviral vectors containing VHH-MV-HA fusions targeting the human cell surface proteins αCD7, αCD8, or αCD4 efficiently and selectively infected primary human cells activated in vitro, see FIG. 9 where specifically infected cells fall within the upper-right quadrants (i.e., high GFP expression and high surface-expression of the target polypeptide). 10E5 human PBMCs were stimulated with αCD3/αCD28 beads and hIL-2, and infected 3-days post-stimulation with Lentivirus. Expression levels were measured using flow cytometry five days post-infection.

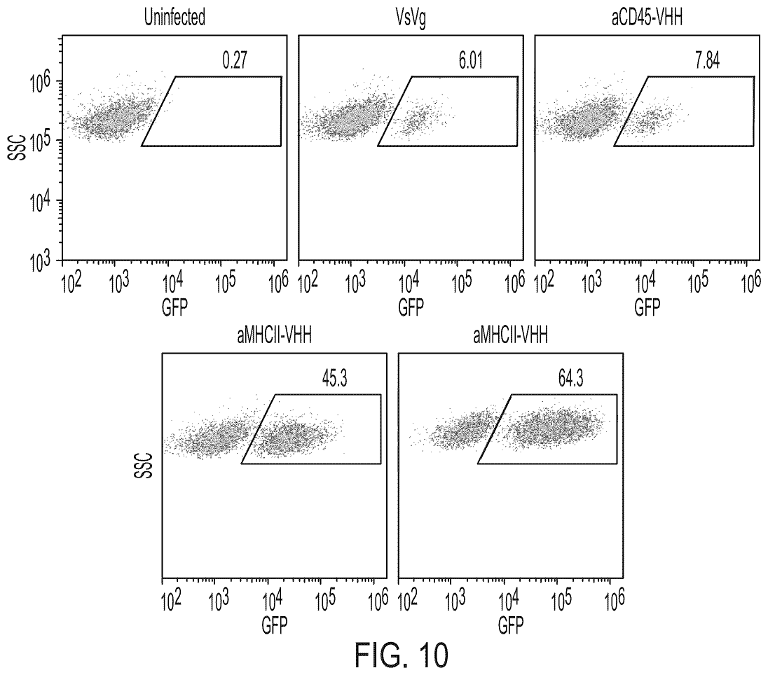

Next, the ability of lentiviral vectors containing VHH-MV-HA fusions to infect murine B cell lymphoma cells was evaluated. Lentiviral vectors containing VHH-MV-HA fusions targeting murine αMHCII or αCD45 were used to infect A20 cells (A20 mouse B cell lymphoma model, which is CD45+and MHCII+) (FIG. 10). The VHH-MV-HA fusions infected the A20 cells more efficiently than VsVg-pseudotyped Lentivirus (FIG. 10). Not being bound by theory, A20 cells express low levels of the low-density lipoprotein-receptor (LDLR) targeted by the VsVg-pseudotyped Lentivirus and, therefore, the VsVg-pseudotyped Lentivirus was unable to infect the A20 cells.

Efficient lentiviral infection of immune cells was accomplished by engineering target-specific lentiviral particle containing VHH-MV-HA fusions. The VHH-MV-HA fusions were superior to scFv-MV-HA fusions and enabled cell-specific targeting in vitro. Successful CRISPR editing was carried out using lentiviral particles retargeted using VHH-MV-HA fusions.

### Example 3: Using VHH-MV-HA Fusions for Targeting Lentiviral Vectors In Vivo

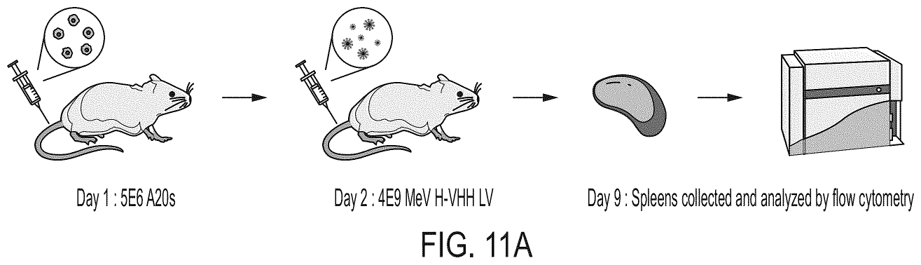

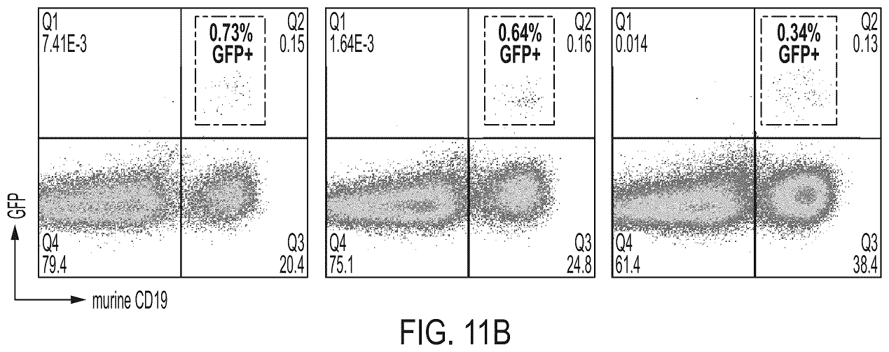

Given the in vitro ability of lentiviral vectors pseudotyped with VHH-MV-HA fusions targeting αMHCII to specifically infect A20 cells, experiments were next undertaken to evaluate the efficacy of the lentiviral vectors in infecting A20 cells in vivo. The lentiviral vectors pseudotyped with VH-MV-HA fusions targeting αMHCII were introduced by intravenous injection to immunocompromised mice (NOD scid gamma (NSG) mice) injected one day before with A20 cells (A20 mouse B cell lymphoma model, which is CD45+and MHCII+). A20 cells within the spleens of the mice were then evaluated on day 9 post-injection with the A20 cells by flow cytometry (FIG. 11A). Cells successfully infected with the Lentivirus expressed GFP. It was found that the A20 cells homed toward the mouse spleen. CD19 was used as a general marker for the cells becauseall, or nearly all, of the A20 cells surface-expressed CD19. The pseudotyped Lentivirus had high selectivity for the target cells, as can be seen by the high number of cells falling within the second quadrant (Q2) of each of the plots of FIG. 11B and the relatively low number of cells falling within the first quadrant (Q1) of the plots (i.e., low CD19 expression and low/no GFP expression).

### Methods

The following methods were employed in the above examples.

### Generation of Retargeted MeV Envelope Proteins

Codon optimized polynucleotides encoding MeV Fc30 (where the “c30” designates a cytoplasmic tail truncation of 30 amino acids) and MeV Hc18 WT (where “c18” designates a cytoplasmic tail truncation of 18 amino acids: alternatively, Hwtc18) were synthesized at GenScript. The MeV H protein contained an N481A mutation (Hmut) to prevent activation from murine TLR2 (CITE). Commercially available hybridomas were cultured and the gDNA synthesized and sequenced for VH and VL sequences at GenScript. scFvs were designed by adding a (G4S)3 linker between the VH and VL domains. The resulting scFvs were cloned into a pCG-Hwtc18 plasmid (which contained a pCG plasmid backbone) through either infusion cloning or Notl and Spel RE sites. Codon-optimized nanobody polynucleotides were synthesized at GenScript and similarly cloned into pCG-Hwtc18.

### Surface Expression Assay

1E6 HEK293T cells were seeded in 6-well plates. 24-hours later, the media was changed with fresh pre-warmed complete DMEM (Dulbecco's modified eagle medium). 1 μg of envelope plasmid was diluted in 100 μL Opti-MEM (optimized minimal essential medium) and incubated with 5 μL PEI (polyethylenimine buffer) for 20 minutes at room temperature. The Opti-MEM, plasmid, PEI mixture was then added dropwise to the cells.

24-hours later the cells were collected via trypsinization and washed with MACS buffer (phosphate-buffered saline (PBS) +1% fetal bovine serum (FBS) 4 mM ethylenediamine tetraacetic acid (EDTA)). 1E6 cells were stained with 10 μg/mL of CL55 anti MeV H mIgG2a antibody or 10 μg/mL Y503 anti MeV F antibody (from French biobank) for 1 hour at 4 C. Cells were washed three times with MACS buffer then incubated with a PE anti-mIgG2a antibody (Biolegend) for 3 minutes. Cells were washed with MACS then analyzed on a CytoflexLX flow cytometer. Alternatively, transfected 293T cells were stained with an anti HA-tag or anti FLAG-tag antibody (Biolegend).

### Lentivirus Production

For Lentivirus generation 18×10E6 HEK293T cells were seeded into a T175 flask with 25 mL of Dulbecco's Modified Eagle Medium (DMEM) (Supplemented with 10% fetal bovine serum (FBS) Pen/Strep and Genatmicin). 24-hours later, media was replaced with warm DMEM. For generation of re-targeted Lentivirus about 2.7 μg of pCG-Hwtc18-VHH, 1.8 μg pCG-Fc30, 13.5 μg psPAX2 (Addgene), and 18 μg of an EFS-GFP transfer vector were diluted in 1.8 mL Opti-MEM (optimized minimal essential medium) to which 144 μL of polyethylenimine (PEI) was added and incubated for 20 minutes at room temperature (for traditional VSV-G pseudotyped Lentivirus 4.5 μg of pMD2.G was added instead of pCG-Fc30 and pCG-Hwtc18-VHH). The mixture was then added dropwise to the cells. 12-16 hours post-transfection, the media was replaced with fresh pre-warmed DMEM. 48-60 hours later the media was collected and filtered through 0.45 μM surfactant-free cellulose acetate (SFCA) membrane to remove cell debris.

To concentrate Lentivirus LentiX was added to virus-containing supernatant at 1 1:3 lentiX:supernatant ratio and incubated at 4 C for 24-72 hours then spun at 1500×g for 45 minutes and resuspended in PBS or HBSS. Lentivirus was also concentrated via ultracentrifugation at 72,000×g for 2 hours and resuspended in PBS (phosphate-buffered saline) or HBSS (Hank's balanced salt solution).

### In Vitro Virus Transduction

For transduction of cancer cell lines, a 96 well plate, 10E3 hNECTIN4 MC38 overexpression cells or A20s were seeded. 1-20 uL of 100× LentiX or Ultracentrifuge-concentrated GFP reporter virus was added per well. 2-3 days later cells were collected and washed with MACS buffer (phosphate-buffered saline (PBS) +1% fetal bovine serum (FBS) 4 mM ethylenediamine tetraacetic acid (EDTA)). Cells were stained with antibodies for the requisite targets (ex/hNECTIN4 for or mMHCII) and then analyzed for GFP expression by flow cytometry. GFP expression was measured every 2-3 days after to access signal stability.

For ex vivo transduction of primary splenocytes and T cells, spleens from 6-10 week old mice were excised and mechanically separated then filtered through 0.45 μm filters. Splenocytes were washed with PBS and then lysed with ACK (ammonium-chloride-potassium) buffer and a pan T cell or CD8 T cells tissue isolation kits (available from Miltenyi Biotech) were used to purify cell populations. Cells were then plated onto anti mCD3 coated 96 well plate with IL2 and anti mCD28 antibody and stimulated for 2 days. Following stimulation 100K cells were plated into a 96 well plates with 1-20 μL of 100× virus and incubated for 2 days. Cells were stained for surface receptors and markers then analyzed with flow cytometry and analyzed every 2-3 days to determine signal stability.

### Other Embodiments

From the foregoing description, it will be apparent that variations and modifications may be made to the invention described herein to adapt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. A pseudotyped viral particle comprising:
(a) an envelope comprising a fusion protein, wherein the fusion protein comprises a viral envelope glycoprotein domain or fragment thereof fused to a single variable domain on a heavy chain (VHH) antibody (VHH) domain or antigen binding fragment thereof, wherein the VHH domain or antigen binding fragment thereof specifically binds an antigen present on a target cell; and
(b) a heterologous polynucleotide.

2. The viral particle of claim 1, wherein the viral envelope glycoprotein domain or fragment thereof comprises a viral hemagglutinin domain or fragment thereof.

3. The viral particle of claim 1, wherein the viral envelope glycoprotein domain or fragment thereof is derived from a hemagglutinin polypeptide of the measles virus.

4. The viral particle of claim 1, wherein the viral envelope glycoprotein domain or fragment thereof comprises a sequence with at least 85% amino acid sequence identity to the following sequence:
MeV-Hwtc18 polypeptide
MGSRIVINREHLMIDRPYVLLAVLFVMFLSLIGLLAIAGIRLHRAAIYT
AEIHKSLSTNLDVTNSIEHQVKDVLTPLFKIIGDEVGLRTPQRFTDLVK
FISDKIKFLNPDREYDERDLTWCINPPERIKLDYDQYCADVAAEELMNA
LVNSTLLEARATNQFLAVSKGNCSGPTTIRGQFSNMSLSLLDLYLSRGY
NVSSIVTMTSQGMYGGTYLVGKPNLSSKGSELSQLSMHRVFEVGVIRNP
GLGAPVFHMTNYFEQPVSNDFSNCMVALGELKFAALCHREDSITIPYQG
SGKGVSFQLVKLGVWKSPTDMRSWVPLSTDDPVIDRLYLSSHRGVIADN
QAKWAVPTTRTDDKLRMETCFQQACKGKNQALCENPEWAPLKDNRIPSY
GVLSVNLSLTVELKIKIASGFGPLITHGSGMDLYKTNHNNVYWLTIPPM
KNLALGVINTLEWIPRFKVSPALFTVPIKEAGEDCHAPTYLPAEVDGDV
KLSSNLVILPGQDLQYVLATYDTSRVEHAVVYYVYSPSRSFSYFYPFRL
PIKGVPIELQVECFTWDKKLWCRHFCVLADSESGGHITHSGMVGMGVSC
TVTREDGT.

5. The viral particle of claim 1, wherein the VHH domain or antigen binding fragment thereof comprises a VHH amino acid sequence with at least 85% sequence identity to a sequence selected from the group consisting of:
Anti-MHCII VHH (N11)
QVQLVQSGGGLVQPGGSLGLSCAASGNIGSRDNMGWYRQAPGKQREWVA
TISGYGIATYRDSVKGRFTVAKDTAKNIVSLQMNYLTTEDTAVYYCYAY
AVDSRNIFWSQGTQVTVS;
Anti-CD45 (32) VHH
QVQLVQSGGGLVQPGGSLRLSCAASGRAFNSAAMGWYRQAPGSQRELVA
SISAGTASYADAVKGRFTISRDYAKNIIYLQMNSLKPDDTAVYFCNYRT
TYTSGYSEDYWGQGTQVTVS;
Anti-CD7 (VHH10) VHH
DVQLQESGGGSVQAGGSLRLSCAASGYTHSSYCMAWFRQAPGREREGVA
SIDSDGTTSYADSVKGRFTISQDNAKNTLYLQMNSLKPEDTAMYYCAAR
FGPMGCVDLSTLSFGHWGQGTQVTVSIT;
Anti-CD4 (03F11) VHH
EVQLVESGGGSVQPGGSLTLSCGTSGRTFNVMGWFRQAPGKEREFVAAV
RWSSTGIYYTQYADSVKSRFTISRDNAKNTVYLEMNSLKPEDTAVYYCA
ADTYNSNPARWDGYDFRGQGTQVTVSS;
and
Anti-CD8 (R3HCD27) VHH
QVQLQESGGGSVQPGGSLRLSCAASGFTFDDYAMSWVRQVPGKGLEWVS
TINWNGGSAEYAEPVKGRFTISRDNAKNTVYLQMNSLKLEDTAVYYCAK
DADLVWYNLSTGQGTQVTVSS.

6. The viral particle of claim 1, wherein the viral envelope glycoprotein domain or fragment thereof fused to the VHH domain or antigen binding fragment thereof fragment thereof comprises a sequence with at least 85% sequence identity to a sequence selected from the group consisting of:
MeV-Hwtc18-MHCII (N11)
MGSRIVINREHLMIDRPYVLLAVLFVMFLSLIGLLAIAGIRLHRAAIYT
AEIHKSLSTNLDVTNSIEHQVKDVLTPLFKIIGDEVGLRTPQRFTDLVK
FISDKIKFLNPDREYDERDLTWCINPPERIKLDYDQYCADVAAEELMNA
LVNSTLLEARATNQFLAVSKGNCSGPTTIRGQFSNMSLSLLDLYLSRGY
NVSSIVTMTSQGMYGGTYLVGKPNLSSKGSELSQLSMHRVFEVGVIRNP
GLGAPVFHMTNYFEQPVSNDFSNCMVALGELKFAALCHREDSITIPYQG
SGKGVSFQLVKLGVWKSPTDMRSWVPLSTDDPVIDRLYLSSHRGVIADN
QAKWAVPTTRTDDKLRMETCFQQACKGKNQALCENPEWAPLKDNRIPSY
GVLSVNLSLTVELKIKIASGFGPLITHGSGMDLYKTNHNNVYWLTIPPM
KNLALGVINTLEWIPRFKVSPALFTVPIKEAGEDCHAPTYLPAEVDGDV
KLSSNLVILPGQDLQYVLATYDTSRVEHAVVYYVYSPSRSFSYFYPFRL
PIKGVPIELQVECFTWDKKLWCRHFCVLADSESGGHITHSGMVGMGVSC
TVTREDGTGGGGSGGGGSGGGGSAAAQVQLVQSGGGLVQPGGSLGLSCA
ASGNIGSRDNMGWYRQAPGKQREWVATISGYGIATYRDSVKGRFTVAKD
TAKNIVSLQMNYLTTEDTAVYYCYAYAVDSRNIFWSQGTQVTVSGGGSG
GGSYPYDVPDYA;
MeV-Hwtc18-CD45 (32)
MGSRIVINREHLMIDRPYVLLAVLFVMFLSLIGLLAIAGIRLHRAAIYT
AEIHKSLSTNLDVTNSIEHQVKDVLTPLFKIIGDEVGLRTPQRFTDLVK
FISDKIKFLNPDREYDFRDLTWCINPPERIKLDYDQYCADVAAEELMNA
LVNSTLLEARATNQFLAVSKGNCSGPTTIRGQFSNMSLSLLDLYLSRGY
NVSSIVTMTSQGMYGGTYLVGKPNLSSKGSELSQLSMHRVFEVGVIRNP
GLGAPVFHMTNYFEQPVSNDFSNCMVALGELKFAALCHREDSITIPYQG
SGKGVSFQLVKLGVWKSPTDMRSWVPLSTDDPVIDRLYLSSHRGVIADN
QAKWAVPTTRTDDKLRMETCFQQACKGKNQALCENPEWAPLKDNRIPSY
GVLSVNLSLTVELKIKIASGFGPLITHGSGMDLYKTNHNNVYWLTIPPM
KNLALGVINTLEWIPRFKVSPALFTVPIKEAGEDCHAPTYLPAEVDGDV
KLSSNLVILPGQDLQYVLATYDTSRVEHAVVYYVYSPSRSFSYFYPFRL
PIKGVPIELQVECFTWDKKLWCRHFCVLADSESGGHITHSGMVGMGVSC
TVTREDGTGGGGSGGGGSGGGGSAAAQVQLVQSGGGLVQPGGSLRLSCA
ASGRAFNSAAMGWYRQAPGSQRELVASISAGTASYADAVKGRFTISRDY
AKNIIYLQMNSLKPDDTAVYFCNYRTTYTSGYSEDYWGQGTQVTVSGGG
SGGGSYPYDVPDYA;
MeV-Hwtc18-CD7 (humanized VHH10)
MGSRIVINREHLMIDRPYVLLAVLFVMFLSLIGLLAIAGIRLHRAAIYT
AEIHKSLSTNLDVTNSIEHQVKDVLTPLFKIIGDEVGLRTPQRFTDLVK
FISDKIKFLNPDREYDFRDLTWCINPPERIKLDYDQYCADVAAEELMNA
LVNSTLLEARATNQFLAVSKGNCSGPTTIRGQFSNMSLSLLDLYLSRGY
NVSSIVTMTSQGMYGGTYLVGKPNLSSKGSELSQLSMHRVFEVGVIRNP
GLGAPVFHMTNYFEQPVSNDFSNCMVALGELKFAALCHREDSITIPYQG
SGKGVSFQLVKLGVWKSPTDMRSWVPLSTDDPVIDRLYLSSHRGVIADN
QAKWAVPTTRTDDKLRMETCFQQACKGKNQALCENPEWAPLKDNRIPSY
GVLSVNLSLTVELKIKIASGFGPLITHGSGMDLYKTNHNNVYWLTIPPM
KNLALGVINTLEWIPRFKVSPALFTVPIKEAGEDCHAPTYLPAEVDGDV
KLSSNLVILPGQDLQYVLATYDTSRVEHAVVYYVYSPSRSFSYFYPFRL
PIKGVPIELQVECFTWDKKLWCRHFCVLADSESGGHITHSGMVGMGVSC
TVTREDGTGGGGSGGGGSGGGGSAAADVQLQESGGGSVQAGGSLRLSCA
ASGYTHSSYCMAWFRQAPGREREGVASIDSDGTTSYADSVKGRFTISQD
NAKNTLYLQMNSLKPEDTAMYYCAARFGPMGCVDLSTLSFGHWGQGTQV
TVSITGGGSGGGSYPYDVPDYA;
MeV-Hwtc18-CD4 (03F11)
MGSRIVINREHLMIDRPYVLLAVLFVMFLSLIGLLAIAGIRLHRAAIYT
AEIHKSLSTNLDVTNSIEHQVKDVLTPLFKIIGDEVGLRTPQRFTDLVK
FISDKIKFLNPDREYDERDLTWCINPPERIKLDYDQYCADVAAEELMNA
LVNSTLLEARATNQFLAVSKGNCSGPTTIRGQFSNMSLSLLDLYLSRGY
NVSSIVTMTSQGMYGGTYLVGKPNLSSKGSELSQLSMHRVFEVGVIRNP
GLGAPVFHMTNYFEQPVSNDFSNCMVALGELKFAALCHREDSITIPYQG
SGKGVSFQLVKLGVWKSPTDMRSWVPLSTDDPVIDRLYLSSHRGVIADN
QAKWAVPTTRTDDKLRMETCFQQACKGKNQALCENPEWAPLKDNRIPSY
GVLSVNLSLTVELKIKIASGFGPLITHGSGMDLYKTNHNNVYWLTIPPM
KNLALGVINTLEWIPRFKVSPALFTVPIKEAGEDCHAPTYLPAEVDGDV
KLSSNLVILPGQDLQYVLATYDTSRVEHAVVYYVYSPSRSFSYFYPFRL
PIKGVPIELQVECFTWDKKLWCRHFCVLADSESGGHITHSGMVGMGVSC
TVTREDGTGGGGSGGGGGGGGSAAAEVQLVESGGGSVQPGGSLTLSCGT
SGRTFNVMGWFRQAPGKEREFVAAVRWSSTGIYYTQYADSVKSRFTISR
DNAKNTVYLEMNSLKPEDTAVYYCAADTYNSNPARWDGYDFRGQGTQVT
VSSGGGSGGGSYPYDVPDYA;
and
MeV-Hwtc18-CD8a (R3HCD27)
MGSRIVINREHLMIDRPYVLLAVLFVMFLSLIGLLAIAGIRLHRAAIYT
AEIHKSLSTNLDVTNSIEHQVKDVLTPLFKIIGDEVGLRTPQRFTDLVK
FISDKIKFLNPDREYDERDLTWCINPPERIKLDYDQYCADVAAEELMNA
LVNSTLLEARATNQFLAVSKGNCSGPTTIRGQFSNMSLSLLDLYLSRGY
NVSSIVTMTSQGMYGGTYLVGKPNLSSKGSELSQLSMHRVFEVGVIRNP
GLGAPVFHMTNYFEQPVSNDFSNCMVALGELKFAALCHREDSITIPYQG
SGKGVSFQLVKLGVWKSPTDMRSWVPLSTDDPVIDRLYLSSHRGVIADN
QAKWAVPTTRTDDKLRMETCFQQACKGKNQALCENPEWAPLKDNRIPSY
GVLSVNLSLTVELKIKIASGFGPLITHGSGMDLYKTNHNNVYWLTIPPM
KNLALGVINTLEWIPRFKVSPALFTVPIKEAGEDCHAPTYLPAEVDGDV
KLSSNLVILPGQDLQYVLATYDTSRVEHAVVYYVYSPSRSFSYFYPFRL
PIKGVPIELQVECFTWDKKLWCRHFCVLADSESGGHITHSGMVGMGVSC
TVTREDGTGGGGSGGGGSGGGGSAAAQVQLQESGGGSVQPGGSLRLSCA
ASGFTFDDYAMSWVRQVPGKGLEWVSTINWNGGSAEYAEPVKGRFTISR
DNAKNTVYLQMNSLKLEDTAVYYCAKDADLVWYNLSTGQGTQVTVSSAA
AYPYDVPDYGSGGGSGGGSYPYDVPDYA.

7. The viral particle of claim 1, wherein the antigen is selected from the group consisting of BCR/Ig, CD3, CD4, CD7, CD8, CD11, CD19, CD20, CD30, CD34, CD38, CD45, CD133, CD103, CD105, CD110, CD117, CTLA-4, CXCR4, DC-SIGN, EGFR, Emr1, EpCAM, GluA4, Her2/neu, IL3R, IL7R, Mac, MHCII, Mucin 4, NK1.1, P-glycoprotein, TIM3, Thy1, and Thy1.2.

8. The viral particle of claim 1, wherein the VHH or fragment thereof is derived from a VHH selected from the group consisting of 03F11, 6QRM, aCD8 VHH, aCD11b VHH, Anti-CD3 VHH, DC1, DC1.8, DC2.1, DC8, DC14, DC15, hH6, 281F12, mH2, MU375, MU551, MU1053, R2HCD26, R3HCD27, R3HCD129, VHH4, VHH6, VHH6 Humanized 1, VHH6 Humanized 2, VHH7, VHH10, VHH10 Humanized 1, VHH10 Humanized 2, VHH32, VHH49, VHH51, VHH81, VHHDC13, VHHG7, VHHN11, and VHHV36.

9. A lentiviral particle comprising:
(a) an envelope comprising a fusion protein, wherein the fusion protein comprises a VHH domain or antigen binding fragment thereof and a Morbillivirus hemagglutinin domain or fragment thereof, wherein the VHH specifically binds αMHCII; and
(b) a polynucleotide encoding a guide polynucleotide and/or a Cas9 or another component of a genome editing system: or
(a) an envelope comprising a fusion protein, wherein the fusion protein comprises a VHH domain or antigen binding fragment thereof and a Morbillivirus hemagglutinin domain or fragment thereof, wherein the VHH specifically binds αCD7, αCD8, or αCD4; and
(b) a polynucleotide encoding a guide polynucleotide and/or a Cas9

10. A method for delivering a heterologous polynucleotide to a target cell, the method comprising: contacting a target cell with the viral particle of claim 1, thereby delivering the heterologous polynucleotide to the target cell.

11. A method of treating a subject having a cancer, the method comprising administering to the subject a composition comprising the pseudotyped viral particle of claim 1.

12. A method for generating a pseudotyped viral particle for delivering a heterologous polynucleotide to a target cell, the method comprising:
(a) displaying on the cell membrane of a eukaryotic cell a viral envelope glycoprotein domain or fragment thereof fused to a VHH domain or fragment thereof, wherein the VHH domain or fragment thereof specifically binds an antigen present on the target cell:
(b) transfecting the eukaryotic cell with a viral transfer vector and one or more additional vectors encoding one or more viral polypeptides, thereby generating the pseudotyped viral particle for delivering a heterologous polynucleotide to the target cell.

13. A eukaryotic cell for generating a pseudotyped viral particle, the eukaryotic cell comprising:
(a) a cell membrane comprising a viral envelope glycoprotein domain or fragment thereof fused to a VHH domain or fragment thereof, wherein the VHH domain or fragment thereof specifically binds an antigen present on a target cell:
(b) a viral transfer vector; and
(c) one or more additional vectors encoding one or more viral polypeptides.

14. A mammalian expression vector comprising a polynucleotide encoding a polypeptide comprising a viral envelope glycoprotein domain or fragment thereof fused to a VHH domain or fragment thereof, wherein the VHH domain or fragment thereof specifically binds an antigen present on a target cell.

15. The expression vector of claim 14, wherein the viral envelope glycoprotein domain or fragment thereof comprises a sequence with at least 85% sequence identity to the following sequence:
MeV-Hwtc18 polypeptide
MGSRIVINREHLMIDRPYVLLAVLFVMFLSLIGLLAIAGIRLHRAAIYT
AEIHKSLSTNLDVTNSIEHQVKDVLTPLFKIIGDEVGLRTPQRFTDLVK
FISDKIKFLNPDREYDERDLTWCINPPERIKLDYDQYCADVAAEELMNA
LVNSTLLEARATNQFLAVSKGNCSGPTTIRGQFSNMSLSLLDLYLSRGY
NVSSIVTMTSQGMYGGTYLVGKPNLSSKGSELSQLSMHRVFEVGVIRNP
GLGAPVFHMTNYFEQPVSNDFSNCMVALGELKFAALCHREDSITIPYQG
SGKGVSFQLVKLGVWKSPTDMRSWVPLSTDDPVIDRLYLSSHRGVIADN
QAKWAVPTTRTDDKLRMETCFQQACKGKNQALCENPEWAPLKDNRIPSY
GVLSVNLSLTVELKIKIASGFGPLITHGSGMDLYKTNHNNVYWLTIPPM
KNLALGVINTLEWIPRFKVSPALFTVPIKEAGEDCHAPTYLPAEVDGDV
KLSSNLVILPGQDLQYVLATYDTSRVEHAVVYYVYSPSRSFSYFYPFRL
PIKGVPIELQVECFTWDKKLWCRHFCVLADSESGGHITHSGMVGMGVSC
TVTREDGT.

16. The expression vector of claim 14, wherein the VHH domain or fragment thereof comprises a sequence with at least 85% sequence identity to a sequence selected from the group consisting of:
Anti-MHCII VHH (N11)
QVQLVQSGGGLVQPGGSLGLSCAASGNIGSRDNMGWYRQAPGKQREWVA
TISGYGIATYRDSVKGRFTVAKDTAKNIVSLQMNYLTTEDTAVYYCYAY
AVDSRNIFWSQGTQVTVS;
Anti-CD45 (32) VHH
QVQLVQSGGGLVQPGGSLRLSCAASGRAFNSAAMGWYRQAPGSQRELVA
SISAGTASYADAVKGRFTISRDYAKNIIYLQMNSLKPDDTAVYFCNYRT
TYTSGYSEDYWGQGTQVTVS;
Anti-CD7 (VHH10) VHH
DVQLQESGGGSVQAGGSLRLSCAASGYTHSSYCMAWFRQAPGREREGVA
SIDSDGTTSYADSVKGRFTISQDNAKNTLYLQMNSLKPEDTAMYYCAAR
FGPMGCVDLSTLSFGHWGQGTQVTVSIT;
Anti-CD4 (03F11) VHH
EVQLVESGGGSVQPGGSLTLSCGTSGRTFNVMGWFRQAPGKEREFVAAV
RWSSTGIYYTQYADSVKSRFTISRDNAKNTVYLEMNSLKPEDTAVYYCA
ADTYNSNPARWDGYDFRGQGTQVTVSS;
and
Anti-CD8 (R3HCD27) VHH
QVQLQESGGGSVQPGGSLRLSCAASGFTFDDYAMSWVRQVPGKGLEWVS
TINWNGGSAEYAEPVKGRFTISRDNAKNTVYLQMNSLKLEDTAVYYCAK
DADLVWYNLSTGQGTQVTVSS.

17. The expression vector of claim 16, wherein the viral envelope glycoprotein domain or fragment thereof and the VHH domain or fragment thereof are separated by a linker.

18. The expression vector of claim 16, wherein viral envelope glycoprotein domain or fragment thereof fused to the VHH domain or fragment thereof comprises a sequence with at least 85% sequence identity to a sequence selected from the group consisting of:
MeV-Hwtc18-MHCII (N11)
MGSRIVINREHLMIDRPYVLLAVLFVMFLSLIGLLAIAGIRLHRAAIYTAEIHKSLSTNLDVTN
SIEHQVKDVLTPLFKIIGDEVGLRTPQRFTDLVKFISDKIKFLNPDREYDERDLTWCINPPERI
KLDYDQYCADVAAEELMNALVNSTLLEARATNQFLAVSKGNCSGPTTIRGQFSNMSLSLLDLYL
SRGYNVSSIVTMTSQGMYGGTYLVGKPNLSSKGSELSQLSMHRVFEVGVIRNPGLGAPVFHMTN
YFEQPVSNDFSNCMVALGELKFAALCHREDSITIPYQGSGKGVSFQLVKLGVWKSPTDMRSWVP
LSTDDPVIDRLYLSSHRGVIADNQAKWAVPTTRTDDKLRMETCFQQACKGKNQALCENPEWAPL
KDNRIPSYGVLSVNLSLTVELKIKIASGFGPLITHGSGMDLYKTNHNNVYWLTIPPMKNLALGV
INTLEWIPRFKVSPALFTVPIKEAGEDCHAPTYLPAEVDGDVKLSSNLVILPGQDLQYVLATYD
TSRVEHAVVYYVYSPSRSFSYFYPFRLPIKGVPIELQVECFTWDKKLWCRHFCVLADSESGGHI
THSGMVGMGVSCTVTREDGTGGGGSGGGGSGGGGSAAAQVQLVQSGGGLVQPGGSLGLSCAASG
NIGSRDNMGWYRQAPGKQREWVATISGYGIATYRDSVKGRFTVAKDTAKNIVSLQMNYLTTEDT
AVYYCYAYAVDSRNIFWSQGTQVTVSGGGSGGGSYPYDVPDYA;
MeV-Hwtc18-CD45 (32)
MGSRIVINREHLMIDRPYVLLAVLFVMFLSLIGLLAIAGIRLHRAAIYTAEIHKSLSTNLDVTN
SIEHQVKDVLTPLFKIIGDEVGLRTPQRFTDLVKFISDKIKFLNPDREYDERDLTWCINPPERI
KLDYDQYCADVAAEELMNALVNSTLLEARATNQFLAVSKGNCSGPTTIRGQFSNMSLSLLDLYL
SRGYNVSSIVTMTSQGMYGGTYLVGKPNLSSKGSELSQLSMHRVFEVGVIRNPGLGAPVFHMTN
YFEQPVSNDFSNCMVALGELKFAALCHREDSITIPYQGSGKGVSFQLVKLGVWKSPTDMRSWVP
LSTDDPVIDRLYLSSHRGVIADNQAKWAVPTTRTDDKLRMETCFQQACKGKNQALCENPEWAPL
KDNRIPSYGVLSVNLSLTVELKIKIASGFGPLITHGSGMDLYKTNHNNVYWLTIPPMKNLALGV
INTLEWIPRFKVSPALFTVPIKEAGEDCHAPTYLPAEVDGDVKLSSNLVILPGQDLQYVLATYD
TSRVEHAVVYYVYSPSRSFSYFYPFRLPIKGVPIELQVECFTWDKKLWCRHFCVLADSESGGHI
THSGMVGMGVSCTVTREDGTGGGGSGGGGSGGGGSAAAQVQLVQSGGGLVQPGGSLRLSCAASG
RAFNSAAMGWYRQAPGSQRELVASISAGTASYADAVKGRFTISRDYAKNIIYLQMNSLKPDDTA
VYFCNYRTTYTSGYSEDYWGQGTQVTVSGGGSGGGSYPYDVPDYA;
MeV-Hwtc18-CD7 (humanized VHH10)
MGSRIVINREHLMIDRPYVLLAVLFVMFLSLIGLLAIAGIRLHRAAIYTAEIHKSLSTNLDVTN
SIEHQVKDVLTPLFKIIGDEVGLRTPQRFTDLVKFISDKIKFLNPDREYDERDLTWCINPPERI
KLDYDQYCADVAAEELMNALVNSTLLEARATNQFLAVSKGNCSGPTTIRGQFSNMSLSLLDLYL
SRGYNVSSIVTMTSQGMYGGTYLVGKPNLSSKGSELSQLSMHRVFEVGVIRNPGLGAPVFHMTN
YFEQPVSNDFSNCMVALGELKFAALCHREDSITIPYQGSGKGVSFQLVKLGVWKSPTDMRSWVP
LSTDDPVIDRLYLSSHRGVIADNQAKWAVPTTRTDDKLRMETCFQQACKGKNQALCENPEWAPL
KDNRIPSYGVLSVNLSLTVELKIKIASGFGPLITHGSGMDLYKTNHNNVYWLTIPPMKNLALGV
INTLEWIPRFKVSPALFTVPIKEAGEDCHAPTYLPAEVDGDVKLSSNLVILPGQDLQYVLATYD
TSRVEHAVVYYVYSPSRSFSYFYPFRLPIKGVPIELQVECFTWDKKLWCRHFCVLADSESGGHI
THSGMVGMGVSCTVTREDGTGGGGSGGGGSGGGGSAAADVQLQESGGGSVQAGGSLRLSCAASG
YTHSSYCMAWFRQAPGREREGVASIDSDGTTSYADSVKGRFTISQDNAKNTLYLQMNSLKPEDT
AMYYCAARFGPMGCVDLSTLSFGHWGQGTQVTVSITGGGSGGGSYPYDVPDYA;
MeV-Hwtc18-CD4 (03F11)
MGSRIVINREHLMIDRPYVLLAVLFVMFLSLIGLLAIAGIRLHRAAIYTAEIHKSLSTNLDVTN
SIEHQVKDVLTPLFKIIGDEVGLRTPQRFTDLVKFISDKIKFLNPDREYDERDLTWCINPPERI
KLDYDQYCADVAAEELMNALVNSTLLEARATNQFLAVSKGNCSGPTTIRGQFSNMSLSLLDLYL
SRGYNVSSIVTMTSQGMYGGTYLVGKPNLSSKGSELSQLSMHRVFEVGVIRNPGLGAPVFHMTN
YFEQPVSNDFSNCMVALGELKFAALCHREDSITIPYQGSGKGVSFQLVKLGVWKSPTDMRSWVP
LSTDDPVIDRLYLSSHRGVIADNQAKWAVPTTRTDDKLRMETCFQQACKGKNQALCENPEWAPL
KDNRIPSYGVLSVNLSLTVELKIKIASGFGPLITHGSGMDLYKTNHNNVYWLTIPPMKNLALGV
INTLEWIPRFKVSPALFTVPIKEAGEDCHAPTYLPAEVDGDVKLSSNLVILPGQDLQYVLATYD
TSRVEHAVVYYVYSPSRSFSYFYPFRLPIKGVPIELQVECFTWDKKLWCRHFCVLADSESGGHI
THSGMVGMGVSCTVTREDGTGGGGSGGGGSGGGGSAAAEVQLVESGGGSVQPGGSLTLSCGTSG
RTFNVMGWFRQAPGKEREFVAAVRWSSTGIYYTQYADSVKSRFTISRDNAKNTVYLEMNSLKPE
DTAVYYCAADTYNSNPARWDGYDFRGQGTQVTVSSGGGSGGGSYPYDVPDYA;
and
MeV-Hwtc18-CD8a (R3HCD27)
MGSRIVINREHLMIDRPYVLLAVLFVMFLSLIGLLAIAGIRLHRAAIYTAEIHKSLSTNLDVTN
SIEHQVKDVLTPLFKIIGDEVGLRTPQRFTDLVKFISDKIKFLNPDREYDFRDLTWCINPPERI
KLDYDQYCADVAAEELMNALVNSTLLEARATNQFLAVSKGNCSGPTTIRGQFSNMSLSLLDLYL
SRGYNVSSIVTMTSQGMYGGTYLVGKPNLSSKGSELSQLSMHRVFEVGVIRNPGLGAPVFHMTN
YFEQPVSNDFSNCMVALGELKFAALCHREDSITIPYQGSGKGVSFQLVKLGVWKSPTDMRSWVP
LSTDDPVIDRLYLSSHRGVIADNQAKWAVPTTRTDDKLRMETCFQQACKGKNQALCENPEWAPL
KDNRIPSYGVLSVNLSLTVELKIKIASGFGPLITHGSGMDLYKTNHNNVYWLTIPPMKNLALGV
INTLEWIPRFKVSPALFTVPIKEAGEDCHAPTYLPAEVDGDVKLSSNLVILPGQDLQYVLATYD
TSRVEHAVVYYVYSPSRSFSYFYPFRLPIKGVPIELQVECFTWDKKLWCRHFCVLADSESGGHI
THSGMVGMGVSCTVTREDGTGGGGSGGGGSGGGGSAAAQVQLQESGGGSVQPGGSLRLSCAASG
FTFDDYAMSWVRQVPGKGLEWVSTINWNGGSAEYAEPVKGRFTISRDNAKNTVYLQMNSLKLED
TAVYYCAKDADLVWYNLSTGQGTQVTVSSAAAYPYDVPDYGSGGGSGGGSYPYDVPDYA.

19. A pharmaceutical composition comprising the pseudotyped viral particle of claim 1, and a pharmaceutically acceptable excipient.

20. A kit comprising the pseudotyped viral particle of claim 1, wherein the pseudotyped viral particle comprises a heterologous polynucleotide comprising a polypeptide-encoding sequence under the control of a promoter, and instructions for the use of the kit.

21. A fusion protein suitable for pseudotyping a viral particle, wherein the fusion protein comprises a viral envelope glycoprotein domain fused to a VHH domain, wherein the VHH domain or fragment thereof specifically binds an antigen present on a target cell, the fusion protein comprising a sequence with at least 85% sequence identity to a sequence selected from the group consisting of:
MeV-Hwtc18-MHCII (N11)
MGSRIVINREHLMIDRPYVLLAVLFVMFLSLIGLLAIAGIRLHRAAIYTAEIHKSLSTNLDVTN
SIEHQVKDVLTPLFKIIGDEVGLRTPQRFTDLVKFISDKIKFLNPDREYDERDLTWCINPPERI
KLDYDQYCADVAAEELMNALVNSTLLEARATNQFLAVSKGNCSGPTTIRGQFSNMSLSLLDLYL
SRGYNVSSIVTMTSQGMYGGTYLVGKPNLSSKGSELSQLSMHRVFEVGVIRNPGLGAPVFHMTN
YFEQPVSNDFSNCMVALGELKFAALCHREDSITIPYQGSGKGVSFQLVKLGVWKSPTDMRSWVP
LSTDDPVIDRLYLSSHRGVIADNQAKWAVPTTRTDDKLRMETCFQQACKGKNQALCENPEWAPL
KDNRIPSYGVLSVNLSLTVELKIKIASGFGPLITHGSGMDLYKTNHNNVYWLTIPPMKNLALGV
INTLEWIPRFKVSPALFTVPIKEAGEDCHAPTYLPAEVDGDVKLSSNLVILPGQDLQYVLATYD
TSRVEHAVVYYVYSPSRSFSYFYPFRLPIKGVPIELQVECFTWDKKLWCRHFCVLADSESGGHI
THSGMVGMGVSCTVTREDGTGGGGSGGGGSGGGGSAAAQVQLVQSGGGLVQPGGSLGLSCAASG
NIGSRDNMGWYRQAPGKQREWVATISGYGIATYRDSVKGRFTVAKDTAKNIVSLQMNYLTTEDT
AVYYCYAYAVDSRNIFWSQGTQVTVSGGGSGGGSYPYDVPDYA;
MeV-Hwtc18-CD45 (32)
MGSRIVINREHLMIDRPYVLLAVLFVMFLSLIGLLAIAGIRLHRAAIYTAEIHKSLSTNLDVTN
SIEHQVKDVLTPLFKIIGDEVGLRTPQRFTDLVKFISDKIKFLNPDREYDERDLTWCINPPERI
KLDYDQYCADVAAEELMNALVNSTLLEARATNQFLAVSKGNCSGPTTIRGQFSNMSLSLLDLYL
SRGYNVSSIVTMTSQGMYGGTYLVGKPNLSSKGSELSQLSMHRVFEVGVIRNPGLGAPVFHMTN
YFEQPVSNDFSNCMVALGELKFAALCHREDSITIPYQGSGKGVSFQLVKLGVWKSPTDMRSWVP
LSTDDPVIDRLYLSSHRGVIADNQAKWAVPTTRTDDKLRMETCFQQACKGKNQALCENPEWAPL
KDNRIPSYGVLSVNLSLTVELKIKIASGFGPLITHGSGMDLYKTNHNNVYWLTIPPMKNLALGV
INTLEWIPRFKVSPALFTVPIKEAGEDCHAPTYLPAEVDGDVKLSSNLVILPGQDLQYVLATYD
TSRVEHAVVYYVYSPSRSFSYFYPFRLPIKGVPIELQVECFTWDKKLWCRHFCVLADSESGGHI
THSGMVGMGVSCTVTREDGTGGGGGGGGSGGGGSAAAQVQLVQSGGGLVQPGGSLRLSCAASG
RAFNSAAMGWYRQAPGSQRELVASISAGTASYADAVKGRFTISRDYAKNIIYLQMNSLKPDDTA
VYFCNYRTTYTSGYSEDYWGQGTQVTVSGGGSGGGSYPYDVPDYA;
MeV-Hwtc18-CD7 (humanized VHH10)
MGSRIVINREHLMIDRPYVLLAVLFVMFLSLIGLLAIAGIRLHRAAIYTAEIHKSLSTNLDVTN
SIEHQVKDVLTPLFKIIGDEVGLRTPQRFTDLVKFISDKIKFLNPDREYDERDLTWCINPPERI
KLDYDQYCADVAAEELMNALVNSTLLEARATNQFLAVSKGNCSGPTTIRGQFSNMSLSLLDLYL
SRGYNVSSIVTMTSQGMYGGTYLVGKPNLSSKGSELSQLSMHRVFEVGVIRNPGLGAPVFHMTN
YFEQPVSNDFSNCMVALGELKFAALCHREDSITIPYQGSGKGVSFQLVKLGVWKSPTDMRSWVP
LSTDDPVIDRLYLSSHRGVIADNQAKWAVPTTRTDDKLRMETCFQQACKGKNQALCENPEWAPL
KDNRIPSYGVLSVNLSLTVELKIKIASGFGPLITHGSGMDLYKTNHNNVYWLTIPPMKNLALGV
INTLEWIPRFKVSPALFTVPIKEAGEDCHAPTYLPAEVDGDVKLSSNLVILPGQDLQYVLATYD
TSRVEHAVVYYVYSPSRSFSYFYPFRLPIKGVPIELQVECFTWDKKLWCRHFCVLADSESGGHI
THSGMVGMGVSCTVTREDGTGGGGSGGGGSGGGGSAAADVQLQESGGGSVQAGGSLRLSCAASG
YTHSSYCMAWFRQAPGREREGVASIDSDGTTSYADSVKGRFTISQDNAKNTLYLQMNSLKPEDT
AMYYCAARFGPMGCVDLSTLSFGHWGQGTQVTVSITGGGSGGGSYPYDVPDYA;
MeV-Hwtc18-CD4 (03F11)
MGSRIVINREHLMIDRPYVLLAVLFVMFLSLIGLLAIAGIRLHRAAIYTAEIHKSLSTNLDVTN
SIEHQVKDVLTPLFKIIGDEVGLRTPQRFTDLVKFISDKIKFLNPDREYDFRDLTWCINPPERI
KLDYDQYCADVAAEELMNALVNSTLLEARATNQFLAVSKGNCSGPTTIRGQFSNMSLSLLDLYL
SRGYNVSSIVTMTSQGMYGGTYLVGKPNLSSKGSELSQLSMHRVFEVGVIRNPGLGAPVFHMTN
YFEQPVSNDFSNCMVALGELKFAALCHREDSITIPYQGSGKGVSFQLVKLGVWKSPTDMRSWVP
LSTDDPVIDRLYLSSHRGVIADNQAKWAVPTTRTDDKLRMETCFQQACKGKNQALCENPEWAPL
KDNRIPSYGVLSVNLSLTVELKIKIASGFGPLITHGSGMDLYKTNHNNVYWLTIPPMKNLALGV
INTLEWIPRFKVSPALFTVPIKEAGEDCHAPTYLPAEVDGDVKLSSNLVILPGQDLQYVLATYD
TSRVEHAVVYYVYSPSRSFSYFYPFRLPIKGVPIELQVECFTWDKKLWCRHFCVLADSESGGHI
THSGMVGMGVSCTVTREDGTGGGGSGGGGSGGGGSAAAEVQLVESGGGSVQPGGSLTLSCGTSG
RTFNVMGWFRQAPGKEREFVAAVRWSSTGIYYTQYADSVKSRFTISRDNAKNTVYLEMNSLKPE
DTAVYYCAADTYNSNPARWDGYDFRGQGTQVTVSSGGGSGGGSYPYDVPDYA;
and
MeV-Hwtc18-CD8a (R3HCD27)
MGSRIVINREHLMIDRPYVLLAVLFVMFLSLIGLLAIAGIRLHRAAIYTAEIHKSLSTNLDVTN
SIEHQVKDVLTPLFKIIGDEVGLRTPQRFTDLVKFISDKIKFLNPDREYDERDLTWCINPPERI
KLDYDQYCADVAAEELMNALVNSTLLEARATNQFLAVSKGNCSGPTTIRGQFSNMSLSLLDLYL
SRGYNVSSIVTMTSQGMYGGTYLVGKPNLSSKGSELSQLSMHRVFEVGVIRNPGLGAPVFHMTN
YFEQPVSNDFSNCMVALGELKFAALCHREDSITIPYQGSGKGVSFQLVKLGVWKSPTDMRSWVP
LSTDDPVIDRLYLSSHRGVIADNQAKWAVPTTRTDDKLRMETCFQQACKGKNQALCENPEWAPL
KDNRIPSYGVLSVNLSLTVELKIKIASGFGPLITHGSGMDLYKTNHNNVYWLTIPPMKNLALGV
INTLEWIPRFKVSPALFTVPIKEAGEDCHAPTYLPAEVDGDVKLSSNLVILPGQDLQYVLATYD
TSRVEHAVVYYVYSPSRSFSYFYPFRLPIKGVPIELQVECFTWDKKLWCRHFCVLADSESGGHI
THSGMVGMGVSCTVTREDGTGGGGSGGGGSGGGGSAAAQVQLQESGGGSVQPGGSLRLSCAASG
FTFDDYAMSWVRQVPGKGLEWVSTINWNGGSAEYAEPVKGRFTISRDNAKNTVYLQMNSLKLED
TAVYYCAKDADLVWYNLSTGQGTQVTVSSAAAYPYDVPDYGSGGGSGGGSYPYDVPDYA.
